(19) United States
(12) Patent Application Publication
(10) Pub. No.: US 2021/0101881 A1
(43) Pub. Date: Apr. 8, 2021

(54) PYRIMIDINE COMPOUND, PREPARATION METHOD THEREOF AND MEDICAL USE THEREOF

(71) Applicant: Ancureall Pharmaceutical (Shanghai) Co., Ltd., Shanghai (CN)

(72) Inventors: Jutong Si, Shanghai (CN); Meifeng Jiang, Shanghai (CN); Zhihe Yang, Shanghai (CN); Liyun Zhang, Shanghai (CN)

(21) Appl. No.: 15/733,500

(22) PCT Filed: Jan. 29, 2019
(86) PCT No.: PCT/CN2019/073646

§ 371 Date: Aug. 12, 2020

(30) Foreign Application Priority Data
Feb. 12, 2018 (CN) 201810144972.6

(51) Int. Cl. C07D 403/04; A61P 35/02; C07D 401/14; C07D 471/04; C07D 405/14; C07D 403/14

(52) U.S. Cl. CPC C07D 403/04; A61P 35/02; C07D 403/14; C07D 471/04; C07D 405/14; C07D 401/14

(57) ABSTRACT

The present invention discloses a pyrimidine compound, a preparation method thereof and a medical use thereof. Specifically, the present invention discloses a pyrimidine compound represented by formula (I), pharmaceutically acceptable salts thereof, a preparation method thereof, and a use thereof as a cyclin-dependent kinase 9 (CDK9) inhibitor, particularly for the treatment of cancer. The definition of each group in formula (I) is the same as that in the specification.

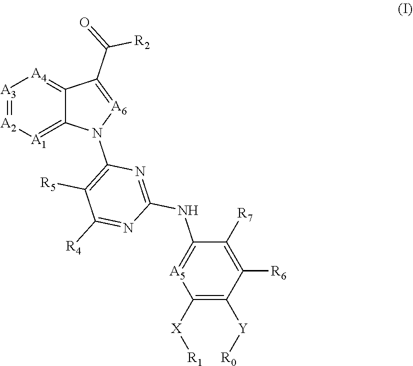

### FIELD OF THE INVENTION

The present invention belongs to the field of medicine, and relates to a novel pyrimidine compound, a method for preparing the same, a pharmaceutical composition comprising the same, and a use thereof as a cyclin dependent kinase 9 (CDK9) inhibitor in the treatment of human diseases including cancer.

### BACKGROUND OF THE INVENTION

The mammalian cell cycle is a highly organized and precisely regulated cell mitosis process. During this process, the genetic material of the cell is replicated and distributed equally between the two proliferating daughter cells. The cell growth factor and cell cycle regulator play an important role in the cell cycle. The cell cycle regulator is a type of intracellular protein synthesized by the cell itself. The abnormal activity of various cell cycle regulators often causes the abnormalities in the normal cell cycle, leading to various types of diseases. For example, when the cell proliferation is out of control, cell transformation may occur which leads to the formation of the cancer cell.

Cyclin dependent kinase (CDK) is a group of serine/threonine protein kinases, which cooperates with Cyclin and is a key regulator of cell cycle progression and transcription. CDK is the catalytic subunit and Cyclin is the regulatory subunit, a heterodimer is formed by the CDK binding to the Cyclin. Various Cyclin-CDK complexes can phosphorylate different substrates in cell via the CDK activity, ultimately achieving the promotion and transformation of different phases of the cell cycle. So far, twenty-one CDK genes (CDK1-CDK20, wherein CDK11 has two genes of CDK11A and CDK11B) and five CDK-like genes CDKL (CDKL1-CDKL5) have been discovered and identified (https://www.genecards.org/). In the functional domain of these CDK protein kinases, the amino acid sequence has a high evolutionary conservation. According to its mechanism and function, CDK can be classified into direct cell cycle regulation CDKs (such as CDK, CDK2, CDK3, CDK 4 and CDK 6) and transcription function CDKs (such as CDK7, CDK 8, CDK 9, CDK 11, CDK 12 and CDK 13). Direct cell cycle regulation CDKs directly regulate the progression of cell cycle stages, and their phosphorylation substrates are cell cycle related proteins. Transcription function CDKs regulate gene transcription by phosphorylating RNA polymerase II complex. It is discovered in clinical data that in the samples of patients diagnosed with different types of malignant tumors and leukemia, such as skin cancer, melanoma, lung cancer, gastric cancer, breast cancer, pancreatic cancer, liver cancer or colon cancer, and acute myeloid leukemia, the different CDKs frequently undergo gene mutation, amplification and overexpression. These mutations are closely related to the occurrence and development of cancer, and/or maintenance of malignant cell phenotypes, as well as patient survival and drug resistance. Meanwhile, basic studies have found that the abnormality of CDK can drive the occurrence of a tumor, and the inhibition of CDK can effectively inhibit/eliminate the growth of tumor cell in vivo and in vitro. CDK has been widely used as a good target for testing and applying cancer treatment. In particular, CDK4/6 selective inhibitors Palbociclib, Ribociclib and Abemaciclib have been successfully used in clinic (Otto T et al., (2017) Nat Rev Cancer 17(2):93-115; Kwapisz D (2017) Breast Cancer Res Treat. 166(1):41-54; Vijayaraghavan S et al., (2017) Target Oncol. 2017 Dec. 7; Ingham M et al., (2017) J Clin Oncol. 35(25):2949-2959; Abou Zahr A et al., (2017) Expert Opin Emerg Drugs. 22(2):137-148; O'Leary B et al., (2016), Nat Rev Clin Oncol. 13(7):417-30); Coin F et al., (2015) Mol Cell. 59(4):513-4; Pozo K et al., (2016) Trends Cancer. 2(10):606-618). Recent studies have found that CDK4/6 and CDK5 have a tumor immunomodulatory function, and selective inhibition of CDK4/6 or CDK5 can enhance the effect of tumor immunotherapy, further proving that CDKs are important target proteins for tumor therapy (Dorand R D et al., (2016) Science. 353(6297): 399-403; Goel S et al., (2017) Nature. 548(7668): 471-475; Deng J et al., (2017) Cancer Discov. 8(2); 216-33; Zhang J et al., (2018) Nature. 553(7686):91-95).

For the years, many different types of CDK inhibitors have undergone extensive preclinical and clinical research. To date, CDK4/6 highly selective inhibitors Palbociclib, Ribociclib and Abemaciclib have been successfully used in the clinical treatment of estrogen receptor-positive, HER2-negative advanced or recurrent breast cancer: Palbociclib and Ribociclib need to be administered in combination with Letrozole (Letrozole); and Abemaciclib can be administered alone or in combination with Fulvestrant. Pan-CDK inhibitors (first-generation CDK inhibitors) such as Alvocidib and Seliciclib are flavonoids. Alvocidib compete with ATP to inhibit CDK1, CDK2, CDK4 and CDK6, with an IC₅₀ value of approximately 40 nM. Seliciclib can inhibit CDK5, Cdc2 and CDK2, with IC₅₀ of 0.2 μM, 0.65 μM and 0.7 μM, respectively, but it has not shown promising antitumor activity in preclinical and clinical studies. The second-generation pan-CDK inhibitors such as Dinaciclib, AT7519, Milciclib, TG02, CYC065 and RGB-286638 can simultaneously inhibit multiple CDKs with high activity. Despite entering multiple phases of clinical trials, these inhibitors administered alone did not show positive therapeutic effects instead showed high clinical side effects. Recently, selective CDK9 inhibitors AZD4573 and BAY-1251152 have entered phase I clinical trial respectively. Although these compounds have shown certain anti-tumor activity in preclinical trial (Lucking U et al., (2017) ChemMedChem. 12(21):1776-1793; Kwiatkowski N et al., (2014) Nature. 511(7511):616-20), there is still an urgent need in clinic for selective CDK9 inhibitors with high efficacy, high specificity and low toxicity for the treatment of cancer. During the long-term research and development of novel selective CDK9 inhibitor, the inventor has discovered a novel pyrimidine compound that can effectively inhibit the in vitro growth of CDK9-positive tumor cells while having its IC₅₀ value can reach a sub-nanomolar level.

### SUMMARY OF THE INVENTION

The object of the present invention is to provide a novel small molecule compound with good specificity, high activity and low toxicity, which can be used as a cyclin dependent kinase 9 (CDK9) inhibitor for preventing and/or treating human diseases including cancer.

The present invention relates to a novel pyrimidine compound. The compound can effectively inhibit the in vitro growth of CDK9-positive leukemia cell MOLM-13 and many different types of tumor cells while having its IC₅₀ value can reach a sub-nanomolar level.

Firstly, the present invention provides a compound of formula (I) or a pharmaceutically acceptable salt thereof,

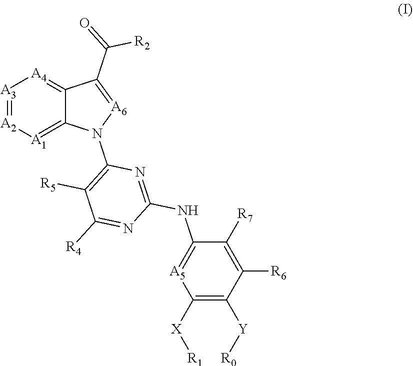

wherein:

A₁, A₂, A₃, A₄ and A₅ are identical or different and are each independently selected from the group consisting of N and CQ;

A₆ is selected from the group consisting of CR₃ and N;

R₂ is selected from the group consisting of alkoxy, hydroxy and amino, wherein the amino is optionally substituted with one or two alkyl(s);

R₃, R₄, R₅, R, and R₇ are each independently selected from group Q;

X and Y are identical or different and are each independently selected from the group consisting of —NR₈—, —O—, —S—, —CH₂—, —C(O)—, —S(O)ₙ— and group Q;

when X and Y are each independently selected from —NR₈—, R₁ and R₀ are identical or different and are each independently selected from the group consisting of hydrogen, alkyl, alkenyl, alkynyl, cycloalkyl, heterocyclyl, aryl, heteroaryl, —R^{u}OR^{x}, —R^{u}N(R^{y})(R^{z})—R^{u}C(O)OR^{N}, —C(O)N(R^{y})(R^{z}), —R^{u}S(O)ₙN(R^{y})(R^{z}) and —R^{u}S(O)R, wherein the alkyl, alkenyl, alkynyl, cycloalkyl, heterocyclyl, aryl and heteroaryl are each independently optionally substituted with one or more substituents selected from the group consisting of halogen, cyano, amino, hydroxy, alkyl, alkoxy, amido, cycloalkyl, heterocyclyl, aryl, haloaryl and heteroaryl; R₈ is selected from the group consisting of hydrogen, alkyl, alkenyl, alkynyl and heterocyclyl; or, R₁ and R₈ or R₀ and R₈ together with the nitrogen attached to them form a heterocyclyl or heteroaryl, wherein the heterocyclyl and heteroaryl are each independently optionally substituted with one or more substituents selected from the group consisting of halogen, alkyl, haloalkyl, alkoxy, haloalkoxy, —C(O)-alkenyl, —C(O)-alkyl, hydroxyalkyl, -alkylene-O-alkyl, heterocyclyl, -alkylene-heterocyclyl, —C(O)-heterocyclyl, —C(O)-cycloalkyl, —C(O)—N(R^{y})(R^{z}) and —R^{u}N(R^{y})(R^{z});

when X and Y are each independently selected from the group consisting of —O—, —S—, —CH₂—, —C(O)— and —S(O)ₙ—, R₁ and R₀ are identical or different and are each independently selected from the group consisting of —R^{u}N(R^{y})(R^{z}), —C(O)N(R^{y})(R^{z}) and —R^{u}S(O)ₙN(R^{y})(R^{z});

when X is selected from group Q, R₁ is absent;

when Y is selected from group Q, R₀ is absent;

each R^{u} is independently selected from the group consisting of a bond, alkylene, alkenylene and alkynylene;

each R^{z} is independently selected from the group consisting of hydrogen, alkyl, hydroxyalkyl, haloalkyl, alkenyl and alkynyl; or

the oxygen in —R^{u}OR^{x}— together with the attached R^{u} and R^{x} form a 3 to 7 membered oxygen-containing heterocyclyl, wherein the heterocyclyl is optionally substituted with one or more group Q;

R^{y} and R^{z} are identical or different and are each independently selected from the group consisting of hydrogen, alkyl, alkoxy, alkenyl, alkynyl, cycloalkyl, haloalkyl and haloalkoxy; or

R^{y} and R^{z} together with the nitrogen attached to them form a heterocyclyl or heteroaryl, wherein the heterocyclyl and heteroaryl are each independently optionally substituted with one or more substituents selected from the group consisting of halogen, alkyl, haloalkyl, alkoxy, haloalkoxy, —C(O)-alkyl, alkyl, alkenyl and alkynyl; each group Q is independently selected from the group consisting of hydrogen, halogen, hydroxy, alkyl, amino, alkoxy, cycloalkyl, alkenyl, alkynyl, cyano, nitro, amido, aryl, heterocyclyl, heteroaryl, —O-(alkylene)-O-alkyl and —O-(alkylene)-heterocyclyl, wherein the alkyl, amino, alkoxy, cycloalkyl, alkenyl, alkynyl, amido, aryl, heterocyclyl and heteroaryl are each independently optionally substituted with one or more substituents selected from the group consisting of hydroxy, halogen and alkyl; and

n is 0, 1 or 2.

In a preferred embodiment of the present invention, the compound of formula (I) or a pharmaceutically acceptable salt thereof according to the present invention, wherein A₁, A₂, A₃, A₄ and A₂ are identical or different and are each independently selected from the group consisting of N and CQ; each group Q is independently selected from the group consisting of hydrogen, halogen, nitro, hydroxy, C₁-C₆ alkyl, C₁-C₆ alkoxy, C₁-C₆ haloalkyl, C₁-C₆ haloalkoxy, C₃-C₆ cycloalkyl, amido, —O—(C₁-C₆ alkylene)-O—C₁-C₆ alkyl and —O—(C₁-C₆ alkylene)-3 to 7 membered heterocyclyl.

In a preferred embodiment of the present invention, the compound of formula (I) or a pharmaceutically acceptable salt thereof according to the present invention, wherein A₁, A₂, A₃ and A₄ are CH.

In a preferred embodiment of the present invention, the compound of formula (I) or a pharmaceutically acceptable salt thereof according to the present invention, wherein A is N, and A₂, A₃ and A₄ are CH.

In a preferred embodiment of the present invention, the compound of formula (I) or a pharmaceutically acceptable salt thereof according to the present invention, wherein A₅ is selected from the group consisting of N and CH.

In a preferred embodiment of the present invention, the compound of formula (I) or a pharmaceutically acceptable salt thereof according to the present invention, wherein A₆ is selected from the group consisting of N and CH.

In a preferred embodiment of the present invention, the compound of formula (I) or a pharmaceutically acceptable salt thereof according to the present invention, wherein:

X is selected from —NR₈—; R⁸ is selected from the group consisting of hydrogen and alkyl; and R₁ is selected from the group consisting of hydrogen, C₁-C₆ alkyl, C₃-C₆ cycloalkyl, 3 to 7 membered heterocyclyl, —R^{u}OR^{x} and —R^{u}N(R^{y})(R^{z}), wherein the C₁-C₆ alkyl, C₃-C₆ cycloalkyl and 3 to 7 membered heterocyclyl are each independently optionally substituted with one or more substituents selected from the group consisting of halogen, cyano, hydroxy, C₁-C₆ alkoxy, 3 to 7 membered heterocyclyl (preferably 3 to 7 membered oxygen-containing or nitrogen-containing heterocyclyl). C₅-C₇ aryl (preferably phenyl), C₅-C₇ haloaryl (preferably halophenyl), 5 to 7 membered heteroaryl and C₃-C₆ cycloalkyl;

Y is selected from group Q; and R₀ is absent;

R^{u}, R^{y}, R^{z} and Q are as defined in formula (I).

In a preferred embodiment of the present invention, the compound of formula (I) or a pharmaceutically acceptable salt thereof according to the present invention, wherein:

X is selected from —NR₈—; and R₁ and R₈ together with the nitrogen attached to them form a heterocyclyl, wherein the heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)-alkenyl, —C(O)-alkyl, hydroxyalkyl, -alkylene-O-alkyl, heterocyclyl, -alkylene-heterocyclyl, —C(O)-heterocyclyl, —C(O)-cycloalkyl, —C(O)—N(R^{y})(R^{z}) and —R^{u}N(R^{y})(R^{z});

Y is selected from group Q; and R₀ is absent;

R^{u}, R^{y}, R^{z} and Q are as defined in formula (I).

In a preferred embodiment of the present invention, the compound of formula (I) or a pharmaceutically acceptable salt thereof according to the present invention, wherein:

X is selected from the group consisting of —O—, —S—, —CH₂—, —C(O)— and —S(O)ₙ—; and R₁ is selected from —R^{u}N(R^{y})(R^{z});

Y is selected from group Q; and R₀ is absent;

R^{u}, R^{y}, R^{z}, n and Q are as defined in formula (I).

In a preferred embodiment of the present invention, the compound of formula (I) or a pharmaceutically acceptable salt thereof according to the present invention, wherein:

Y is selected from —NR₈—; R₈ is selected from the group consisting of hydrogen and alkyl; and R₀ is selected from the group consisting of hydrogen, C₁-C₆ alkyl, C₃-C₆ cycloalkyl, 3 to 7 membered heterocyclyl, —R^{u}OR^{x} and —R^{u}N(R^{y})(R^{z}), wherein the C₁-C₆ alkyl, C₃-C₆ cycloalkyl and 3 to 7 membered heterocyclyl are each independently optionally substituted with one or more substituents selected from the group consisting of halogen, cyano, hydroxy, C₁-C₆ alkoxy, 3 to 7 membered heterocyclyl (preferably 3 to 7 membered oxygen-containing or nitrogen-containing heterocyclyl), C₅-C₇ aryl (preferably phenyl), C₅-C₇ haloaryl (preferably halophenyl), 5 to 7 membered heteroaryl and C₃-C₆ cycloalkyl;

X is selected from group Q; and R₁ is absent;

R^{u}, R^{y}, R^{z} and Q are as defined in formula (I).

In a preferred embodiment of the present invention, the compound of formula (I) or a pharmaceutically acceptable salt thereof according to the present invention, wherein:

Y is selected from —NR₈—; and R₀ and R₈ together with the nitrogen attached to them form a heterocyclyl, wherein the heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)-alkenyl, —C(O)-alkyl, hydroxyalkyl, -alkylene-O-alkyl, heterocyclyl, -alkylene-heterocyclyl, —C(O)-heterocyclyl, —C(O)-cycloalkyl, —C(O)—N(R^{y})(R^{z}) and —R^{u}N(R^{y})(R^{z});

X is selected from group Q, and R₁ is absent;

R^{u}, R^{y}, R^{z} and Q are as defined in formula (I).

In a preferred embodiment of the present invention, the compound of formula (I) or a pharmaceutically acceptable salt thereof according to the present invention, wherein:

Y is selected from the group consisting of —O—, —S—, —CH₂—, —C(O)— and —S(O)ₙ—; and R₀ is selected from —R^{u}N(R^{y})(R^{z});

X is selected from group Q; and R₁ is absent;

R^{u}, R^{y}, R^{z}, n and Q are as defined in formula (I).

In a preferred embodiment of the present invention, the compound of formula (I) or a pharmaceutically acceptable salt thereof according to the present invention, wherein:

X is selected from the group consisting of hydrogen, halogen, hydroxy, alkyl, haloalkyl, amino, alkoxy, haloalkoxy, cycloalkyl, cyano and nitro; and R₁ is absent;

Y is selected from the group consisting of hydrogen, halogen, hydroxy, alkyl, haloalkyl, amino, alkoxy, haloalkoxy, cycloalkyl, cyano and nitro; and R₀ is absent.

In a preferred embodiment of the present invention, the compound of formula (I) or a pharmaceutically acceptable salt thereof according to the present invention, wherein:

X is selected from —NR₈—; and R₁ is selected from the group consisting of hydrogen, C₁-C₆ alkyl and —R^{u}N(R^{y})(R^{z});

Y is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₀ is absent;

R₈ is selected from the group consisting of hydrogen and C₁-C₆ alkyl;

R^{u} is selected from C₁-C₆ alkylene;

R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or

R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

In a preferred embodiment of the present invention, the compound of formula (I) or a pharmaceutically acceptable salt thereof according to the present invention, wherein:

X is selected from —NR₈—; and R₁ and R₈ together with the nitrogen attached to them form a 5 to 7 membered heterocyclyl, preferably a morpholinyl, piperidinyl, piperazinyl, pyrrolidinyl or azepanyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)—C₂-C₆ alkenyl, —C(O)—C₁-C₆ alkyl, C₁-C₆ hydroxyalkyl, —C₁-C₆ alkylene-O—C₁-C₆ alkyl, 3 to 7 membered heterocyclyl, —C₁-C₆ alkylene-3 to 7 membered heterocyclyl, —C(O)-3 to 7 membered heterocyclyl, —C(O)—C₃-C₆ cycloalkyl, —C(O)—N(R^{y})(R^{z}) and —R^{u}N(R^{y})(R^{z});

Y is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₀ is absent;

R^{u} is selected from C₁-C₆ alkylene;

R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or

R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

In a preferred embodiment of the present invention, the compound of formula (I) or a pharmaceutically acceptable salt thereof according to the present invention, wherein:

X is selected from the group consisting of —O—, —S—, —CH₂—, —C(O)— and —S(O)₂—; and R₁ is selected from —R^{u}N(R^{y})(R^{z});

Y is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₀ is absent;

R^{u} is selected from the group consisting of a bond and C₁-C₆ alkylene; R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or

R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

In a preferred embodiment of the present invention, the compound of formula (I) or a pharmaceutically acceptable salt thereof according to the present invention, wherein:

Y is selected from —NR₈—; and R₀ is selected from the group consisting of hydrogen, C₁-C₆ alkyl and —R^{u}N(R^{y})(R^{z});

X is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₁ is absent;

R₈ is selected from the group consisting of hydrogen and C₁-C₆ alkyl;

R^{u} is selected from C₁-C₆ alkylene;

R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃—C cycloalkyl; or

R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

In a preferred embodiment of the present invention, the compound of formula (I) or a pharmaceutically acceptable salt thereof according to the present invention, wherein:

Y is selected from —NR₈—; and R₀ and R₈ together with the nitrogen attached to them form a 5 to 7 membered heterocyclyl, preferably a morpholinyl, piperidinyl, piperazinyl, pyrrolidinyl or azepanyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)—C₂-C₆ alkenyl, —C(O)—C₁-C₆ alkyl, C₁-C₆ hydroxyalkyl, —C₁-C₆ alkylene-O—C₁-C₆ alkyl, 3 to 7 membered heterocyclyl, —C₁-C₆ alkylene-3 to 7 membered heterocyclyl, —C(O)-3 to 7 membered heterocyclyl, —C(O)—C₁-C₆ cycloalkyl, —C(O)—N(R^{y})(R^{z}) and —R^{u}N(R^{y})(R^{z});

X is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₁ is absent;

R^{u} is selected from C₁-C₆ alkylene;

R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or

R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

In a preferred embodiment of the present invention, the compound of formula (I) or a pharmaceutically acceptable salt thereof according to the present invention, wherein:

Y is selected from the group consisting of —O—, —S—, —CH₂—, —C(O)— and —S(O)₂—; and R₀ is selected from —R^{u}N(R^{y})(R^{z});

X is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₁ is absent;

R^{u} is selected from the group consisting of a bond and C₁-C₆ alkylene;

R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or

R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

In a preferred embodiment of the present invention, the compound of formula (I) or a pharmaceutically acceptable salt thereof according to the present invention, wherein R₂ is selected from the group consisting of hydroxy, amino and methylamino.

In a preferred embodiment of the present invention, the compound of formula (I) or a pharmaceutically acceptable salt thereof according to the present invention, wherein:

R₃, R₄, R₅, R₆ and R₇ are each independently selected from the group consisting of hydrogen, halogen, hydroxy, C₁-C₆ alkyl, C₁-C₆ alkoxy, C₁-C₆ haloalkyl, C₁-C₆ haloalkoxy, C₃-C₆ cycloalkyl, nitro, cyano and amino.

The compound of formula (I) of the present invention includes, but is not limited to:
1-[2-(3-bromo-4-fluoro-phenylamino)-pyrimidin-4-yl]-1H-indole-3-carboxamide;
1-[2-(3-chloro-4-trifluoromethyl-phenylamino)-pyrimidin-4-yl]-1H-indole-3-carboxamide;
1-[2-(2-chloro-3-fluoro-phenylamino)-pyrimidin-4-yl]-1H-indole-3-carboxamide;
1-[2-(3-fluoro-5-trifluoromethyl-phenylamino)-pyrimidin-4-yl]-1H-indole-3-carboxamide;
1-[2-(3,4-methoxy-phenylamino)-pyrimidin-4-yl]-1H-indole-3-carboxamide;
1-[2-(4-fluoro-3-nitro-phenylamino)-pyrimidin-4-yl]-1H-indole-3-carboxamide;
1-[2-(4-fluoro-2-methoxy-5-nitro-phenylamino)-pyrimidin-4-yl]-1H-indole-3-carboxamide;
1-{2-[4-(2-dimethylamino-ethylamino)-phenylamino]-pyrimidin-4-yl}-1H-indole-3-carboxamide;
1-{2-[4-(2-morpholin-4-yl-ethylamino)-phenylamino]-pyrimidin-4-yl}-1H-indole-3-carboxamide;
1-{2-[4-(2-piperidin-1-yl-ethylamino)-phenylamino]-pyrimidin-4-yl}-1H-indole-3-carboxamide;
1-(2-{4-[2-(4-methyl-piperazin-1-yl)-ethylamino]-phenylamino}-pyrimidin-4-yl)-1H-indole-3-carboxamide;
1-{2-[4-(piperidin-4-amino)-phenylamino]-pyrimidin-4-yl}-1H-indole-3-carboxamide;
1-{2-[4-(2-pyrrolidin-1-yl-ethylamino)-phenylamino]-pyrimidin-4-yl}-1H-indole-3-carboxamide;
1-[2-(4-morpholin-4-methyl-phenylamino)-pyrimidin-4-yl]-1H-indole-3-carboxamide;
1-{2-[4-(4-acetyl-piperazin-1-methyl)-phenylamino]-pyrimidin-4-yl}-1H-indole-3-carboxamide;
1-{2-[4-(4-methyl-piperazin-1-carbonyl)-phenylamino]-pyrimidin-4-yl}-1H-indole-3-carboxamide;
1-[2-(4-methoxy-phenylamino)-pyrimidin-4-yl]-1H-indole-3-carboxamide;
1-(2-{4-[2-(4-methyl-piperazin-1-yl)-ethoxy]-phenylamino}-pyrimidin-4-yl)-1H-indole-3-carboxamide;
1-{2-[4-(2-dimethylamino-ethoxy)-phenylamino]-pyrimidin-4-yl}-1H-indole-3-carboxamide;
1-{2-[4-(2-pyrrolidin-1-yl-ethoxy)-phenylamino]-pyrimidin-4-yl}-1H-indole-3-carboxamide;
1-{2-[4-(2-morpholin-4-yl-ethoxy)-phenylamino]-pyrimidin-4-yl}-1H-indole-3-carboxamide;
1-(2-{4-[2-(4-piperazin-1-yl)-ethoxy]-phenylamino]-pyrimidin-4-yl}-1H-indole-3-carboxamide;
1-{2-[4-(2-dimethylamino-ethylthio)-phenylamino]-pyrimidin-4-yl}-1H-indole-3-carboxamide;
1-[2-(4-[1,4′]bipiperidin-1′-yl-phenylamino)-pyrimidin-4-yl]-1H-indole-3-carboxamide;
1-(2-{4-[4-(2-hydroxy-ethyl)-piperazin-1-yl]-phenylamino}-pyrimidin-4-yl)-1H-indole-3-carboxamide;
1-(2-{4-[4-(2-methoxy-ethyl)-piperazin-1-yl]-phenylamino}-pyrimidin-4-yl)-1H-indole-3-carboxamide;
1-[2-(4-piperazin-1-yl-phenylamino)-pyrimidin-4-yl]-1H-indole-3-carboxamide;
1-[2-(3-methyl-4-piperazin-1-yl-phenylamino)-pyrimidin-4-yl]-1H-indole-3-carboxamide;
4-fluoro-1-[2-(4-piperazin-1-yl-phenylamino)-pyrimidin-4-yl]-1H-indole-3-carboxamide;
6-fluoro-1-[2-(4-piperazin-1-yl-phenylamino)-pyrimidin-4-yl]-1H-indole-3-carboxamide;
7-fluoro-1-[2-(4-piperazin-1-yl-phenylamino)-pyrimidin-4-yl]-1H-indole-3-carboxamide;
5-bromo-1-[2-(4-piperazin-1-yl-phenylamino)-pyrimidin-4-yl]-1H-indole-3-carboxamide;
5-methoxy-1-[2-(4-piperazin-1-yl-phenylamino)-pyrimidin-4-yl]-1H-indole-3-carboxamide;
1-[5-chloro-2-(4-piperazin-1-yl-phenylamino)-pyrimidin-4-yl]-1H-indole-3-carboxamide;
1-[5-fluoro-2-(4-piperazin-1-yl-phenylamino)-pyrimidin-4-yl]-1H-indole-3-carboxamide;
1-[5-methoxy-2-(4-piperazin-1-yl-phenylamino)-pyrimidin-4-yl]-1H-indole-3-carboxamide;
1-[5-methyl-2-(4-piperazin-1-yl-phenylamino)-pyrimidin-4-yl]-1H-indole-3-carboxamide;
1-[6-methyl-2-(4-piperazin-1-yl-phenylamino)-pyrimidin-4-yl]-1H-indole-3-carboxamide;
1-[5-fluoro-2-(3-methyl-4-piperazin-1-yl-phenylamino)-pyrimidin-4-yl]-1H-indole-3-carboxamide;
1-[5-fluoro-2-(3-methoxy-4-piperazin-1-yl-phenylamino)-pyrimidin-4-yl]-1H-indole-3-carboxamide;
1-[5-fluoro-2-(3-fluoro-4-piperazin-1-yl-phenylamino)-pyrimidin-4-yl]-1H-indole-3-carboxamide;
1-[5-chloro-2-(3-methyl-4-piperazin-1-yl-phenylamino)-pyrimidin-4-yl]-1H-indole-3-carboxamide;
1-[5-chloro-2-(3-methoxy-4-piperazin-1-yl-phenylamino)-pyrimidin-4-yl]-1H-indole-3-carboxamide;
1-[5-chloro-2-(3-fluoro-4-piperazin-1-yl-phenylamino)-pyrimidin-4-yl]-1H-indole-3-carboxamide;
1-{2-[4-(4-methyl-piperazin-1-yl)-phenylamino]-pyrimidin-4-yl}-1H-indole-3-carboxamide;
1-{2-[3-fluoro-4-(4-methyl-piperazin-1-yl)-phenylamino]-pyrimidin-4-yl}-1H-indole-3-carboxamide;
1-{2-[3,5-difluoro-4-(4-methyl-piperazin-1-yl)-phenylamino]-pyrimidin-4-yl}-1H-indole-3-carboxamide;
1-{2-[3-methoxy-4-(4-methyl-piperazin-1-yl)-phenylamino]-pyrimidin-4-yl}-1H-indole-3-carboxamide;
1-{2-[3-cyano-4-(4-methyl-piperazin-1-yl)-phenylamino]-pyrimidin-4-yl}-1H-indole-3-carboxamide;
1-{2-[3-methyl-4-(4-methyl-piperazin-1-yl)-phenylamino]-pyrimidin-4-yl}-1H-indole-3-carboxamide;
1-{2-[4-(4-isopropyl-piperazin-1-yl)-3-methyl-phenylamino]-pyrimidin-4-yl}-1H-indole-3-carboxamide;
1-{5-fluoro-2-[4-(4-methyl-piperazin-1-yl)-phenylamino]-pyrimidin-4-yl}-1H-indole-3-carboxamide;
1-{5-chloro-2-[4-(4-methyl-piperazin-1-yl)-phenylamino]-pyrimidin-4-yl}-1H-indole-3-carboxamide;
1-{5-fluoro-2-[3-fluoro-4-(4-methyl-piperazin-1-yl)-phenylamino]-pyrimidin-4-yl}-1H-indole-3-carboxamide;
1-{5-fluoro-2-[3-methyl-4-(4-methyl-piperazin-1-yl)-phenylamino]-pyrimidin-4-yl}-1H-indole-3-carboxamide;
1-{5-chloro-2-[3-methyl-4-(4-methyl-piperazin-1-yl)-phenylamino]-pyrimidin-4-yl}-1H-indole-3-carboxamide;
1-{2-[4-(4-ethyl-piperazin-1-yl)-3-methyl-phenylamino]-5-fluoro-pyrimidin-4-yl}-1H-indole-3-carboxamide;
1-{5-chloro-2-[4-(4-ethyl-piperazin-1-yl)-3-methyl-phenylamino]-pyrimidin-4-yl}-1H-indole-3-carboxamide;
1-{5-fluoro-2-[4-(4-isopropyl-piperazin-1-yl)-3-methyl-phenylamino]-pyrimidin-4-yl}-1H-indole-3-carboxamide;
1-{5-chloro-2-[4-(4-isopropyl-piperazin-1-yl)-3-methyl-phenylamino]-pyrimidin-4-yl}-1H-indole-3-carboxamide;
1-{5-chloro-2-[3-fluoro-4-(4-methyl-piperazin-1-yl)-phenylamino]-pyrimidin-4-yl}-1H-indole-3-carboxamide;
1-(2-{3-fluoro-4-[4-(1-methyl-piperidin-4-yl)-piperazin-1-yl]-phenylamino}-pyrimidin-4-yl)-1H-indole-3-carboxamide;
1-(2-{-4-[4-(2-dimethylamino-ethyl)-piperazin-1-yl]-3-fluoro-phenylamino}-pyrimidin-4-yl)-1H-indole-3-carboxamide;
1-{2-[4-(4-acryloyl-piperazin-1-yl)-3-methyl-phenylamino]-pyrimidin-4-yl}-1H-indole-3-carboxamide;
1-{2-[3-methyl-4-(4-propionyl-piperazin-1-yl)-phenylamino]-pyrimidin-4-yl}-1H-indole-3-carboxamide;
1-{2-[4-(4-acetyl-piperazin-1-yl)-3-fluoro-phenylamino]-pyrimidin-4-yl}-1H-indole-3-carboxamide;
1-{2-[4-(4-methoxy-piperidin-1-yl)-phenylamino]-pyrimidin-4-yl}-1H-indole-3-carboxamide;
1-{2-[4-(4-dimethylamino-piperidin-1-yl)-3-methyl-phenylamino]-pyrimidin-4-yl}-1H-indole-3-carboxamide;
1-{2-[4-(4-methyl-[1,4]homopiperazin-1-yl)-phenylamino]-pyrimidin-4-yl}-1H-indole-3-carboxamide;
1-[2-(4-morpholin-4-yl-phenylamino)-pyrimidin-4-yl]-1H-indole-3-carboxamide;
1-[2-(3-fluoro-4-morpholin-4-yl-phenylamino)-pyrimidin-4-yl]-1H-indole-3-carboxamide;
1-(2-{4-[methyl-(2-morpholin-4-ethyl)-amino]-phenylamino}-pyrimidin-4-yl)-1H-indole-3-carboxamide;
1-[2-(4-{methyl-[2-(4-methyl-piperazin-1-yl)-ethyl]-amino}-phenylamino)-pyrimidin-4-yl]-1H-indole-3-carboxamide;
1-[2-(4-acetyl-piperazin-1-yl)-ethyl]-methyl-amino)-phenylamino)-pyrimidin-4-yl]-1H-indole-3-carboxamide;
1-[2-(4-dimethylamino-phenylamino)-pyrimidin-4-yl]-1H-indole-3-carboxamide;
1-(2-{4-[(3-dimethylamino-propyl)-methyl-amino]-phenylamino}-pyrimidin-4-yl)-1H-indole-3-carboxamide;
1-(2-{4-[(2-dimethylamino-ethyl)-methyl-amino]-phenylamino}-pyrimidin-4-yl)-1H-indole-3-carboxamide;
1-(2-{2-bromo-4-[(2-dimethylamino-ethyl)-methyl-amino]-phenylamino}-pyrimidin-4-yl)-1H-indole-3-carboxamide;
1-(2-{4-[(2-dimethylamino-ethyl)-methyl-amino]-3-methyl-phenylamino}-pyrimidin-4-yl)-1H-indole-3-carboxamide;
1-(2-{4-[(2-dimethylamino-ethyl)-methyl-amino]-3-methoxy-phenylamino}-pyrimidin-4-yl)-1H-indole-3-carboxamide;
1-(2-{4-[(2-dimethylamino-ethyl)-methyl-amino]-3-isopropoxy-phenylamino}-pyrimidin-4-yl)-1H-indole-3-carboxamide;
1-(2-{3-chloro-4-[(2-dimethylamino-ethyl)-methyl-amino]-phenylamino}-pyrimidin-4-yl)-1H-indole-3-carboxamide;
1-(2-{3-chloro-4-[(3-dimethylamino-propyl)-methyl-amino]-phenylamino}-pyrimidin-4-yl)-1H-indole-3-carboxamide;
1-(2-{4-[(2-dimethylamino-ethyl)-methyl-amino]-phenylamino}-5-fluoro-pyrimidin-4-yl)-1H-indole-3-carboxamide;
1-(5-chloro-2-{4-[(2-dimethylamino-ethyl)-methyl-amino]-phenylamino}-pyrimidin-4-yl)-1H-indole-3-carboxamide;
1-(2-{4-[(2-dimethylamino-ethyl)-methyl-amino]-3-methoxy-phenylamino}-5-fluoro-pyrimidin-4-yl)-1H-indole-3-carboxamide:
1-(2-{4-[(2-dimethylamino-ethyl)-methyl-amino]-3-methoxy-phenylamino}-5-chloro-pyrimidin-4-yl)-1H-indole-3-carboxamide;
1-(2-{4-[(2-dimethylamino-ethyl)-methyl-amino]-3-methyl-phenylamino}-5-fluoro-pyrimidin-4-yl)-1H-indole-3-carboxamide;
1-(5-chloro-2-{4-[(2-dimethylamino-ethyl)-methyl-amino]-3-methyl-phenylamino}-pyrimidin-4-yl)-1H-indole-3-carboxamide;
1-(2-{4-[methyl-(2-pyrrolidin-1-ylethyl)-amino]-phenylamino}-pyrimidin-4-yl)-1H-indole-3-carboxamide;
1-(2-((3-methoxy-4-(methyl(2-(pyrrolidin-1-yl)ethyl)amino)phenylamino)pyrimidin-4-yl)-1H-indole-3-carboxamide;
1-(2-{3-fluoro-4-[methyl-(2-pyrrolidin-1-yl-ethyl)-amino]-phenylamino}-pyrimidin-4-yl)-1H-indole-3-carboxamide;
1-(5-fluoro-2-{4-[methyl-(2-pyrrolidin-1-yl-ethyl)-amino]-phenylamino}-pyrimidin-4-yl)-1H-indole-3-carboxamide;
1-(5-chloro-2-{4-[methyl-(2-pyrrolidin-1-yl-ethyl)-amino]-phenylamino}-pyrimidin-4-yl)-1H-indole-3-carboxamide;
1-(5-fluoro-2-{3-fluoro-4-[methyl-(2-pyrolidin-1-yl-ethyl)-amino]-phenylamino}-pyrimidin-4-yl)-1H-indole-3-carboxamide;
1-(5-fluoro-2-{3-methyl-4-[methyl-(2-pyrrolidin-1-yl-ethyl)-amino]-phenylamino}-pyrimidin-4-yl)-1H-indole-3-carboxamide;
1-(5-chloro-2-{3-methyl-4-[methyl-(2-pyrrolidin-1-yl-ethyl)-amino]-phenylamino}-pyrimidin-4-yl)-1H-indole-3-carboxamide;
1-(5-fluoro-2-{3-methoxy-4-[methyl-(2-pyrrolidin-1-yl-ethyl)-amino]-phenylamino}-pyrimidin-4-yl)-1H-indole-3-carboxamide;
1-(5-chloro-2-{3-methoxy-4-[methyl-(2-pyrrolidin-1-yl-ethyl)-amino]-phenylamino}-pyrimidin-4-yl)-1H-indole-3-carboxamide;
1-(2-{3-methoxy-4-[methyl-(2-pyrrolidin-1-yl-ethyl)-amino]-phenylamino}-5-methyl-pyrimidin-4-yl)-1H-indole-3-carboxamide;
1-[2-(4-piperazin-1-yl-phenylamino)-pyrimidin-4-yl]-1H-indole-3-carboxmethylamide;
1-[2-(2-chloro-4-piperazin-1-yl-phenylamino)-pyrimidin-4-yl]-1H-indole-3-carboxmethylamide;
1-[2-(3-chloro-4-piperazin-1-yl-phenylamino)-pyrimidin-4-yl]-1H-indole-3-carboxmethylamide;
1-{2-[4-(1-methyl-piperidin-4-yl)-phenylamino]-pyrimidin-4-yl}-1H-indole-3-carboxmethylamide;
1-[2-(4-piperidin-4-yl-phenylamino)-pyrimidin-4-yl]-1H-indole-3-carboxmethylamide;
1-{2-[3-(4-methyl-piperazin-1-yl)-phenylamino]-pyrimidin-4-yl}-1H-indole-3-carboxmethylamide;
1-[2-(3-piperazin-1-yl-phenylamino)-pyrimidin-4-yl]-1H-indole-3-carboxmethylamide;
1-[2-(4-sulfamoyl-phenylamino)-pyrimidin-4-yl]-1H-indole-3-carboxmethylamide;
1-[2-(3-dimethylamino-phenylamino)-pyrimidin-4-yl]-1H-indole-3-carboxamide;
1-(2-{3-[(2-dimethylamino-ethyl)-methyl-amino]-phenylamino}-pyrimidin-4-yl)-1H-indole-3-carboxamide;
1-[2-(3-piperazin-1-yl-phenylamino)-pyrimidin-4-yl]-1H-indole-3-carboxamide;
1-{2-[3-(4-methyl-[1,4]homopiperazin-1-yl)-phenylamino]-pyrimidin-4-yl}-1H-indole-3-carboxamide;
1-{2-[3-(4-methyl-piperazin-1-yl)-phenylamino]-pyrimidin-4-yl}-1H-indole-3-carboxamide;
1-{2-[3-fluoro-5-(4-methyl-piperazin-1-yl)-phenylamino]-pyrimidin-4-yl}-1H-indole-3-carboxamide;
1-{5-fluoro-2-[3-(4-methyl-piperazin-1-yl)-phenylamino]-pyrimidin-4-yl}-1H-indole-3-carboxamide;
1-{5-fluoro-2-[4-fluoro-3-(4-methyl-piperazin-1-yl)-phenylamino]-pyrimidin-4-yl}-1H-indole-3-carboxamide;
1-{5-fluoro-2-[4-methyl-3-(4-methyl-piperazin-1-yl)-phenylamino]-pyrimidin-4-yl}-1H-indole-3-carboxamide;
1-{5-chloro-2-[3-(4-methyl-piperazin-1-yl)-phenylamino]-pyrimidin-4-yl}-1H-indole-3-carboxamide;
1-{5-chloro-2-[4-fluoro-3-(4-methyl-piperazin-1-yl)-phenylamino]-pyrimidin-4-yl}-1H-indole-3-carboxamide;
1-{5-chloro-2-[4-chloro-3-(4-methyl-piperazin-1-yl)-phenylamino]-pyrimidin-4-yl}-1H-indole-3-carboxamide;
1-{5-chloro-2-[4-methyl-3-(4-methyl-piperazin-1-yl)-phenylamino]-pyrimidin-4-yl}-1H-indole-3-carboxamide;
1-{5-chloro-2-[4-methoxy-3-(4-methyl-piperazin-1-yl)-phenylamino]-pyrimidin-4-yl}-1H-indole-3-carboxamide;
1-{5-methoxy-2-[3-(4-methyl-piperazin-1-yl)-phenylamino]-pyrimidin-4-yl}-1H-indole-3-carboxamide;
1-{5-methyl-2-[3-(4-methyl-piperazin-1-yl)-phenylamino]-pyrimidin-4-yl}-1H-indole-3-carboxamide;
1-(2-{4-[4-(2-hydroxy-ethyl)-piperazin-1-yl]-phenylamino}-pyrimidin-4-yl)-1H-indole-3-carboxamide;
1-(2-{3-[4-(2-methoxy-ethyl)-piperazin-1-yl]-phenylamino]-pyrimidin-4-yl}-1H-indole-3-carboxamide;
1-{2-[3-(4-acryloyl-piperazin-1-yl)-phenylamino]-pyrimidin-4-yl}-1H-indole-3-carboxamide;
1-{2-[3-(4-propionyl-piperazin-1-yl)-phenylamino]-pyrimidin-4-yl}-1H-indole-3-carboxamide;
1-{2-[3-(2-dimethylamino-ethoxy)-phenylamino]-pyrimidin-4-yl}-1H-indole-3-carboxamide;
1-[2-(pyridin-2-ylamino)-pyrimidin-4-yl]-1H-indole-3-carboxamide;
1-[2-(4-methoxy-pyridin-2-ylamino)-pyrimidin-4-yl]-1H-indole-3-carboxamide;
1-[2-(4,6-dimethyl-pyridin-2-ylamino)-pyrimidin-4-yl]-1H-indole-3-carboxamide;
1-[2-(5-piperazin-1-yl-pyridin-2-ylamino)-pyrimidin-4-yl]-1H-indole-3-carboxamide;
1-[5-chloro-2-(5-piperazin-1-yl-pyridin-2-ylamino)-pyrimidin-4-yl]-1H-indole-3-carboxamide;
1-[2-(4-piperazin-1-yl-phenylamino)-pyrimidin-4-yl]-1H-pyrrolo[2,3-b]pyridine-3-carboxamide;
1-[2-(4-piperazin-1-yl-phenylamino)-pyrimidin-4-yl]-1H-pyrrolo[2,3-b]pyridine-3-carboxamide;
1-[5-fluoro-2-(3-morpholin-4-yl-phenylamino)-pyrimidin-4-yl]-1H-indole-3-carboxamide;
1-[2-(3-piperazin-1-yl-phenylamino)-pyrimidin-4-yl]-1H-indazole-3-carboxamide;
1-{5-fluoro-2-[3-(4-morpholin-4-yl-piperidin-1-yl)-phenylamino]-pyrimidin-4-yl}-1H-indole-3-carboxamide;
1-(5-fluoro-2-{3-[4-(tetrahydro-pyran-4-yl)-piperazin-1-yl]-phenylamino}-pyrimidin-4-yl)-1H-indole-3-carboxamide;
1-{5-fluoro-2-[3-(4-methyl-piperazin-1-yl)-phenylamino]-pyrimidin-4-yl}-5-methoxy-1H-indole-3-carboxamide;
1-{2-[3-(4-ethyl-piperazin-1-yl)-phenylamino]-5-fluoro-pyrimidin-4-yl}-1H-indole-3-carboxamide;
1-{5-fluoro-2-[3-(4-methyl-piperazin-1-ylmethyl)-phenylamino]-pyrimidin-4-yl}-1H-indole-3-carboxamide;
1-{5-fluoro-2-[3-(4-isopropyl-piperazin-1-yl)-phenylamino]-pyrimidin-4-yl}-1H-indole-3-carboxamide;
1-{2-[3-(4-sec-butyl-piperazin-1-yl)-phenylamino]-5-fluoro-pyrimidin-4-yl}-1H-indole-3-carboxamide;
1-[5-fluoro-2-(3-piperazin-1-yl-phenylamino)-pyrimidin-4-yl]-1H-indole-3-carboxamide;
1-{2-[3-(4-tert-butyl-piperazin-1-yl)-phenylamino]-5-fluoro-pyrimidin-4-yl}-1H-indole-3-carboxamide;
1-(5-fluoro-2-{3-[4-(1-methyl-piperidin-4-yl)-piperazin-1-yl]-phenylamino}-pyrimidin-4-yl)-1H-indole-3-carboxamide;
1-(5-fluoro-2-{3-[4-(tetrahydro-pyran-4-yl)-piperazin-1-yl]-phenylamino}-pyrimidin-4-yl)-5-methoxy-1H-indole-3-carboxamide;
1-(5-fluoro-2-{3-[4-(tetrahydro-furan-3-yl)-piperazin-1-yl]-phenylamino}-pyrimidin-4-yl)-1H-indole-3-carboxamide;
1-(5-fluoro-2-{3-[4-(tetrahydro-furan-3-yl)-piperazin-1-yl]-phenylamino}-pyrimidin-4-yl)-5-methoxy-1H-indole-3-carboxamide;
1-{2-[3-(4-ethyl-piperazin-1-yl)-phenylamino]-5-fluoro-pyrimidin-4-yl}-5-methoxy-1H-indole-3-carboxamide;
1-[5-fluoro-2-(3-pyrazol-1-yl-phenylamino)-pyrimidin-4-yl]-1H-indole-3-carboxamide;
1-{5-fluoro-2-[3-(4-methyl-pyrazol-1-yl)-phenylamino]-pyrimidin-4-yl}-1H-indole-3-carboxamide;
1-(5-fluoro-2-{3-[4-(2-hydroxy-propyl)-piperazin-1-yl]-phenylamino}-pyrimidin-4-yl)-1H-indole-3-carboxamide;
1-{5-fluoro-2-[3-(4-oxiranylmethyl-piperazin-1-yl)-phenylamino]-pyrimidin-4-yl}-1H-indole-3-carboxamide;
1-(5-fluoro-2-{3-[4-(tetrahydro-pyran-4-ylmethyl)-piperazin-1-yl]-phenylamino}-pyrimidin-4-yl)-1H-indole-3-carboxamide;
1-(5-fluoro-2-{3-[4-(tetrahydro-furan-2-carbonyl)-piperazin-1-yl]-phenylamino}-pyrimidin-4-yl)-1H-indole-3-carboxamide;
1-{5-fluoro-2-[3-(4-isopropyl-piperazin-1-yl)-phenylamino]-pyrimidin-4-yl}-5-methoxy-1H-indole-3-carboxamide;
1-(5-fluoro-2-{3-[4-(tetrahydro-furan-2-ylmethyl)-piperazin-1-yl]-phenylamino}-pyrimidin-4-yl)-1H-indole-3-carboxamide;
1-{2-[3-(4-ethyl-piperazin-1-yl)-phenylamino]-5-fluoro-pyrimidin-4-yl}-5-fluoro-1H-indole-3-carboxamide;
5-amino-1-{2-[3-(4-ethyl-piperazin-1-yl)-phenylamino]-5-fluoro-pyrimidin-4-yl}-1H-indole-3-carboxamide;
1-{2-[3-(4-ethyl-piperazin-1-yl)-phenylamino]-5-fluoro-pyrimidin-4-yl}-5-hydroxy-1H-indole-3-carboxamide;
1-{2-[3-(4-ethyl-piperazin-1-yl)-phenylamino]-5-fluoro-pyrimidin-4-yl}-5-(2-meth oxy-ethoxy)-1H-indole-3-carboxamide;
1-{2-[3-(4-acetyl-piperazin-1-yl)-phenylamino]-5-fluoro-pyrimidin-4-yl}-1H-indole-3-carboxamide;
1-(5-fluoro-2-{3-[4-(tetrahydro-pyran-4-carbonyl)-piperazin-1-yl]-phenylamino}-pyrimidin-4-yl)-1H-indole-3-carboxamide;
1-(5-fluoro-2-{3-[4-(morpholin-4-carbonyl)-piperazin-1-yl]-phenylamino}-pyrimidin-4-yl)-1H-indole-3-carboxamide;
1-{2-[3-(4-ethyl-piperazin-1-yl)-phenylamino]-5-fluoro-pyrimidin-4-yl}-5-(2-morpholin-4-yl-ethoxy)-1H-indole-3-carboxamide;
1-(5-fluoro-2-{3-[4-(pyrrolidin-2-carbonyl)-1-piperazin-1-yl]-phenylamino}-pyrimidin-4-yl)-1H-indole-3-carboxamide;
1-(5-fluoro-2-{3-[4-(tetrahydro-furan-3-carbonyl)-piperazin-1-yl]-phenylamino}-pyrimidin-4-yl)-1H-indole-3-carboxamide;
1-{2-[3-(4-cyclopentanecarbonyl-piperazin-1-yl)-phenylamino]-5-fluoro-pyrimidin-4-yl}-1H-indole-3-carboxamide;
1-{5-fluoro-2-[3-(4-methylcarbamoyl-piperazin-1-yl)-phenylamino]-pyrimidin-4-yl}-1H-indole-3-carboxamide.

In another aspect, the present invention provides a method for preparing the compound of formula (I) or a pharmaceutically acceptable salt thereof, comprising the following steps of:

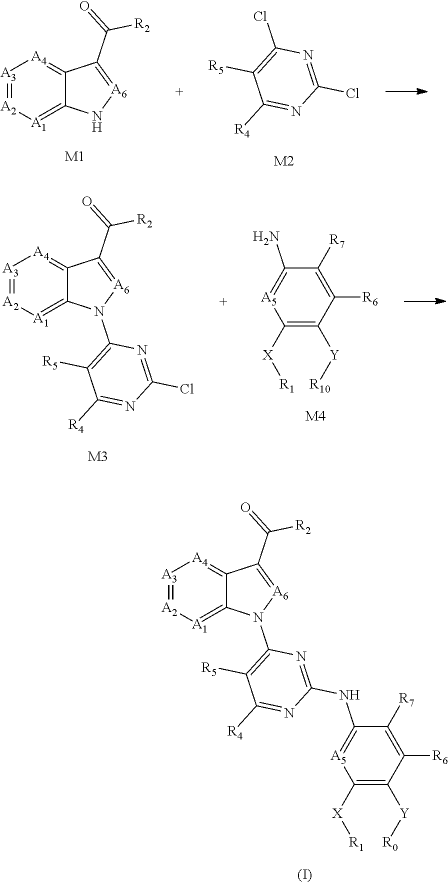

intermediate M1 is reacted with intermediate M2 in a solvent in the presence of a base and a catalyst to give intermediate M3; said solvent is preferably N,N dimethylformamide (DMF) or N-methylpyrrolidone (NMP); said base is preferably potassium carbonate or cesium carbonate; and said catalyst is preferably 1-hydroxybenzotriazole (HOBT);

intermediate M3 is reacted with intermediate M4 in a solvent under acid catalysis to give the compound of formula (I); said solvent is preferably isopropanol, isopentanol, sec-pentanol or dioxane; and said acid is preferably hydrochloric acid, sulfuric acid, methanesulfonic acid, p-toluenesulfonic acid or benzenesulfonic acid; wherein X, Y, A₁, A₂, A₃, A₄, A₅, A₆, R₀, R₁, R₂, R₄, R₅, R₆ and R₇ are as defined in formula (I) above.

The present invention further relates to a pharmaceutical composition comprising a therapeutically effective amount of the compound of formula (I) or a pharmaceutically acceptable salt thereof according to the present invention as the active ingredient, and a pharmaceutically acceptable carrier.

The present invention further relates to a use of the compound of formula (I) or a pharmaceutically acceptable salt thereof or the pharmaceutical composition comprising the same in the preparation of a CDK9 inhibitor.

The present invention further relates to a use of the compound of formula (I) or a pharmaceutically acceptable salt thereof or the pharmaceutical composition comprising the same in the preparation of a medicament for the treatment of cancer in mammals including human. The cancer includes, but is not limited to, non-solid tumors such as leukemia, and solid tumors such as skin cancer, melanoma, lung cancer, gastric cancer, breast cancer, pancreatic cancer, liver cancer or colon cancer.

The present invention further relates to a method for inhibiting CDK9 comprising administering an inhibitory effective amount of the compound of formula (I) or a pharmaceutically acceptable salt thereof or the pharmaceutical composition comprising the same to a patient in need thereof.

The present invention further relates to a method for treating cancers in mammals, including human, comprising administering a therapeutically effective amount of the compound of formula (I) or a pharmaceutically acceptable salt thereof or the pharmaceutical composition comprising the same to a patient in need thereof. The cancer includes, but is not limited to, non-solid tumors such as leukemia, and solid tumors such as skin cancer, melanoma, lung cancer, gastric cancer, breast cancer, pancreatic cancer, liver cancer or colon cancer.

The present invention further relates to a compound of formula (I), a pharmaceutically acceptable salt thereof, a metabolite thereof, a prodrug thereof or a pharmaceutical composition comprising the same for use as a drug.

The present invention further relates to a compound of formula (I), a pharmaceutically acceptable salt thereof or a pharmaceutical composition comprising the same for use as a CDK9 inhibitor.

The present invention further relates to a compound of formula (I), a pharmaceutically acceptable salt thereof, a metabolite thereof or a pharmaceutical composition comprising the same for use in treating cancers, wherein the cancer includes, but is not limited to, non-solid tumors such as leukemia, and solid tumors such as skin cancer, melanoma, lung cancer, gastric cancer, breast cancer, pancreatic cancer, liver cancer or colon cancer.

The present invention further relates to a compound of formula (I), a pharmaceutically acceptable salt thereof, a metabolite thereof or a pharmaceutical composition comprising the same for use in treating cancers in combination with other drugs or cancer therapies.

### DETAILED DESCRIPTION OF THE INVENTION

Unless otherwise specified, all technical and scientific terms used herein have the same meaning as commonly understood by the person skilled in the art. All the patents, applications, published applications, and other publications are incorporated herein by reference in their entirety. If there are multiple definitions for the terms used herein, unless otherwise indicated, the terms in this section shall prevail. If the number of any given substituent is not specified, one or more substituents may be present. For example, “haloalkyl” may contain one or more of the same or different halogens. In the description herein, if the chemical structure is inconsistent with the chemical name, the chemical structure shall prevail. As used herein, abbreviations for any protecting groups, amino acids, and other compounds are indicated by their commonly accepted abbreviations or indicated according to the IUPAC-IUB Commission on Biochemical Nomenclature (refer to Biochem. 1972, 77:942-944), unless otherwise specified.

Unless there is a contrary statement, the following terms used in the specification and claims have the following meanings.

The term “alkyl” refers to a saturated aliphatic hydrocarbon group, which is a straight or branched chain group comprising 1 to 20 carbon atoms. The straight or branched alkyl has 1 to 18 carbon atoms, preferably 1 to 10 carbon atoms, more preferably 1 to 6 carbon atoms, and even more preferably 1 to 4 carbon atoms. Non-limiting examples include, but are not limited to, methyl, ethyl, n-propyl, isopropyl, n-butyl, isobutyl, sec-butyl, tert-butyl, n-pentyl, iso-pentyl, neopentyl, hexyl, isohexyl, n-heptyl, isoheptyl, n-octyl, isooctyl, n-nonyl, n-decyl and the like. In the present description, “alkyl” further includes a cyclicalkyl group having 3 to 10 carbon atoms, preferably 3 to 8 carbon atoms, more preferably 4 to 6 carbon atoms, such as cyclopropyl, cyclobutyl, cyclopentyl, cyclohexyl, cycloheptyl, cyclooctyl, cyclononyl, cyclodecyl, decahydronaphthalenyl, norbomane and adamantyl. The alkyl may be substituted or unsubstituted. When substituted, the substituents may be substituted at any available point, the substituent is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, amino, haloalkyl, hydroxyalkyl, carboxy and carboxylic ester groups.

The term “alkylene” refers to a saturated linear or branched aliphatic hydrocarbon group having two residues derived from the removal of two hydrogen atoms from the same carbon atom or two different carbon atoms of the parent alkane. The linear or branched alkylene has 1 to 20 carbon atoms, preferably 1 to 12 carbon atoms, and more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), 1,5-pentylene (—CH₂CH₂CH₂CH₂CH₂—) and the like.

The term “alkenyl” refers to a straight or branched hydrocarbon group consisting of carbon and hydrogen atoms which comprises at least one double bond and connected to the remaining part of the molecule by a single bond or double bond. The alkenyl preferably has 2 to 10 carbon atoms, more preferably 2 to 6 carbon atoms, even more preferably 2 to 4 carbon atoms. Non-limiting examples include vinyl, propenyl, butenyl, pentenyl, pentadienyl, hexenyl. The alkenyl group can be substituted or unsubstituted. When substituted, the substituent is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, amino, haloalkyl, hydroxyalkyl, carboxy and carboxylic ester groups.

The term “alkynyl” refers to a straight or branched hydrocarbon chain group consisting of carbon and hydrogen atoms which comprises at least one triple bond, and connected to the remaining part of the molecule by a single bond or triple bond. The alkynyl preferably has 2 to 10 carbon atoms, more preferably 2 to 6 carbon atoms, even more preferably 2 to 4 carbon atoms. Non-limiting examples include ethynyl, propynyl, butynyl, pentynyl, hexynyl. The alkynyl group can be substituted or unsubstituted. When substituted, the substituent is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, amino, haloalkyl, hydroxyalkyl, carboxy and carboxylic ester groups.

The term “cycloalkyl” refers to saturated or partially unsaturated monocyclic or polycyclic ring hydrocarbon group containing 3 to 20 carbon atoms, preferably 3 to 12 carbon atoms, more preferably 3 to 10 carbon atoms, and most preferably 3 to 7 carbon atoms. Non-limiting examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like, preferably cyclopropyl, cyclohexenyl. Polycyclic cycloalkyl groups include spiro, fused and bridged cycloalkyl groups. The cycloalkyl can be optionally substituted or unsubstituted. When substituted, the substituent is preferably one or more of groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, amino, haloalkyl, hydroxyalkyl, carboxy and carboxylic ester groups.

The term “heterocyclyl” refers to a saturated or partially unsaturated monocyclic or polycyclic ring hydrocarbon group containing 3 to 20 ring atoms, wherein one or more ring atoms are heteroatoms selected from the group consisting of N, O and S(O)ₘ (wherein m is an integer of 0 to 2), but excludes —O—O—, —O—S— or —S—S— moiety, and the remaining ring atoms are carbon atoms. Preferably, the heterocyclyl has 3 to 12 ring atoms wherein 1 to 4 atoms are heteroatoms; more preferably, 3 to 10 ring atoms; more preferably 3 to 7 ring atoms; even more preferably 4 to 6 ring atoms; and most preferably 5 to 6 ring atoms. Non-limited examples of monocyclic heterocyclyl include oxiranyl, pyrrolidinyl, piperidinyl, piperazinyl, morpholinyl, thiomorpholinyl, homopiperazinyl, pyranyl, tetrahydrofuranyl, azepanyl and the like. Polycyclic heterocyclyl include spiro, fused and bridged heterocyclyl. The heterocyclyl group can be optionally substituted or unsubstituted. When substituted, the substituent is preferably one or more of groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, amino, haloalkyl, hydroxyalkyl, carboxy and carboxylic ester groups.

The term “aryl” refers to an all-carbon monocyclic or fused polycyclic (i.e., rings that share adjacent pairs of carbon atoms) groups having a conjugated π-electron system, preferably a 5 to 10 membered aryl, more preferably a 5 to 7 membered aryl, even more preferably phenyl and naphthyl, and most preferably phenyl. The aryl group may be completely aromatic, such as phenyl, naphthyl, anthryl, phenanthryl and the like. The aryl group may also be a combination of an aromatic ring and a non-aromatic ring, for example, indene, fluorene, acenaphthene and the like. The aryl ring may be fused to a heteroaryl, heterocyclyl or cycloalkyl ring, wherein the ring bound to the parent structure is the aryl ring. Non-limiting examples include:

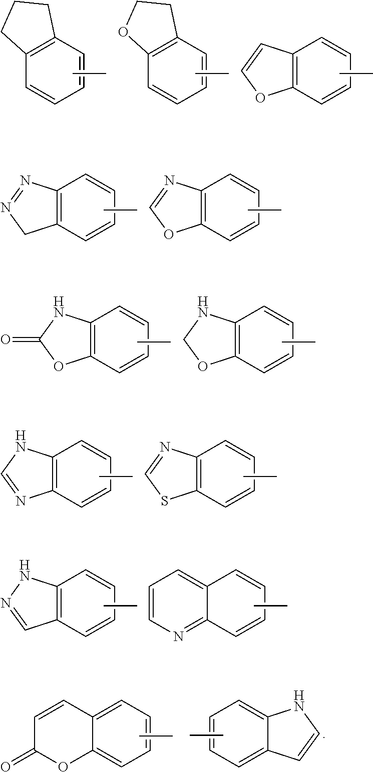

The aryl group can be substituted or unsubstituted. When substituted, the substituent is preferably one or more of groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, amino, haloalkyl, hydroxyalkyl, carboxy and carboxylic ester groups.

The term “heteroaryl” refers to a 5 to 14 membered heteroaromatic system having 1 to 4 heteroatoms selected from the group consisting of O, S and N. The heteroaryl is preferably a 5 to 10 membered heteroaryl, more preferably a 5 to 7 membered heteroaryl, even more preferably a 5 or 6 membered heteroaryl, such as thiadiazolyl, pyrazolyl, oxazolyl, oxadiazolyl, imidazolyl, triazolyl, thiazolyl, furyl, thienyl, pyridyl, pyrrolyl, N-alkylpyrrolyl, pyrimidyl, pyrazinyl, imidazolyl, tetrazolyl and the like. The heteroaryl ring may be fused to an aryl, heterocyclyl or cycloalkyl ring, where the ring bound to the parent structure is the heteroaryl ring. Non-limiting examples include:

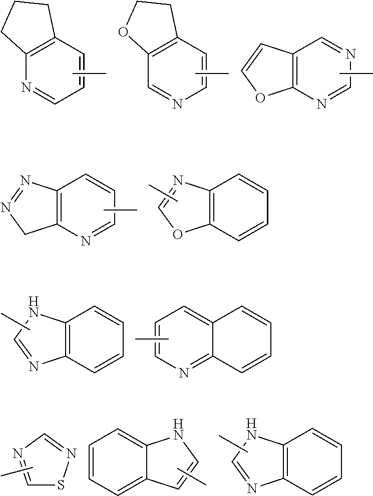

The heteroaryl can be optionally substituted or unsubstituted. When substituted, the substituent is preferably one or more groups independently selected from the group consisting of alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxyl, heterocycloalkoxyl, cycloalkylthio, heterocyclylthio, amino, haloalkyl, hydroxyalkyl, carboxy and carboxylic ester groups.

The term “alkoxy” refers to —O-(alkyl) and —O-(unsubstituted cycloalkyl), wherein the alkyl and cycloalkyl are as defined above. Non-limiting examples include methoxy, ethoxy, propoxy, butoxy, cyclopropyloxy, cyclobutyloxy, cyclopentyloxy, cyclohexyloxy and on the like. The alkoxy can be optionally substituted or unsubstituted. When substituted, the substituent is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, amino, haloalkyl, hydroxyalkyl, carboxy and carboxylic ester groups.

The term “haloalkyl” refers to an alkyl group in which one or more hydrogen atoms is replaced by a halogen, wherein the alkyl is as defined above. Non-limiting examples include chloromethyl, trifluoromethyl, 1-chloro-2-fluoroethyl, 2,2-difluoroethyl, 2-fluoropropyl, 2-fluoropropan-2-yl, 2,2,2-trifluoroethyl, 1,1-difluoroethyl, 1,3-difluoro-2-methylpropyl, 2,2-difluorocyclopropyl, (trifluoromethyl)cyclopropyl, 4,4-difluorocyclohexyl and 2,2,2-trifluoro-1,1-dimethyl-ethyl.

The term “haloalkoxy” refers to an alkoxy group in which one or more hydrogen atoms is replaced by a halogen, wherein the alkoxy is as defined above.

The term “halogen” includes fluorine, chlorine, bromine and iodine.

The term “amino” refers to a —NH₂ group.

The term “nitro” refers to a —NO₂ group.

The term “cyano” refers to a —CN group.

The term “hydroxy” refers to an —OH group.

The term “hydroxyalkyl” refers to an alkyl group substituted with hydroxy(s), wherein the alkyl is as defined above.

The term “hydroxyalkoxy” refers to an alkoxy group substituted with hydroxy(s), wherein the alkoxy is as defined above.

The term “acyl” refers to a —C(O)R group, wherein R refers to an alkyl, cycloalkyl, alkenyl, alkynyl, wherein the alkyl, cycloalkyl, alkenyl and alkynyl are as defined above.

Non-limiting examples include acetyl, propionyl, butyryl, pentanoyl, hexanoyl, vinylacyl and acryloyl.

The term “amido” refers to a —NHC(O)OR or —C(O)NH₂ group, where R refers to an alkyl, alkenyl, alkynyl, wherein the alkyl, alkenyl and alkynyl are as defined above.

Non-limiting examples include carboxamido, acetamido, propionamido, butyrylamino, pentanoylamino, hexanoylamino, vinylacylamino and acryloylamino.

The term “ester group” refers to a —C(O)OR group, where R refers to an alkyl or cycloalkyl, wherein the alkyl and cycloalkyl are as defined above. Non-limiting examples include ethyl ester group, propyl ester group, butyl ester group, pentyl ester group, cyclopropyl ester group, cyclobutyl ester group, cyclopentyl ester group and cyclohexyl ester group.

“Optionally substituted with” in the present description means that a group is unsubstituted or substituted with one or more (e.g. 2, 3 or 4) substituents, wherein the substituent is selected from the group consisting of halogen, alkyl, alkenyl, alkynyl, haloalkyl, alkoxy, aryl, haloaryl, aryloxy, arylalkyl, arylalkoxy, heterocycloalkyloxy, haloarylalkyloxy, alkylamino, alkylacyl, cyano, heterocyclyl and the like. These substituents can be further substituted. For example, the alkyl as a substituent is also optionally substituted with one or more groups selected from the group consisting of halogen, hydroxy, alkoxy, alkylamino, pyrrolidinyl, phenyl, pyridyl and halophenyl. The heterocyclyl group as a substituent is also optionally substituted with one or more groups selected from the group consisting of halogen, alkyl and alkoxy.

A “pharmaceutical composition” refers to a mixture of one or more of the compounds according to the present invention or physiologically/pharmaceutically acceptable salts or prodrugs thereof with other chemical components, and other components such as physiologically/pharmaceutically acceptable carriers and excipients. The purpose of the pharmaceutical composition is to facilitate administration of a compound to an organism, which is conducive to the absorption of the active ingredient so as to exert biological activity.

Method for Preparing the Compound of Formula (I) of the Present Invention

In order to achieve the purpose of the present invention, the present invention mainly applies the following synthetic schemes and technical solutions.

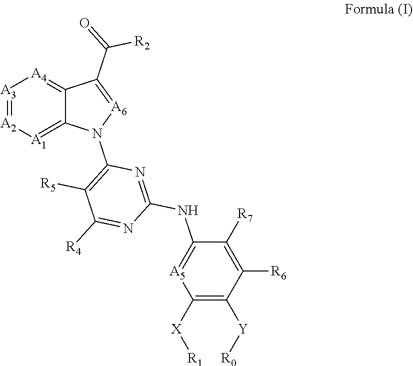

The synthesis of the compound of the present invention is mainly divided into two parts:

Part 1: Synthesis of intermediate M3 from intermediate M1 and pyrimidine intermediate M2

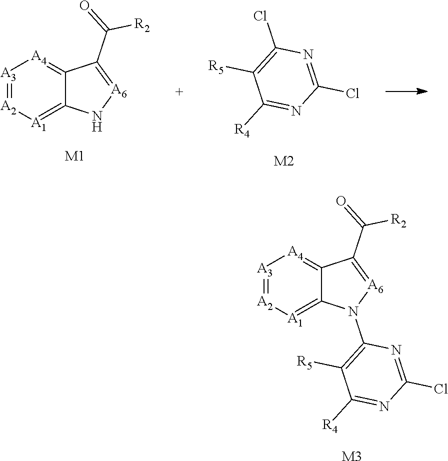

Intermediate M1 and pyrimidine intermediate M2 are subjected to a substitution reaction at an appropriate temperature in the presence of a base and a catalyst in an appropriate solvent to obtain intermediate product M3; the base can be, for example, potassium carbonate, cesium carbonate and the like, the solvent can be, for example, DMF, NMP and the like, and the catalyst can be, for example, 1-hydroxybenzotriazole (HOBT).

Synthesis of Indole Intermediate M1:

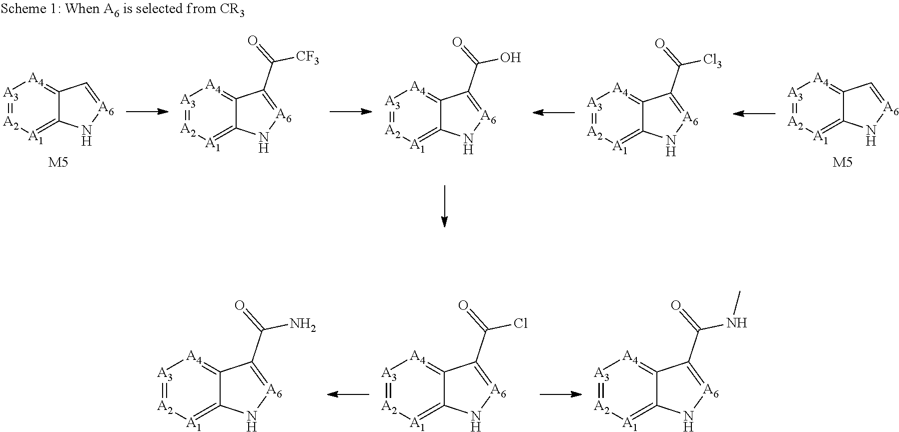

Firstly, a trifluoroacetyl or trichloroacetyl group is introduced into the 3-position of the indole ring of indole intermediate M5 in an appropriate solvent at an appropriate temperature in the presence of trifluoroacetic anhydride or trichloroacetyl chloride. The solvent can be, for example, tetrahydrofuran, dichloromethane and the like.

Secondly, the trifluoroacetyl or trichloroacetyl group is hydrolyzed into a carboxy group in the presence of a base solution. The base solution can be, for example, an aqueous solution of sodium hydroxide, an aqueous solution of potassium hydroxide and the like.

Thirdly, the carboxylic acid is reacted in an appropriate solvent in the presence of an appropriate chlorinating reagent and catalyst to obtain an acyl chloride. The solvent can be, for example, tetrahydrofuran, dichloromethane and the like, the chlorinating reagent can be, for example, oxalyl chloride, sulfoxide chloride, phosphorus oxychloride and the like, and the catalyst can be, for example, DMF and the like.

Finally, the acyl chloride is reacted with methylamine hydrochloride or ammonia in an appropriate solvent in the presence of a base to obtain an indole amide intermediate. The solvent can be, for example, tetrahydrofuran, dichloromethane, DMF and the like, and the base can be, for example, potassium carbonate, triethylamine, pyridine, ammonia and the like.

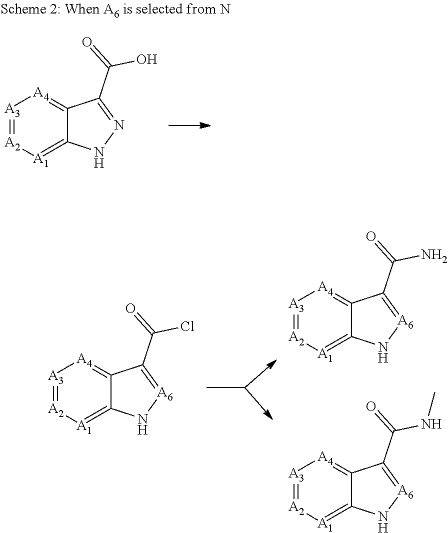

Firstly, an indazole carboxylic acid is reacted in an appropriate solvent in the presence of an appropriate chlorinating reagent and catalyst to obtain an acyl chloride. The solvent can be, for example, tetrahydrofuran, dichloromethane and the like, the chlorinating reagent can be, for example, oxalyl chloride, sulfoxide chloride, phosphorus oxychloride and the like, and the catalyst can be, for example, DMF and the like.

Then, the acyl chloride is reacted with methylamine hydrochloride or ammonia in an appropriate solvent in the presence of a base to obtain an indazole amide intermediate. The solvent can be, for example, tetrahydrofuran, dichloromethane, DMF and the like, and the base can be, for example, potassium carbonate, triethylamine, pyridine, ammonia and the like.

Synthesis of Pyrimidine Intermediate M2:

Substituted pyrimidine intermediates are generally commercially available.

Part 2: Synthesis of the compound of formula (I) from pyrimidine intermediate M3 and aniline intermediate M4

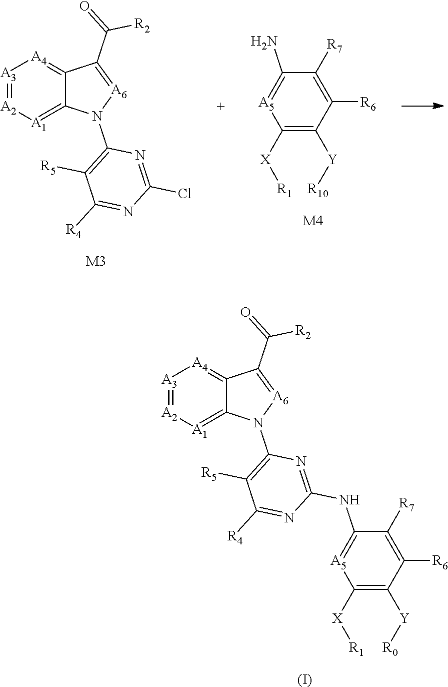

Intermediate M3 and aniline intermediate M4 are reacted under acid catalysis at an appropriate temperature in an appropriate solvent to obtain the compound of formula (I).

The solvent can be, for example, isopropanol, isopentanol, sec-pentanol, dioxane and the like, and the acid can be, for example, hydrochloric acid, sulfuric acid, methanesulfonic acid, p-toluenesulfonic acid, benzenesulfonic acid and the like.

Synthesis of Aniline Intermediate M4: Two Schemes as Follows:

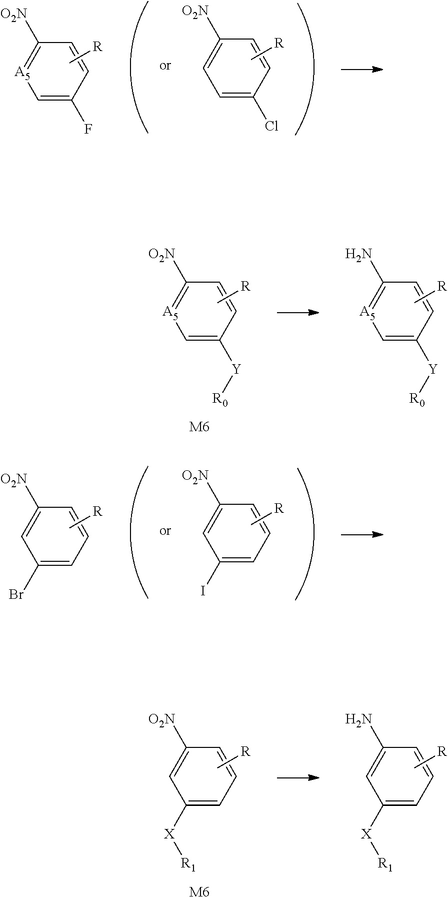

Firstly, nitrobenzene compounds are used as the starting material. If the halogen group is at the para position of the nitro group, intermediate product M6 is obtained by a nucleophilic substitution reaction at an appropriate temperature and pH under base catalysis in an appropriate solvent; the base can be, for example, potassium carbonate, cesium carbonate and the like, and the solvent can be, for example, DMF, acetonitrile and the like. If the halogen group is at the meta position of the nitro group, intermediate M6 is obtained by a Buchwald reaction in an appropriate solvent in the presence of a base, a catalyst and a ligand; the solvent is preferably dioxane and toluene, the base is preferably sodium tert-butoxide, potassium tert-butoxide and cesium carbonate, the catalyst is preferably (pd)₂(dba)₃, palladium acetate and pd (dba)₂, and the ligand is preferably Xphos and BINAP.

Then, the nitro group of intermediate product M6 is reduced to an amino group to obtain intermediate M4; the reduction of the nitro group can be carried out in, for example, an iron powder/ammonium chloride system or H₂/palladium-carbon system.

Wherein, X, Y, A₁, A₂, A₃, A₄, A₅, A₆, R₀, R₁, R₂, R₃, R₄, R₅, R₆ and R₇ are as defined in formula (I) above, and R has the same definition as that of group Q.

The pharmaceutically acceptable salt of the compound of formula (I) of the present invention can be an acid addition salt or a base addition salt. The acid can be an inorganic acid, including but not limited to hydrochloric acid, sulfuric acid, phosphoric acid, hydrobromic acid; or can be an organic acid, including but not limited to citric acid, maleic acid, oxalic acid, formic acid, acetic acid, propionic acid, valeric acid, glycolic acid, benzoic acid, fumaric acid, trifluoroacetic acid, succinic acid, tartaric acid, lactic acid, glutamic acid, aspartic acid, salicylic acid, pyruvic acid, methanesulfonic acid, benzenesulfonic acid, p-toluenesulfonic acid. The base can be an inorganic base, including but not limited to sodium hydroxide, potassium hydroxide, magnesium hydroxide, calcium hydroxide; or can be an organic base, including but not limited to ammonium hydroxide, triethylamine, N,N-dibenzyl ethylenediamine, chloroprocaine, choline, ammonia, diethanolamine and other hydroxyalkyl amines, ethylene diamine, N-methyl glucosamine, procaine, N-benzyl phenylethylamine, arginine or lysine; or can be an alkaline metal salt, including but not limited to lithium, potassium or sodium salts; or can be an alkaline earth metal salt, including but not limited to barium, calcium or magnesium salts; or can be a transition metal salt, including but not limited to zinc salt; or can be other metal salts, including but not limited to sodium hydrogen phosphate or disodium hydrogen phosphate.

In another aspect of the present invention, the compound of formula (I) or a pharmaceutically acceptable salt or prodrug thereof is prepared into a clinically acceptable pharmaceutical composition. According to clinical indications, administration route and way, such pharmaceutical formulations include, but are not limited to, oral formulations such as tablets, gels, soft/hard capsules, emulsions, dispersible powders, granules, water/oil emulsions; injections including intravenous injections, intramuscular injections, intraperitoneal injections, rectal administration suppositories, intracranial injections, which can be aqueous solutions or oil solutions; topical formulations including creams, ointments, gels, water/oil solutions and inclusion formulations; inhalation dosage forms including fine powders, liquid aerosols, and various dosage forms suitable for implanting in body.

A pharmaceutically acceptable carrier, diluent or excipient can be added to the pharmaceutical composition of the present invention as needed. These carriers, diluents, or excipients should comply with the rules of the pharmaceutical preparation process and be compatible with the active ingredients. Carriers for solid oral formulations include, but are not limited to, mannitol, lactose, starch, magnesium stearate, cellulose, glucose, sucrose, cyclodextrin and vitamin E-PEG 1000 which is a molecular carrier for facilitating intestinal absorption. Suitable colorants, sweeteners, flavoring agents and preservatives can be added to the oral formulation.

The compound of formula (I) of the present invention or a pharmaceutically acceptable salt or prodrug thereof is administered to a warm-blooded animal with a unit dose of 0.01-400 mg/kg.

The compound of formula (I) of the present invention or the pharmaceutically acceptable salt or prodrug thereof can be used alone in the above cancer therapy, or as a combination therapy with one or more of the following conventional therapies: radiation therapy, chemotherapy, immunotherapy, tumor vaccine, oncolytic virus therapy, RNAi, cancer adjuvant therapy, bone marrow transplantation and stem cell transplantation, including but not limited to the following anti-tumor drugs and therapies:

1) Alkylating agents, such as cisplatin, oxaliplatin, chlorambucil, cyclophosphamide, nitrogen mustard, melphalan, temozolomide, busulfan, nitrosoureas.

2) Anti-tumor antibiotics, such as doxorubicin, bleomycin, doxorubicin, daunorubicin, epirubicin, idarubicin, mitomycin C, actinomycin, mithramycin; anti-mitotic drugs such as vincristine, vinblastine, vindesine, vinorelbine, paclitaxel, taxotere, Polo kinase inhibitors.

3) Antimetabolites and antifolates, such as fluoropyrimidine, methotrexate, cytarabine, azacitidine, decitabine, raltitrexed, hydroxyurea, IDH1/IDH2 mutant inhibitor.

4) Topoisomerase inhibitors such as epipodophyllotoxin, camptothecin, irinotecan.

5) Cell growth inhibitors, such as anti-estrogen/anti-androgen drugs, such as tamoxifen, fulvestrant, toremifene, raloxifene, droloxifene, idoxifene, bicalutamide, flutamide, nilutamide, cyproterone acetate;

LHRH antagonists or LHRH agonists, such as goserelin, leuprolide, buserelin; progestogens such as megestrol acetate;

Aromatase inhibitors, such as anastrozole, letrozole, vorozole, exemestane; 5a-reductase inhibitors such as finasteride.

6) Anti-invasive agents, such as c-Src kinase family inhibitors, metalloproteinase inhibitors, inhibitors of urokinase plasminogen activator receptor function, and heparinase-like antibodies.

7) Growth function inhibitors, such as growth factor antibodies and growth factor receptor antibodies such as anti-HER2 antibody Trastuzumab, anti-EGFR antibody Panitumumab, anti-EGFR antibody Cetuximab; such inhibitors also include other tyrosine kinase inhibitors and serine/threonine kinase inhibitors, such as Ras/Raf signal conduction inhibitor, cell signaling inhibitors of MEK and/or AKT kinase, c-kit inhibitor, abl kinase inhibitor, PI3 kinase inhibitor, JAKs and STAT3 inhibitor, FLT3 kinase inhibitor, CSF-1R kinase inhibitor, IGF receptor kinase inhibitor, aurora kinase inhibitor, NTRKA/B/C kinase inhibitor.

8) Antiangiogenic agents, such as Bevacizumab, a drug that inhibits vascular endothelial growth factor, and VEGF receptor tyrosine kinase inhibitor.

9) Epigenetic inhibitors such as histone deacetylase inhibitors (HDACi), DNA methyltransferase inhibitors (DNMTi), histone acetyltransferase inhibitors, histone demethylase inhibitors, histone methyltransferase inhibitors and the like.

10) Poly ADP-ribose polymerase inhibitors (PARPi) such as Olaparib, Rucaparib and Niraparib.

11) Tumor immunotherapy, including any in vitro and in vivo methods to increase the immunogenicity of the patient's tumor cell. For example, transfections of cytokines IL-2, IL-4 or GM-CSF; methods of reducing the ineffectiveness of T cells such as anti-PD-1/PD-L mAbs; methods of using transfected immune cells such as dendritic cells transfected with cytokines; methods of using the tumor cell lines transfected with cytokines; methods of reducing the functions of immunosuppressive cells such as regulatory T cells, myeloid-derived suppressor cells, or dendritic cells expressing indoleamine 2,3-deoxygenase; agonist that increases immune cell activity such as STING; as well as methods of cancer vaccines consisting of tumor-associated antigen proteins or peptides.

12) Chimeric antigen receptor T-cell immunotherapy (CART).

13) Oncogene therapy such as CRISPR-Cas 9, RNAi, gene transduction.

### EXAMPLES

The present invention will be further described with reference to the following examples, but the examples should not be considered as limiting the scope of the present invention.

The structures of the compounds are identified by nuclear magnetic resonance (NMR) and/or mass spectrometry (MS). NMR shifts (8) are given in parts per million (ppm). NMR is determined by a Bruker AVANCE-400 machine. The solvents for determination are deuterated-dimethyl sulfoxide (DMSO-d₆), deuterated-chloroform (CDCl₃) and deuterated-methanol (CD₃OD), and the internal standard is tetramethylsilane (TMS).

MS is determined using a liquid chromatography mass spectrometer (Thermo, Ultimate 3000/MSQ).

HPLC is determined using a high-pressure liquid chromatograph (Agilent 1260 Infinity, Gemini C18 250×4.6 mm, 5u column).

The silica gel plate HSGF245 used for thin layer chromatography (TLC) has a specification of 0.15 mm to 0.2 mm. The specifications for the separation and purification of the product by thin-layer chromatography are 0.9 mm to 1.0 mm (Yantai Huanghai).

Column chromatography generally applies 200 to 300 mesh silica gel as the carrier (Yantai Huanghai silica gel).

The known starting materials of the present invention can be synthesized using or in accordance with methods known in the prior art, or purchased from Shanghai Darui Fine Chemicals Co., Ltd., Shanghai Titan Technology Co., Ltd., Shanghai Runjie Chemical Reagent Co., Ltd., TCI, or Aldrich Chemical Company. If the experimental conditions are not specified in the examples, usually the conventional conditions or conditions recommended by the raw material or product manufacturers are adopted.

Reagents that are not specified the sources are conventional reagents purchased from the market.

Unless otherwise specified in the examples, the reactions can be carried out under argon or nitrogen atmosphere. The argon or nitrogen atmosphere means that the reaction flask is connected to an argon or nitrogen balloon of approximately 1 L in volume.

Unless otherwise specified in the examples, the solution refers to an aqueous solution.

Unless otherwise specified in the examples, the reaction temperature is room temperature from 20° C. to 30° C.

### Example 1

### Preparation of 1-[2-(3-bromo-4-fluoro-phenylamino)-pyrimidin-4-yl]-1H-indole-3-carboxamide (Compound 1)

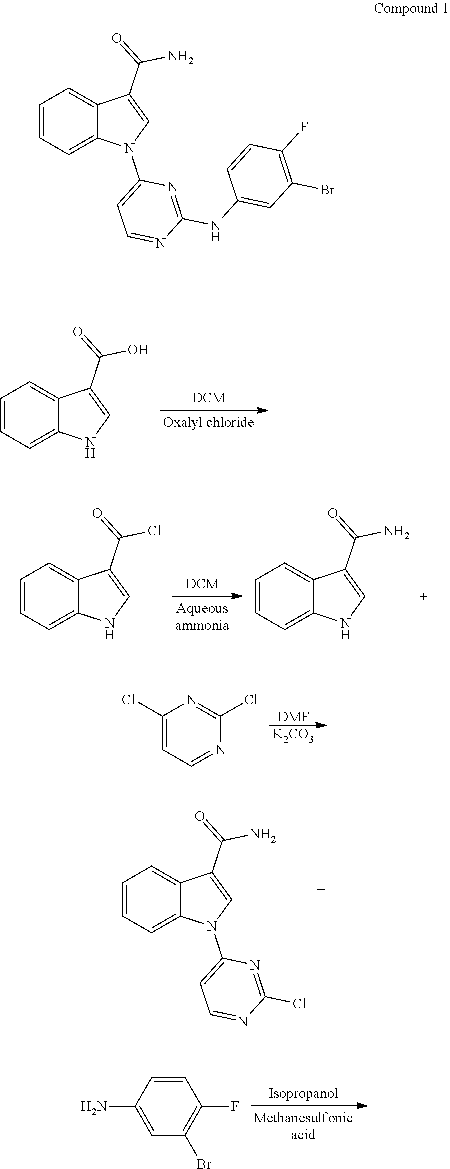

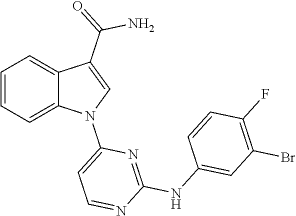 [SMILES: NC(=O)c1cn(-c2ccnc(Nc3ccc(F)c(Br)c3)n2)c2ccccc12]

### Step 1: Preparation of 1H-indole-3-carbonyl chloride

3-Indolecarboxylic acid (30 g, 0.186 mol) was stirred in 500 ml of dichloromethane at room temperature, and failed to dissolve completely. 0.5 ml of DMF was added to the solution, then oxalyl chloride (71.0 g, 0.56 mol) was slowly added dropwise to the solution at room temperature. The addition was completed after 30 minutes, and the reaction solution was reacted at room temperature for 2 hours. TLC showed that the reaction was completed. The reaction solution was concentrated under reduced pressure to obtain the crude product 1H-indole-3-carbonyl chloride (yellow solid). The product was used directly in the next step without purification.

### Step 2: Preparation of 1H-indole-3-carboxamide

1H-indole-3-carbonyl chloride (0.186 mol, theoretical yield) obtained in step 1 was added to 500 ml of DCM, and stirred at room temperature for 30 minutes. It could not dissolve completely, and a turbid dispersion system was obtained. 350 ml of aqueous ammonia and 200 ml of DCM were added to a 2 L three-necked flask and stirred vigorously. The turbid dispersion system of 1H-indole-3-carbonyl chloride in dichloromethane was slowly added dropwise to the 2 L three-necked flask at room temperature. The addition was completed after 20 minutes, and the reaction solution was reacted at room temperature for 1 hour. TLC showed that the reaction was completed. The reaction solution was filtrated. The resulting solid was washed with a small amount of ethanol, and dried by blowing (60° C.) for 8 hours to obtain 20 g of the crude product 1H-indole-3-carboxamide (yellow solid). The product was used directly in the next step without purification.

### Step 3: Preparation of 1-(2-chloro-pyrimidin-4-yl)-1H-indole-3-carboxamide

30 ml of DMF was added to a 100 ml three-necked flask, then 2,4-dichloropyrimidine (9.8 g, 0.06 mol), HOBT (1.2 g, 0.008 mol), potassium carbonate (18.1 g, 0.12 mol) and 1H-indole-3-carboxamide (7 g, 0.04 mol) were added successively at room temperature under stirring. The reaction solution was reacted at room temperature for 1 hour, then heated to 80° C. and reacted for 2 hours. TLC showed that the reaction was completed. The reaction solution was cooled to room temperature, then 60 ml of water was added dropwise to precipitate a solid. The solution was filtrated to obtain the crude product. The resulting crude product was added to 25 ml ethanol, and stirred at room temperature for 30 minutes. The solution was filtrated. The resulting solid was washed with a small amount of ethanol, and dried by blowing at 60° C. for 8 hours to obtain 10 g of 1-(2-chloro-pyrimidin-4-yl)-1H-indole-3-carboxamide as solid.

### Step 4: Preparation of 1-(4-benzimidazol-1-yl-phenyl)-3-isoxazol-3-yl-urea

1-(2-Chloro-pyrimidin-4-yl)-1H-indole-3-carboxamide (100 mg, 0.367 mmol) obtained in Step 3, 3-bromo-4-fluoroaniline (69.5 mg, 0.367 mmol) and methanesulfonic acid (52.8 mg, 0.55 mmol) were dispersed in 10 ml of isopropanol, and reacted under reflux for 4 hours. TLC showed that the reaction was substantially completed. The reaction solution was cooled, then 10 ml of methyl tert-butyl ether was added. The solution was stirred at room temperature for 10 minutes and then filtrated, and the resulting solid was washed with a small amount of methyl tert-butyl ether. The resulting solid was dissolved in 50 ml of dichloromethane/methanol (dichloromethane:methanol=5:1), to which 10 ml of aqueous sodium hydroxide solution (0.5 mol/L) was added, and the resulting solution was extracted with dichloromethane. The organic phase was washed with saturated NaCl solution twice, dried over anhydrous sodium sulfate, filtrated and concentrated under reduced pressure. The resulting residues were purified by column chromatography (eluent: dichloromethane/methanol) to obtain 20 mg of 1-[2-(3-bromo-4-fluoro-phenylamino)-pyrimidin-4-yl]-1H-indole-3-carboxamide as solid.

¹HNMR (DMSO-d6, 400 MHz) δ: 10.00 (1H, s), 8.78 (1H, s), 8.72 (1H, d), 8.64 (1H, d), 8.27 (2H, d), 7.71 (2H, br), 7.40-7.29 (3H, m), 7.18 (2H, d).

LC-MS (ESI): 428.0 (M+H)⁺.

### Example 2

### Preparation of 1-[2-(3-chloro-4-trifluoromethyl-phenylamino)-pyrimidin-4-yl]-1H-indole-3-carboxamide (Compound 2)

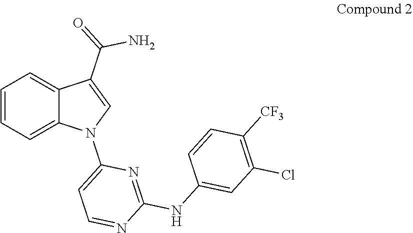 [SMILES: NC(=O)c1cn(-c2ccnc(Nc3ccc(C(F)(F)F)c(Cl)c3)n2)c2ccccc12]

1-[2-(3-Chloro-4-trifluoromethyl-phenylamino)-pyrimidin-4-yl]-1H-indole-3-carboxamide was obtained in accordance with the same preparation method of Example 1 except for replacing the 3-bromo-4-fluoroaniline in Step 4 with 3-chloro-4-trifluoromethylaniline (TCI).

¹HNMR (DMSO-d6, 400 MHz) δ: 10.42 (1H, s), 8.81 (1H, s), 8.77 (1H, d), 8.73 (1H, d), 8.34 (1H, s), 8.28 (1H, d), 7.90 (1H, d), 7.82 (1H, d), 7.69 (s), 7.40-7.31 (2H, m), 7.29 (1H, d), 7.21 (1H, s).

LC-MS (ESI): 432.0 (M+H)⁺.

### Example 3

### Preparation of 1-[2-(2-chloro-3-fluoro-phenylamino)-pyrimidin-4-yl]-1H-indole-3-carboxamide (Compound 3)

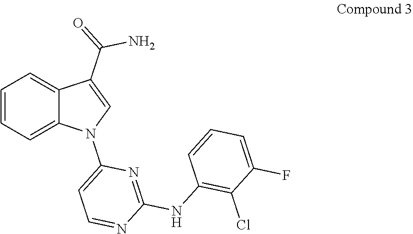 [SMILES: NC(=O)c1cn(-c2ccnc(Nc3cccc(F)c3Cl)n2)c2ccccc12]

1-[2-(2-chloro-3-fluoro-phenylamino)-pyrimidin-4-yl]-1H-indole-3-carboxamide was obtained in accordance with the same preparation method of Example 1 except for replacing the 3-bromo-4-fluoroaniline in Step 4 with 2-chloro-3-fluoroaniline (TCI).

¹HNMR (DMSO-d6, 400 MHz) δ: 9.55 (1H, s), 8.78 (1H, m), 8.55 (1H, d), 8.36 (1H, d), 8.22 (1H, d), 7.65 (1H, s), 7.57 (1H, d), 7.46 (1H, q), 7.34 (1H, t), 7.25 (1H, t), 7.18-7.12 (3H, br).

LC-MS (ESI): 382.0 (M+H)⁺.

### Example 4

### Preparation of 1-[2-(3-fluoro-5-trifluoromethyl-phenylamino)-pyrimidin-4-yl]-1H-indole-3-carboxamide (Compound 4)

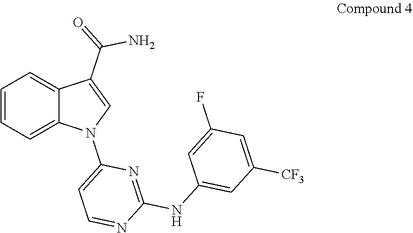 [SMILES: NC(=O)c1cn(-c2ccnc(Nc3cc(F)cc(C(F)(F)F)c3)n2)c2ccccc12]

1-[2-(3-Fluoro-5-trifluoromethyl-phenylamino)-pyrimidin-4-yl]-1H-indole-3-carboxamide was obtained in accordance with the same preparation method of Example 1 except for replacing the 3-bromo-4-fluoroaniline in Step 4 with 3-fluoro-5-trifluoromethylaniline (TCI).

¹HNMR (DMSO-d6, 400 MHz) δ: 10.38 (1H, s), 8.81 (1H, s), 8.76 (1H, d), 8.72 (1H, d), 8.26 (1H, d), 8.13 (1H, d), 8.04 (1H, s), 7.67 (1H, s), 7.34 (2H, m), 7.27-7.22 (3H, m).

LC-MS (ESI): 416.0 (M+H)⁺.

### Example 5

### Preparation of 1-[2-(3,4-methoxy-phenylamino)-pyrimidin-4-yl]-1H-indole-3-carboxamide (Compound 5)

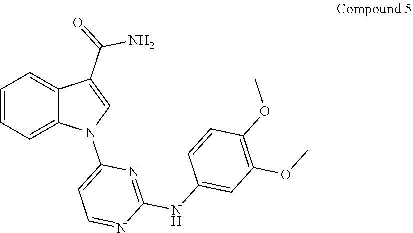 [SMILES: COc1ccc(Nc2nccc(-n3cc(C(N)=O)c4ccccc43)n2)cc1OC]

1-[2-(3,4-Methoxy-phenylamino)-pyrimidin-4-yl]-1H-indole-3-carboxamide was obtained in accordance with the same preparation method of Example 1 except for replacing the 3-bromo-4-fluoroaniline in Step 4 with 3,4-dimethoxyaniline (TCI).

¹HNMR (DMSO-d6, 400 MHz) δ: 9.64 (1H, s), 8.85 (1H, s), 8.78 (1H, s), 8.67 (1H, br), 8.56 (1H, d), 8.26 (1H, d), 7.66 (1H, br), 7.43 (1H, d), 7.29 (2H, br), 7.17 (1H, s), 7.07 (1H, d), 6.95 (1H, d), 3.75 (3H, s), 3.73 (3H, s).

LC-MS (ESI): 390.1 (M+H)⁺.

### Example 6

### Preparation of 1-[2-(4-fluoro-3-nitro-phenylamino)-pyrimidin-4-yl]-1H-indole-3-carboxamide (Compound 6)

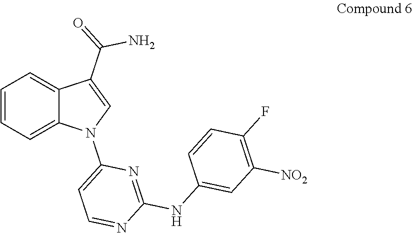 [SMILES: NC(=O)c1cn(-c2ccnc(Nc3ccc(F)c([N+](=O)[O-])c3)n2)c2ccccc12]

1-[2-(4-Fluoro-3-nitro-phenylamino)-pyrimidin-4-yl]-1H-indole-3-carboxamide was obtained in accordance with the same preparation method of Example 1 except for replacing the 3-bromo-4-fluoroaniline in Step 4 with 3-nitro-4-fluoroaniline (TCI).

¹HNMR (DMSO-d6, 400 MHz) δ: 8.83 (1H, s), 8.77-8.72 (2H, m), 8.68 (1H, d), 8.28 (1H, dd), 8.13-8.10 (1H, br), 7.71 (1H, s), 7.61-7.56 (1H, m), 7.37-7.30 (2H, m), 7.25 (2H, d).

LC-MS (ESI): 393.1 (M+H)⁺.

### Example 7

### Preparation of 1-[2-(4-fluoro-2-methoxy-5-nitro-phenylamino)-pyrimidin-4-yl]-1H-indole-3-carboxamide (Compound 7)

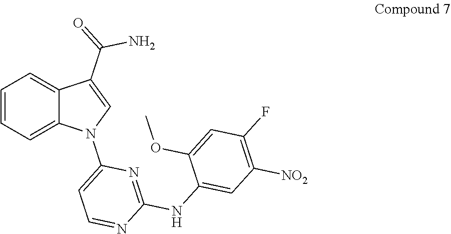 [SMILES: COc1cc(F)c([N+](=O)[O-])cc1Nc1nccc(-n2cc(C(N)=O)c3ccccc32)n1]

1-[2-(4-Fluoro-2-methoxy-5-nitro-phenylamino)-pyridin-4-yl]-1H-indole-3-carboxamide was obtained in accordance with the same preparation method of Example 1 except for replacing the 3-bromo-4-fluoroaniline in Step 4 with 2-methoxy-4-fluoro-5-nitroaniline (TCI).

¹HNMR (DMSO-d6, 400 MHz) δ: 8.79 (1H, s), 8.72 (1H, d), 8.59 (1H, d), 8.52 (1H, d), 8.25 (1H, d), 7.67 (1H, s), 7.42 (1H, d), 7.30-7.15 (4H, m), 3.99 (3H, s).

LC-MS (ESI): 423.0 (M+H)⁺.

### Example 8

### Preparation of 1-{2-[4-(2-dimethylamino-ethylamino)-phenylamino]-pyrimidin-4-yl}-1H-indole-3-carboxamide (Compound 8)

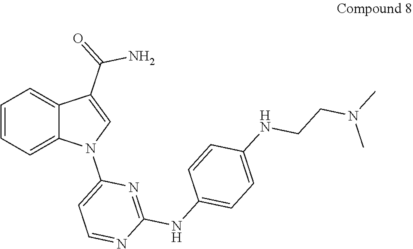 [SMILES: CN(C)CCNc1ccc(Nc2nccc(-n3cc(C(N)=O)c4ccccc43)n2)cc1]

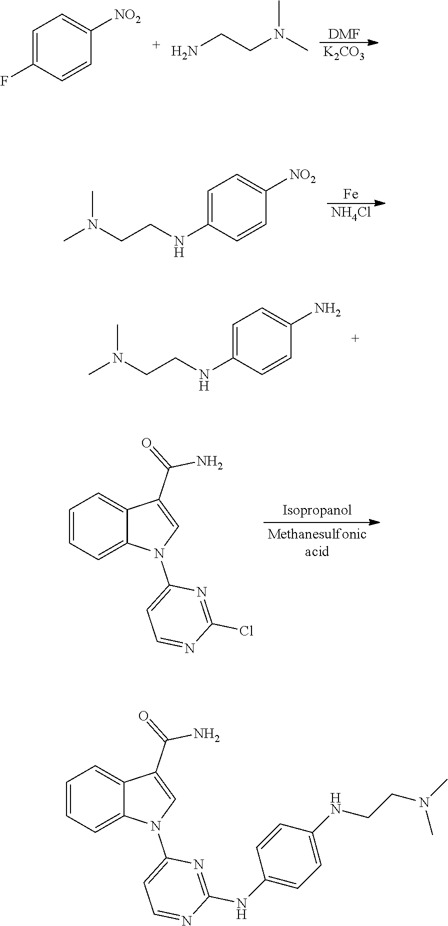

Step 1: Preparation of N,N-dimethyl-N′-(4-nitro-phenyl)-ethane-1,2-diamine

4-Fluoronitrobenzene (423 mg, 3 mmol) and N,N-dimethylethylenediamine (400 mg, 4.5 mmol) were dissolved in DMF (5 ml). Potassium carbonate (1.24 g, 9 mmol) was added at room temperature, and the resulting mixture was heated to 80° C. and reacted for 2 hours. TLC showed that the reaction was completed. The reaction solution was cooled to room temperature, slowly poured into water (50 ml), and extracted with ethyl acetate (50 ml×2). The organic phase was washed with saturated NaCl solution twice, dried over anhydrous sodium sulfate, filtrated, and concentrated under reduced pressure to obtain the crude product N,N-dimethyl-N′-(4-nitro-phenyl)-ethane-1,2-diamine (600 mg, yellow oil). The product was used directly in the next step without purification.

### Step 2: Preparation of N-(2-dimethylamino-ethyl)-benzene-1,4-diamine

The product N,N-dimethyl-N′-(4-nitro-phenyl)-ethane-1,2-diamine (600 mg, 2.87 mmol) obtained in Step 1, reduced iron powder (480 mg, 9 mmol) and ammonium chloride (670 mg, 12 mmol) were added into ethanol (20 ml)/water (5 ml). The resulting mixture was heated to 90° C. and reacted for 1 hour. The reaction solution was cooled to room temperature, slowly poured into saturated aqueous sodium bicarbonate solution (50 ml), and extracted with ethyl acetate (50 ml×2). The organic phase was washed with saturated NaCl solution twice, dried over anhydrous sodium sulfate, filtrated, and concentrated under reduced pressure to obtain the crude product N-(2-dimethylamino-ethyl)-benzene-1,4-diamine (400 mg, yellow oil). The product was used directly in the next step without purification.

### Step 3: Preparation of 1-{2-[4-(2-dimethylamino-ethylamino)-phenylamino]-pyrimidin-4-yl}-1H-indole-3-carboxamide

1-{2-[4-(2-Dimethylamino-ethylamino)-phenylamino]-pyrimidin-4-yl}-1H-indole-3-carboxamide was obtained in accordance with the same preparation method of Step 4 of Example 1 except for replacing the 3-bromo-4-fluoroaniline in Step 4 of Example 1 with N-(2-dimethylamino-ethyl)-benzene-1,4-diamine (prepared in Step 2).

¹HNMR (DMSO-d6, 400 MHz) δ: 9.43 (1H, s), 8.81 (1H, s), 8.69 (1H, s), 8.53 (1H, d), 8.30 (1H, d), 7.73 (1H, s), 7.44 (2H, d), 7.32 (2H, m), 7.22 (1H, s) 7.03 (1H, d), 6.68 (1H, d), 5.27 (1H, s), 3.16 (2H, t), 2.55 (2H, t), 2.27 (6H, s).

LC-MS (ESI): 416.1 (M+H)⁺.

### Example 9

### Preparation of 1-{2-[4-(2-morpholin-4-yl-ethylamino)-phenylamino]-pyrimidin-4-yl}-1H-indole-3-carboxamide (Compound 9)

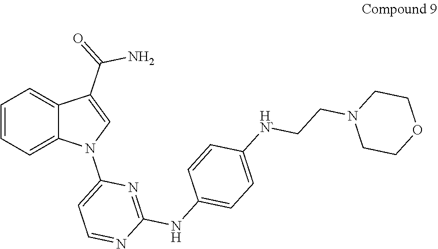

1-{2-[4-(2-Morpholin-4-yl-ethylamino)-phenylamino]-pyrimidin-4-yl}-1H-indole-3-carboxamide was obtained in accordance with the same preparation method of Example 8 except for replacing the N,N-dimethylethylenediamine in Step 1 with N-(2-aminoethyl)morpholine (Darui).

¹HNMR (DMSO-d6, 400 MHz) δ: 9.37 (1H, s), 8.76 (1H, s), 8.65 (1H, s), 8.48 (1H, d), 8.24 (1H, dd), 7.67 (1H, s), 7.39 (2H, d), 7.29 (2H, m), 7.16 (1H, s), 6.98 (1H, d), 6.63 (2H, d), 5.25 (1H, s), 3.61 (4H, t), 3.15 (2H, t), 2.53 (2H, t), 2.44 (4H, t).

LC-MS (ESI): 458.2 (M+H)⁺.

### Example 10

### Preparation of 1-{2-[4-(2-piperidin-1-yl-ethylamino)-phenylamino]-pyrimidin-4-yl}-1H-indole-3-carboxamide (Compound 10)

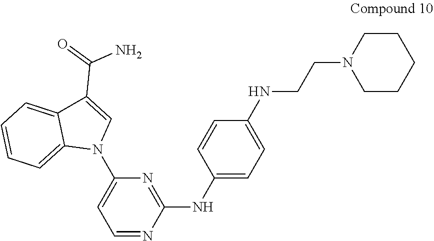 [SMILES: NC(=O)c1cn(-c2ccnc(Nc3ccc(NCCN4CCCCC4)cc3)n2)c2ccccc12]

1-{2-[4-(2-Piperidin-1-yl-ethylamino)-phenylamino]-pyrimidin-4-yl}-1H-indole-3-carboxamide was obtained in accordance with the same preparation method of Example 8 except for replacing the N,N-dimethylethylenediamine in Step 1 with 1-(2-aminoethyl)piperidine (Darui).

¹HNMR (DMSO-d6, 400 MHz) δ: 9.39 (1H, s), 8.76 (1H, s), 8.65 (1H, s), 8.49 (1H, d), 8.26 (1H, d), 7.69 (1H, s), 7.39 (2H, d), 7.28 (2H, d), 7.19 (1H, s), 6.98 (1H, d), 6.62 (2H, d), 5.22 (1H, s), 3.17 (2H, t), 2.47 (2H, t), 2.40 (4H, t), 1.52 (4H, m), 1.40 (2H, m).

LC-MS (ESI): 456.1 (M+H)⁺.

### Example 11

### Preparation of 1-(2-{4-[2-(4-methyl-piperazin-1-yl)-ethylamino]-phenylamino}-pyrimidin-4-yl)-1H-indole-3-carboxamide (Compound 11)

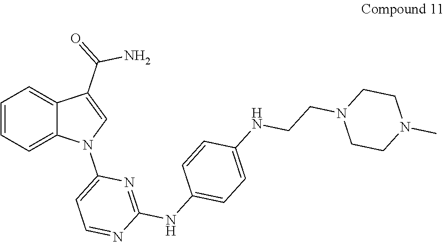 [SMILES: CN1CCN(CCNc2ccc(Nc3nccc(-n4cc(C(N)=O)c5ccccc54)n3)cc2)CC1]

1-(2-{4-[2-(4-Methyl-piperazin-1-yl)-ethylamino]-phenylamino}-pyrimidin-4-yl)-1H-indole-3-carboxamide was obtained in accordance with the same preparation method of Example 8 except for replacing the N,N-dimethylethylenediamine in Step 1 with 4-methyl-1-piperazinyl ethylamine (Darui).

¹HNMR (DMSO-d6, 400 MHz) δ: 9.38 (1H, s), 8.76 (1H, s), 8.65 (1H, s), 8.49 (1H, d), 8.25 (1H, d), 7.68 (1H, s), 7.39 (2H, d), 7.29 (2H, m), 7.18 (1H, s), 6.98 (1H, d), 6.63 (2H, d), 5.23 (1H, s), 3.13 (2H, t), 2.54-2.36 (10H, m), 2.18 (3H, s).

LC-MS (ESI): 471.1 (M+H)⁺.

### Example 12

### Preparation of 1-{2-[4-(piperidin-4-amino)-phenylamino]-pyrimidin-4-yl}-1H-indole-3-carboxamide (Compound 12)

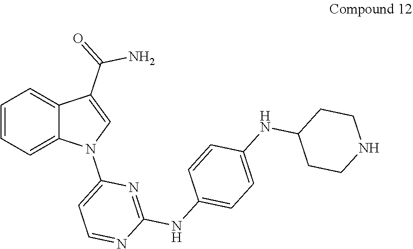 [SMILES: NC(=O)c1cn(-c2ccnc(Nc3ccc(NC4CCNCC4)cc3)n2)c2ccccc12]

1-{2-[4-(Piperidin-4-amino)-phenylamino]-pyrimidin-4-yl}-1H-indole-3-carboxamide was obtained in accordance with the same preparation method of Example 8 except for replacing the N,N-dimethylethylenediamine in Step 1 with 1-Boc-4-aminopiperidine (Darui).

¹HNMR (DMSO-d6, 400 MHz) δ: 1.15-1.27 (2H, m), 1.88 (2H, d), 2.54 (2H, t), 2.95 (2H, d), 3.23 (1H, m), 5.25 (1H, d), 6.60 (2H, d), 6.97 (1H, d), 7.14-7.35 (5H, m), 7.72 (1H, br), 8.24 (1H, d), 8.46 (1H, d), 8.64 (1H, br), 8.78 (1H, s), 9.34 (1H, s).

LC-MS (ESI): 428.1 (M+H)⁺.

### Example 13

### Preparation of 1-{2-[4-(2-pyrrolidin-1-yl-ethylamino)-phenylamino]-pyrimidin-4-yl}-1H-indole-3-carboxamide (Compound 13)

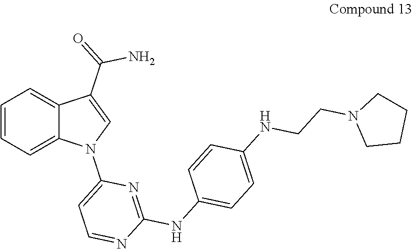 [SMILES: NC(=O)c1cn(-c2ccnc(Nc3ccc(NCCN4CCCC4)cc3)n2)c2ccccc12]

1-{2-[4-(2-Pyrrolidin-1-yl-ethylamino)-phenylamino]-pyrimidin-4-yl}-1H-indole-3-carboxamide was obtained in accordance with the same preparation method of Example 8 except for replacing the N,N-dimethylethylenediamine in Step 1 with 1-(2-aminoethyl)pyrrolidine (Darui).

¹HNMR (DMSO-d6, 400 MHz) δ: 9.36 (1H, s), 8.76 (1H, s), 8.65 (1H, s), 8.49 (1H, d), 8.25 (1H, d), 7.67 (1H, s), 7.39 (2H, d), 7.28 (2H, m), 7.16 (1H, s) 6.98 (1H, d), 6.63 (2H, d), 6.42 (1H, d), 3.16 (2H, t), 3.02 (2H, t), 2.67 (4H, m), 1.72 (4H, m).

LC-MS (ESI): 442.2 (M+H)⁺.

### Example 14

### Preparation of 1-[2-(4-morpholin-4-methyl-phenylamino)-pyrimidin-4-yl]-1H-indole-3-carboxamide (Compound 14)

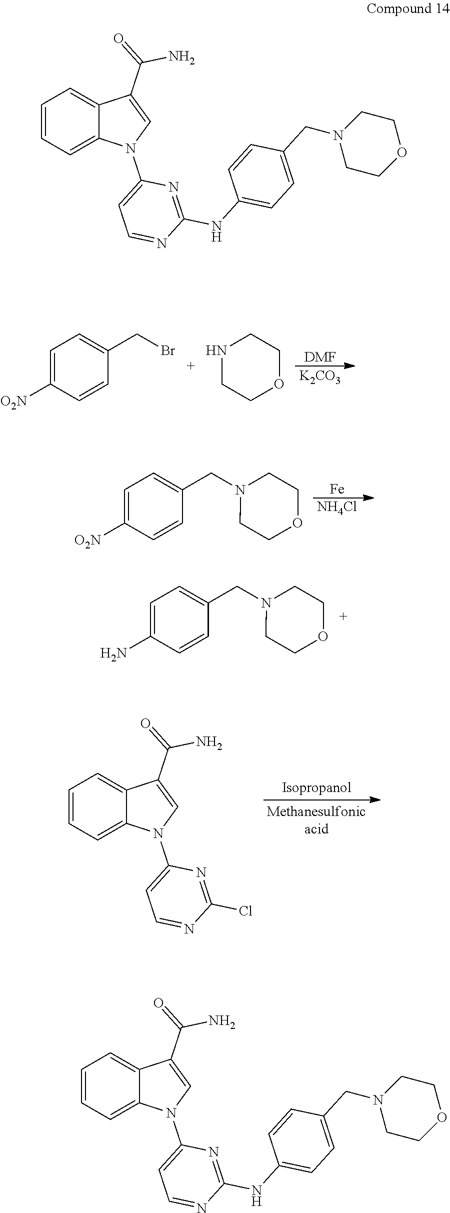

### Step 1: Preparation of 4-(4-nitro-benzyl)-morpholine

p-Nitrobenzyl bromide (430 mg, 2 mmol) and morpholine (261 mg, 3 mmol) were dissolved in DMF (5 ml). Potassium carbonate (828 mg, 6 mmol) was added at room temperature, and the resulting mixture was heated to 80° C. and reacted for 2 hours. TLC showed that the reaction was completed. The reaction solution was cooled to room temperature, slowly poured into water (50 ml), and extracted with ethyl acetate (50 ml×2). The organic phase was washed with saturated NaCl solution twice, dried over anhydrous sodium sulfate, filtrated, and concentrated under reduced pressure to obtain the crude product 4-(4-nitro-benzyl)-morpholine (350 mg). The product was used directly in the next step without purification.

Step 2: Preparation of 4-morpholino-4-ylmethyl-aniline

The product 4-(4-nitro-benzyl)-morpholine (350 mg, 1.58 mmol) obtained in Step 1, reduced iron powder (441 mg, 7.89 mmol) and ammonium chloride (676 mg, 12.6 mmol) were added into ethanol (20 ml)/water (5 ml). The resulting mixture was heated to 90° C. and reacted for 1 hour. The reaction solution was cooled to room temperature, slowly poured into saturated aqueous sodium bicarbonate solution (50 ml), and extracted with ethyl acetate (50 ml×2). The organic phase was washed with saturated NaCl solution twice, dried over anhydrous sodium sulfate, filtrated, and concentrated under reduced pressure to obtain the crude product 4-morpholino-4-ylmethyl-aniline (200 mg). The product was used directly in the next step without purification.

### Step 3: Preparation of 1-[2-(4-morpholin-4-methyl-phenylamino)-pyrimidin-4-yl]-1H-indole-3-carboxamide

1-[2-(4-Morpholin-4-methyl-phenylamino)-pyrimidin-4-yl]-1H-indole-3-carboxamide was obtained in accordance with the same preparation method of Step 4 of Example 1 except for replacing the 3-bromo-4-fluoroaniline in Step 4 of Example 1 with 4-morpholino-4-ylmethyl-aniline (prepared in Step 2).

¹HNMR (DMSO-d6, 400 MHz) δ: 2.36 (4H, br), 2.44 (2H, s), 3.58 (4H, br), 7.10 (1H, d), 7.18 (1H, br), 7.25-7.33 (4H, m), 7.62-7.72 (3H, m), 8.24-8.27 (1H, m), 8.58 (1H, d), 8.71 (1H, br), 8.78 (1H, s), 9.80 (1H, s).

LC-MS (ESI): 429.1 (M+H)⁺.

### Example 15

### Preparation of 1-{2-[4-(4-acetyl-piperazin-1-methyl)-phenylamino]-pyrimidin-4-yl}-1H-indole-3-carboxamide (Compound 15)

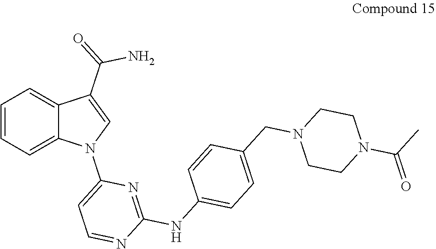 [SMILES: CC(=O)N1CCN(Cc2ccc(Nc3nccc(-n4cc(C(N)=O)c5ccccc54)n3)cc2)CC1]

1-{2-[4-(4-Acetyl-piperazin-1-methyl)-phenylamino]-pyrimidin-4-yl}-1H-indole-3-carboxamide was obtained in accordance with the same preparation method of Example 14 except for replacing the morpholine in Step 1 of Example 14 with 4-acetylpiperazine.

¹HNMR (DMSO-d6, 400 MHz) δ: 1.99 (3H, s), 2.34 (2H, br), 2.40 (2H, br), 3.49 (4H, br), 7.12 (1H, d), 7.19-7.34 (5H, m), 7.71-7.75 (3H, m), 8.26 (1H, m), 8.59 (1H, d), 8.73 (1H, br), 8.79 (1H, s), 9.84 (1H, s).

LC-MS (ESI): 470.2 (M+H)⁺.

### Example 16

### Preparation of 1-{2-[4-(4-methyl-piperazin-1-carbonyl)-phenylamino]-pyrimidin-4-yl}-1H-indole-3-carboxamide (Compound 16)

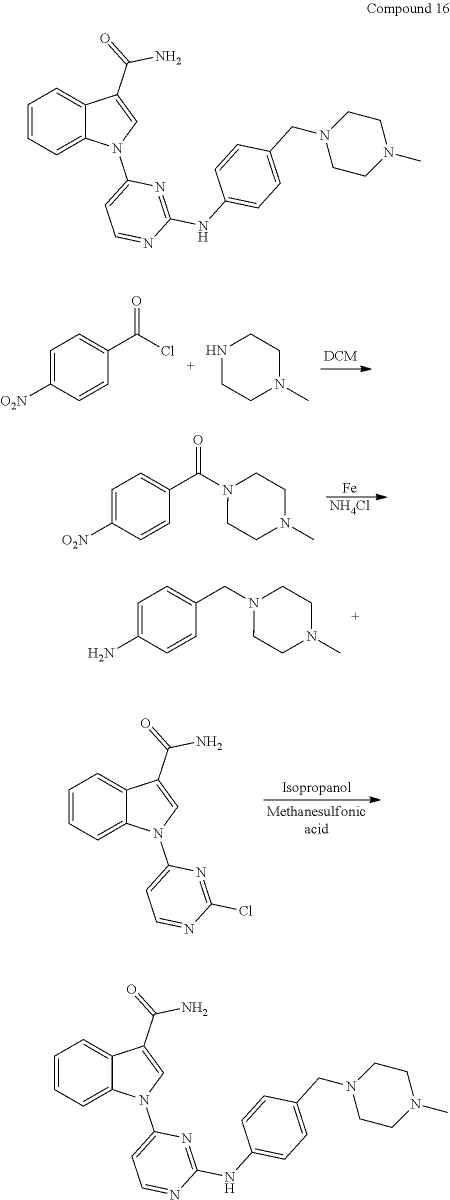

### Step 1: Preparation of (4-methyl-piperazin-1-yl)-(4-nitro-phenyl)-methanone

4-Nitrobenzoyl chloride (555 mg, 3 mmol) was dissolved in DCM (20 ml), and the resulting solution was cooled in an ice water bath. N-methylpiperazine (900 mg, 9 mol) was slowly added dropwise, and the reaction solution was stirred at room temperature for 30 minutes after completion of the addition. TLC showed that the reaction was completed. The reaction solution was slowly poured into 100 ml of water, and extracted with dichloromethane (50 ml×2). The organic phase was washed with saturated NaCl solution twice, dried over anhydrous sodium sulfate, filtrated, and concentrated under reduced pressure to obtain the crude product (4-methyl-piperazin-1-yl)-(4-nitro-phenyl)-methanone (695 mg). The product was used directly in the next step without purification.

### Step 2: Preparation of (4-amino-phenyl)-(4-methyl-piperazin-1-yl)-methanone

The product (4-methyl-piperazin-1-yl)-(4-nitro-phenyl)-methanone (695 mg, 2.80 mmol) obtained in Step 1, reduced iron powder (784 mg, 14.0 mmol) and ammonium chloride (1.2 g, 22.4 mmol) were added into ethanol (40 ml)/water (1 ml). The resulting mixture was heated to 90° C. and reacted for 1 hour. The reaction solution was cooled to room temperature, slowly poured into saturated aqueous sodium bicarbonate solution (150 ml), and extracted with ethyl acetate (100 ml×2). The organic phase was washed with saturated NaCl solution twice, dried over anhydrous sodium sulfate, filtrated, and concentrated under reduced pressure to obtain the crude product (4-amino-phenyl)-(4-methyl-piperazin-1-yl)-methanone (510 mg). The product was used directly in the next step without purification.

### Step 3: Preparation of 1-{2-[4-(4-methyl-piperazin-1-carbonyl)-phenylamino]-pyrimidin-4-yl}-1H-indole-3-carboxamide

1-{2-[4-(4-Methyl-piperazin-1-carbonyl)-phenylamino]-pyrimidin-4-yl}-1H-indole-3-carboxamide was obtained in accordance with the same preparation method of Step 4 of Example 1 except for replacing the 3-bromo-4-fluoroaniline in Step 4 of Example 1 with (4-amino-phenyl)-(4-methyl-piperazin-1-yl)-methanone (prepared in Step 2).

¹HNMR (DMSO-d₆, 400 MHz) δ: 10.06 (1H, s), 8.80 (1H, s), 8.76 (1H, d), 8.65 (1H, d), 8.26 (1H, d), 7.88 (2H, d), 7.70 (1H, s), 7.42 (2H, d), 7.33 (2H, m), 7.19 (2H, d), 3.54 (4H, s), 2.39 (4H, s), 2.25 (3H, s).

LC-MS (ESI): 456.1 (M+H)⁺.

### Example 17

### Preparation of 1-[2-(4-methoxy-phenylamino)-pyrimidin-4-yl]-1H-indole-3-carboxamide (Compound 17)

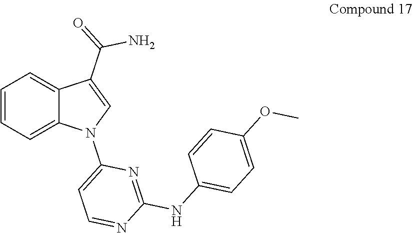 [SMILES: COc1ccc(Nc2nccc(-n3cc(C(N)=O)c4ccccc43)n2)cc1]

1-[2-(4-Methoxy-phenylamino)-pyrimidin-4-yl]-1H-indole-3-carboxamide was obtained in accordance with the same preparation method of Step 4 of Example 1 except for replacing the 3-bromo-4-fluoroaniline in Step 4 of Example 1 with 4-methoxyaniline.

¹HNMR (DMSO-d6, 400 MHz) δ: 3.77 (3H, s), 6.95 (2H, d), 7.06 (1H, d), 7.19 (1H, s), 7.28-7.31 (2H, m), 7.64 (2H, d), 7.71 (1H, s), 8.24-8.26 (1H, m), 8.54 (1H, d), 8.68 (1H, br), 8.79 (1H, s), 9.65 (1H, s).

LC-MS (ESI): 360.2 (M+H)⁺.

### Example 18

### Preparation of 1-(2-{4-[2-(4-methyl-piperazin-1-yl)-ethoxy]-phenylamino}-pyrimidin-4-yl)-1H-indole-3-carboxamide (Compound 18)

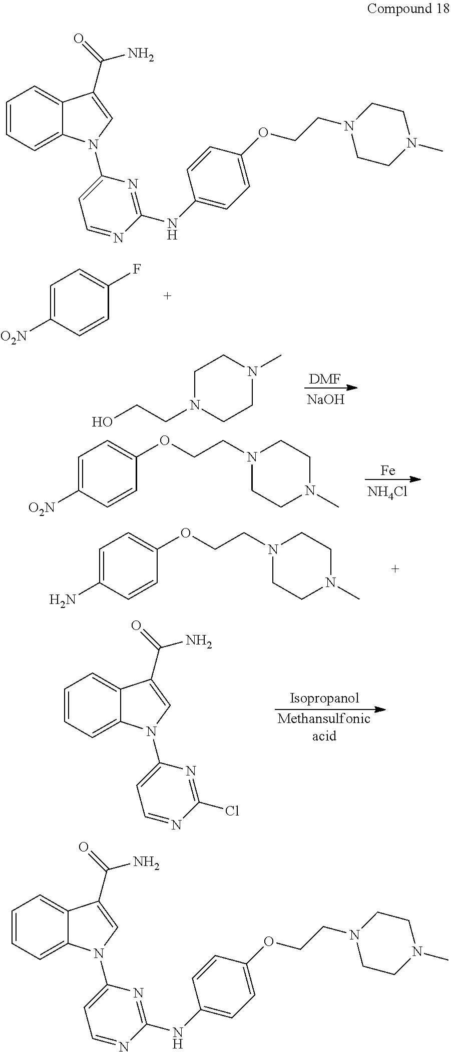

### Step 1: Preparation of 1-methyl-4-[2-(4-nitro-phenoxy)-ethyl]-piperazine

4-Fluoronitrobenzene (500 mg, 3.5 mmol) and 1-(2-hydroxyethyl)-4-methylpiperazine (760 mg, 5.3 mmol) were dissolved in DMF (10 ml). Sodium hydroxide (400 mg, 10.5 mmol) was added at room temperature, and the reaction solution was reacted at room temperature for 3 hours after completion of the addition. TLC showed that the reaction was completed. The reaction solution was slowly poured into 50 ml of water, and extracted with ethyl acetate (50 ml×2). The organic phase was washed with saturated NaCl solution twice, dried over anhydrous sodium sulfate, filtrated, and concentrated under reduced pressure to obtain the crude product 1-methyl-4-[2-(4-nitro-phenoxy)-ethyl]-piperazine (1.1 g, oil). The product was used directly in the next step without purification.

### Step 2: Preparation of 4-[2-(4-methyl-piperazin-1-yl)-ethoxy]-aniline

The product 1-methyl-4-[2-(4-nitro-phenoxy)-ethyl]-piperazine (1.1 g, 4 mmol) obtained in Step 1, reduced iron powder (900 mg, 16 mmol) and ammonium chloride (1.5 g, 28 mmol) were added into ethanol (80 ml)/water (20 ml). The resulting mixture was heated to 90° C. and reacted for 1 hour. The reaction solution was cooled to room temperature, slowly poured into saturated aqueous sodium bicarbonate solution (150 ml), and extracted with ethyl acetate (100 ml×2). The organic phase was washed with saturated NaCl solution twice, dried over anhydrous sodium sulfate, filtrated, and concentrated under reduced pressure to obtain the crude product 4-[2-(4-methyl-piperazin-1-yl)-ethoxy]-aniline (720 mg, oil). The product was used directly in the next step without purification.

### Step 3: Preparation of 1-(2-{4-[2-(4-methyl-piperazin-1-yl)-ethoxy]-phenylamino}-pyrimidin-4-yl)-1H-indole-3-carboxamide

1-(2-{4-[2-(4-Methyl-piperazin-1-yl)-ethoxy]-phenylamino}-pyrimidin-4-yl)-1H-indole-3-carboxamide was obtained in accordance with the same preparation method of Step 4 of Example 1 except for replacing the 3-bromo-4-fluoroaniline in Step 4 of Example 1 with 4-[2-(4-methyl-piperazin-1-yl)-ethoxy]-aniline (prepared in Step 2).

¹HNMR (DMSO-d6, 400 MHz) δ: 9.65 (1H, s), 8.78 (1, s), 8.68 (1H, s), 8.55 (1H, d), 8.25 (1K, dd), 7.69 (1H, s), 7.63 (2H, d), 7.30 (2H, m), 7.19 (1H, s) 7.06 (1H, d), 6.96 (2H, d), 4.07 (2H, t), 2.69 (2H, t), 3.36 (4H, t), 2.17 (3H, s).

LC-MS (ESI): 472.3 (M+H)⁺.

### Example 19

### Preparation of 1-{2-[4-(2-dimethylamino-ethoxy)-phenylamino]-pyrimidin-4-yl}-1H-indole-3-carboxamide (Compound 19)

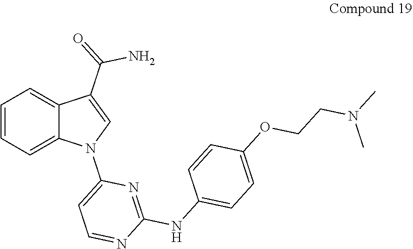 [SMILES: CN(C)CCOc1ccc(Nc2nccc(-n3cc(C(N)=O)c4ccccc43)n2)cc1]

1-{2-[4-(2-Dimethylamino-ethoxy)-phenylamino]-pyrimidin-4-yl}-1H-indole-3-carboxamide was obtained in accordance with the same preparation method of Example 18 except for replacing the 1-(2-hydroxyethyl)-4-methylpiperazine in Step 1 of Example 18 with N,N-dimethylethanolamine.

¹HNMR (DMSO-d6, 400 MHz) δ: 2.23 (6H, s), 2.63 (2H, t), 4.05 (2H, t), 6.94 (2H, d), 7.05 (1H, d), 7.16 (1H, br), 7.27-7.30 (2H, m), 7.60-7.69 (3H, m), 8.23-8.26 (1H, m), 8.53 (1H, d), 8.67 (1H, br), 8.76 (1H, s), 9.63 (1H, s).

LC-MS (ESI): 417.1 (M+H)⁺.

### Example 20

### Preparation of 1-{2-[4-(2-pyrrolidin-1-yl-ethoxy)-phenylamino]-pyrimidin-4-yl}-1H-indole-3-carboxamide (Compound 20)

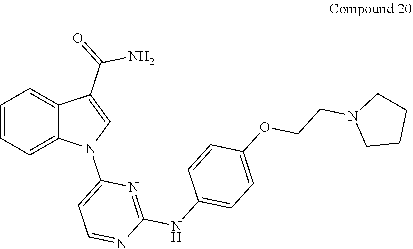 [SMILES: NC(=O)c1cn(-c2ccnc(Nc3ccc(OCCN4CCCC4)cc3)n2)c2ccccc12]

1-{2-[4-(2-Pyrrolidin-1-yl-ethoxy)-phenylamino]-pyrimidin-4-yl}-1H-indole-3-carboxamide was obtained in accordance with the same preparation method of Example 18 except for replacing the 1-(2-hydroxyethyl)-4-methylpiperazine in Step 1 of Example 18 with N-(2-hydroxyethyl)pyrrolidine.

¹HNMR (DMSO-d6, 400 MHz) δ: 9.65 (1H, s), 8.77 (1H, s), 8.68 (1H, s), 8.55 (1H, d), 8.25 (1H, dd), 7.69 (1H, s), 7.64 (2H, d), 7.29 (2H, m), 7.20 (1H, s), 7.06 (1H, d), 6.96 (2H, d), 4.06 (2H, t), 2.80 (2H, t), 2.53 (4H, t), 1.70 (4H, m).

LC-MS (ESI): 443.2 (M+H)⁺.

### Example 21

### Preparation of 1-{2-[4-(2-morpholin-4-yl-ethoxy)-phenylamino]-pyrimidin-4-yl}-1H-indole-3-carboxamide (Compound 21)

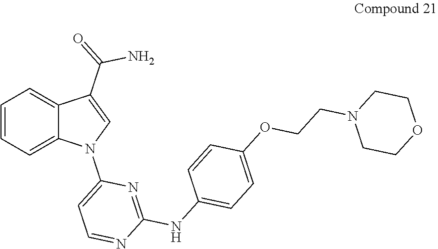 [SMILES: NC(=O)c1cn(-c2ccnc(Nc3ccc(OCCN4CCOCC4)cc3)n2)c2ccccc12]

1-{2-[4-(2-Morpholin-4-yl-ethoxy)-phenylamino]-pyrimidin-4-yl}-1H-indole-3-carboxamide was obtained in accordance with the same preparation method of Example 18 except for replacing the 1-(2-hydroxyethyl)-4-methylpiperazine in Step 1 of Example 18 with N-(2-hydroxyethyl)morpholine.

¹HNMR (DMSO-d6, 400 MHz) δ: 9.65 (1H, s), 8.77 (1H, s), 8.68 (1H, s), 8.55 (1H, d), 8.25 (1H, dd), 7.69 (1H, s), 7.64 (2H, d), 7.29 (2H, m), 7.19 (1H, s), 7.06 (11H, d), 6.97 (2H, d), 4.09 (2H, t), 3.60 (4H, t), 2.70 (2H, t).

LC-MS (ESI): 459.2 (M+H)⁺.

### Example 22

### Preparation of 1-(2-{4-[2-(4-piperazin-1-yl)-ethoxy]-phenylamino]-pyrimidin-4-yl}-1H-indole-3-carboxamide (Compound 22)

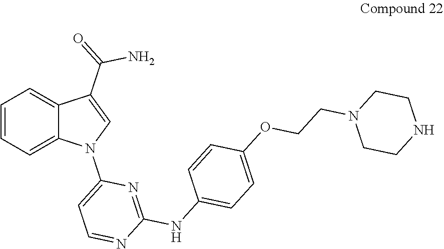 [SMILES: NC(=O)c1cn(-c2ccnc(Nc3ccc(OCCN4CCNCC4)cc3)n2)c2ccccc12]

1-(2-{4-[2-(4-Piperazin-1-yl)-ethoxy]-penylamino]-pyrimidin-4-yl}-1H-indole-3-carboxamide was obtained in accordance with the same preparation method of Example 18 except for replacing the 1-(2-hydroxyethyl)-4-methylpiperazine in Step 1 of Example 18 with N-(2-hydroxyethyl)piperazine.

¹HNMR (DMSO-d6, 400 MHz) δ: 9.64 (1H, s), 8.78 (1H, s), 8.68 (1H, s), 8.55 (1H, d), 8.25 (1H, dd), 7.68 (1H, s), 7.64 (2H, d), 7.30 (2H, m), 7.19 (1H, s), 7.06 (1H, d), 6.96 (2H, d), 4.07 (2H, t), 3.18 (1H, s), 2.72 (4H, t), 2.69 (2H, t), 2.42 (4H, t).

LC-MS (ESI): 458.2 (M+H)⁺.

### Example 23

### Preparation of 1-{2-[4-(2-dimethylamino-ethylthio)-phenylamino]-pyrimidin-4-yl}-1H-indole-3-carboxamide (Compound 23)

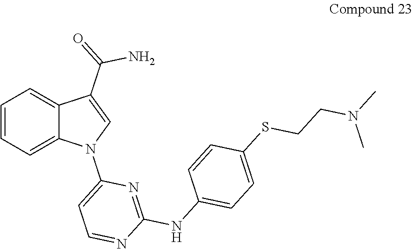 [SMILES: CN(C)CCSc1ccc(Nc2nccc(-n3cc(C(N)=O)c4ccccc43)n2)cc1]

1-{2-[4-(2-Dimethylamino-ethylthio)-phenylamino]-pyrimidin-4-yl}-1H-indole-3-carboxamide was obtained in accordance with the same preparation method of Example 18 except for replacing the 1-(2-hydroxyethyl)-4-methylpiperazine in Step 1 of Example 18 with 2-dimethylaminoethanethiol.

¹HNMR (DMSO-d6, 400 MHz) δ: 9.90 (1H, s), 8.79 (1H, s), 8.73 (1H, s), 8.61 (1H, d), 8.25 (1H, dd), 7.78 (2H, d), 7.70 (1H, s), 7.31-7.37 (4H, m), 7.20 (1H, s) 7.14 (1H, d), 3.02 (2H, t), 2.46 (2H, t), 2.18 (6H, s).

LC-MS (ESI): 433.2 (M+H)⁺.

### Example 24

### Preparation of 1-[2-(4-[1,4′]bipiperidin-1′-yl-phenylamino)-pyrimidin-4-yl]-1H-indole-3-carboxamide (Compound 24)

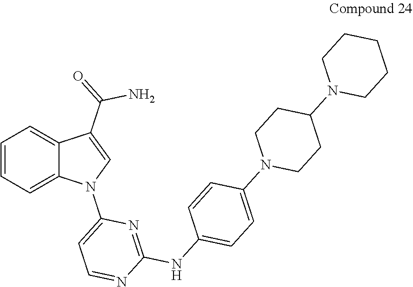 [SMILES: NC(=O)c1cn(-c2ccnc(Nc3ccc(N4CCC(N5CCCCC5)CC4)cc3)n2)c2ccccc12]

1-[2-(4-[1,4′]Bipiperidin-1′-yl-phenylamino)-pyrimidin-4-yl]-1H-indole-3-carboxamide was obtained in accordance with the same preparation method of Example 8 except for replacing the N,N-dimethylethylenediamine in Step 1 of Example 8 with 4-piperidinylpiperidine.

¹HNMR (DMSO-d6, 400 MHz) δ: 1.36-1.42 (2H, m), 1.45-1.62 (6H, m), 1.76-1.83 (2H, m), 2.27-2.35 (1H, m), 2.47 (4H, t), 2.56-2.63 (2H, m), 3.68 (2H, d), 6.94 (2H, d), 7.03 (1H, d), 7.19 (1H, br), 7.27-7.29 (2H, m), 7.54 (2H, d), 7.70 (1H, br), 8.23-8.26 (1H, m), 8.52 (1H, d), 8.70 (1H, br), 8.78 (1H, s), 9.57 (1H, s).

LC-MS (ESI): 496.2 (M+H)⁺.

### Example 25

### Preparation of 1-(2-{4-[4-(2-hydroxy-ethyl)-piperazin-1-yl]-phenylamino}-pyrimidin-4-yl)-1H-indole-3-carboxamide (Compound 25)

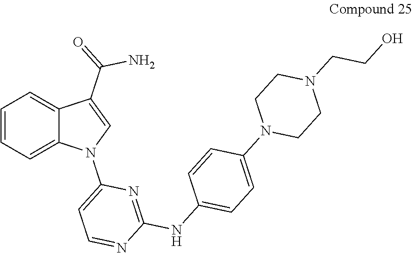 [SMILES: NC(=O)c1cn(-c2ccnc(Nc3ccc(N4CCN(CCO)CC4)cc3)n2)c2ccccc12]

1-(2-{4-[4-(2-Hydroxy-ethyl)-piperazin-1-yl]-phenylamino}-pyrimidin-4-yl)-1H-indole-3-carboxamide was obtained in accordance with the same preparation method of Example 8 except for replacing the N,N-dimethylethylenediamine in Step 1 of Example 8 with 1-(2-hydroxyethyl)piperazine.

¹HNMR (DMSO-d6, 400 MHz) δ: 9.57 (1H, s), 8.76 (1H, s), 8.67 (1H, s), 8.53 (1H, d), 8.24 (1H, t), 7.69 (1H, s), 7.55 (2H, d), 7.30 (2H, m), 7.18 (1H, s), 7.02 (1H, d), 6.94 (2H, d), 4.53 (1H, s), 3.56 (2H, m), 3.12 (4H, s), 2.62 (4H, s).

LC-MS (ESI): 458.2 (M+H)⁺.

### Example 26

### Preparation of 1-(2-{4-[4-(2-methoxy-ethyl)-piperazin-1-yl]-phenylamino}-pyrimidin-4-yl)-1H-indole-3-carboxamide (Compound 26)

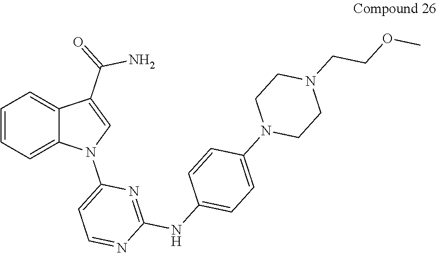 [SMILES: COCCN1CCN(c2ccc(Nc3nccc(-n4cc(C(N)=O)c5ccccc54)n3)cc2)CC1]

1-(2-{4-[4-(2-Methoxy-ethyl)-piperazin-1-yl]-phenylamino}-pyrimidin-4-yl)-1H-indole-3-carboxamide was obtained in accordance with the same preparation method of Example 8 except for replacing the N,N-dimethylethylenediamine in Step 1 of Example 8 with 1-(2-methoxyethyl)piperazine.

¹HNMR (DMSO-d6, 400 MHz) δ: 9.58 (1H, s), 8.77 (1H, s), 8.70 (1H, s), 8.53 (1H, d), 8.25 (1H, dd), 7.69 (1H, s), 7.58 (2H, d), 7.30 (2H, m), 7.19 (1H, s) 7.04 (11H, d), 6.95 (2H, d), 3.48 (2H, t), 3.26 (3H, s), 3.10 (4H, t), 2.58 (4H, t), 2.53 (2H, t).

LC-MS (ESI): 472.2 (M+H)⁺.

### Example 27

### Preparation of 1-[2-(4-piperazin-1-yl-phenylamino)-pyrimidin-4-yl]-1H-indole-3-carboxamide (Compound 27)

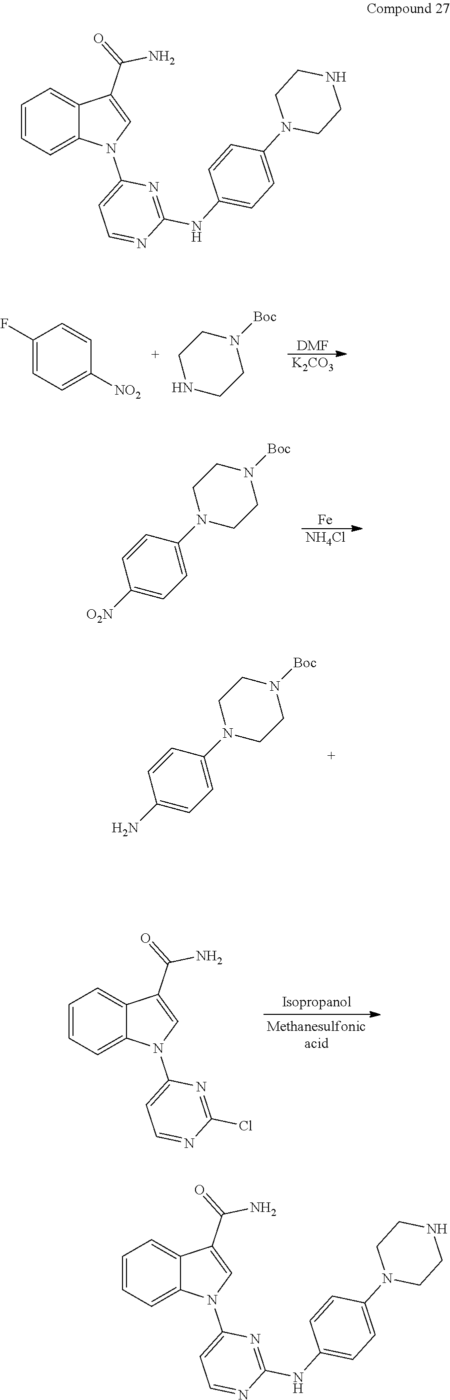

### Step 1: Preparation of tert-butyl 4-(4-nitro-phenyl)-piperazine-1-carboxylate

4-Fluoronitrobenzene (5 g, 35.5 mmol) and N-Boc-piperazine (7.9 g, 42.6 mmol) were dissolved in DMF (80 ml). Potassium carbonate (7.4 g, 53.3 mmol) was added at room temperature, and the resulting mixture was heated to 90° C. and reacted for 4 hours. TLC showed that the reaction was completed. The reaction solution was cooled to room temperature, and slowly poured into water (200 ml). The solution was stirred at room temperature for 30 minutes and filtrated. The solid was washed with water, and dried by blowing (60° C.) for 8 hours to obtain tert-butyl 4-(4-nitro-phenyl)-piperazine-1-carboxylate (9.5 g, yellow solid). The product was used directly in the next step without purification.

### Step 2: Preparation of tert-butyl 4-(4-amino-phenyl)-piperazine-1-carboxylate

The product tert-butyl 4-(4-nitro-phenyl)-piperazine-1-carboxylate (9.5 g, 31 mmol) obtained in Step 1, reduced iron powder (6.9 g, 124 mmol) and ammonium chloride (11.6 g, 217 mmol) were added into ethanol (100 ml)/water (25 ml). The resulting mixture was heated to 90° C. and reacted for 2 hours. The reaction solution was cooled to room temperature, slowly poured into saturated aqueous sodium bicarbonate solution (200 ml), and extracted with ethyl acetate (100 ml×2). The organic phase was washed with saturated NaCl solution twice, dried over anhydrous sodium sulfate, filtrated, and concentrated under reduced pressure to obtain tert-butyl 4-(4-amino-phenyl)-piperazine-1-carboxylate (5.1 g, yellow solid). The product was used directly in the next step without purification.

### Step 3: Preparation of 1-[2-(4-piperazin-1-yl-phenylamino)-pyrimidin-4-yl]-1H-indole-3-carboxamide

1-[2-(4-Piperazin-1-yl-phenylamino)-pyrimidin-4-yl]-1H-indole-3-carboxamide was obtained in accordance with the same preparation method of Step 4 of Example 1 except for replacing the 3-bromo-4-fluoroaniline in Step 4 of Example 1 with tert-butyl 4-(4-amino-phenyl)-piperazine-1-carboxylate (prepared in Step 2).

¹HNMR (DMSO-d6, 400 MHz) δ: 9.58 (1H, s), 8.78 (1H, s), 8.69 (1H, br), 8.52 (1H, d), 8.25 (1H, m), 7.70 (1H, s), 7.56 (2H, d), 7.30 (2H, m), 7.19 (1H, br), 7.04 (1H, d), 6.93-6.94 (2H, d), 3.02 (4H, m), 2.86 (4H, m).

LC-MS (ESI): 414.2 (M+H)⁺.

### Example 28

### Preparation of 1-[2-(3-methyl-4-piperazin-1-yl-phenylamino)-pyrimidin-4-yl]-1H-indole-3-carboxamide (Compound 28)

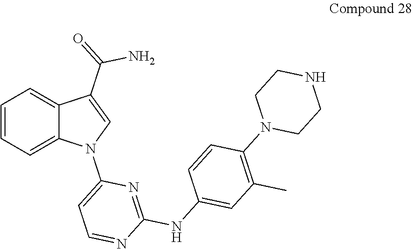 [SMILES: Cc1cc(Nc2nccc(-n3cc(C(N)=O)c4ccccc43)n2)ccc1N1CCNCC1]

1-[2-(3-Methyl-4-piperazin-1-yl-phenylamino)-pyrimidin-4-yl]-1H-indole-3-carboxamide was obtained in accordance with the same preparation method of Example 27 except for replacing the 4-fluoronitrobenzene in Step 1 of Example 27 with 2-fluoro-5-nitrotoluene (Darui).

¹HNMR (DMSO-d6, 400 MHz) δ: 9.63 (s, 1H), 8.78 (s, 1H), 8.70 (br, 1H), 8.54-8.55 (d, 1H), 8.23-8.26 (m, 1H), 7.68 (br, 1H), 7.57-7.58 (d, 1H), 7.46-7.48 (m, 1H), 7.27-7.31 (m, 2H), 7.17 (br, 1H), 7.05-7.06 (d, 1H), 6.99-7.01 (d, 1H), 2.89-2.92 (m, 4H), 2.76-2.80 (m, 4H), 2.26 (s, 3H).

LC-MS (ESI): 428.2 (M+H)⁺.

### Example 29

### Preparation of 4-fluoro-1-[2-(4-piperazin-1-yl-phenylamino)-pyrimidin-4-yl]-1H-indole-3-carboxamide (Compound 29)

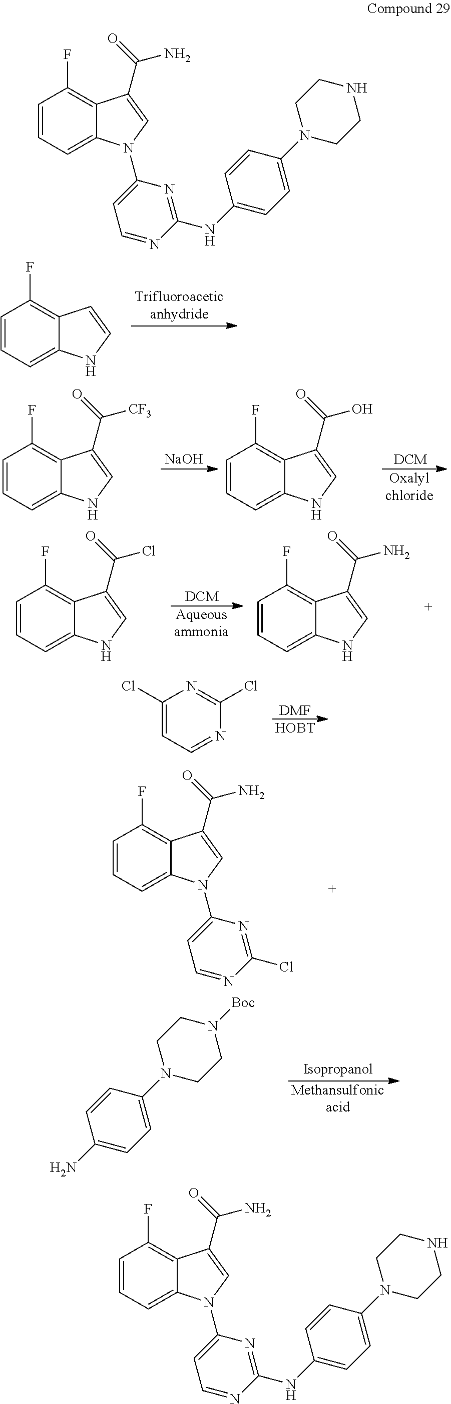

### Step 1: Preparation of 2,2,2-trifluoro-1-(4-fluoro-1H-indol-3-yl)-ethanone

4-Fluoroindole (2.7 g, 0.02 mol) was dissolved in 30 ml of DMF, and the resulting solution was cooled in an ice water bath to 0-5° C. Trifluoroacetic anhydride (6.3 g, 0.03 mol) was slowly add dropwise at this temperature, and the reaction solution was warmed to room temperature and reacted for 2 hours after completion of the addition. TLC showed that the reaction was completed. The reaction solution was poured into water (150 ml), stirred at room temperature for 20 minutes and filtrated. The solid was washed with water, and dried by blowing (60° C.) for 4 hours to obtain 2,2,2-trifluoro-1-(4-fluoro-1H-indol-3-yl)-ethanone (4 g, solid).

### Step 2: Preparation of 4-fluoro-1H-indole-3-carboxylic acid

2,2,2-Trifluoro-O-(4-fluoro-H-indol-3-yl)-ethanone (4 g, 0.017 mmol) obtained in Step 1 and sodium hydroxide (6.9 g, 0.17 mmol) were dissolved in 80 ml of water. The reaction solution was heated to 100° C. and reacted for 3 hours. The reaction solution was cooled to room temperature. 50 ml of water was added, and the solution was extracted with ethyl acetate twice. The pH of the aqueous phase was adjusted to 5-6 with 1 mol/L dilute hydrochloric acid, and solid was precipitated during the process. The solution was filtrated, the solid was washed with water, and dried by blowing (60° C.) for 8 hours to obtain 4-fluoro-H-indole-3-carboxylic acid (840 mg, solid).

### Step 3: Preparation of 4-fluoro-1H-indole-3-carbonyl chloride

4-Fluoro-1H-indole-3-carbonyl chloride was obtained in accordance with the same preparation method of Step 1 of Example 1 except for replacing the 3-indole carboxylic acid in Step 1 of Example 1 with 4-fluoro-H-indole-3-carboxylic acid (prepared in Step 2).

### Step 4: Preparation of 4-fluoro-H-indole-3-carboxamide

4-Fluoro-1H-indole-3-carboxamide was obtained in accordance with the same preparation method of Step 2 of Example 1 except for replacing the 1H-indole-3-carbonyl chloride in Step 2 of Example 1 with 4-fluoro-1H-indole-3-carbonyl chloride (prepared in Step 3).

### Step 5: Preparation of 1-(2-chloro-pyrimidin-4-yl)-4-fluoro-1H-indole-3-carboxamide

1-(2-Chloro-pyrimidin-4-yl)-4-fluoro-1H-indole-3-carboxamide was obtained in accordance with the same preparation method of Step 3 of Example 1 except for replacing the 1H-indole-3-carboxamide in Step 3 of Example 1 with 4-fluoro-1H-indole-3-carboxamide (prepared in Step 4).

### Step 6: Preparation of 4-fluoro-1-[2-(4-piperazin-1-yl-phenylamino)-pyrimidin-4-yl]-1H-indole-3-carboxamide

1-(2-Chloro-pyrimidin-4-yl)-4-fluoro-1H-indole-3-carboxamide (120 mg, 0.4 mmol) obtained in Step 5, tert-butyl 4-(4-amino-phenyl)-piperazine-1-carboxylate (120 mg, 0.4 mmol) (prepared in Step 2 of Example 27) and methanesulfonic acid (60 mg, 0.6 mmol) was dispersed in 10 ml of isopropanol. The reaction solution was reacted at 80° C. for 4 hours. TLC showed that the reaction was completed. The reaction solution was cooled to room temperature, and solid was precipitated. The solution was left to stand at room temperature for 1 hour, and the supernatant was removed. The resulting solid was dissolved in 50 ml of dichloromethane/methanol (dichloromethane:methanol=5:1). 10 ml of aqueous sodium hydroxide solution (0.5 mol/L) was added, and the solution was extracted with dichloromethane. The organic phase was washed with saturated NaCl solution twice, dried over anhydrous sodium sulfate, filtrated and concentrated under reduced pressure. The resulting residues were purified by column chromatography (eluent: dichloromethane/methanol) to obtain 4-fluoro-1-[2-(4-piperazin-1-yl-phenylamino)-pyrimidin-4-yl]-1H-indole-3-carboxamide (26 mg, solid).

¹HNMR (DMSO-d6, 400 MHz) δ: 9.58 (1H, s), 8.51 (3H, m), 7.55 (3H, m), 7.28 (2H, br), 7.17 (1H, d), 7.09 (1H, t), 6.94 (1H, s), 6.91 (1H, s), 3.01 (4H, t), 2.86 (4H, t).

LC-MS (ESI): 432.1 (M+H)⁺.

### Example 30

### Preparation of 6-fluoro-1-[2-(4-piperazin-1-yl-phenylamino)-pyrimidin-4-yl]-1H-indole-3-carboxamide (Compound 30)

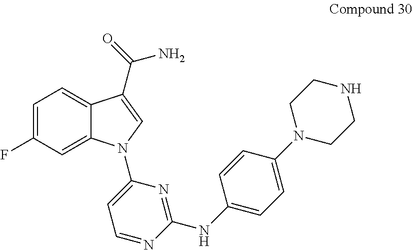 [SMILES: NC(=O)c1cn(-c2ccnc(Nc3ccc(N4CCNCC4)cc3)n2)c2cc(F)ccc12]

6-Fluoro-1-[2-(4-piperazin-1-yl-phenylamino)-pyrimidin-4-yl]-1H-indole-3-carboxamide was obtained in accordance with the same preparation method of Example 29 except for replacing the 4-fluoroindole in Step 1 of Example 29 with 6-fluoroindole.

¹HNMR (DMSO-d6, 400 MHz) δ: 9.64 (1H, s), 8.80 (1H, s), 8.54 (0H, d), 8.22 (1H, m), 7.70 (1H, s), 7.54 (2H, br), 7.24 (1H, s), 7.17 (1H, m), 7.01 (1H, d), 6.96 (1H, s), 6.94 (1H, s), 3.04 (4H, t), 2.88 (4H, t).

LC-MS (ESI): 432.1 (M+H)⁺.

### Example 31

### Preparation of 7-fluoro-1-[2-(4-piperazin-1-yl-phenylamino)-pyrimidin-4-yl]-1H-indole-3-carboxamide (Compound 31)

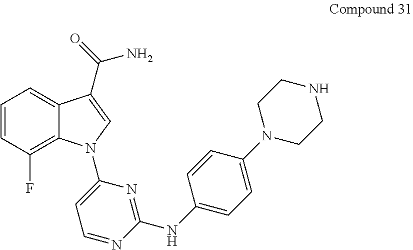 [SMILES: NC(=O)c1cn(-c2ccnc(Nc3ccc(N4CCNCC4)cc3)n2)c2c(F)cccc12]

7-Fluoro-1-[2-(4-piperazin-1-yl-phenylamino)-pyrimidin-4-yl]-1H-indole-3-carboxamide was obtained in accordance with the same preparation method of Example 29 except for replacing the 4-fluoroindole in Step 1 of Example 29 with 7-fluoroindole.

¹HNMR (DMSO-d6, 400 MHz) δ: 9.68 (1H, s), 8.58 (2H, m), 8.16 (1H, d), 7.83 (1H, s), 7.62 (2H, d), 7.30 (1H, m), 7.19 (2H, m), 6.96 (1H, t), 6.89 (1H, br), 6.87 (1H, br), 2.99 (4H, br), 2.85 (4H, br).

LC-MS (ESI): 432.1 (M+H)⁺.

### Example 32

### Preparation of 5-bromo-1-[2-(4-piperazin-1-yl-phenylamino)-pyrimidin-4-yl]-1H-indole-3-carboxamide (Compound 32)

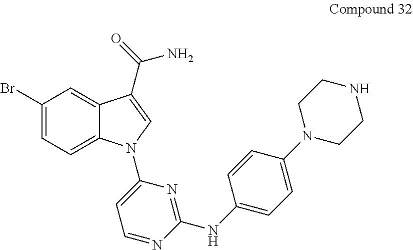 [SMILES: NC(=O)c1cn(-c2ccnc(Nc3ccc(N4CCNCC4)cc3)n2)c2ccc(Br)cc12]

5-Bromo-1-[2-(4-piperazin-1-yl-phenylamino)-pyrimidin-4-yl]-1H-indole-3-carboxamide was obtained in accordance with the same preparation method of Steps 3 to 6 of Example 29 except for replacing the 4-fluoro-1H-indole-3-carboxylic acid in Step 3 of Example 29 with 5-bromo-1H-indole-3-carboxylic acid.

¹HNMR (DMSO-d6, 400 MHz) δ: 3.00 (4H, t), 3.13 (4H, t), 6.97 (2H, d), 7.02 (1H, d), 7.29 (1H, br), 7.40 (1H, d), 7.57 (2H, d), 7.76 (1H, br), 8.41 (1H, d), 8.55 (1H, d), 8.65 (1H, br), 8.86 (1H, s), 9.64 (1H, s).

LC-MS (ESI): 492.0 (M+H)⁺.

### Example 33

### Preparation of 5-methoxy-1-[2-(4-piperazin-1-yl-phenylamino)-pyrimidin-4-yl]-1H-indole-3-carboxamide (Compound 33)

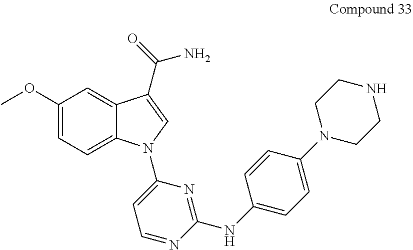 [SMILES: COc1ccc2c(c1)c(C(N)=O)cn2-c1ccnc(Nc2ccc(N3CCNCC3)cc2)n1]

5-Methoxy-1-[2-(4-piperazin-1-yl-phenylamino)-pyrimidin-4-yl]-1H-indole-3-carboxamide was obtained in accordance with the same preparation method of Steps 3 to 6 of Example 29 except for replacing the 4-fluoro-1H-indole-3-carboxylic acid in Step 3 of Example 29 with 5-methoxy-H-indole-3-carboxylic acid.

¹HNMR (DMSO-d6, 400 MHz) δ: 3.23 (4H, br), 3.35 (4H, br), 3.82 (3H, s), 6.87 (1H, d), 7.01 (2H, d), 7.06 (1H, d), 7.17 (1H, br), 7.62 (2H, d), 7.70-7.78 (2H, m), 8.51 (1H, d), 8.62 (1H, br), 8.85 (1H, s), 9.63 (1H, s).

LC-MS (ESI): 444.2 (M+H)⁺.

### Example 34

### Preparation of 1-[5-chloro-2-(4-piperazin-1-yl-phenylamino)-pyrimidin-4-yl]-1H-indole-3-carboxamide (Compound 34

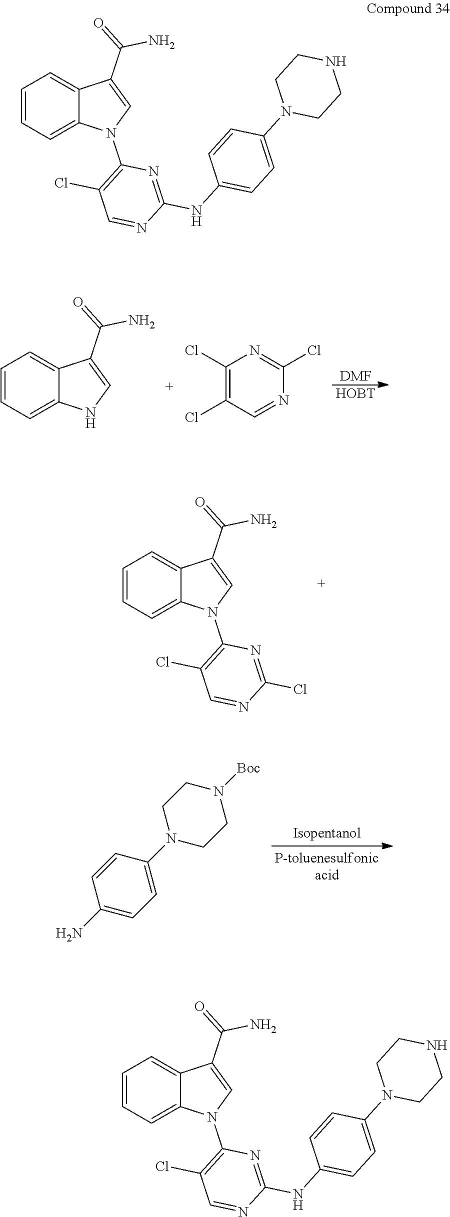

### Step 1: Preparation of 1-(2,5-dichloro-pyrimidin-4-yl)-1H-indole-3-carboxamide

2,4,5-Trichloropyrimidine (1.5 g, 8.43 mmol) and HOBT (152 mg, 1.12 mmol) were dissolved in 15 ml of DMF. Potassium carbonate (2.3 g, 16.8 mmol) was added at room temperature, and the resulting solution was stirred at room temperature for 10 minutes. 1H-Indole-3-carboxamide (900 mg, 5.62 mmol) (prepared in Step 2 of Example 1) was added, the reaction solution was warmed to 80° C. and stirred for 4 hours. TLC showed that the reaction was completed. The reaction solution was cooled to room temperature, and slowly poured into water (60 ml). The solution was stirred at room temperature for 30 minutes and filtrated. The solid was washed with water, and dried by blowing (60° C.) for 8 hours to obtain 1-(2,5-dichloro-pyrimidin-4-yl)-1H-indole-3-carboxamide (1.4 g, yellow solid), which was used directly in the next step without purification.

### Step 2: Preparation of 1-[5-chloro-2-(4-piperazin-1-yl-phenylamino)-pyrimidin-4-yl]-1H-indole-3-carboxamide

1-(2,5-Dichloro-pyrimidin-4-yl)-1H-indole-3-carboxamide (100 mg, 0.36 mmol) obtained in Step 1, tert-butyl 4-(4-amino-phenyl)-piperazine-1-carboxylate (110 mg, 0.36 mmol) (prepared in Step 2 of Example 27) and p-toluenesulfonic acid (68 mg, 0.39 mmol) were dissolved in 10 ml of isopentanol, and reacted at 120° C. overnight. TLC showed that the reaction was substantially completed. The reaction solution was cooled to room temperature, and 5 ml of aqueous sodium hydroxide solution (1 mol/L) and 15 ml of petroleum ether were added successively. The solution was stirred at room temperature for 30 minutes, and solid was precipitated which was then filtrated. The resulting solid was dissolved in a small amount of dichloromethane/methanol (dichloromethane:methanol=2:1), and purified by preparative TLC (developing solvent: dichloromethane/methanol) to obtain 1-[5-chloro-2-(4-piperazin-1-yl-phenylamino)-pyrimidin-4-yl]-1H-indole-3-carboxamide (18 mg, solid).

¹HNMR (DMSO-d6, 400 MHz) δ: 9.98 (s, 1H), 9.22 (br, 1H), 8.75 (s, 1H), 8.51 (s, 1H), 8.26-8.28 (m, 1H), 7.68-7.74 (m, 2H), 7.57-7.59 (d, 2H), 7.28 (m, 2H), 6.92-6.94 (d, 2H), 3.29 (m, 4H), 3.18 (m, 4H).

LC-MS (ESI): 448.1 (M+H)⁺.

### Example 35

### Preparation of 1-[5-fluoro-2-(4-piperazin-1-yl-phenylamino)-pyrimidin-4-yl]-1H-indole-3-carboxamide (Compound 35)

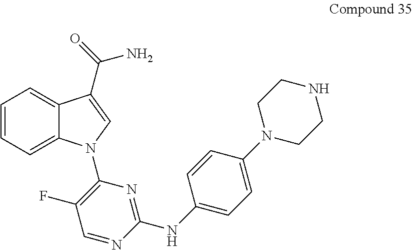 [SMILES: NC(=O)c1cn(-c2nc(Nc3ccc(N4CCNCC4)cc3)ncc2F)c2ccccc12]

1-[5-Fluoro-2-(4-piperazin-1-yl-phenylamino)-pyrimidin-4-yl]-1H-indole-3-carboxamide was obtained in accordance with the same preparation method of Example 34 except for replacing the 2,4,5-trichloropyrimidine in Step 1 of Example 34 with 2,4-dichloro-5-fluoropyrimidine.

¹HNMR (DMSO-d6, 400 MHz) δ: 9.71 (s, 1H), 8.71-8.72 (d, 1H), 8.55 (s, 1H), 8.28-8.30 (m, 1H), 8.23 (br, 1H), 7.87 (br, 1H), 7.56-7.58 (d, 2H), 7.30-7.32 (m, 2H), 7.17 (br, 1H), 6.92-6.94 (d, 2H), 3.14 (m, 4H), 3.03 (m, 4H).

LC-MS (ESI): 432.2 (M+H)⁺.

### Example 36

### Preparation of 1-[5-methoxy-2-(4-piperazin-1-yl-phenylamino)-pyrimidin-4-yl]-1H-indole-3-carboxamide (Compound 36)

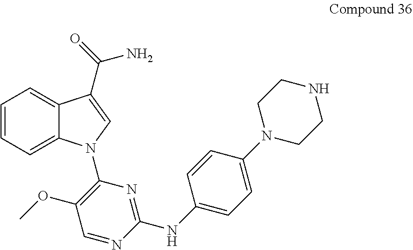 [SMILES: COc1cnc(Nc2ccc(N3CCNCC3)cc2)nc1-n1cc(C(N)=O)c2ccccc21]

1-[5-Methoxy-2-(4-piperazin-1-yl-phenylamino)-pyrimidin-4-yl]-1H-indole-3-carboxamide was obtained in accordance with the same preparation method of Example 34 except for replacing the 2,4,5-trichloropyrimidine in Step 1 of Example 34 with 2,4-dichloro-5-methoxypyrimidine.

¹HNMR (DMSO-d6, 400 MHz) δ: 9.40 (s, 1H), 8.58 (s, 1H), 8.56 (s, 1H), 8.25-8.27 (m, 1H), 8.02-8.04 (m, 1H), 7.77 (br, 1H), 7.57-7.59 (d, 2H), 7.24-7.29 (m, 2H), 7.10 (br, 1H), 6.86-6.88 (d, 2H), 3.02-3.04 (m, 4H), 2.90-2.93 (m, 4H).

LC-MS (ESI): 444.2 (M+H)⁺.

### Example 37

### Preparation of 1-[5-methyl-2-(4-piperazin-1-yl-phenylamino)-pyrimidin-4-yl]-1H-indole-3-carboxamide (Compound 37)

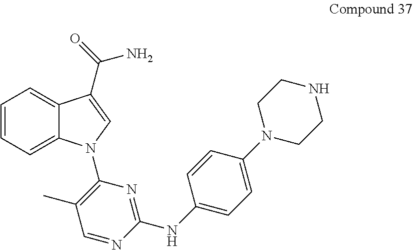 [SMILES: Cc1cnc(Nc2ccc(N3CCNCC3)cc2)nc1-n1cc(C(N)=O)c2ccccc21]

1-[5-Methyl-2-(4-piperazin-1-yl-phenylamino)-pyrimidin-4-yl]-1H-indole-3-carboxamide was obtained in accordance with the same preparation method of Example 34 except for replacing the 2,4,5-trichloropyrimidine in Step 1 of Example 34 with 2,4-dichloro-5-methylpyrimidine.

¹HNMR (DMSO-d6, 400 MHz) δ: 9.57 (s, 1H), 8.55 (d, 1H), 8.40 (s, 1H), 8.28-8.30 (m, 1H), 7.65-7.70 (m, 2H), 7.56-7.58 (d, 2H), 7.23-7.30 (m, 2H), 7.09 (br, 1H), 6.83-685 (d, 2H), 2.98-3.00 (m, 4H), 2.87-2.90 (m, 4H), 2.16 (s, 3H).

LC-MS (ESI): 428.2 (M+H)⁺.

### Example 38

### Preparation of 1-[6-methyl-2-(4-piperazin-1-yl-phenylamino)-pyrimidin-4-yl]-1H-indole-3-carboxamide (Compound 38)

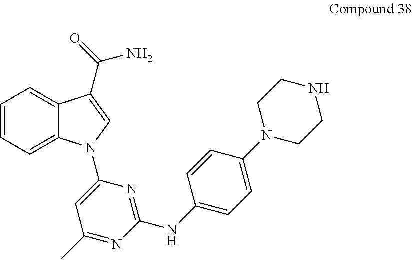 [SMILES: Cc1cc(-n2cc(C(N)=O)c3ccccc32)nc(Nc2ccc(N3CCNCC3)cc2)n1]

1-[6-Methyl-2-(4-piperazin-1-yl-phenylamino)-pyrimidin-4-yl]-1H-indole-3-carboxamide was obtained in accordance with the same preparation method of Example 34 except for replacing the 2,4,5-trichloropyrimidine in Step 1 of Example 34 with 2,4-dichloro-6-methylpyrimidine.

¹HNMR (DMSO-d6, 400 MHz) δ: 9.54 (s, 1H), 8.62 (br, 1H), 8.23-8.25 (m, 1H), 7.69 (br, 1H), 7.59-7.61 (d, 2H), 7.26-7.30 (m, 2H), 7.15 (br, 1H), 6.94-6.97 (m, 2H), 3.11 (m, 4H), 2.98 (m, 4H), 2.43 (s, 3H).

LC-MS (ESI): 428.2 (M+H)⁺.

### Example 39

### Preparation of 1-[5-fluoro-2-(3-methyl-4-piperazin-1-yl-phenylamino)-pyrimidin-4-yl]-1H-indole-3-carboxamide (Compound 39)

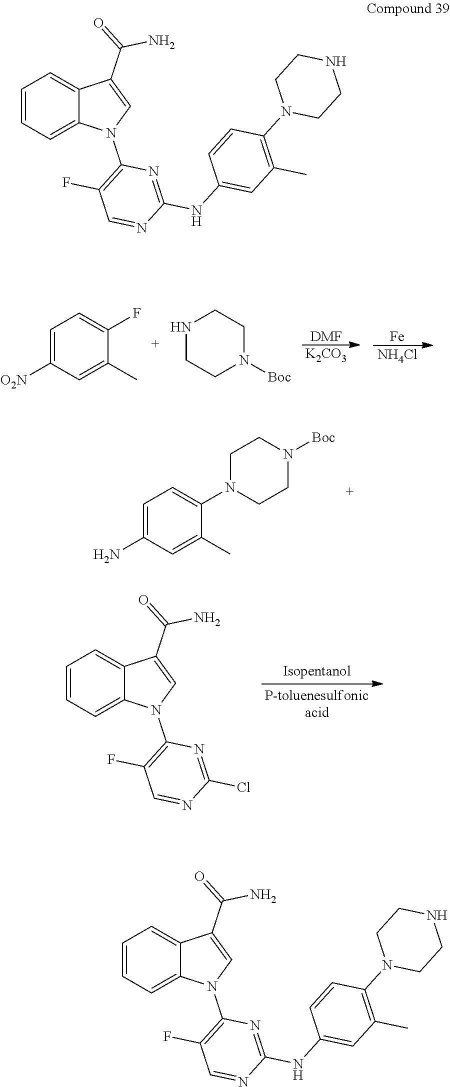

### Step 1: Preparation of Tert-Butyl

4-(4-amino-2-methyl-phenyl)-piperazine-1-carboxylate 2-Fluoro-5-nitrotoluene (1.55 g, 0.01 mmol) and N-Boc-piperazine (2.23 g, 0.012 mmol) were dissolved in DMF (20 ml). Potassium carbonate (2.0 g, 0.015 mmol) was added at room temperature, and the resulting mixture was heated to 90° C. and reacted for 2 hours. TLC showed that the reaction was completed. The reaction solution was cooled to room temperature, slowly poured into water (10 ml), and extracted with ethyl acetate (50 ml×2). The organic phase was washed with saturated NaCl solution twice, dried over anhydrous sodium sulfate, filtrated, and concentrated under reduced pressure. Reduced iron powder (1.84 g, 0.04 mol), ammonium chloride (3.75 g, 0.07 mol), 60 ml of ethanol and 20 ml of water were added to the resulting product. The resulting mixture was heated to 90° C. and reacted for 1 hour. TLC showed that the reaction was completed. The reaction solution was cooled to room temperature, slowly poured into saturated aqueous sodium bicarbonate solution (100 ml), and extracted with ethyl acetate (50 ml×2). The organic phase was washed with saturated NaCl solution twice, dried over anhydrous sodium sulfate, filtrated, and concentrated under reduced pressure to obtain tert-butyl 4-(4-amino-2-methyl-phenyl)-piperazine-1-carboxylate (1.6 g). The product was used directly in the next step without purification.

### Step 2: Preparation of 1-[5-fluoro-2-(3-methyl-4-piperazin-1-yl-phenylamino)-pyrimidin-4-yl]-1H-indole-3-carboxamide

Tert-butyl 4-(4-amino-2-methyl-phenyl)-piperazine-1-carboxylate (100 mg, 0.345 mmol) obtained in Step 1, 1-(2-chloro-5-fluoro-pyrimidin-4-yl)-1H-indole-3-carboxamide (100 mg, 0.345 mmol) (prepared in Example 35) and p-toluenesulfonic acid (71 mg, 0.414 mmol) were dissolved in 10 ml of isopentanol, and reacted at 120° C. overnight. TLC showed that the reaction was substantially completed. The reaction solution was cooled to room temperature, and 15 ml of methyl tert-butyl ether was added. The solution was stirred at room temperature for 30 minutes, and the solid was precipitated which was then filtrated. The resulting solid was dissolved in 50 ml of dichloromethane/methanol (dichloromethane:methanol=2:1), and 10 ml of aqueous sodium hydroxide solution (0.5 mol/L) was added. The solution was stirred at room temperature for 20 minutes, and extracted with dichloromethane (50 ml×2). The organic phase was washed with saturated NaCl solution twice, dried over anhydrous sodium sulfate, filtrated, and concentrated under reduced pressure. The resulting residues were purified by preparative TLC (developing solvent: dichloromethane/methanol) to obtain 1-[5-fluoro-2-(3-methyl-4-piperazin-1-yl-phenylamino)-pyrimidin-4-yl]-1H-indole-3-carboxamide (9 mg, solid).

¹HNMR (DMSO-d6, 400 MHz) δ: 9.79 (s, 1H), 8.74-8.75 (d, 1H), 8.56 (d, 1H), 8.25-8.30 (m, 2H), 7.86 (br, 1H), 7.60-7.61 (d, 1H), 7.47-7.50 (m, 1H), 7.30-7.34 (m, 2H), 7.15 (br, 1H), 6.99-7.01 (d, 1H), 3.19 (m, 4H), 3.00 (m, 4H), 2.23 (s, 3H).

LC-MS (ESI): 446.3 (M+H)⁺.

### Example 40

### Preparation of 1-[5-fluoro-2-(3-methoxy-4-piperazin-1-yl-phenylamino)-pyrimidin-4-yl]-1H-indole-3-carboxamide (Compound 40)

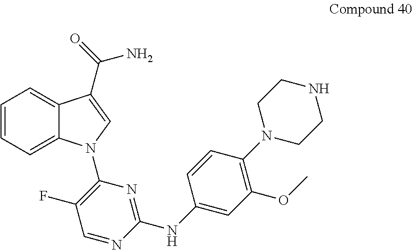 [SMILES: COc1cc(Nc2ncc(F)c(-n3cc(C(N)=O)c4ccccc43)n2)ccc1N1CCNCC1]

1-[5-Fluoro-2-(3-methoxy-4-piperazin-1-yl-phenylamino)-pyrimidin-4-yl]-1H-indole-3-carboxamide was obtained in accordance with the same preparation method of Example 39 except for replacing the 1-fluoro-5-nitrotoluene in Step 1 of Example 39 with 1-fluoro-5-nitroanisole.

¹HNMR (DMSO-d6, 400 MHz) δ: 9.81 (s, 1H), 8.75-8.76 (d, 1H), 8.57 (d, 1H), 8.28-8.31 (m, 1H), 8.23 (br, 1H), 7.87 (br, 1H), 7.43-7.44 (d, 1H), 7.30-7.32 (m, 2H), 7.25-7.27 (m, 1H), 7.17 (br, 1H), 6.85-6.87 (d, 1H), 3.70 (s, 3H), 3.05 (m, 4H), 3.01 (m, 4H).

LC-MS (ESI): 462.2 (M+H)⁺.

### Example 41

### Preparation of 1-[5-fluoro-2-(3-fluoro-4-piperazin-1-yl-phenylamino)-pyrimidin-4-yl]-1H-indole-3-carboxamide (Compound 41)

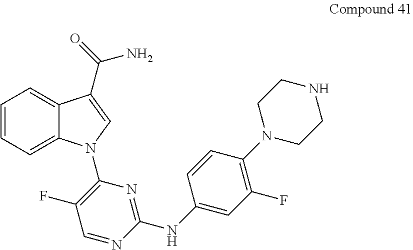 [SMILES: NC(=O)c1cn(-c2nc(Nc3ccc(N4CCNCC4)c(F)c3)ncc2F)c2ccccc12]

1-[5-Fluoro-2-(3-fluoro-4-piperazin-1-yl-phenylamino)-pyrimidin-4-yl]-1H-indole-3-carboxamide was obtained in accordance with the same preparation method of Example 39 except for replacing the 1-fluoro-5-nitrotoluene in Step 1 of Example 39 with 3,4-difluoronitrobenzene.

¹HNMR (DMSO-d6, 400 MHz) δ: 10.00 (s, 1H), 8.78-8.79 (d, 1H), 8.55-8.56 (d, 1H), 8.25-8.31 (m, 2H), 7.86 (br, 1H), 7.71-7.76 (dd, 1H), 7.39-7.72 (m, 1H), 7.30-7.34 (m, 2H), 7.16 (br, 1H), 7.03-7.07 (t, 1H), 3.13 (m, 8H).

LC-MS (ESI): 450.2 (M+H)⁺.

### Example 42

### Preparation of 1-[5-chloro-2-(3-methyl-4-piperazin-1-yl-phenylamino)-pyrimidin-4-yl]-1H-indole-3-carboxamide (Compound 42)

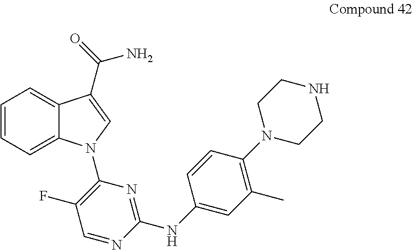 [SMILES: Cc1cc(Nc2ncc(F)c(-n3cc(C(N)=O)c4ccccc43)n2)ccc1N1CCNCC1]

1-[5-Chloro-2-(3-methyl-4-piperazin-1-yl-phenylamino)-pyrimidin-4-yl]-1H-indole-3-carboxamide was obtained in accordance with the same preparation method of Example 39 except for replacing the 1-(2-chloro-5-fluoro-pyrimidin-4-yl)-1H-indole-3-carboxamide in Step 2 of Example 39 with 1-(2,5-dichloro-pyrimidin-4-yl)-1H-indole-3-carboxamide (prepared in Step 1 of Example 34).

¹HNMR (DMSO-d6, 400 MHz) δ: 10.00 (s, 1H), 8.78 (s, 1H), 8.54 (s, 1H), 8.27-8.29 (m, 1H), 7.77-7.80 (m, 2H), 7.60 (s, 1H), 7.46 (m, 1H), 7.29-7.33 (m, 2H), 7.13 (br, 1H), 6.95-6.97 (d, 1H), 3.20 (m, 4H), 2.98 (m, 4H), 2.19 (s, 3H).

LC-MS (ESI): 462.1 (M+H)⁺.

### Example 43

### Preparation of 1-[5-chloro-2-(3-methoxy-4-piperazin-1-yl-phenylamino)-pyrimidin-4-yl]-1H-indole-3-carboxamide (Compound 43

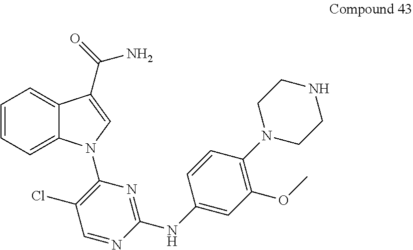 [SMILES: COc1cc(Nc2ncc(Cl)c(-n3cc(C(N)=O)c4ccccc43)n2)ccc1N1CCNCC1]

1-[5-Chloro-2-(3-methoxy-4-piperazin-1-yl-phenylamino)-pyrimidin-4-yl]-1H-indole-3-carboxamide was obtained in accordance with the same preparation method of Example 40 except for replacing the 1-(2-chloro-5-fluoro-pyrimidin-4-yl)-1H-indole-3-carboxamide in Example 40 with 1-(2,5-dichloro-pyrimidin-4-yl)-1H-indole-3-carboxamide (prepared in Step 1 of Example 34).

¹HNMR (DMSO-d6, 400 MHz) δ: 10.09 (s, 1H), 8.80 (s, 1H), 8.55 (s, 1H), 8.26-8.29 (m, 1H), 7.74-7.76 (m, 2H), 7.51 (br, 1H), 7.26-7.31 (m, 2H), 7.21-7.22 (m, 1H), 7.13 (br, 1H), 6.85-6.87 (d, 1H), 3.62 (s, 3H), 3.18 (m, 4H), 3.12 (m, 4H).

LC-MS (ESI): 478.1 (M+H)⁺.

### Example 44

### Preparation of 1-[5-chloro-2-(3-fluoro-4-piperazin-1-yl-phenylamino)-pyrimidin-4-yl]-1H-indole-3-carboxamide (Compound 44)

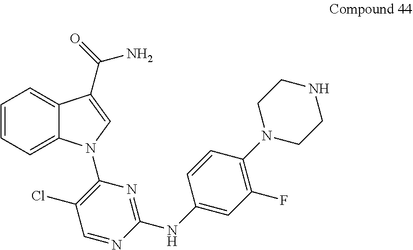 [SMILES: NC(=O)c1cn(-c2nc(Nc3ccc(N4CCNCC4)c(F)c3)ncc2Cl)c2ccccc12]

1-[5-Chloro-2-(3-fluoro-4-piperazin-1-yl-phenylamino)-pyrimidin-4-yl]-1H-indole-3-carboxamide was obtained in accordance with the same preparation method of Example 41 except for replacing the 1-(2-chloro-5-fluoro-pyrimidin-4-yl)-1H-indole-3-carboxamide in Example 41 with 1-(2,5-dichloro-pyrimidin-4-yl)-1H-indole-3-carboxamide (prepared in Step 1 of Example 34).

¹HNMR (DMSO-d6, 400 MHz) δ: 10.26 (s, 1H), 8.83 (s, 1H), 8.55 (s, 1H), 8.28-8.30 (m, 1H), 7.77-7.80 (m, 2H), 7.71-7.75 (m, 1H), 7.37-7.42 (m, 1H), 7.27-7.33 (m, 2H), 7.13 (br, 1H), 7.01-7.05 (t, 1H), 3.23 (m, 4H), 3.16 (m, 4H).

LC-MS (ESI): 466.1 (M+H)⁺.

### Example 45

### Preparation of 1-{2-[4-(4-methyl-piperazin-1-yl)-phenylamino]-pyrimidin-4-yl}-1H-indole-3-carboxamide (Compound 45)

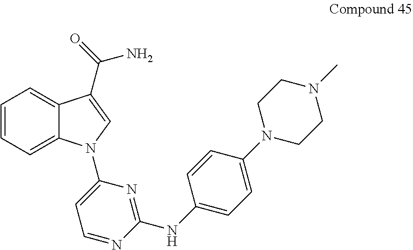 [SMILES: CN1CCN(c2ccc(Nc3nccc(-n4cc(C(N)=O)c5ccccc54)n3)cc2)CC1]

1-{2-[4-(4-Methyl-piperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylpiperazine.

¹HNMR (DMSO-d6, 400 MHz) δ: 9.58 (1H, s), 8.77 (1H, s), 8.69 (1H, s), 8.53 (1H, d), 8.26 (1H, t), 7.69 (1H, s), 7.58 (2H, d), 7.29 (2H, t), 7.18 (1H, br), 7.04 (1H, d), 6.96 (2H, d), 3.11 (4H, t), 2.47 (4H, d), 2.24 (3H, s).

LC-MS (ESI): 428.1 (M+H)⁺.

### Example 46

### Preparation of 1-{2-[3-fluoro-4-(4-methyl-piperazin-1-yl)-phenylamino]-pyrimidin-4-yl}-1H-indole-3-carboxamide (Compound 46)

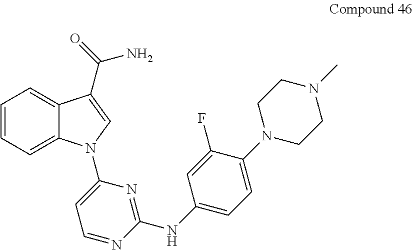 [SMILES: CN1CCN(c2ccc(Nc3nccc(-n4cc(C(N)=O)c5ccccc54)n3)cc2F)CC1]

1-{2-[3-Fluoro-4-(4-methyl-piperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylpiperazine and replacing the 4-fluoronitrobenzene in Step 1 of Example 27 with 3,4-difluoronitrobenzene.

¹HNMR (DMSO-d6, 400 MHz) δ: 9.85 (1H, s), 8.78 (1H, s), 8.73 (1H, s), 8.60 (1H, d), 8.26 (1H, dd), 7.77 (2H, m), 7.42 (1H, dd), 7.33 (2H, m), 7.19 (1H, s), 7.12 (1H, d), 7.03 (1H, t), 2.99 (4H, s), 2.25 (3H, s).

LC-MS (ESI): 446.2 (M+H)⁺.

### Example 47

### Preparation of 1-{2-[3,5-difluoro-4-(4-methyl-piperazin-1-yl)-phenylamino]-pyrimidin-4-yl}-1H-indole-3-carboxamide (Compound 47)

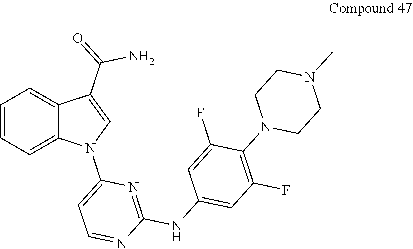 [SMILES: CN1CCN(c2c(F)cc(Nc3nccc(-n4cc(C(N)=O)c5ccccc54)n3)cc2F)CC1]

1-{2-[3,5-difluoro-4-(4-methyl-piperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylpiperazine and replacing the 4-fluoronitrobenzene in Step 1 of Example 27 with 3,4,5-trifluoronitrobenzene.

¹HNMR (DMSO-d6, 400 MHz) δ: 10.04 (1H, s), 8.78 (1H, s), 8.71-8.73 (1H, d), 8.63-8.64 (1H, d), 8.25-8.27 (1H, d), 7.68 (1H, br), 7.51-7.54 (2, m), 7.30-7.37 (2, m), 7.17-7.20 (2H, m), 3.06 (4, m), 2.42 (4H, m), 2.22 (3H, s).

LC-MS (ESI): 464.2 (M+H)⁺.

### Example 48

### Preparation of 1-{2-[3-methoxy-4-(4-methyl-piperazin-1-yl)-phenylamino]-pyrimidin-4-yl}-1H-indole-3-carboxamide (Compound 48)

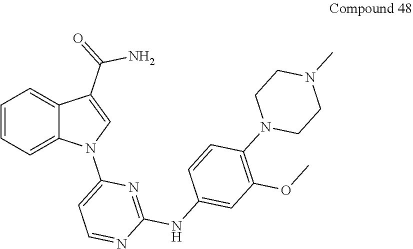 [SMILES: COc1cc(Nc2nccc(-n3cc(C(N)=O)c4ccccc43)n2)ccc1N1CCN(C)CC1]

1-{2-[3-Methoxy-4-(4-methyl-piperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylpiperazine and replacing the 4-fluoronitrobenzene in Step 1 of Example 27 with 1-fluoro-5-nitroanisole.

¹HNMR (DMSO-d6, 400 MHz) δ: 8.75 (1H, s), 8.62 (1H, s), 8.45 (1H, d), 8.38 (1H, s), 8.22 (1H, d), 7.67 (1H, s), 7.65 (1H, s), 7.40 (1H, d), 7.24 (1H, d), 7.14 (2H, s), 6.96 (1H, d), 6.70 (1H, s), 6.56 (1H, d), 3.77 (3H, s), 3.21 (4H, t), 2.52 (4H, t), 2.28 (3H, s).

LC-MS (ESI): 458.3 (M+H)⁺.

### Example 49

### Preparation of 1-{2-[3-cyano-4-(4-methyl-piperazin-1-yl)-phenylamino]-pyrimidin-4-yl}-1H-indole-3-carboxamide (Compound 49)

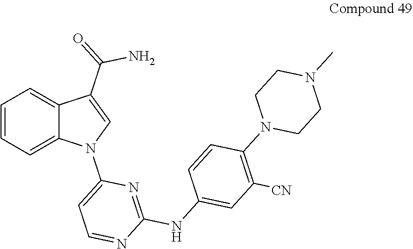

1-{2-[3-Cyano-4-(4-methyl-piperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylpiperazine and replacing the 4-fluoronitrobenzene in Step 1 of Example 27 with 3-cyano-4-fluoronitrobenzene.

¹HNMR (DMSO-d6, 400 MHz) δ: 9.98 (1H, s), 8.80 (1H, s), 8.71 (1H, s), 8.61 (1H, d), 8.26 (1H, d), 8.21 (1H, s), 7.88 (1H, s), 7.70 (1H, d), 7.33 (2H, m), 7.23 (2H, d), 7.16 (1H, d), 3.12 (4H, t), 2.54 (4H, t), 2.27 (3H, s).

LC-MS (ESI): 453.2 (M+H)⁺.

### Example 50

### Preparation of 1-{2-[3-methyl-4-(4-methyl-piperazin-1-yl)-phenylamino]-pyrimidin-4-yl}-1H-indole-3-carboxamide (Compound 50)

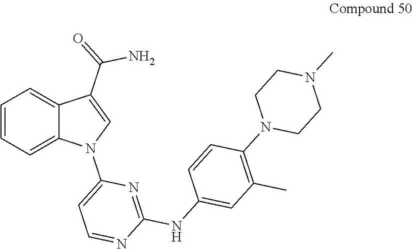 [SMILES: Cc1cc(Nc2nccc(-n3cc(C(N)=O)c4ccccc43)n2)ccc1N1CCN(C)CC1]

1-{2-[3-Methyl-4-(4-methyl-piperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylpiperazine and replacing the 4-fluoronitrobenzene in Step 1 of Example 27 with 2-fluoro-5-nitrotoluene.

¹HNMR (DMSO-d6, 400 MHz) δ: 9.66 (1H, s), 8.79 (1H, s), 8.72 (1H, s), 8.56 (1H, d), 8.26 (1H, dd), 7.70 (1H, s), 7.59 (1H, d), 7.48 (1H, d), 7.30 (2H, dd), 7.21 (1H, s), 7.06 (2H, dd), 2.84 (4H, t), 2.51 (4H, t), 2.25 (6H, d).

LC-MS (ESI): 442.2 (M+H)⁺.

### Example 51

### Preparation of 1-{2-[4-(4-isopropyl-piperazin-1-yl)-3-methyl-phenylamino]-pyrimidin-4-yl}-1H-indole-3-carboxamide (Compound 51)

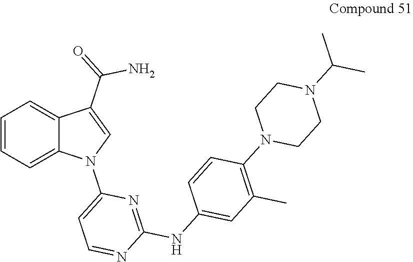 [SMILES: Cc1cc(Nc2nccc(-n3cc(C(N)=O)c4ccccc43)n2)ccc1N1CCN(C(C)C)CC1]

1-{2-[4-(4-Isopropyl-piperazin-1-yl)-3-methyl-phenylamino]-pyrimidin-4-yl}-1H-indole-3-carboxamide was obtained in accordance with the same preparation method of Example 27 except for replacing the N-Boc-piperazine in Step 1 of Example 27 with N-isopropylpiperazine and replacing the 4-fluoronitrobenzene in Step 1 of Example 27 with 2-fluoro-5-nitrotoluene.

¹HNMR (DMSO-d6, 400 MHz) δ: 9.67 (1H, s), 8.83 (1H, s), 8.75 (1H, s), 8.60 (1H, d), 8.31 (1H, dd), 7.73 (1H, s), 7.62 (1H, d), 7.53 (1H, d), 7.34 (2H, dd), 7.22 (1H, s), 7.11 (1H, d), 7.08 (H d), 2.88 (4H, br), 2.76 (1H, br), 2.67 (4H, br), 2.31 (3H, s), 1.08 (6H, d).

LC-MS (ESI): 470.3 (M+H)⁺.

### Example 52

### Preparation of 1-{5-fluoro-2-[4-(4-methyl-piperazin-1-yl)-phenylamino]-pyrimidin-4-yl}-1H-indole-3-carboxamide (Compound 52)

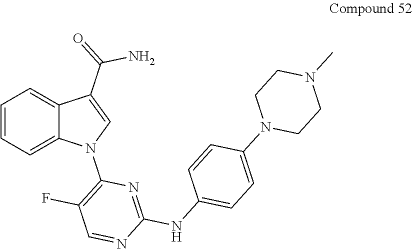 [SMILES: CN1CCN(c2ccc(Nc3ncc(F)c(-n4cc(C(N)=O)c5ccccc54)n3)cc2)CC1]

1-{5-Fluoro-2-[4-(4-methyl-piperazin-1-yl)-phenylamino]-pyrimidin-4-yl}-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 4-(4-methyl-piperazin-1-yl)-aniline (prepared in Example 45).

¹HNMR (DMSO-d6, 400 MHz) δ: 9.77 (s, 1H), 8.72-8.73 (d, 1H), 8.56 (d, 1H), 8.28-8.31 (m, 1H), 8.23 (br, 1H), 7.90 (br, 1H), 7.60-7.62 (d, 2H), 7.30-7.33 (m, 2H), 7.18 (br, 1H), 6.97-6.99 (d, 2H), 3.12 (m, 8H), 2.79 (s, 3H).

LC-MS (ESI): 446.2 (M+H)⁺.

### Example 53

### Preparation of 1-{5-chloro-2-[4-(4-methyl-piperazin-1-yl)-phenylamino]-pyrimidin-4-yl}-1H-indole-3-carboxamide (Compound 53)

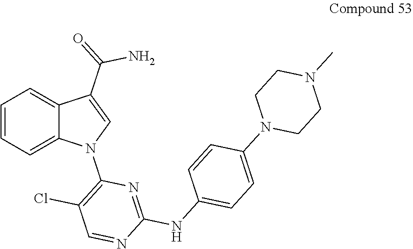 [SMILES: CN1CCN(c2ccc(Nc3ncc(Cl)c(-n4cc(C(N)=O)c5ccccc54)n3)cc2)CC1]

1-{5-Chloro-2-[4-(4-methyl-piperazin-1-yl)-phenylamino]-pyrimidin-4-yl}-1H-indole-3-carboxamide was obtained in accordance with the same preparation method of Step 2 of Example 34 except for replacing the tert-butyl 4-(4-amino-2-methyl-phenyl)-piperazine-1-carboxylate in Step 2 of Example 34 with 4-(4-methyl-piperazin-1-yl)-aniline (prepared in Example 45).

¹HNMR (DMSO-d6, 400 MHz) δ: 10.01 (s, 1H), 8.77 (s, 1H), 8.51 (s, 1H), 8.27-8.29 (m, 1H), 7.78 (br, 1H), 7.73-7.75 (m, 1H), 7.58-7.60 (d, 2H), 7.28-7.32 (m, 2H), 7.15 (br, 1H), 6.93-6.95 (d, 2H), 3.22 (m, 8H), 2.77 (s, 3H).

LC-MS (ESI): 462.2 (M+H)⁺.

### Example 54

### Preparation of 1-{5-fluoro-2-[3-fluoro-4-(4-methyl-piperazin-1-yl)-phenylamino]-pyrimidin-4-yl}-1H-indole-3-carboxamide (Compound 54)

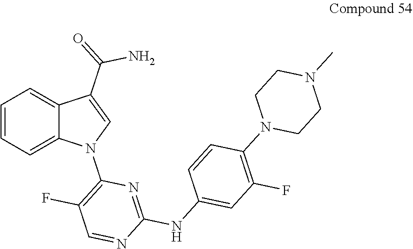 [SMILES: CN1CCN(c2ccc(Nc3ncc(F)c(-n4cc(C(N)=O)c5ccccc54)n3)cc2F)CC1]

1-{5-Fluoro-2-[3-fluoro-4-(4-methyl-piperazin-1-yl)-phenylamino]-pyrimidin-4-yl}-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 3-fluoro-4-(4-methyl-piperazin-1-yl)-aniline (prepared in Example 46).

¹HNMR (DMSO-d6, 400 MHz) δ: 9.94 (s, 1H), 8.76-8.77 (d, 1H), 8.55 (d, 1H), 8.26-8.31 (m, 2H), 7.85 (br, 1H), 7.67-7.70 (m, 1H), 7.31-7.38 (m, 3H), 7.16 (br, 1H), 6.97-7.01 (t, 1H), 2.98 (m, 4H), 2.52 (m, 4H), 2.26 (s, 3H).

LC-MS (ESI): 464.2 (M+H)⁺.

### Example 55

### Preparation of 1-{5-fluoro-2-[3-methyl-4-(4-methyl-piperazin-1-yl)-phenylamino]-pyrimidin-4-yl}-1H-indole-3-carboxamide (Compound 55)

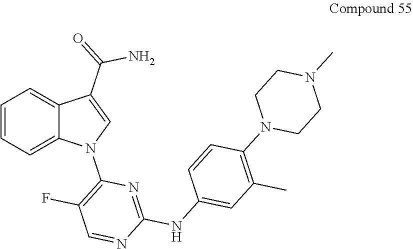 [SMILES: Cc1cc(Nc2ncc(F)c(-n3cc(C(N)=O)c4ccccc43)n2)ccc1N1CCN(C)CC1]

1-{5-Fluoro-2-[3-methyl-4-(4-methyl-piperazin-1-yl)-phenylamino]-pyrimidin-4-yl}-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 3-methyl-4-(4-methyl-piperazin-1-yl)-aniline (prepared in Example 50).

¹HNMR (DMSO-d6, 400 MHz) δ: 9.82 (s, 1H), 8.77 (d, 1H), 8.58 (s, 1H), 8.29-8.31 (m, 2H), 7.91 (br, 1H), 7.63 (s, 1H), 7.48-7.49 (d, 1H), 7.31-7.33 (m, 2H), 7.18 (m, 1H), 7.00-7.02 (d, 1H), 3.11-3.17 (m, 8H), 2.80 (s, 3H), 2.24 (s, 3H).

LC-MS (ESI): 460.2 (M+H)⁺.

### Example 56

### Preparation of 1-{5-chloro-2-[3-methyl-4-(4-methyl-piperazin-1-yl)-phenylamino]-pyrimidin-4-yl}-1H-indole-3-carboxamide (Compound 56)

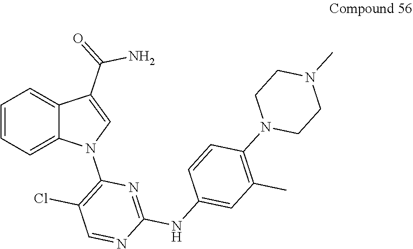 [SMILES: Cc1cc(Nc2ncc(Cl)c(-n3cc(C(N)=O)c4ccccc43)n2)ccc1N1CCN(C)CC1]

1-{5-Chloro-2-[3-methyl-4-(4-methyl-piperazin-1-yl)-phenylamino]-pyrimidin-4-yl}-1H-indole-3-carboxamide was obtained in accordance with the same preparation method of Step 2 of Example 34 except for replacing the tert-butyl 4-(4-amino-2-methyl-phenyl)-piperazine-1-carboxylate in Step 2 of Example 34 with 3-methyl-4-(4-methyl-piperazin-1-yl)-aniline (prepared in Example 50).

¹HNMR (DMSO-d6, 400 MHz) δ: 10.01 (s, 1H), 8.77 (s, 1H), 8.54 (s, 1H), 8.27-8.29 (m, 1H), 7.77-7.79 (m, 2H), 7.59 (s, 1H), 7.44-7.46 (d, 1H), 7.27-7.32 (m, 2H), 7.13 (m, 1H), 6.95-6.97 (d, 1H), 3.00-3.06 (m, 8H), 2.64 (s, 3H), 2.18 (s, 3H).

LC-MS (ESI): 476.1 (M+H)⁺.

### Example 57

### Preparation of 1-{2-[4-(4-ethyl-piperazin-1-yl)-3-methyl-phenylamino]-5-fluoro-pyrimidin-4-yl}-1H-indole-3-carboxamide (Compound 57)

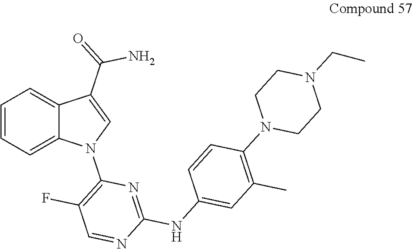 [SMILES: CCN1CCN(c2ccc(Nc3ncc(F)c(-n4cc(C(N)=O)c5ccccc54)n3)cc2C)CC1]

1-{2-[4-(4-Ethyl-piperazin-1-yl)-3-methyl-phenylamino]-5-fluoro-pyrimidin-4-yl}-1H-indole-3-carboxamide was obtained in accordance with the same preparation method of Example 39 except for replacing the N-Boc-piperazine in Step 1 of Example 39 with N-ethylpiperazine.

¹HNMR (DMSO-d6, 400 MHz) δ: 9.77 (s, 1H), 8.77-8.78 (d, 1H), 8.60 (s, 1H), 8.33-8.35 (m, 2H), 7.90 (br, 1H), 7.61 (s, 1H), 7.49-7.51 (d, 1H), 7.35-7.37 (m, 2H), 7.20 (br, 1H), 7.03-7.05 (d, 1H), 2.89 (m, 4H), 2.45-2.80 (m, 6H), 2.27 (s, 3H), 1.11-1.12 (m, 3H).

LC-MS (ESI): 474.2 (M+H)⁺.

### Example 58

### Preparation of 1-{5-chloro-2-[4-(4-ethyl-piperazin-1-yl)-3-methyl-phenylamino]-pyrimidin-4-yl}-1H-indole-3-carboxamide (Compound 58)

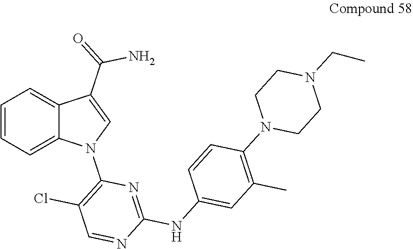 [SMILES: CCN1CCN(c2ccc(Nc3ncc(Cl)c(-n4cc(C(N)=O)c5ccccc54)n3)cc2C)CC1]

1-{5-Chloro-2-[4-(4-ethyl-piperazin-1-yl)-3-methyl-phenylamino]-pyrimidin-4-yl}-1H-indole-3-carboxamide was obtained in accordance with the same preparation method of Example 34 except for replacing the N-Boc-piperazine in Step 1 of Example 34 with N-ethylpiperazine.

¹HNMR (DMSO-d6, 400 MHz) δ: 10.00 (s, 1H), 8.77 (d, 1H), 8.53 (s, 1H), 8.27-8.29 (m, 1H), 7.78-7.80 (m, 2H), 7.57 (s, 1H), 7.43-7.45 (m, 1H), 7.27-7.33 (m, 2H), 7.10 (br, 1H), 6.95-6.97 (d, 1H), 2.57-3.01 (m, 10H), 1.12 (m, 3H).

LC-MS (ESI): 490.1 (M+H)⁺.

### Example 59

### Preparation of 1-{5-fluoro-2-[4-(4-isopropyl-piperazin-1-yl)-3-methyl-phenylamino]-pyrimidin-4-yl}-1H-indole-3-carboxamide Compound 59)

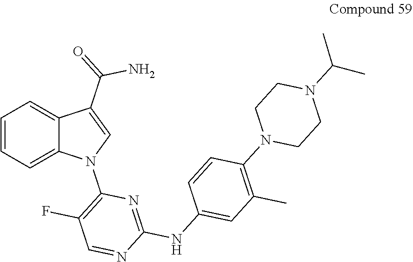 [SMILES: Cc1cc(Nc2ncc(F)c(-n3cc(C(N)=O)c4ccccc43)n2)ccc1N1CCN(C(C)C)CC1]

1-{5-Fluoro-2-[4-(4-isopropyl-piperazin-1-yl)-3-methyl-phenylamino]-pyrimidin-4-yl}-1H-indole-3-carboxamide was obtained in accordance with the same preparation method of Example 39 except for replacing the N-Boc-piperazine in Step 1 of Example 39 with N-isopropylpiperazine.

¹HNMR (DMSO-d6, 400 MHz) δ: 9.72 (s, 1H), 8.72-8.73 (d, 1H), 8.55 (s, 1H), 8.28-8.30 (m, 2H), 7.85 (br, 1H), 7.56 (s, 1H), 7.43-7.45 (d, 1H), 7.30-7.32 (m, 2H), 7.15 (br, 1H), 6.97-6.99 (d, 1H), 2.60-2.90 (m, 9H), 1.05-1.06 (d, 6H).

LC-MS (ESI): 488.2 (M+H)⁺.

### Example 60

### Preparation of 1-{5-chloro-2-[4-(4-isopropyl-piperazin-1-yl)-3-methyl-phenylamino]-pyrimidin-4-yl}-1H-indole-3-carboxamide (Compound 60)

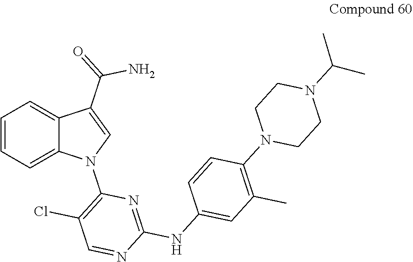 [SMILES: Cc1cc(Nc2ncc(Cl)c(-n3cc(C(N)=O)c4ccccc43)n2)ccc1N1CCN(C(C)C)CC1]

1-{5-Chloro-2-[4-(4-isopropyl-piperazin-1-yl)-3-methyl-phenylamino]-pyrimidin-4-yl}-1H-indole-3-carboxamide was obtained in accordance with the same preparation method of Example 34 except for replacing the N-Boc-piperazine in Step 1 of Example 34 with N-isopropylpiperazine.

¹HNMR (DMSO-d6, 400 MHz) δ: 10.00 (s, 1H), 8.77 (s, 1H), 8.53 (s, 1H), 8.27-8.29 (m, 1H), 7.78-7.80 (m, 2H), 7.57 (s, 1H), 7.44-7.46 (m, 1H), 7.27-7.33 (m, 2H), 7.13 (br, 1H), 6.95-6.97 (d, 1H), 2.65-3.11 (m, 9H), 1.12-1.13 (d, 6H).

LC-MS (ESI): 504.1 (M+H)⁺.

### Example 61

### Preparation of 1-{5-chloro-2-[3-fluoro-4-(4-methyl-piperazin-1-yl)-phenylamino]-pyrimidin-4-yl}-1H-indole-3-carboxamide (Compound 61)

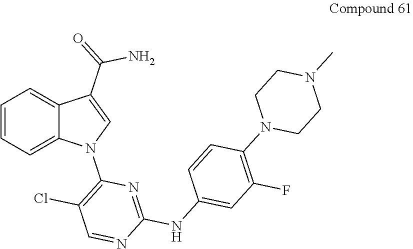 [SMILES: CN1CCN(c2ccc(Nc3ncc(Cl)c(-n4cc(C(N)=O)c5ccccc54)n3)cc2F)CC1]

1-{5-Chloro-2-[3-fluoro-4-(4-methyl-piperazin-1-yl)-phenylamino]-pyrimidin-4-yl}-1H-indole-3-carboxamide was obtained in accordance with the same preparation method of Step 2 of Example 34 except for replacing the tert-butyl 4-(4-amino-2-methyl-phenyl)-piperazine-1-carboxylate in Step 2 of Example 34 with 3-fluoro-4-(4-methyl-piperazin-1-yl)-aniline (prepared in Example 54).

¹HNMR (DMSO-d6, 400 MHz) δ: 10.25 (s, 1H), 8.83 (d, 1H), 8.54 (s, 1H), 8.27-8.29 (m, 1H), 7.77-7.79 (m, 2H), 7.70-7.74 (m, 1H), 7.38-7.41 (m, 1H), 7.27-7.33 (m, 2H), 7.15 (br, 1H), 7.00-7.05 (m, 1H), 3.07-3.17 (m, 8H), 2.64 (s, 3H).

LC-MS (ESI): 480.1 (M+H)⁺.

### Example 62

### Preparation of 1-(2-{3-fluoro-4-[4-(1-methyl-piperidin-4-yl)-piperazin-1-yl]-phenylamino}-pyrimidin-4-yl)-1H-indole-3-carboxamide (Compound 62)

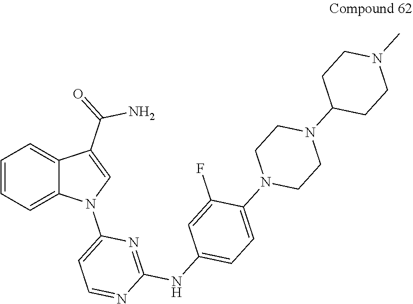 [SMILES: CN1CCC(N2CCN(c3ccc(Nc4nccc(-n5cc(C(N)=O)c6ccccc65)n4)cc3F)CC2)CC1]

1-(2-{3-Fluoro-4-[4-(1-methyl-piperidin-4-yl)-piperazin-1-yl]-phenylamino}-pyrimidin-4-yl)-H-indole-3-carboxamide was obtained in accordance with the same preparation method of Example 27 except for replacing the 4-fluoronitrobenzene in Step of Example 27 with 3,4-difluoronitrobenzene and replacing the N-Boc-piperazine in Step 1 of Example 27 with 1-(1-methyl-4-piperidinyl)piperazine.

¹HNMR (DMSO-d6, 400 MHz) δ: 9.85 (1H, s), 8.79 (1H, s), 8.73 (1H, s), 8.60 (1H, d), 8.28 (1H, d), 7.72 (2H, m), 7.42 (1H, d), 7.32 (2H, d), 7.20 (1H, s), 7.12 (1H, d), 7.02 (1H, t), 2.98 (4H, s), 2.82 (2H, d), 2.63 (4H, s), 2.19-2.15 (4H, m), 1.86 (2H, t), 1.76 (2H, d), 1.45 (2H, t).

LC-MS (ESI): 529.2 (M+H)⁺.

### Example 63

### Preparation of 1-(2-{4-[4-(2-dimethylamino-ethyl)-piperazin-1-yl]-3-fluoro-phenylamino}-pyrimidin-4-yl)-1H-indole-3-carboxamide (Compound 63

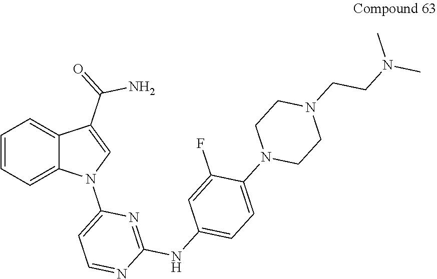 [SMILES: CN(C)CCN1CCN(c2ccc(Nc3nccc(-n4cc(C(N)=O)c5ccccc54)n3)cc2F)CC1]

1-(2-{-4-[4-(2-Dimethylamino-ethyl)-piperazin-1-yl]-3-fluoro-phenylamino}-pyrimidin-4-yl)-1H-indole-3-carboxamide was obtained in accordance with the same preparation method of Example 27 except for replacing the 4-fluoronitrobenzene in Step 1 of Example 27 with 3,4-difluoronitrobenzene and replacing the N-Boc-piperazine in Step 1 of Example 27 with 1-(2-dimethylaminoethyl)piperazine.

¹HNMR (DMSO-d6, 400 MHz) δ: 2.52-2.58 (6H, m), 3.12 (4H, br), 3.25 (3H, s), 3.47 (2H, t), 6.63 (1H, d), 7.10 (1H, d), 7.14-7.23 (2H, m), 7.24-7.27 (1H, m), 7.29-7.35 (2H, m), 7.39 (1H, s), 7.69 (1H, br), 8.24-8.27 (1H, m), 8.59 (1H, d), 8.72 (1H, br), 8.79 (1H, s), 9.69 (1H, s).

LC-MS (ESI): 472.2 (M+H)⁺.

### Example 64

### Preparation of 1-{2-[4-(4-acryloyl-piperazin-1-yl)-3-methyl-phenylamino]-pyrimidin-4-yl}-1H-indole-3-carboxamide (Compound 64)

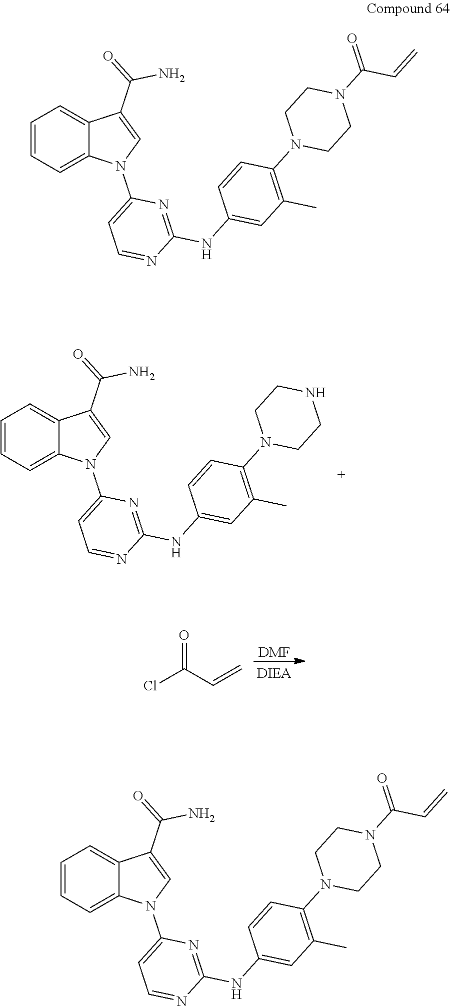

1-[2-(3-Methyl-4-piperazin-1-yl-phenylamino)-pyrimidin-4-yl]-1H-indole-3-carboxamide (Compound 28) (100 mg, 0.234 mmol) and N,N-diisopropylethylamine (90.5 mg, 0.702 mol) were dissolved in DMF (10 ml). Potassium carbonate (32.3 mg, 0.234 mmol) was added at room temperature, and then acryloyl chloride (25.4 mg, 0.281 mmol) was slowly added dropwise under an ice bath. After completion of the addition, the ice bath was removed, and the reaction solution was slowly warmed to room temperature and reacted for 1 hour. TLC showed that the reaction was completed. The reaction solution was slowly poured into water, and extracted with ethyl acetate (30 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol) to obtain 1-{2-[4-(4-acryloyl-piperazin-1-yl)-3-methyl-phenylamino]-pyrimidin-4-yl}-1H-indole-3-carboxamide (33 mg, white solid).

¹HNMR (DMSO-d6, 400 MHz) δ: 9.69 (s, 1H), 8.78 (s, 1H), 8.70 (br, 1H), 8.55-8.57 (d, 1H), 8.24-8.26 (m, 1H), 7.70 (br, 1H), 7.61-7.62 (d, 1H), 7.48-7.50 (d, 1H), 7.29-7.31 (m, 2H), 7.21 (br, 1H), 7.06-7.07 (d, 1H), 7.02-7.04 (d, 1H), 6.83-6.90 (m, 1H), 6.13-6.18 (m, 1H), 5.70-5.74 (m, 1H), 3.71 (m, 4H), 2.82 (m, 4H), 2.30 (s, 3H).

LC-MS (ESI): 482.2 (M+H)⁺.

### Example 65

### Preparation of 1-{2-[3-methyl-4-(4-propionyl-piperazin-1-yl)-phenylamino]-pyrimidin-4-yl}-1H-indole-3-carboxamide (Compound 65)

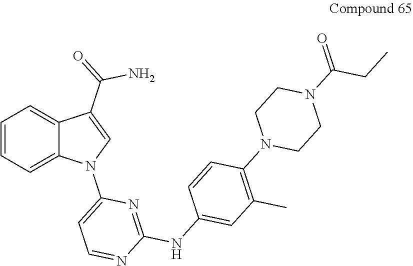 [SMILES: CCC(=O)N1CCN(c2ccc(Nc3nccc(-n4cc(C(N)=O)c5ccccc54)n3)cc2C)CC1]

1-{2-[3-Methyl-4-(4-propionyl-piperazin-1-yl)-phenylamino]-pyrimidin-4-yl}-1H-indole-3-carboxamide was obtained in accordance with the same preparation method of Example 64 except for replacing the acryloyl chloride in Example 64 with propionyl chloride.

¹HNMR (DMSO-d6, 400 MHz) δ: 9.69 (s, 1H), 8.79 (s, 1H), 8.71 (br, 1H), 8.55-8.57 (d, 1H), 8.24-8.26 (m, 1H), 7.70 (br, 1H), 7.61-7.62 (d, 1H), 7.48-7.50 (d, 1H), 7.29-7.31 (m, 2H), 7.21 (br, 1H), 7.06-7.07 (d, 1H), 7.01-7.03 (d, 1H), 3.58-3.61 (m, 4H), 2.77-2.83 (m, 4H), 2.34-2.40 (q, 2H), (m, 1H), 2.29 (s, 3H), 1.00-1.04 (t, 3H).

LC-MS (ESI): 484.2 (M+H)⁺.

### Example 66

### Preparation of 1-{2-[4-(4-acetyl-piperazin-1-yl)-3-fluoro-phenylamino]-pyrimidin-4-yl}-1H-indole-3-carboxamide (Compound 66)

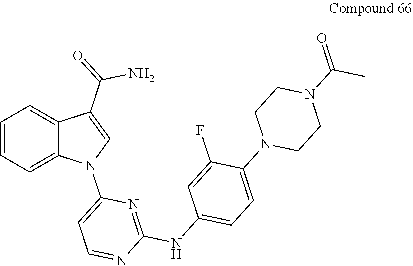 [SMILES: CC(=O)N1CCN(c2ccc(Nc3nccc(-n4cc(C(N)=O)c5ccccc54)n3)cc2F)CC1]

1-{2-[4-(4-Acetyl-piperazin-1-yl)-3-fluoro-phenylamino]-pyrimidin-4-yl}-1H-indole-3-carboxamide was obtained in accordance with the same preparation method of Example 27 except for replacing the 4-fluoronitrobenzene in Step 1 of Example 27 with 3,4-difluoronitrobenzene and replacing the N-Boc-piperazine in Step 1 of Example 27 with N-acetylpiperazine.

¹HNMR (DMSO-d6, 400 MHz) δ: 9.90 (1H, s), 8.80 (1H, s), 8.74 (1H, s), 8.62 (1H, d), 8.28 (1H, d), 7.81 (1H, d), 7.71 (1H, s), 7.45 (1H, d), 7.33 (2H, d), 7.22 (1H, s), 7.14 (1H, d), 7.05 (1H, t), 3.61 (4H, t), 2.99 (4H, d), 2.06 (3H, s).

LC-MS (ESI): 474.1 (M+H)⁺.

### Example 67

### Preparation of 1-{2-[4-(4-methoxy-piperidin-1-yl)-phenylamino]-pyrimidin-4-yl}-1H-indole-3-carboxamide (Compound 67)

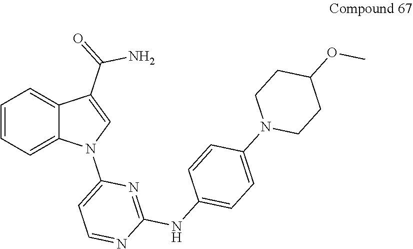 [SMILES: COC1CCN(c2ccc(Nc3nccc(-n4cc(C(N)=O)c5ccccc54)n3)cc2)CC1]

1-{2-[4-(4-Methoxy-piperidin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with 4-methoxypiperidine.

¹HNMR (DMSO-d6, 400 MHz) δ: 9.57 (1H, s), 8.77 (1H, s), 8.69 (1H, s), 8.53 (1H, d), 8.25 (1H, t), 7.68 (1H, s), 7.56 (2H, d), 7.28 (2H, br), 7.19+(1H, br), 7.03 (1H, d), 6.97 (2H, d), 3.46 (2H, br), 3.28 (3H, s), 2.85 (2H, br), 1.96 (3H, m), 1.56 (2H, br).

LC-MS (ESI): 443.2 (M+H)⁺.

### Example 68

### Preparation of 1-{2-[4-(4-dimethylamino-piperidin-1-yl)-3-methyl-phenylamino]-pyrimidin-4-yl}-1H-indole-3-carboxamide (Compound 68)

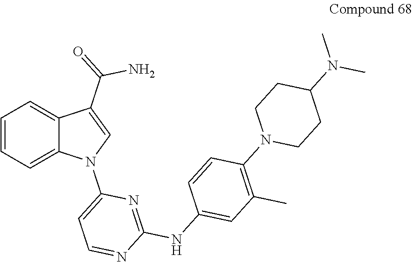 [SMILES: Cc1cc(Nc2nccc(-n3cc(C(N)=O)c4ccccc43)n2)ccc1N1CCC(N(C)C)CC1]

1-{2-[4-(4-Dimethylamino-piperidin-1-yl)-3-methyl-phenylamino]-pyrimidin-4-yl}-1H-indole-3-carboxamide was obtained in accordance with the same preparation method of Example 27 except for replacing the 4-fluoronitrobenzene in Step 1 of Example 27 with 2-fluoro-5-nitrotoluene and replacing the N-Boc-piperazine in Step 1 of Example 27 with 4-dimethyl aminopiperidine.

¹HNMR (DMSO-d6, 400 MHz) δ: 9.61 (1H, s), 8.77 (1H, s), 8.70 (1H, s), 8.55 (1H, d), 8.25 (1H, dd), 7.66 (1H, s), 7.57 (1H, d), 7.47 (1H, d), 7.29 (2H, dd), 7.16 (1H, s), 7.06 (1H, d), 7.02 (1H, d), 3.18 (1H, d), 3.10 (2H, br), 2.60 (2H, t), 2.29 (6H, s), 2.25 (3H, s), 1.89 (2H, br), 1.56 (2H, br).

LC-MS (ESI): 470.2 (M+H)⁺.

### Example 69

### Preparation of 1-{2-[4-(4-methyl-[1,4]homopiperazin-1-yl)-phenylamino]-pyrimidin-4-yl}-1H-indole-3-carboxamide (Compound 69)

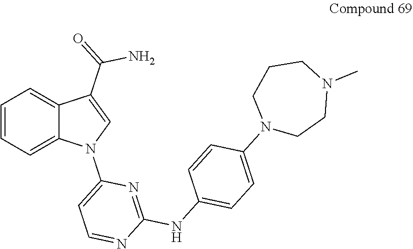 [SMILES: CN1CCCN(c2ccc(Nc3nccc(-n4cc(C(N)=O)c5ccccc54)n3)cc2)CC1]

1-{2-[4-(4-Methyl-[1,4]homopiperazin-1-yl)-phenylamino]-pyrimidin-4-yl}-1H-indole-3-carboxamide was obtained in accordance with the same preparation method of Example 27 except for replacing the N-Boc-piperazine in Step 1 of Example 27 with N-methylhomopiperazine.

¹HNMR (DMSO-d6, 400 MHz) δ: 1.96-2.04 (2H, m), 2.46 (3H, s), 2.73 (2H, br), 2.86 (2H, br), 3.59 (2H, br), 3.46 (2H, br), 6.74 (2H, d), 6.99 (1H, d), 7.13-7.31 (3H, m), 7.48 (2H, d), 7.69 (1H, br), 8.23-8.26 (1H, m), 8.50 (1H, d), 8.68 (1H, br), 8.78 (1H, s), 9.44 (1H, s).

LC-MS (ESI): 442.2 (M+H)⁺.

### Example 70

### Preparation of 1-[2-(4-morpholin-4-yl-phenylamino)-pyrimidin-4-yl]-1H-indole-3-carboxamide (Compound 70)

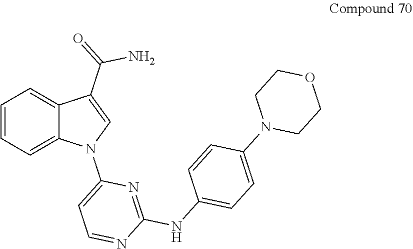 [SMILES: NC(=O)c1cn(-c2ccnc(Nc3ccc(N4CCOCC4)cc3)n2)c2ccccc12]

1-[2-(4-Morpholin-4-yl-phenylamino)-pyrimidin-4-yl]-1H-indole-3-carboxamide was obtained in accordance with the same preparation method of Example 27 except for replacing the N-Boc-piperazine in Step 1 of Example 27 with morpholine.

¹HNMR (DMSO-d6, 400 MHz) δ:3.08 (4H, t), 3.76 (4H, t), 6.96 (2H, d), 7.05 (1H, d), 7.18 (1H, br), 7.28-7.30 (2H, m), 7.59 (2H, d), 7.72 (1H, br), 8.24-8.26 (1H, m), 8.53 (1H, d), 8.71 (1H, br), 8.80 (1H, s), 9.61 (1H, s).

LC-MS (ESI): 415.1 (M+H)⁺.

### Example 71

### Preparation of 1-[2-(3-fluoro-4-morpholin-4-yl-phenylamino)-pyrimidin-4-yl]-1H-indole-3-carboxamide (Compound 71)

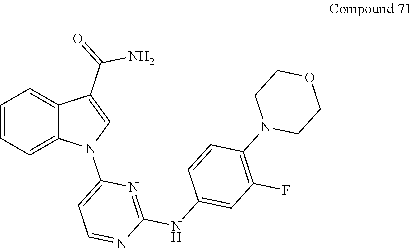 [SMILES: NC(=O)c1cn(-c2ccnc(Nc3ccc(N4CCOCC4)c(F)c3)n2)c2ccccc12]

1-[2-(3-Fluoro-4-morpholin-4-yl-phenylamino)-pyrimidin-4-yl]-1H-indole-3-carboxamide was obtained in accordance with the same preparation method of Example 27 except for replacing the 4-fluoronitrobenzene in Step 1 of Example 27 with 3,4-difluoronitrobenzene and replacing the N-Boc-piperazine in Step 1 of Example 27 with morpholine.

¹HNMR (DMSO-d6, 400 MHz) δ: 9.87 (1H, s), 8.79 (1H, s), 8.72 (1H, s), 8.61 (1H, d), 8.26 (1H, dd), 7.79 (1H, d), 7.69 (1H, s), 7.44 (1H, dd), 7.33 (2H, m), 7.19 (1H, s), 7.13 (1H, d), 7.04 (1H, t), 3.76 (4H, t), 2.98 (4H, t).

LC-MS (ESI): 433.1 (M+H)⁺.

### Example 72

### Preparation of 1-(2-{4-[methyl-(2-morpholin-4-ethyl)-amino]-phenylamino}-pyrimidin-4-yl)-1H-indole-3-carboxamide (Compound 72)

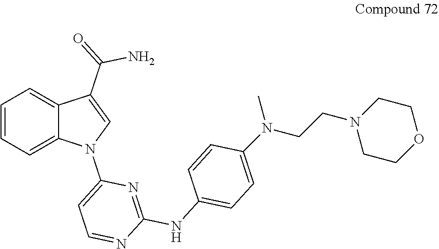 [SMILES: CN(CCN1CCOCC1)c1ccc(Nc2nccc(-n3cc(C(N)=O)c4ccccc43)n2)cc1]

1-(2-{4-[Methyl-(2-morpholin-4-ethyl)-amino]-phenylamino}-pyrimidin-4-yl)-1H-indole-3-carboxamide was obtained in accordance with the same preparation method of Example 27 except for replacing the N-Boc-piperazine in Step 1 of Example 27 with N-methyl-2-morpholinoethylamine.

¹HNMR (DMSO-d6, 400 MHz) δ: 9.45 (1H, s), 8.76 (1H, s), 8.69 (1H, s), 8.50 (1H, d), 8.25 (1H, dd), 7.68 (1H, s), 7.48 (2H, d), 7.30 (2H, m), 7.18 (1H, s), 6.99 (1H, d), 6.74 (2H, d), 3.57 (4H, t), 3.46 (2H, t), 2.91 (3H, s), 2.45 (6H, m).

LC-MS (ESI): 472.2 (M+H)⁺.

### Example 73

### Preparation of 1-[2-(4-{methyl-[2-(4-methyl-piperazin-1-yl)-ethyl]-amino}-phenylamino)-pyrimidin-4-yl]-1H-indole-3-carboxamide (Compound 73)

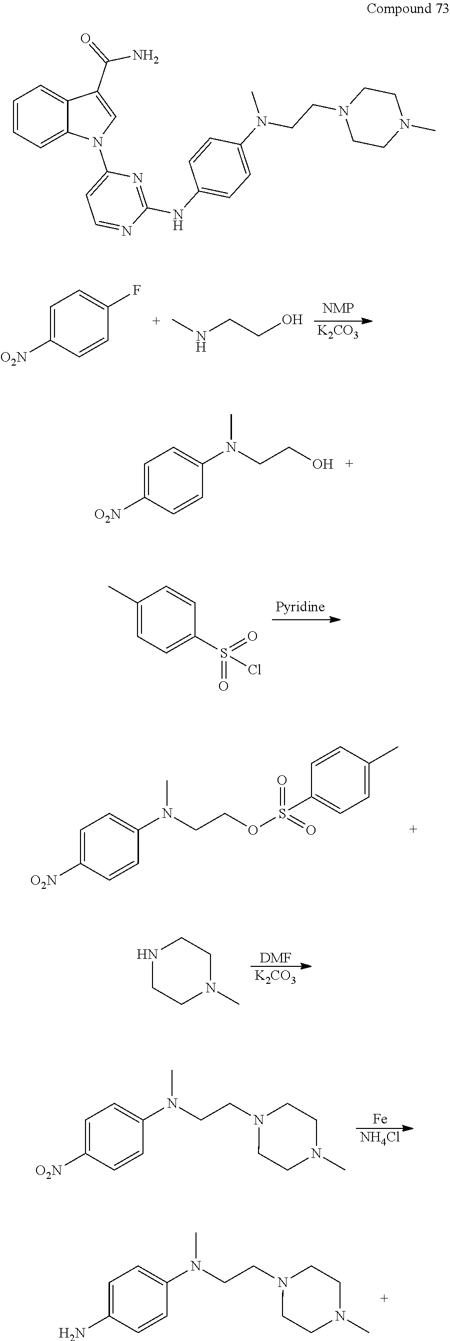

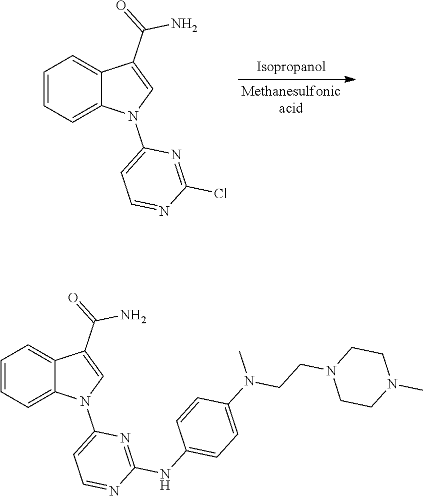 [SMILES: NC(=O)c1cn(-c2ccnc(Cl)n2)c2ccccc12 ; CN1CCN(CCN(C)c2ccc(Nc3nccc(-n4cc(C(N)=O)c5ccccc54)n3)cc2)CC1]

### Step 1: Preparation of 2-[methyl-(4-nitro-phenyl)-amino]-ethanol

4-Fluoronitrobenzene (22.1 g, 0.157 mol) and N-methyl-2-hydroxyethylamine (15.3 g, 0.204 mol) were dissolved in NMP (150 ml). Potassium carbonate (43.3 g, 0.314 mol) was added at room temperature, and the resulting solution was heated to 100° C. and reacted for 8 hours. TLC showed that the reaction was completed. The reaction solution was cooled to room temperature, slowly poured into 500 ml of water, and extracted with ethyl acetate (150 ml×2). The organic phase was washed with saturated NaCl solution twice, dried over anhydrous sodium sulfate, filtrated, and concentrated under reduced pressure to obtain 2-[methyl-(4-nitro-phenyl)-amino]-ethanol (25.2 g, yellow solid).

### Step 2: Preparation of 2-[methyl-(4-nitro-phenyl)-amino]-ethyl 4-methylbenzenesulfonate

The product 2-[methyl-(4-nitro-phenyl)-amino]-ethanol (25.2 g, 0.128 mol) obtained in Step 1 was dissolved in 160 ml of pyridine, and the resulting solution was cooled in an ice water bath. P-toluenesulfonyl chloride (36.6 g, 0.192 mol) was slowly added dropwise, and the reaction solution was warmed to room temperature and reacted for 12 hours after completion of the addition. TLC showed that the reaction was completed. The reaction solution was slowly poured into 1500 ml of water to precipitate a solid, stirred at room temperature for 30 minutes and filtrated. The solid was washed with water, and dried by blowing (60° C.) for 12 hours to obtain 2-[methyl-(4-nitro-phenyl)-amino]-ethyl 4-methylbenzenesulfonate (35.5 g, yellow solid). The product was used directly in the next step without purification.

### Step 3: Preparation of methyl-[2-(4-methyl-piperazin-1-yl)-ethyl]-(4-nitro-phenyl)-amine

The product 2-[methyl-(4-nitro-phenyl)-amino]-ethyl 4-methylbenzenesulfonate (518 mg, 1.48 mmol) obtained in Step 2 and N-methylpiperazine (1.5 g, 14.8 mmol) were dissolved in DMF (8 ml). Potassium carbonate (210 mg, 1.52 mmol) was added at room temperature, and the resulting mixture was heated to 100° C. and reacted for 12 hours. TLC showed that the reaction was completed. The reaction solution was cooled to room temperature, slowly poured into 30 ml of water, and extracted with ethyl acetate (50 ml×2). The organic phase was washed with saturated NaCl solution twice, dried over anhydrous sodium sulfate, filtrated, and concentrated under reduced pressure to obtain methyl-[2-(4-methyl-piperazin-1-yl)-ethyl]-(4-nitro-phenyl)-amine (370 mg, solid). The product was used directly in the next step without purification.

### Step 4: Preparation of N-methyl-N-[2-(4-methyl-piperazin-1-yl)-ethyl]-benzene-1,4-diamine

The product methyl-[2-(4-methyl-piperazin-1-yl)-ethyl]-(4-nitro-phenyl)-amine (370 mg, 1.33 mmol) obtained in Step 3, reduced iron powder (300 mg, 5.36 mmol) and ammonium chloride (500 mg, 9.35 mmol) were added into ethanol (50 ml)/water (12.5 ml). The resulting mixture was heated to 90° C. and reacted for 1 hour. The reaction solution was cooled to room temperature, slowly poured into saturated aqueous sodium bicarbonate solution (150 ml), and extracted with ethyl acetate (100 ml×2). The organic phase was washed with saturated NaCl solution twice, dried over anhydrous sodium sulfate, filtrated, and concentrated under reduced pressure to obtain N-methyl-N-[2-(4-methyl-piperazin-1-yl)-ethyl]-benzene-1,4-diamine (280 mg, solid). The product was used directly in the next step without purification.

### Step 5: Preparation of 1-[2-(4-{methyl-[2-(4-methyl-piperazin-1-yl)-ethyl]-amino}-phenylamino)-pyrimidin-4-yl]-1H-indole-3-carboxamide

1-[2-(4-{Methyl-[2-(4-methyl-piperazin-1-yl)-ethyl]-amino}-phenylamino)-pyrimidin-4-yl]-1H-indole-3-carboxamide was obtained in accordance with the same preparation method of Step 4 of Example 1 except for replacing the 3-bromo-4-fluoroaniline in Step 4 of Example 1 with N-methyl-N-[2-(4-methyl-piperazin-1-yl)-ethyl]-benzene-1,4-diamine (prepared in Step 3).

¹HNMR (DMSO-d6, 400 MHz) δ: 2.21 (3H, s), 2.33-2.54 (10H, m), 2.95 (3H, s), 3.48 (2H, t), 6.76 (2H, d), 7.03 (1H, d), 7.21-7.34 (3H, m), 7.52 (2H, d), 7.73 (1H, s), 8.28-8.30 (1H, m), 8.54 (1H, d), 8.71 (1H, br), 8.81 (1H, s), 9.49 (1H, s).

LC-MS (ESI): 485.2 (M+H)⁺.

### Example 74

### Preparation of 1-[2-(4-acetyl-piperazin-1-yl)-ethyl]-methyl-amino}-phenylamino)-pyrimidin-4-yl]-1H-indole-3-carboxamide (Compound 74)

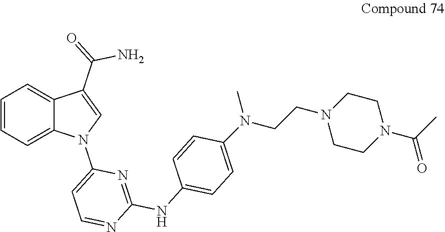 [SMILES: CC(=O)N1CCN(CCN(C)c2ccc(Nc3nccc(-n4cc(C(N)=O)c5ccccc54)n3)cc2)CC1]

1-[2-(4-Acetyl-piperazin-1-yl)-ethyl]-methyl-amino}-phenylamino)-pyrimidin-4-yl]-1H-indole-3-carboxamide was obtained in accordance with the same preparation method of Example 73 except for replacing the N-methylpiperazine in Step 1 of Example 73 with N-acetylpiperazine.

¹HNMR (DMSO-d6, 400 MHz) δ: 1.99 (3H, s), 2.38-2.51 (6H, m), 2.91 (3H, s), 3.39-3.48 (6H, m), 6.71-6.74 (2H, d), 6.77-6.98 (1H, d), 7.15 (1H, br), 7.25-7.29 (2H, m), 7.46-748 (2H, d), 7.66 (1H, br), 8.22-8.25 (1H, m), 8.48-8.49 (1H, d), 8.67 (1H, br), 8.75 (1H, s), 9.43 (1H, s).

LC-MS (ESI): 513.3 (M+H)⁺.

### Example 75

### Preparation of 1-[2-(4-dimethylamino-phenylamino)-pyrimidin-4-yl]-1H-indole-3-carboxamide (Compound 75)

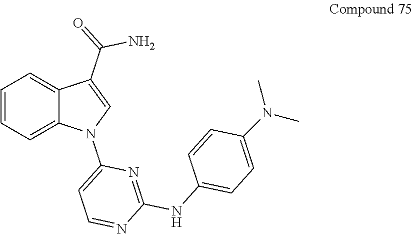 [SMILES: CN(C)c1ccc(Nc2nccc(-n3cc(C(N)=O)c4ccccc43)n2)cc1]

1-[2-(4-Dimethylamino-phenylamino)-pyrimidin-4-yl]-1H-indole-3-carboxamide was obtained in accordance with the same preparation method of Example 27 except for replacing the N-Boc-piperazine in Step 1 of Example 27 with dimethylamine hydrochloride.

¹HNMR (DMSO-d6, 400 MHz) δ: 2.89 (6H, s), 6.77 (2H, d), 7.01 (1H, d), 7.18 (1H, br), 7.26-7.31 (2H, m), 7.51 (2H, d), 7.71 (1H, br), 8.23-8.26 (1H, m), 8.50 (1H, d), 8.68 (1H, br), 8.79 (1H, s), 9.48 (1H, s).

LC-MS (ESI): 373.1 (M+H)⁺.

### Example 76

### Preparation of 1-(2-{4-[(3-dimethylamino-propyl)-methyl-amino]-phenylamino}-pyrimidin-4-yl)-1H-indole-3-carboxamide (Compound 76)

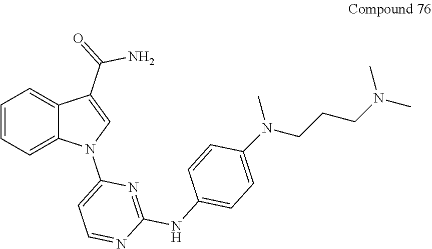 [SMILES: CN(C)CCCN(C)c1ccc(Nc2nccc(-n3cc(C(N)=O)c4ccccc43)n2)cc1]

1-(2-{4-[(3-Dimethylamino-propyl)-methyl-amino]-phenylamino}-pyrimidin-4-yl)-1H-indole-3-carboxamide was obtained in accordance with the same preparation method of Example 27 except for replacing the N-Boc-piperazine in Step 1 of Example 27 with N,N,N′-trimethyl-1,3-propanediamine.

¹HNMR (DMSO-d6, 400 MHz) δ: 9.44 (1H, s), 8.76 (1H, s), 8.66 (1H, s), 8.49 (1H, d), 8.24 (1H, dd), 7.67 (1H, s), 7.48 (2H, d), 7.27 (2H, m), 7.17 (1H, s), 6.99 (1H, d), 6.74 (2H, d), 3.33 (2H, t), 2.88 (3H, s), 2.29 (2H, t), 2.18 (6H, s), 1.66 (2H, m).

LC-MS (ESI): 444.2 (M+H)⁺.

### Example 77

### Preparation of 1-(2-{4-[(2-dimethylamino-ethyl)-methyl-amino]-phenylamino}-pyrimidin-4-yl)-1H-indole-3-carboxamide (Compound 77)

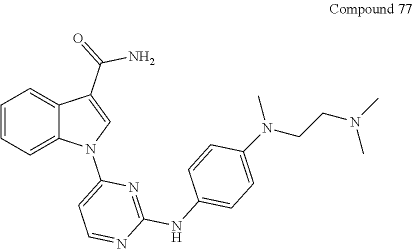 [SMILES: CN(C)CCN(C)c1ccc(Nc2nccc(-n3cc(C(N)=O)c4ccccc43)n2)cc1]

1-(2-{4-[(2-Dimethylamino-ethyl)-methyl-amino]-phenylamino}-pyrimidin-4-yl)-1H-indole-3-carboxamide was obtained in accordance with the same preparation method of Example 27 except for replacing the N-Boc-piperazine in Step 1 of Example 27 with N,N,N′-trimethylethylenediamine.

¹HNMR (DMSO-d6, 400 MHz) δ: 9.44 (1H, s), 8.76 (1H, s), 8.66 (1H, s), 8.50 (1H, d), 8.25 (1H, d), 7.67 (11H, s), 7.49 (2H, d), 7.30 (3H, m), 6.99 (1H, d), 6.73 (2H, d), 3.44 (2H, t), 2.91 (3H, s), 2.44 (2H, t), 2.22 (6H, s).

LC-MS (ESI): 430.2 (M+H)⁺.

### Example 78

### Preparation of 1-(2-{2-bromo-4-[(2-dimethylamino-ethyl)-methyl-amino]-phenylamino}-pyrimidin-4-yl)-1H-indole-3-carboxamide (Compound 78)

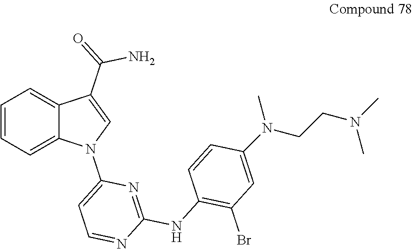 [SMILES: CN(C)CCN(C)c1ccc(Nc2nccc(-n3cc(C(N)=O)c4ccccc43)n2)c(Br)c1]

1-(2-{2-Bromo-4-[(2-dimethylamino-ethyl)-methyl-amino]-phenylamino}-pyrimidin-4-yl)-1H-indole-3-carboxamide was obtained in accordance with the same preparation method of Example 27 except for replacing the 4-fluoronitrobenzene in Step 1 of Example 27 with 2-bromo-4-fluoronitrobenzene and replacing the N-Boc-piperazine in Step 1 of Example 27 with N,N,N′-trimethylethylenediamine.

¹HNMR (DMSO-d6, 400 MHz) δ: 9.11 (1H, s), 8.75 (1H, s), 8.45 (1H, d), 8.20 (1H, d), 7.64 (1H, s), 7.29 (1H, d), 7.21 (2H, m), 7.03 (1H, s), 6.97 (2H, m), 6.80 (1H, dd), 3.48 (2H, t), 2.97 (3H, s), 2.43 (2H, t), 2.21 (6H, s).

LC-MS (ESI): 508.0 (M+H)⁺.

### Example 79

### Preparation of 1-(2-{4-[(2-dimethylamino-ethyl)-methyl-amino]-3-methyl-phenylamino}-pyrimidin-4-yl)-1H-indole-3-carboxamide (Compound 79)

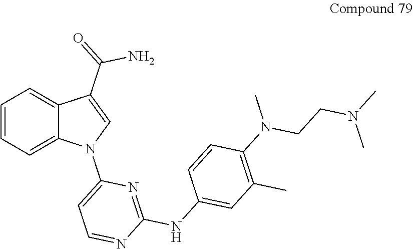 [SMILES: Cc1cc(Nc2nccc(-n3cc(C(N)=O)c4ccccc43)n2)ccc1N(C)CCN(C)C]

1-(2-{4-[(2-Dimethylamino-ethyl)-methyl-amino]-3-methyl-phenylamino}-pyrimidin-4-yl)-1H-indole-3-carboxamide was obtained in accordance with the same preparation method of Example 27 except for replacing the 4-fluoronitrobenzene in Step 1 of Example 27 with 2-fluoro-5-nitrotoluene and replacing the N-Boc-piperazine in Step 1 of Example 27 with N,N,N′-trimethylethylenediamine.

¹HNMR (DMSO-d6, 400 MHz) δ: 9.64 (1H, s), 8.80 (1H, s), 8.71 (1H, s), 8.56 (1H, d), 8.26 (1H, dd), 7.70 (1H, s), 7.57 (1H, d), 7.48 (1H, d), 7.30 (2H, m) 7.20 (1H, s), 7.07 (2H, t), 2.95 (2H, t), 2.64 (3H, s), 2.40 (2H, t), 2.26 (3H, s), 2.16 (6H, s).

LC-MS (ESI): 444.2 (M+H)⁺.

### Example 80

### Preparation of 1-(2-{4-[(2-dimethylamino-ethyl)-methyl-amino]-3-methoxy-phenylamino}-pyrimidin-4-yl)-1H-indole-3-carboxamide (Compound 80)

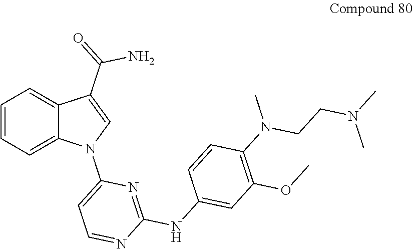 [SMILES: COc1cc(Nc2nccc(-n3cc(C(N)=O)c4ccccc43)n2)ccc1N(C)CCN(C)C]

1-(2-{4-[(2-Dimethylamino-ethyl)-methyl-amino]-3-methoxy-phenylamino}-pyrimidin-4-yl)-1H-indole-3-carboxamide was obtained in accordance with the same preparation method of Example 27 except for replacing the 4-fluoronitrobenzene in Step 1 of Example 27 with 2-fluoro-5-nitroanisole and replacing the N-Boc-piperazine in Step 1 of Example 27 with N,N,N′-trimethylethylenediamine.

¹HNMR (DMSO-d6, 400 MHz) δ: 9.65 (1H, s), 8.81 (1H, s), 8.70 (1H, s), 8.55 (1H, d), 8.26 (1H, dd), 7.71 (1H, s), 7.39 (1H, d), 7.30-7.20 (4H, m), 7.08 (1H, d), 6.88 (1H, d), 3.75 (3H, s), 3.08 (2H, t), 2.71 (3H, s), 2.39 (2H, t), 2.15 (6H, s).

LC-MS (ESI): 460.2 (M+H)⁺.

### Example 81

### Preparation of 1-(2-{4-[(2-dimethylamino-ethyl)-methyl-amino]-3-isopropoxy-phenylamino}-pyrimidin-4-yl)-1H-indole-3-carboxamide (Compound 81)

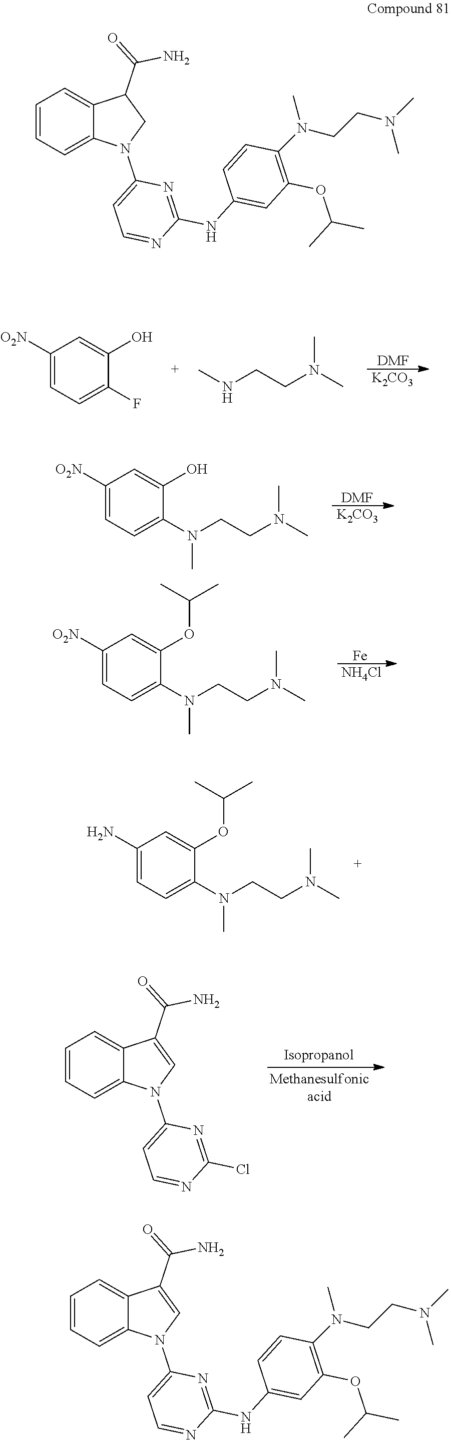

### Step 1: Preparation of 2-[(2-dimethylamino-ethyl)-methyl-amino]-5-nitro-phenol

2-Fluoro-5-nitrophenol (3.2 g, 0.02 mol) and N,N,N′-trimethylethylenediamine (6.2 g, 0.06 mol) were dissolved in DMF (25 ml). Potassium carbonate (8.3 g, 0.06 mol) was added at room temperature, and the resulting solution was heated to 90° C. and reacted for 8 hours. TLC showed that the reaction was substantially completed. The reaction solution was cooled to room temperature, slowly poured into 100 ml of water, and extracted with ethyl acetate (60 ml×3). The organic phase was washed with saturated NaCl solution twice, dried over anhydrous sodium sulfate, filtrated, and concentrated under reduced pressure to obtain the crude product 2-[(2-dimethylamino-ethyl)-methyl-amino]-5-nitro-phenol (2.2 g).

### Step 2: Preparation of N-(2-isopropyl-4-nitro-phenyl)-N,N,N′-trimethyl-ethane-1,2-diamine

The product 2-[(2-dimethylamino-ethyl)-methyl-amino]-5-nitro-phenol (1 g, 4 mmol) obtained in Step 2 and bromoisopropane (740 mg, 6 mmol) were dissolved in DMF (10 ml). Potassium carbonate (1.6 g, 12 mmol) and a catalytic amount of potassium iodide were added at room temperature, and the resulting mixture was heated to 100° C. and reacted for 62 hours. TLC showed that the reaction was substantially completed. The reaction solution was cooled to room temperature, slowly poured into 50 ml of water, and extracted with ethyl acetate (50 ml×2). The organic phase was washed with saturated NaCl solution twice, dried over anhydrous sodium sulfate, filtrated, and concentrated under reduced pressure to obtain N-(2-isopropyl-4-nitro-phenyl)-N,N′,N′-trimethyl-ethane-1,2-diamine (1.2 g, oil). The product was used directly in the next step without purification.

### Step 3: Preparation of N-methyl-N-[2-(4-methyl-piperazin-1-yl)-ethyl]-benzene-1,4-diamine

The product N-(2-isopropyl-4-nitro-phenyl)-N,N′,N′-trimethyl-ethane-1,2-diamine (1.2 g, 4.1 mmol) obtained in Step 2, reduced iron powder (918 mg, 16.4 mmol) and ammonium chloride (1.5 g, 28.7 mmol) were added into ethanol (50 ml)/water (12.5 ml). The resulting mixture was heated to 90° C. and reacted for 1 hour. The reaction solution was cooled to room temperature, slowly poured into saturated aqueous sodium bicarbonate solution (150 ml), and extracted with ethyl acetate (100 ml×2). The organic phase was washed with saturated NaCl solution twice, dried over anhydrous sodium sulfate, filtrated, and concentrated under reduced pressure to obtain N-methyl-N-[2-(4-methyl-piperazin-1-yl)-ethyl]-benzene-1,4-diamine (600 mg, oil). The product was used directly in the next step without purification.

### Step 4: Preparation of 1-(2-{4-[(2-dimethylamino-ethyl)-methyl-amino]-3-isopropoxy-phenylamino}-pyrimidin-4-yl)-1H-indole-3-carboxamide

1-(2-{4-[(2-Dimethylamino-ethyl)-methyl-amino]-3-isopropoxy-phenylamino}-pyrimidin-4-yl)-1H-indole-3-carboxamide was obtained in accordance with the same preparation method of Step 4 of Example 1 except for replacing the 3-bromo-4-fluoroaniline in Step 4 of Example 1 with N-methyl-N-[2-(4-methyl-piperazin-1-yl)-ethyl]-benzene-1,4-diamine (prepared in Step 3).

¹HNMR (DMSO-d6, 400 MHz) δ: 9.60 (1H, s), 8.78 (1H, s), 8.69 (1H, s), 8.56 (1H, d), 8.26 (1H, dd), 7.69 (1H, s), 7.36 (1H, d), 7.30 (2H, br), 7.22 (2H, m), 7.06 (1H, d), 6.88 (1H, d), 3.18 (1H, d), 3.09 (2H, t), 2.72 (3H, s), 2.40 (2H, t), 2.14 (6H, s).

LC-MS (ESI): 488.2 (M+H)⁺.

### Example 82

### Preparation of 1-(2-{3-chloro-4-[(2-dimethylamino-ethyl)-methyl-amino]-phenylamino}-pyrimidin-4-yl)-1H-indole-3-carboxamide (Compound 82)

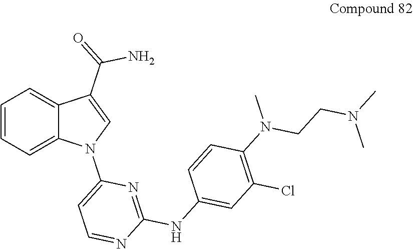 [SMILES: CN(C)CCN(C)c1ccc(Nc2nccc(-n3cc(C(N)=O)c4ccccc43)n2)cc1Cl]

1-(2-{3-Chloro-4-[(2-dimethylamino-ethyl)-methyl-amino]-phenylamino}-pyrimidin-4-yl)-1H-indole-3-carboxamide was obtained in accordance with the same preparation method of Example 27 except for replacing the 4-fluoronitrobenzene in Step 1 of Example 27 with 3-chloro-4-fluoronitrobenzene and replacing the N-Boc-piperazine in Step 1 of Example 27 with N,N,N′-trimethylethylenediamine.

¹HNMR (DMSO-d6, 400 MHz) δ: 9.85 (1H, s), 8.80 (1H, s), 8.73 (1H, s), 8.61 (1H, d), 8.26 (1H, dd), 7.96 (1H, s), 7.70 (1H, s), 7.60 (1H, dd), 7.32 (2H, m), 7.22 (2H, d), 7.13 (1H, d), 3.07 (2H, t), 2.73 (3H, s), 2.45 (2H, t), 2.17 (6H, s).

LC-MS (ESI): 464.2 (M+H)⁺.

### Example 83

### Preparation of 1-(2-{3-chloro-4-[(3-dimethylamino-propyl)-methyl-amino]-phenylamino}-pyrimidin-4-yl)-1H-indole-3-carboxamide (Compound 83)

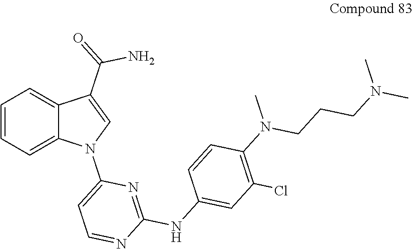 [SMILES: CN(C)CCCN(C)c1ccc(Nc2nccc(-n3cc(C(N)=O)c4ccccc43)n2)cc1Cl]

1-(2-{3-Chloro-4-[(3-dimethylamino-propyl)-methyl-amino]-phenylamino}-pyrimidin-4-yl)-1H-indole-3-carboxamide was obtained in accordance with the same preparation method of Example 27 except for replacing the 4-fluoronitrobenzene in Step 1 of Example 27 with 3-chloro-4-fluoronitrobenzene and replacing the N-Boc-piperazine in Step 1 of Example 27 with N,N,N′-trimethyl-1,3-propanediamine.

¹HNMR (DMSO-d6, 400 MHz) δ: 9.84 (1H, s), 8.79 (1H, s), 8.72 (1H, s), 8.60 (1H, d), 8.26 (1H, dd), 7.95 (1H, s), 7.68 (1H, d), 7.59 (1H, dd), 7.32 (2H, m), 7.21 (2H, d), 7.13 (1H, d), 2.97 (2H, t), 2.68 (3H, s), 2.27 (2H, t), 2.12 (6H, s), 1.62 (2H, m).

LC-MS (ESI): 478.2 (M+H)⁺.

### Example 84

### Preparation of 1-(2-{4-[(2-dimethylamino-ethyl)-methyl-amino]-phenylamino}-5-fluoro-pyrimidin-4-yl)-1H-indole-3-carboxamide (Compound 84)

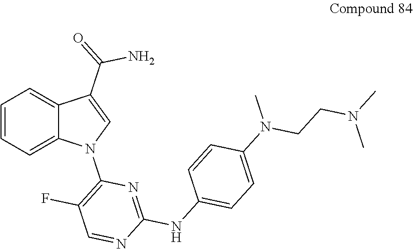 [SMILES: CN(C)CCN(C)c1ccc(Nc2ncc(F)c(-n3cc(C(N)=O)c4ccccc43)n2)cc1]

1-(2-{4-[(2-Dimethylamino-ethyl)-methyl-amino]-phenylamino}-5-fluoro-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with N-(2-dimethylamino-ethyl)-N-methyl-benzene-1,4-diamine (prepared in Example 77).

¹HNMR (DMSO-d6, 400 MHz) δ: 9.56 (s, 1H), 8.67-8.68 (d, 1H), 8.54 (d, 1H), 8.27-8.30 (m, 1H), 8.23 (br, 1H), 7.87 (br, 1H), 7.46-7.48 (d, 2H), 7.26-7.32 (m, 2H), 7.17 (br, 1H), 6.70-6.73 (d, 2H), 3.44-3.47 (t, 2H), 2.88 (s, 3H), 2.60 (t, 2H), 2.36 (s, 6H).

LC-MS (ESI): 448.2 (M+H)⁺.

### Example 85

### Preparation of 1-(5-chloro-2-{4-[(2-dimethylamino-ethyl)-methyl-amino]-phenylamino}-pyrimidin-4-yl)-1H-indole-3-carboxamide (Compound 85

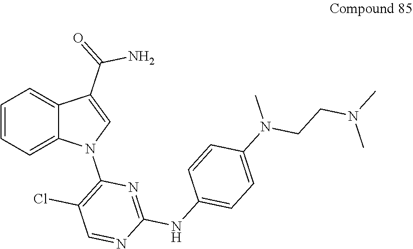 [SMILES: CN(C)CCN(C)c1ccc(Nc2ncc(Cl)c(-n3cc(C(N)=O)c4ccccc43)n2)cc1]

1-(5-Chloro-2-{4-[(2-dimethylamino-ethyl)-methyl-amino]-phenylamino}-pyrimidin-4-yl)-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with N-(2-dimethylamino-ethyl)-N-methyl-benzene-1,4-diamine (prepared in Example 77).

¹HNMR (DMSO-d6, 400 MHz) δ: 9.81 (s, 1H), 8.71 (s, 1H), 8.51 (s, 1H), 8.26-8.28 (m, 1H), 7.72-7.76 (m, 2H), 7.45-7.47 (d, 2H), 7.25-7.29 (m, 2H), 7.13 (br, 1H), 6.64-6.66 (d, 2H), 3.38-3.42 (t, 2H), 2.85 (s, 3H), 2.43-2.46 (t, 2H), 2.24 (s, 6H).

LC-MS (ESI): 464.2 (M+H)⁺.

### Example 86

### Preparation of 1-(2-{4-[(2-dimethylamino-ethyl)-methyl-amino]-3-methoxy-phenylamino}-5-fluoro-pyrimidin-4-yl)-1H-indole-3-carboxamide (Compound 86)

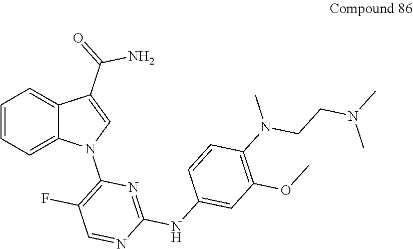 [SMILES: COc1cc(Nc2ncc(F)c(-n3cc(C(N)=O)c4ccccc43)n2)ccc1N(C)CCN(C)C]

1-(2-{4-[(2-Dimethylamino-ethyl)-methyl-amino]-3-methoxy-phenylamino}-5-fluoro-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with N¹-(2-dimethylamino-ethyl)-2-methoxy-N¹-methyl-benzene-1,4-diamine (prepared in Example 80).

¹HNMR (DMSO-d6, 400 MHz) δ: 9.76 (s, 1H), 8.74-8.75 (d, 1H), 8.56 (s, 1H), 8.28-8.30 (m, 1H), 8.22 (br, 1H), 7.82 (br, 1H), 7.41 (s, 1H), 7.29-7.31 (m, 2H), 7.14-7.23 (m, 2H), 6.88-6.90 (d, 1H), 3.70 (s, 3H), 3.08-3.11 (t, 2H), 2.68 (m, 5H), 2.41 (s, 6H).

LC-MS (ESI): 478.2 (M+H)⁺.

### Example 87

### Preparation of 1-(2-{4-[(2-dimethylamino-ethyl)-methyl-amino]-3-methoxy-phenylamino}-5-chloro-pyrimidin-4-yl)-1H-indole-3-carboxamide (Compound 87)

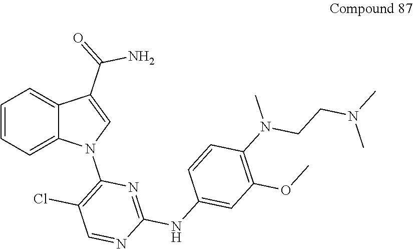 [SMILES: COc1cc(Nc2ncc(Cl)c(-n3cc(C(N)=O)c4ccccc43)n2)ccc1N(C)CCN(C)C]

1-(2-{4-[(2-Dimethylamino-ethyl)-methyl-amino]-3-methoxy-phenylamino}-5-chloro-pyrimidin-4-yl)-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with N¹-(2-dimethylamino-ethyl)-2-methoxy-N¹-methyl-benzene-1,4-diamine (prepared in Example 80).

¹HNMR (DMSO-d6, 400 MHz) δ: 10.07 (s, 1H), 8.80 (s, 1H), 8.54 (s, 1H), 8.27-8.29 (m, 1H), 7.74-7.76 (m, 2H), 7.50 (br, 1H), 7.26-7.30 (m, 2H), 7.18-7.20 (m, 1H), 7.13 (br, 1H), 6.91-6.94 (d, 1H), 3.63 (s, 3H), 3.12-3.14 (m, 2H), 3.06-3.08 (m, 2H), 2.71 (s, 6H), 2.66 (s, 3H).

LC-MS (ESI): 494.2 (M+H)⁺.

### Example 88

### Preparation of 1-(2-{4-[(2-dimethylamino-ethyl)-methyl-amino]-3-methyl-phenylamino}-5-fluoro-pyrimidin-4-yl)-1H-indole-3-carboxamide (Compound 88)

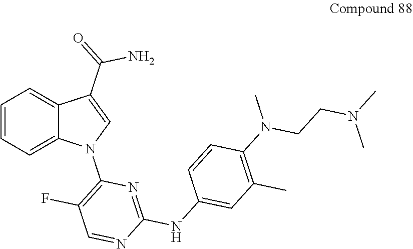 [SMILES: Cc1cc(Nc2ncc(F)c(-n3cc(C(N)=O)c4ccccc43)n2)ccc1N(C)CCN(C)C]

1-(2-{4-[(2-Dimethylamino-ethyl)-methyl-amino]-3-methyl-phenylamino}-5-fluoro-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with N¹-(2-dimethylamino-ethyl)-2-methyl-N¹-methyl-benzene-1,4-diamine (prepared in Example 79).

¹HNMR (DMSO-d6, 400 MHz) δ: 9.75 (s, 1H), 8.73-8.74 (d, 1H), 8.56 (d, 1H), 8.26-8.31 (m, 2H), 7.88 (br, 1H), 7.56 (d, 1H), 7.44-7.46 (dd, 1H), 7.30-7.34 (m, 2H), 7.17 (br, 1H), 7.05-7.07 (d, 1H), 2.98-3.01 (t, 2H), 2.60 (m, 5H), 2.32 (s, 6H), 2.22 (s, 3H).

LC-MS (ESI): 462.2 (M+H)⁺.

### Example 89

### Preparation of 1-(5-chloro-2-{4-[(2-dimethylamino-ethyl)-methyl-amino]-3-methyl-phenylamino}-pyrimidin-4-yl)-1H-indole-3-carboxamide (Compound 89)

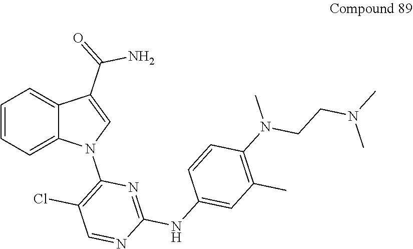 [SMILES: Cc1cc(Nc2ncc(Cl)c(-n3cc(C(N)=O)c4ccccc43)n2)ccc1N(C)CCN(C)C]

1-(5-Chloro-2-{4-[(2-dimethylamino-ethyl)-methyl-amino]-3-methyl-phenylamino}-pyrimidin-4-yl)-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with N¹-(2-dimethylamino-ethyl)-2-methyl-N¹-methyl-benzene-1,4-diamine (prepared in Example 79).

¹HNMR (DMSO-d6, 400 MHz) δ: 10.02 (s, 1H), 8.78 (s, 1H), 8.55 (s, 1H), 8.27-8.29 (m, 1H), 7.78-7.80 (m, 2H), 7.57 (s, 1H), 7.44-7.47 (m, 1H), 7.27-7.33 (m, 2H), 7.14 (br, 1H), 7.04-7.06 (d, 1H), 3.07-3.12 (t, 2H), 2.86-2.89 (t, 2H), 2.57 (s, 3H), 2.52 (s, 6H), 2.19 (s, 3H).

LC-MS (ESI): 478.1 (M+H)⁺.

### Example 90

### Preparation of 1-(2-{4-[methyl-(2-pyrrolidin-1-ylethyl)-amino]-phenylamino}-pyrimidin-4-yl)-1H-indole-3-carboxamide (Compound 90)

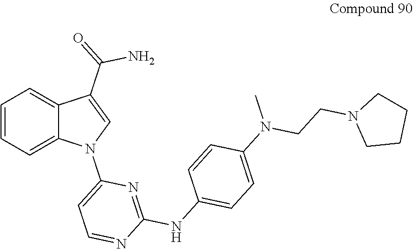 [SMILES: CN(CCN1CCCC1)c1ccc(Nc2nccc(-n3cc(C(N)=O)c4ccccc43)n2)cc1]

1-(2-{4-[Methyl-(2-pyrrolidin-1-ylethyl)-amino]-phenylamino}-pyrimidin-4-yl)-1H-indole-3-carboxamide was obtained in accordance with the same preparation method of Example 73 except for replacing the N-methylpiperazine in Step 3 of Example 73 with pyrrolidine.

¹HNMR (DMSO-d6, 400 MHz) δ: 1.95 (4H, br), 2.93 (3H, s), 3.03-3.11 (4H, m), 3.54 (2H, br), 3.72 (2H, t), 6.88 (2H, d), 7.06 (1H, d), 7.17 (1H, br), 7.25-7.31 (2H, m), 7.56 (2H, d), 7.76 (1H, br), 8.24-8.27 (1H, m), 8.51 (1H, d), 8.70 (1H, br), 8.87 (1H, s), 9.53 (1H, s), 10.26 (1H, br), 11.10 (1H, s).

LC-MS (ESI): 456.2 (M+H)⁺.

### Example 91

### Preparation of 1-(2-((3-methoxy-4-(methyl(2-(pyrrolidin-1-yl)ethyl)amino)phenylamino)pyrimidin-4-yl)-1H-indole-3-carboxamide (Compound 91)

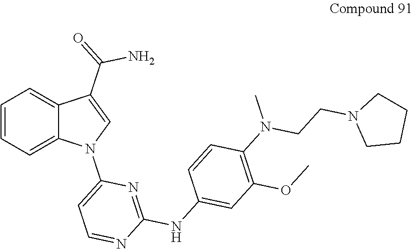 [SMILES: COc1cc(Nc2nccc(-n3cc(C(N)=O)c4ccccc43)n2)ccc1N(C)CCN1CCCC1]

1-(2-((3-Methoxy-4-(methyl(2-(pyrrolidin-1-yl)ethyl)amino)phenylamino)pyrimidin-4-yl)-1H-indole-3-carboxamide was obtained in accordance with the same preparation method of Example 73 except for replacing the 4-fluoronitrobenzene in Step 1 of Example 73 with 2-fluoro-5-nitroanisole and replacing the N-methylpiperazine in Step 3 of Example 73 with pyrrolidine.

¹HNMR (DMSO-d6, 400 MHz) δ: 9.64 (1H, s), 8.80 (1H, s), 8.70 (1H, s), 8.57 (1H, d), 8.26 (1H, dd), 7.69 (1H, s), 7.38 (1H, s), 7.30-7.19 (4H, br), 7.08 (1H, d), 6.89 (1H, d), 3.75 (3H, s), 3.12 (2H, t), 2.72 (3H, s), 2.56 (2H, t), 2.43 (4H, br), 1.66 (4H, br).

LC-MS (ESI): 486.2 (M+H)⁺.

### Example 92

### Preparation of 1-(2-{3-fluoro-4-[methyl-(2-pyrrolidin-1-yl-ethyl)-amino]-phenylamino}-pyrimidin-4-yl)-1H-indole-3-carboxamide (Compound 92)

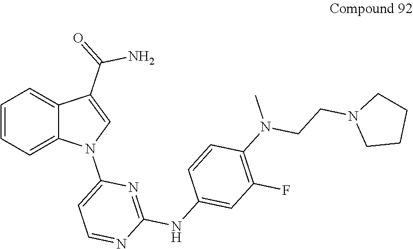 [SMILES: CN(CCN1CCCC1)c1ccc(Nc2nccc(-n3cc(C(N)=O)c4ccccc43)n2)cc1F]

1-(2-{3-Fluoro-4-[methyl-(2-pyrrolidin-1-yl-ethyl)-amino]-phenylamino}-pyrimidin-4-yl)-1H-indole-3-carboxamide was obtained in accordance with the same preparation method of Example 73 except for replacing the 4-fluoronitrobenzene in Step 1 of Example 73 with 3,4-difluoronitrobenzene and replacing the N-methylpiperazine in Step 3 of Example 73 with pyrrolidine.

¹HNMR (DMSO-d6, 400 MHz) δ: 9.77 (s, 1H), 8.77 (d, 1H), 8.71 (br, 1H), 8.57-8.58 (d, 1H), 8.25-8.26 (m, 1H), 7.66-7.69 (m, 2H), 7.36-7.38 (d, 1H), 7.30 (m, 2H), 7.17 (br, 1H), 7.08-7.10 (m, 1H), 6.96-7.01 (t, 1H), 3.19 (m, 2H), 2.79 (s, 3H), 2.59 (m, 2H), 2.45 (m, 4H), 1.66 (m, 4H).

LC-MS (ESI): 474.2 (M+H)⁺.

### Example 93

### Preparation of 1-(5-fluoro-2-{4-[methyl-(2-pyrrolidin-1-yl-ethyl)-amino]-phenylamino}-pyrimidin-4-yl)-1H-indole-3-carboxamide (Compound 93

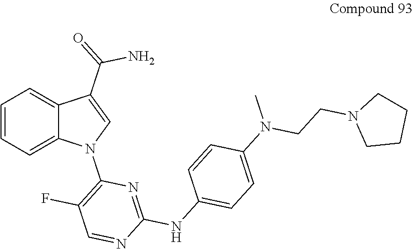 [SMILES: CN(CCN1CCCC1)c1ccc(Nc2ncc(F)c(-n3cc(C(N)=O)c4ccccc43)n2)cc1]

1-(5-Fluoro-2-{4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with N-methyl-N-(2-pyrrolidin-1-yl-ethyl)-benzene-1,4-diamine (prepared in Example 90).

¹HNMR (DMSO-d6, 400 MHz) δ: 9.62 (s, 1H), 8.73-8.74 (d, 1H), 8.59 (d, 1H), 8.33-8.35 (m, 1H), 8.28 (br, 1H), 7.91 (br, 1H), 7.54-7.56 (d, 2H), 7.34-7.38 (m, 2H), 7.20 (br, 1H), 6.81-6.83 (d, 2H), 3.60-3.63 (t, 2H), 3.03 (m, 4H), 2.95 (s, 3H), 2.50 (m, 2H), 1.88 (m, 4H).

LC-MS (ESI): 474.2 (M+H)⁺.

### Example 94

### Preparation of 1-(5-chloro-2-{4-[methyl-(2-pyrrolidin-1-yl-ethyl)-amino]-phenylamino}-pyrimidin-4-yl)-1H-indole-3-carboxamide (Compound 94)

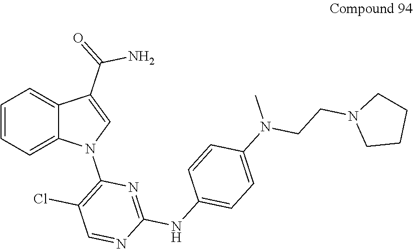 [SMILES: CN(CCN1CCCC1)c1ccc(Nc2ncc(Cl)c(-n3cc(C(N)=O)c4ccccc43)n2)cc1]

1-(5-Chloro-2-{4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with N-methyl-N-(2-pyrrolidin-1-yl-ethyl)-benzene-1,4-diamine (prepared in Example 90).

¹HNMR (DMSO-d6, 400 MHz) δ: 9.84 (s, 1H), 8.72 (s, 1H), 8.50 (s, 1H), 8.26-8.28 (m, 1H), 7.72-0.74 (m, 2H), 7.48-7.50 (d, 2H), 7.26-7.32 (m, 2H), 7.13 (br, 1H), 6.71-6.74 (m, 2H), 3.54 (m, 2H), 2.95 (m, 4H), 2.87 (s, 3H), 2.51 (m, 2H), 1.81 (m, 4H).

LC-MS (ESI): 490.1 (M+H)⁺.

### Example 95

### Preparation of 1-(5-fluoro-2-{3-fluoro-4-[methyl-(2-pyrrolidin-1-yl-ethyl)-amino]-phenylamino}-pyrimidin-4-yl)-1H-indole-3-carboxamide (Compound 95)

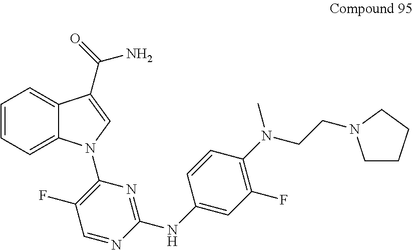 [SMILES: CN(CCN1CCCC1)c1ccc(Nc2ncc(F)c(-n3cc(C(N)=O)c4ccccc43)n2)cc1F]

1-(5-Fluoro-2-{3-fluoro-4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 2-fluoro-N′-methyl-N′-(2-pyrrolidin-1-yl-ethyl)-benzene-1,4-diamine (prepared in Example 92).

¹HNMR (DMSO-d6, 400 MHz) δ: 9.93 (s, 1H), 8.77-8.78 (d, 1H), 8.56 (d, 1H), 8.28-8.31 (m, 2H), 7.89 (br, 1H), 7.64-7.68 (m, 1H), 7.31-7.37 (m, 3H), 7.19 (br, 1H), 6.98-7.02 (t, 1H), 3.19-3.32 (t, 2H), 2.76 (s, 3H), 2.67 (m, 6H), 1.72 (m, 4H).

LC-MS (ESI): 492.2 (M+H)⁺.

### Example 96

### Preparation of 1-(5-fluoro-2-{3-methyl-4-[methyl-(2-pyrrolidin-1-yl-ethyl)-amino]-phenylamino}-pyrimidin-4-yl)-1H-indole-3-carboxamide (Compound 96)

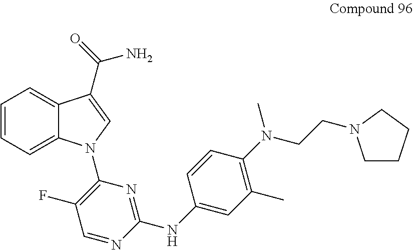 [SMILES: Cc1cc(Nc2ncc(F)c(-n3cc(C(N)=O)c4ccccc43)n2)ccc1N(C)CCN1CCCC1]

1-(5-Fluoro-2-{3-methyl-4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 2-methyl-N¹-methyl-N¹-(2-pyrrolidin-1-yl-ethyl)-benzene-1,4-diamine (prepared in accordance with Example 73).

¹HNMR (DMSO-d6, 400 MHz) δ: 9.81 (s, 1H), 8.75-8.76 (d, 1H), 8.56 (d, 1H), 8.29-8.31 (m, 2H), 7.89 (br, 1H), 7.60 (m, 1H), 7.47-7.49 (m, 1H), 7.31-7.34 (m, 2H), 7.19 (br, 1H), 7.08-7.11 (d, 1H), 3.15-3.42 (m, 8H), 2.60 (s, 3H), 2.25 (s, 3H), 1.90 (m, 4H).

LC-MS (ESI): 488.2 (M+H)⁺.

### Example 97

### Preparation of 1-(5-chloro-2-{3-methyl-4-[methyl-(2-pyrrolidin-1-yl-ethyl)-amino]-phenylamino}-pyrimidin-4-yl)-1H-indole-3-carboxamide (Compound 97)

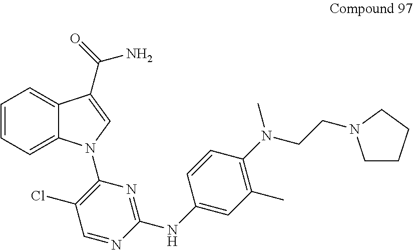 [SMILES: Cc1cc(Nc2ncc(Cl)c(-n3cc(C(N)=O)c4ccccc43)n2)ccc1N(C)CCN1CCCC1]

1-(5-Chloro-2-{3-methyl-4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with 2-methyl-N′-methyl-N′-(2-pyrrolidin-1-yl-ethyl)-benzene-1,4-diamine (prepared in accordance with Example 73).

¹HNMR (DMSO-d6, 400 MHz) δ: 10.04 (s, 1H), 8.79 (s, 1H), 8.53 (s, 1H), 8.27-8.29 (d, 1H), 7.78-7.81 (m, 2H), 7.58 (s, 1H), 7.45-7.47 (m, 1H), 7.30-7.34 (m, 2H), 7.15 (br, 1H), 7.05-7.07 (d, 1H), 3.32 (m, 2H), 3.18-3.26 (m, 6H), 2.57 (s, 3H), 2.20 (s, 3H), 1.90 (m, 4H).

LC-MS (ESI): 504.2 (M+H)⁺.

### Example 98

### Preparation of 1-(5-fluoro-2-{3-methoxy-4-[methyl-(2-pyrrolidin-1-yl-ethyl)-amino]-phenylamino}-pyrimidin-4-yl)-1H-indole-3-carboxamide (Compound 98)

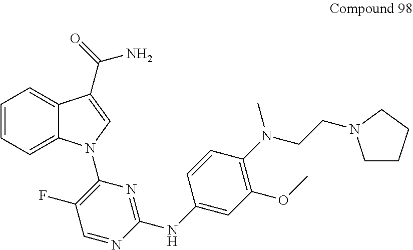 [SMILES: COc1cc(Nc2ncc(F)c(-n3cc(C(N)=O)c4ccccc43)n2)ccc1N(C)CCN1CCCC1]

1-(5-Fluoro-2-{3-methoxy-4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 2-methoxy-N′-methyl-N′-(2-pyrrolidin-1-yl-ethyl)-benzene-1,4-diamine (prepared in Example 91).

¹HNMR (DMSO-d6, 400 MHz) δ: 9.82 (s, 1H), 8.75-8.76 (d, 1H), 8.57 (d, 1H), 8.28-8.30 (m, 1H), 8.22 (br, 1H), 7.88 (br, 1H), 7.74-7.75 (m, 1H), 7.30-7.33 (m, 2H), 7.23-7.25 (m, 1H), 7.18 (br, 1H), 6.93-6.95 (d, 1H), 3.71 (s, 3H), 3.17-3.19 (m, 2H), 3.01 (m, 6H), 2.69 (s, 3H), 1.87 (m, 4H).

LC-MS (ESI): 504.2 (M+H)⁺.

### Example 99

### Preparation of 1-(5-chloro-2-{3-methoxy-4-[methyl-(2-pyrrolidin-1-yl-ethyl)-amino]-phenylamino}-pyrimidin-4-yl)-1H-indole-3-carboxamide (Compound 99

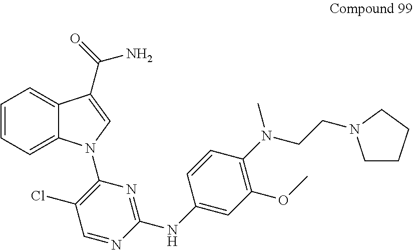 [SMILES: COc1cc(Nc2ncc(Cl)c(-n3cc(C(N)=O)c4ccccc43)n2)ccc1N(C)CCN1CCCC1]

1-(5-Chloro-2-{3-methoxy-4-[methyl-(2-pyrrolidin-1-yl-ethyl)-amino]-phenylamino}-pyrimidin-4-yl)-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with 2-methoxy-N¹-methyl-N¹-(2-pyrrolidin-1-yl-ethyl)-benzene-1,4-diamine (prepared in Example 91).

¹HNMR (DMSO-d6, 400 MHz) δ: 10.08 (s, 1H), 8.80 (s, 1H), 8.54 (s, 1H), 8.27-8.29 (m, 1H), 7.73-7.76 (m, 2H), 7.51 (s, 1H), 7.26-7.32 (m, 2H), 7.15-7.20 (m, 2H), 6.93-6.95 (d, 1H), 3.64 (s, 3H), 3.31 (m, 2H), 3.18-3.26 (m, 6H), 2.66 (s, 3H), 1.92 (m, 4H).

LC-MS (ESI): 520.2 (M+H)⁺.

### Example 100

### Preparation of 1-(2-{3-methoxy-4-[methyl-(2-pyrrolidin-1-yl-ethyl)-amino]-phenylamino}-5-methyl-pyrimidin-4-yl)-1H-indole-3-carboxamide (Compound 100)

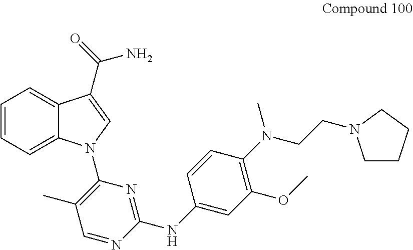 [SMILES: COc1cc(Nc2ncc(C)c(-n3cc(C(N)=O)c4ccccc43)n2)ccc1N(C)CCN1CCCC1]

1-(2-{3-methoxy-4-[methyl-(2-pyrrolidin-1-yl-ethyl)-amino]-phenylamino}-5-methyl-pyrimidin-4-yl)-1H-indole-3-carboxamide was obtained in accordance with the same preparation method of Example 37 except for replacing the tert-butyl 4-(4-amino-phenyl)-piperazine-1-carboxylate in Example 37 with 2-methoxy-N′-methyl-N′-(2-pyrrolidin-1-yl-ethyl)-benzene-1,4-diamine (prepared in Example 91).

¹HNMR (DMSO-d6, 400 MHz) δ: 9.70 (s, 1H), 8.60 (s, 1H), 8.47 (s, 1H), 8.29-8.30 (m, 1H), 7.74 (br, 1H), 7.67-7.68 (m, 1H), 7.54 (s, 1H), 7.25-7.27 (m, 2H), 7.18-7.20 (m, 1H), 7.10 (br, 1H), 6.88-6.86 (d, 1H), 3.61 (s, 3H), 3.13 (m, 2H), 3.01 (m, 6H), 2.65 (s, 3H), 2.20 (s, 3H), 1.83 (m, 4H).

LC-MS (ESI): 500.2 (M+H)⁺.

### Example 101

### Preparation of 1-[2-(4-piperazin-1-yl-phenylamino)-pyrimidin-4-yl]-1H-indole-3-carbo methylamide (Compound 101)

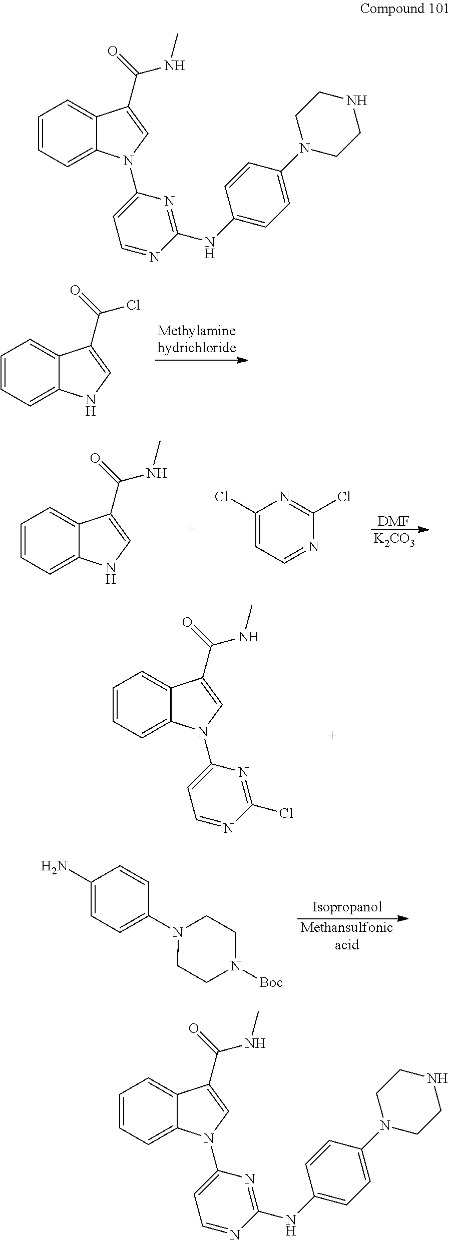

### Step 1: Preparation of 1H-indole-3-carboxmethylamide

Methylamine hydrochloride (1.34 g, 0.02 mol) and triethylamine (3 g, 0.03 mol) were dissolved in 30 ml of dichloromethane, and the resulting solution was cooled in an ice water bath. 1H-Indole-3-carbonyl chloride (1.79 g, 0.01 mol, dissolved in 20 ml of dichloromethane) (prepared in Step 1 of Example 1) was slowly added dropwise, and the reaction solution was warmed to room temperature and reacted for 2 hours after completion of the addition. TLC showed that the reaction was completed. The reaction solution was slowly poured into water, and extracted with dichloromethane (80 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol) to obtain 1H-indole-3-carboxmethylamide (2.1 g, yellow solid).

### Step 2: Preparation of 1-(2-chloro-pyrimidin-4-yl)-1H-indole-3-carboxmethylamide

1-(2-Chloro-pyrimidin-4-yl)-1H-indole-3-carboxmethylamide was obtained in accordance with the same preparation method of Step 3 of Example 1 except for replacing the 1H-indole-3-carboxamide in Step 3 of Example 1 with 1H-indole-3-carboxmethylamide (prepared in Step 1).

### Step 3: Preparation of 1-[2-(4-piperazin-1-yl-phenylamino)-pyrimidin-4-yl]-1H-indole-3-carboxmethylamide

1-(2-Chloro-pyrimidin-4-yl)-1H-indole-3-carboxmethylamide (100 mg, 0.35 mmol) obtained in Step 2, tert-butyl 4-(4-amino-phenyl)-piperazine-1-carboxylate (96 mg, 0.35 mmol) and methanesulfonic acid (100 mg, 1.05 mmol) were dispersed in 10 ml of isopropanol, and reacted under reflux for 12 hours. TLC showed that the reaction was substantially completed. The reaction solution was cooled, then 10 ml of methyl tert-butyl ether was added. The solution was stirred at room temperature for 10 minutes and then filtrated, and the resulting solid was washed with a small amount of methyl tert-butyl ether. The resulting solid was dissolved in 50 ml of dichloromethane/methanol (dichloromethane:methanol=5:1), to which 10 ml of aqueous sodium hydroxide solution (0.5 mol/L) was added, and the resulting solution was extracted with dichloromethane. The organic phase was washed with saturated NaCl solution twice, dried over anhydrous sodium sulfate, filtrated and concentrated under reduced pressure. The resulting residues were purified by column chromatography (eluent: dichloromethane/methanol) to obtain 1-[2-(4-piperazin-1-yl-phenylamino)-pyrimidin-4-yl]-1H-indole-3-carboxmethylamide (35 mg, solid).

¹HNMR (DMSO-d6, 400 MHz) δ: 9.57 (s, 1H), 8.72 (s, 1H), 8.68 (br, 1H), 8.51-8.52 (d, 1H), 8.21-8.23 (m, 2H), 7.56-7.58 (d, 2H), 7.28-7.31 (m, 2H), 7.03-7.05 (d, 1H), 6.93-6.96 (d, 2H), 3.06-3.08 (m, 4H), 2.89-2.94 (m, 4H), 2.82-2.83 (d, 3H).

LC-MS (ESI): 428.2 (M+H)⁺.

### Example 102

### Preparation of 1-[2-(2-chloro-4-piperazin-1-yl-phenylamino)-pyrimidin-4-yl]-1H-indole-3-carboxmethylamide (Compound 102)

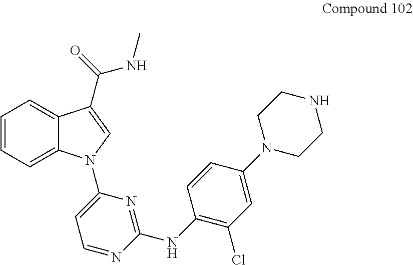 [SMILES: CNC(=O)c1cn(-c2ccnc(Nc3ccc(N4CCNCC4)cc3Cl)n2)c2ccccc12]

1-[2-(2-Chloro-4-piperazin-1-yl-phenylamino)-pyrimidin-4-yl]-1H-indole-3-carboxmethylamide was obtained in accordance with the same preparation method of Example 101 except for replacing the tert-butyl 4-(4-amino-phenyl)-piperazine-1-carboxylate in Step 3 of Example 101 with tert-butyl 4-(4-amino-3-chloro-phenyl)-piperazine-1-carboxylate (prepared in accordance with Example 27).

¹HNMR (DMSO-d6, 400 MHz) δ: 9.18 (s, 1H), 9.67 (s, 1H), 8.45-8.46 (d, 1H), 8.15-8.18 (m, 2H), 7.35-7.37 (d, 1H), 7.21-7.25 (m, 1H), 6.93-7.09 (m, 4H), 3.31-3.37 (m, 4H), 2.88-2.91 (m, 4H), 2.81-2.82 (d, 3H).

LC-MS (ESI): 462.1 (M+H)⁺.

### Example 103

### Preparation of 1-[2-(3-chloro-4-piperazin-1-yl-phenylamino)-pyrimidin-4-yl]-1H-indole-3-carboxmethylamide (Compound 103)

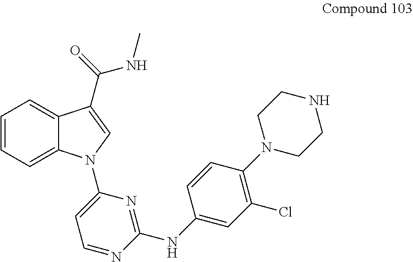 [SMILES: CNC(=O)c1cn(-c2ccnc(Nc3ccc(N4CCNCC4)c(Cl)c3)n2)c2ccccc12]

1-[2-(3-Chloro-4-piperazin-1-yl-phenylamino)-pyrimidin-4-yl]-1H-indole-3-carboxmethylamide was obtained in accordance with the same preparation method of Example 101 except for replacing the tert-butyl 4-(4-amino-phenyl)-piperazine-1-carboxylate in Step 3 of Example 101 with tert-butyl 4-(4-amino-2-chloro-phenyl)-piperazine-1-carboxylate (prepared in accordance with Example 27).

¹HNMR (DMSO-d6, 400 MHz) δ: 9.86 (s, 1H), 8.71-8.73 (m, 2H), 8.59-8.60 (d, 1H), 8.19-8.24 (m, 2H), 7.98 (m, 1H), 7.59-7.62 (m, 1H), 7.31-7.33 (m, 2H), 7.12-7.16 (m, 2H), 2.89 (m, 8H), 2.82-2.84 (d, 3H).

LC-MS (ESI): 462.2 (M+H)⁺.

### Example 104

### Preparation of 1-{2-[4-(1-methyl-piperidin-4-yl)-phenylamino]-pyrimidin-4-yl}-1H-indole-3-carboxmethylamide (Compound 104)

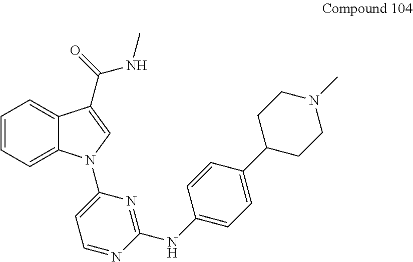 [SMILES: CNC(=O)c1cn(-c2ccnc(Nc3ccc(C4CCN(C)CC4)cc3)n2)c2ccccc12]

1-{2-[4-(1-Methyl-piperidin-4-yl)-phenylamino]-pyrimidin-4-yl}-1H-indole-3-carboxmethylamide was obtained in accordance with the same preparation method of Example 101 except for replacing the tert-butyl 4-(4-amino-phenyl)-piperazine-1-carboxylate in Step 3 of Example 101 with 4-(1-methyl-4-piperidinyl)aniline (Darui).

¹HNMR (DMSO-d6, 400 MHz) δ: 9.79 (s, 1H), 8.79 (s, 1H), 8.72 (br, 1H), 8.56-8.57 (d, 1H), 8.23-8.29 (m, 2H), 7.68-7.70 (d, 2H), 7.29-7.32 (m, 2H), 7.22-7.24 (d, 2H), 7.13-7.14 (d, 1H), 2.62-2.85 (m, 1H), 1.93 (m, 4H).

LC-MS (ESI): 441.3 (M+H)⁺.

### Example 105

### Preparation of 1-[2-(4-piperidin-4-yl-phenylamino)-pyrimidin-4-yl]-1H-indole-3-carboxmethylamide

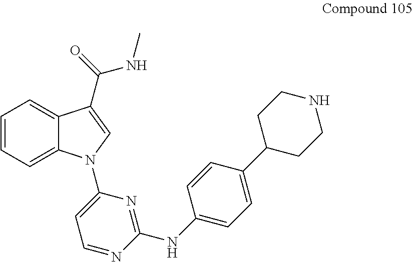 [SMILES: CNC(=O)c1cn(-c2ccnc(Nc3ccc(C4CCNCC4)cc3)n2)c2ccccc12]

1-[2-(4-Piperidin-4-yl-phenylamino)-pyrimidin-4-yl]-1H-indole-3-carboxmethylamide was obtained in accordance with the same preparation method of Example 101 except for replacing the tert-butyl 4-(4-amino-phenyl)-piperazine-1-carboxylate in Step 3 of Example 101 with 1-Boc-4-(4-aminophenyl)piperidine (Darui).

¹HNMR (DMSO-d6, 400 MHz) δ: 9.78 (s, 1H), 8.76 (s, 1H), 8.72 (br, 1H), 8.56-8.57 (d, 1H), 8.22-8.26 (m, 2H), 7.68-7.70 (d, 2H), 7.29-7.33 (m, 2H), 7.19-7.22 (d, 2H), 7.11-7.12 (d, 1H), 2.65-2.83 (m, 8H), 1.63-1.84 (m, 5H).

LC-MS (ESI): 427.2 (M+H)⁺.

### Example 106

### Preparation of 1-{2-[3-(4-methyl-piperazin-1-yl)-phenylamino]-pyrimidin-4-yl}-1H-indole-3-carboxmethylamide (Compound 106)

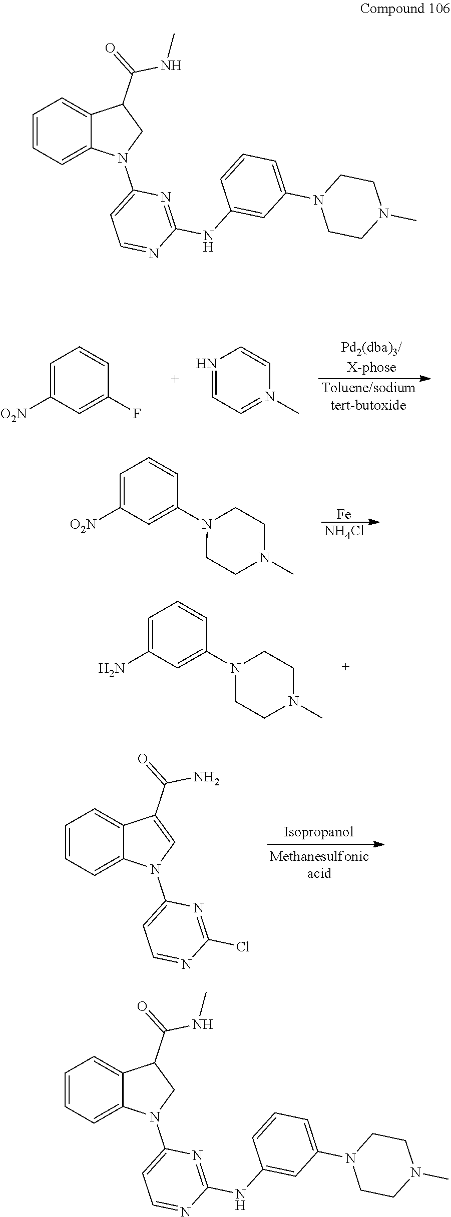

### Step 1: Preparation of 1-methyl-4-(3-nitro-phenyl)-piperazine

M-bromonitrobenzene (3 g, 15 mmol), N-methylpiperazine (1.8 g, 18 mmol), Xphos (1.25 g), Pd₂(dba)₃ (1.37 g) and sodium tert-butoxide (2.88 g, 30 mmol) were dissolved in 30 ml of toluene under a nitrogen atmosphere, and the resulting reaction solution was reacted at 90° C. for 3 hours. The reaction solution was cooled to room temperature, to which 100 ml of dichloromethane was added. The solution was stirred at room temperature for 5 minutes and filtrated, the filtrate was concentrated under reduced pressure. The resulting residues were purified by column chromatography (eluent: methanol/dichloromethane) to obtain the product 1-methyl-4-(3-nitro-phenyl)-piperazine (1.45 g).

### Step 2: Preparation of 3-(4-methyl-piperazin-1-yl)-aniline

The product 1-methyl-4-(3-nitro-phenyl)-piperazine (1.45 g, 6.56 mmol) obtained in Step 1, reduced iron powder (1.47 g, 26.2 mmol) and ammonium chloride (2.452 g, 46 mmol) were added into ethanol (100 ml)/water (30 ml). The resulting mixture was heated to 90° C. and reacted for 2 hours. The reaction solution was cooled to room temperature, slowly poured into saturated aqueous sodium bicarbonate solution (200 ml), and extracted with ethyl acetate (100 ml×2). The organic phase was washed with saturated NaCl solution twice, dried over anhydrous sodium sulfate, filtrated, and concentrated under reduced pressure to obtain 3-(4-methyl-piperazin-1-yl)-aniline (1.0 g). The product was used directly in the next step without purification.

### Step 3: Preparation of 1-{2-[3-(4-methyl-piperazin-1-yl)-phenylamino]-pyrimidin-4-yl}-1H-indole-3-carboxmethylamide

1-{2-[3-(4-Methyl-piperazin-1-yl)-phenylamino]-pyrimidin-4-yl}-1H-indole-3-carboxmethylamide was obtained in accordance with the same preparation method of Example 101 except for replacing the tert-butyl 4-(4-amino-phenyl)-piperazine-1-carboxylate in Step 3 of Example 101 with 3-(4-methyl-piperazin-1-yl)-aniline.

¹HNMR (DMSO-d6, 400 MHz) δ: 9.69 (s, 1H), 8.68-8.71 (m, 2H), 8.57-8.59 (d, 1H), 8.19-8.24 (m, 2H), 7.42 (m, 1H), 7.30-7.32 (m, 2H), 7.23-7.27 (m, 1H), 7.15-7.19 (m, 1H), 7.10-7.12 (d, 1H), 6.64-6.66 (m, 1H), 3.16 (m, 4H), 2.82-2.83 (d, 3H), 2.59 (m, 4H), 2.33 (s, 3H).

LC-MS (ESI): 442.2 (M+H)⁺.

### Example 107

### Preparation of 1-[2-(3-piperazin-1-yl-phenylamino)-pyrimidin-4-yl]-1H-indole-3-carboxmethylamide (Compound 107)

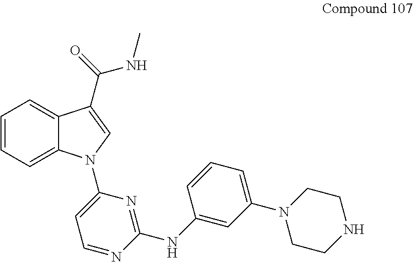 [SMILES: CNC(=O)c1cn(-c2ccnc(Nc3cccc(N4CCNCC4)c3)n2)c2ccccc12]

1-[2-(3-Piperazin-1-yl-phenylamino)-pyrimidin-4-yl]-1H-indole-3-carboxmethylamide was obtained in accordance with the same preparation method of Example 106 except for replacing the N-methylpiperazine in Step 1 of Example 106 with N-Boc-piperazine.

¹HNMR (DMSO-d6, 400 MHz) δ: 9.69 (s, 1H), 8.68-8.71 (m, 2H), 8.58-8.60 (d, 1H), 8.19-8.24 (m, 2H), 7.41 (m, 1H), 7.31-7.33 (m, 2H), 7.23-7.25 (m, 1H), 7.15-7.19 (m, 1H), 7.10-7.12 (d, 1H), 6.62-6.64 (m, 1H), 3.06-3.08 (m, 4H), 2.82-2.87 (m, 7H).

LC-MS (ESI): 428.2 (M+H)⁺.

### Example 108

### Preparation of 1-[2-(4-sulfamoyl-phenylamino)-pyrimidin-4-yl]-1H-indole-3-carboxmethylamide (Compound 108)

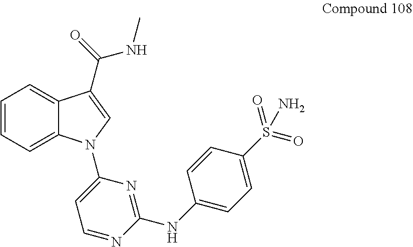 [SMILES: CNC(=O)c1cn(-c2ccnc(Nc3ccc(S(N)(=O)=O)cc3)n2)c2ccccc12]

1-[2-(4-Sulfamoyl-phenylamino)-pyrimidin-4-yl]-1H-indole-3-carboxmethylamide was obtained in accordance with the same preparation method of Example 101 except for replacing the tert-butyl 4-(4-amino-phenyl)-piperazine-1-carboxylate in Step 3 of Example 101 with 4-aminobenzenesulfonamide (Darui).

¹HNMR (DMSO-d6, 400 MHz) δ: 10.30 (s, 1H), 8.80 (s, 1H), 8.73-8.75 (d, 1H), 8.65-8.67 (d, 1H), 8.24-8.28 (m, 2H), 797-7.99 (d, 2H), 7.79-7.81 (d, 2H), 7.31-7.38 (m, 2H), 7.27-7.28 (d, 1H), 2.83 (s, 3H).

LC-MS (ESI): 423.1 (M+H)⁺.

### Example 109

### Preparation of 1-[2-(3-dimethylamino-phenylamino)-pyrimidin-4-yl]-1H-indole-3-carboxamide (Compound 109)

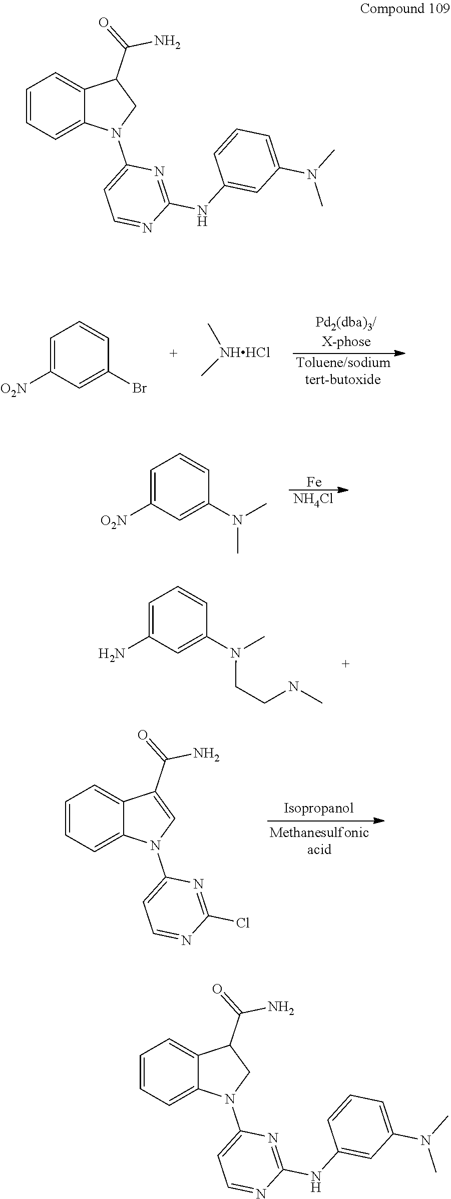

### Step 1: Preparation of dimethyl-(3-nitro-phenyl)-amine

m-Bromonitrobenzene (2 g, 10 mmol), dimethylamine hydrochloride (1.0 g, 12 mmol), Xphos (476 mg), Pd₂(dba)₃ (457 g) and sodium tert-butoxide (2.88 g, 30 mmol) were dissolved in 25 ml of toluene under a nitrogen atmosphere, and the resulting reaction solution was reacted at 90° C. for 3 hours. The reaction solution was cooled to room temperature, to which 100 ml of dichloromethane was added. The solution was stirred at room temperature for 5 minutes and filtrated, the filtrate was concentrated under reduced pressure. The resulting residues were purified by column chromatography (eluent: methanol/dichloromethane) to obtain the product dimethyl-(3-nitro-phenyl)-amine (1.5 g).

### Step 2: Preparation of dimethyl-(3-nitro-phenyl)-amine

The product dimethyl-(3-nitro-phenyl)-amine (1.5 g, 9 mmol) obtained in Step 1, reduced iron powder (2.02 g, 36 mmol) and ammonium chloride (3.37 g, 63 mmol) were added into ethanol (60 ml)/water (20 ml). The resulting mixture was heated to 90° C. and reacted for 2 hours. The reaction solution was cooled to room temperature, slowly poured into saturated aqueous sodium bicarbonate solution (100 ml), and extracted with ethyl acetate (60 ml×2). The organic phase was washed with saturated NaCl solution twice, dried over anhydrous sodium sulfate, filtrated, and concentrated under reduced pressure to obtain dimethyl-(3-nitro-phenyl)-amine (1.1 g). The product was used directly in the next step without purification.

### Step 3: Preparation of 1-[2-(3-dimethylamino-phenylamino)-pyrimidin-4-yl]-1H-indole-3-carboxamide

1-[2-(3-Dimethylamino-phenylamino)-pyrimidin-4-yl]-1H-indole-3-carboxamide was obtained in accordance with the same preparation method of Step 4 of Example 1 except for replacing the 3-bromo-4-fluoroaniline in Step 4 of Example 1 with dimethyl-(3-nitro-phenyl)-amine (prepared in Step 2).

¹HNMR (DMSO-d6, 400 MHz) δ: 2.89 (6H, s), 6.43-6.46 (1H, m), 7.09 (1H, d), 7.11-7.22 (4H, m), 7.29-7.32 (2H, m), 7.68 (1H, br), 8.24-8.27 (1H, m), 8.58 (1H, d), 8.72-8.78 (1H, br), 8.79 (1H, s), 9.65 (1H, s).

LC-MS (ESI): 373.1 (M+H)⁺.

### Example 110

### Preparation of 1-(2-{3-[(2-dimethylamino-ethyl)-methyl-amino]-phenylamino}-pyrimidin-4-yl)-1H-indole-3-carboxamide (Compound 110)

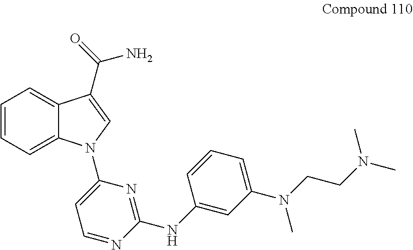 [SMILES: CN(C)CCN(C)c1cccc(Nc2nccc(-n3cc(C(N)=O)c4ccccc43)n2)c1]

1-(2-{3-[(2-Dimethylamino-ethyl)-methyl-amino]-phenylamino}-pyrimidin-4-yl)-1H-indole-3-carboxamide was obtained in accordance with the same preparation method of Example 109 except for replacing the dimethylamine hydrochloride in Step 1 of Example 109 with N,N,N′-trimethylethylenediamine.

¹HNMR (DMSO-d6, 400 MHz) δ: 2.17 (6H, s), 2.40 (2H, t), 2.89 (3H, s), 3.42 (2H, t), 6.38-6.41 (1H, m), 7.07-7.24 (5H, m), 7.29-7.32 (2H, m), 7.68 (1H, br), 8.24-8.27 (1H, m), 8.56 (1H, d), 8.72-8.78 (2H, m), 9.64 (1H, s).

LC-MS (ESI): 430.2 (M+H)⁺.

### Example 111

### Preparation of 1-[2-(3-piperazin-1-yl-phenylamino)-pyrimidin-4-yl]-1H-indole-3-carboxamide (Compound 111)

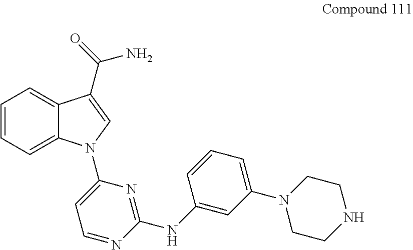 [SMILES: NC(=O)c1cn(-c2ccnc(Nc3cccc(N4CCNCC4)c3)n2)c2ccccc12]

1-[2-(3-Piperazin-1-yl-phenylamino)-pyrimidin-4-yl]-1H-indole-3-carboxamide was obtained in accordance with the same preparation method of Example 109 except for replacing the dimethylamine hydrochloride in Step 1 of Example 109 with N-Boc-piperazine.

¹HNMR (DMSO-d6, 400 MHz) δ: 2.81 (4H, br), 3.03 (4H, br), 6.61-6.63 (1H, m), 7.10-7.34 (6H, m), 7.39 (1H, s), 7.53 (1H, br), 8.24-8.27 (1H, m), 8.59 (1H, d), 8.72 (1H, br), 8.82 (1H, s), 9.69 (1H, s).

LC-MS (ESI): 414.1 (M+H)⁺.

### Example 112

### Preparation of 1-{2-[3-(4-methyl-[1,4]homopiperazin-1-yl)-phenylamino]-pyrimidin-4-yl}-1H-indole-3-carboxamide (Compound 112)

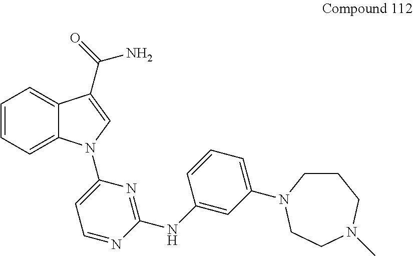 [SMILES: CN1CCCN(c2cccc(Nc3nccc(-n4cc(C(N)=O)c5ccccc54)n3)c2)CC1]

1-{2-[3-(4-Methyl-[1,4]homopiperazin-1-yl)-phenylamino]-pyrimidin-4-yl}-1H-indole-3-carboxamide was obtained in accordance with the same preparation method of Example 109 except for replacing the dimethylamine hydrochloride in Step 1 of Example 109 with N-methyl homopiperazine.

¹HNMR (DMSO-d6, 400 MHz) δ: 9.62 (1H, s), 8.80 (1H, s), 8.73 (1H, s), 8.58 (1H, d), 8.26 (1H, dd), 7.70 (1H, s), 7.31 (2H, d), 7.09-7.18 (5H, m), 6.42 (1H, d), 3.54 (2H, s), 3.42 (4H, t), 2.75 (2H, s), 2.62 (2H, s), 2.37 (3H, s).

LC-MS (ESI): 442.2 (M+H)⁺.

### Example 113

### Preparation of 1-{2-[3-(4-methyl-piperazin-1-yl)-phenylamino]-pyrimidin-4-yl}-1H-indole-3-carboxamide (Compound 113)

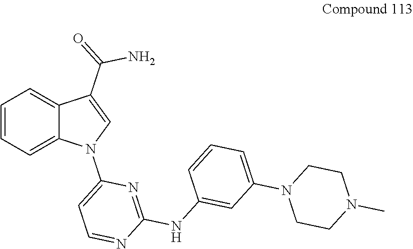 [SMILES: CN1CCN(c2cccc(Nc3nccc(-n4cc(C(N)=O)c5ccccc54)n3)c2)CC1]

1-{2-[3-(4-Methyl-piperazin-1-yl)-phenylamino]-pyrimidin-4-yl}-1H-indole-3-carboxamide was obtained in accordance with the same preparation method of Example 109 except for replacing the dimethylamine hydrochloride in Step 1 of Example 109 with N-methyl piperazine.

¹HNMR (DMSO-d6, 400 MHz) δ: 9.69 (1H, s), 8.81 (1H, s), 8.72 (1H, s), 8.60 (1H, d), 8.26 (1H, t), 7.70 (1H, s), 7.41 (1H, s), 7.31 (2H, t), 7.27 (1H, br), 7.18 (2H, br), 7.12 (1H, d), 6.66 (1H, d), 3.16 (4H, br), 2.54 (4H, br), 2.29 (3H, s).

LC-MS (ESI): 428.3 (M+H)⁺.

### Example 114

### Preparation of 1-{2-[3-fluoro-5-(4-methyl-piperazin-1-yl)-phenylamino]-pyrimidin-4-yl}-1H-indole-3-carboxamide (Compound 114)

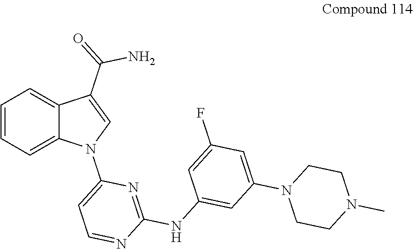 [SMILES: CN1CCN(c2cc(F)cc(Nc3nccc(-n4cc(C(N)=O)c5ccccc54)n3)c2)CC1]

1-{2-[3-Fluoro-5-(4-methyl-piperazin-1-yl)-phenylamino]-pyrimidin-4-yl}-1H-indole-3-carboxamide was obtained in accordance with the same preparation method of Example 109 except for replacing the dimethylamine hydrochloride in Step 1 of Example 109 with N-methyl piperazine and replacing the m-bromonitrobenzene in Step 1 of Example 109 with 3-fluoro-5-bromonitrobenzene.

¹HNMR (DMSO-d6, 400 MHz) δ: 9.84 (s, 1H), 8.81 (s, 1H), 8.73-8.84 (d, 1H), 8.62-8.63 (d, 1H), 8.26-8.28 (m, 1H), 7.70 (br, 1H), 7.15-7.35 (m, 6H), 7.41-7.44 (d, 1H), 3.14 (m, 4H), 2.42 (m, 4H), 2.21 (s, 3H).

LC-MS (ESI): 446.2 (M+H)⁺.

### Example 115

### Preparation of 1-{5-fluoro-2-[3-(4-methyl-piperazin-1-yl)-phenylamino]-pyrimidin-4-yl}-1H-indole-3-carboxamide (Compound 115)

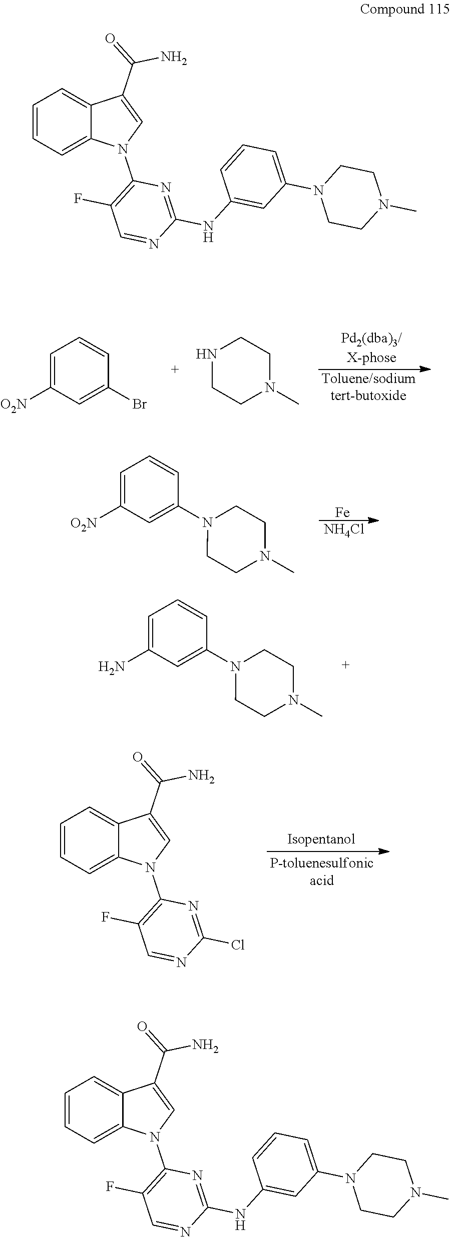

### Step 1: Preparation of 1-methyl-4-(3-nitro-phenyl)-piperazine

m-Bromonitrobenzene (3 g, 15 mmol), N-methylpiperazine (1.8 g, 18 mmol), Xphos (625 mg, 1.5 mmol), Pd₂(dba)₃ (686 mg, 0.75 mmol) and sodium tert-butoxide (2.88 g, 30 mmol) were dissolved in 50 ml of toluene under a nitrogen atmosphere, and the resulting reaction solution was reacted at 90° C. for 8 hours. The reaction solution was cooled to room temperature, to which 100 ml of dichloromethane was added. The solution was stirred at room temperature for 5 minutes and filtrated, the filtrate was concentrated under reduced pressure. The resulting residues were purified by column chromatography (eluent: methanol/dichloromethane) to obtain the product 1-methyl-4-(3-nitro-phenyl)-piperazine (2.8 g).

### Step 2: Preparation of 3-(4-methyl-piperazin-1-yl)-aniline

The product 1-methyl-4-(3-nitro-phenyl)-piperazine (2.8 g, 12.6 mmol) obtained in Step 1, reduced iron powder (2.8 g, 50.46 mmol) and ammonium chloride (4.7 g, 88.2 mmol) were added into ethanol (60 ml)/water (20 ml). The resulting mixture was heated to 90° C. and reacted for 2 hours. The reaction solution was cooled to room temperature, slowly poured into saturated aqueous sodium bicarbonate solution (100 ml), and extracted with ethyl acetate (60 ml×2). The organic phase was washed with saturated NaCl solution twice, dried over anhydrous sodium sulfate, filtrated, and concentrated under reduced pressure to obtain 3-(4-methyl-piperazin-1-yl)-aniline (2.0 g). The product was used directly in the next step without purification.

### Step 3: Preparation of 1-(5-fluoro-2-[3-(4-methyl-piperazin-1-yl)-phenylamino]-pyrimidin-4-yl}-1H-indole-3-carboxamide

1-(5-Fluoro-2-[3-(4-methyl-piperazin-1-yl)-phenylamino]-pyrimidin-4-yl}-1H-indole-3-carboxamide was obtained in accordance with the same preparation method of Step 2 of Example 39 except for replacing the tert-butyl 4-(4-amino-2-methyl-phenyl)-piperazine-1-carboxylate in Step 2 of Example 39 with 3-(4-methyl-piperazin-1-yl)-aniline (prepared in Step 2).

¹HNMR (DMSO-d6, 400 MHz) δ: 9.87 (s, 1H), 8.83-8.784 (d, 1H), 8.62 (d, 1H), 8.34-8.37 (m, 1H), 8.27 (br, 1H), 7.92 (br, 1H), 7.48 (s, 1H), 7.35-7.40 (m, 2H), 7.17-7.25 (m, 3H), 6.66-6.68 (d, 1H), 3.20 (m, 4H), 2.68 (m, 4H), 2.41 (s, 3H).

LC-MS (ESI): 446.2 (M+H)⁺.

### Example 116

### Preparation of 1-{5-fluoro-2-[4-fluoro-3-(4-methyl-piperazin-1-yl)-phenylamino]-pyrimidin-4-yl}-1H-indole-3-carboxamide (Compound 116)

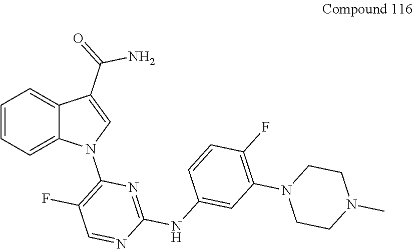 [SMILES: CN1CCN(c2cc(Nc3ncc(F)c(-n4cc(C(N)=O)c5ccccc54)n3)ccc2F)CC1]

1-{5-Fluoro-2-[4-fluoro-3-(4-methyl-piperazin-1-yl)-phenylamino]-pyrimidin-4-yl}-1H-indole-3-carboxamide was obtained in accordance with the same preparation method of Example 115 except for replacing the m-bromonitrobenzene in Step 1 of Example 115 with 3-bromo-4-fluoronitrobenzene.

¹HNMR (DMSO-d6, 400 MHz) δ: 9.89 (s, 1H), 8.77-8.78 (d, 1H), 8.55 (d, 1H), 8.28-8.31 (m, 2H), 8.19 (br, 1H), 7.86 (br, 1H), 7.46-7.48 (m, 1H), 7.31-7.35 (m, 3H), 7.19 (br, 1H), 7.05-7.10 (m, 1H), 3.01 (m, 4H), 2.61 (m, 4H), 2.33 (s, 3H).

LC-MS (ESI): 464.1 (M+H)⁺.

### Example 117

### Preparation of 1-{5-fluoro-2-[4-methyl-3-(4-methyl-piperazin-1-yl)-phenylamino]-pyrimidin-4-yl}-1H-indole-3-carboxamide (Compound 117)

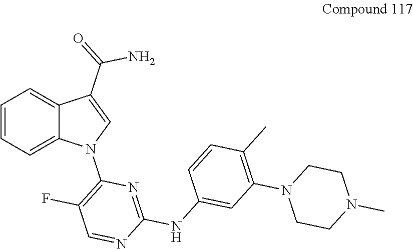 [SMILES: Cc1ccc(Nc2ncc(F)c(-n3cc(C(N)=O)c4ccccc43)n2)cc1N1CCN(C)CC1]

1-{5-Fluoro-2-[4-methyl-3-(4-methyl-piperazin-1-yl)-phenylamino]-pyrimidin-4-yl}-1H-indole-3-carboxamide was obtained in accordance with the same preparation method of Example 115 except for replacing the m-bromonitrobenzene in Step 1 of Example 115 with 2-bromo-4-nitrotoluene.

¹HNMR (DMSO-d6, 400 MHz) δ: 9.83 (s, 1H), 8.76-8.77 (d, 1H), 8.56 (d, 1H), 8.29-8.31 (m, 2H), 8.21 (br, 1H), 7.87 (br, 1H), 7.44 (s, 1H), 7.40-7.42 (d, 1H), 7.31-7.33 (m, 2H), 7.18 (br, 1H), 7.07-7.09 (d, 1H), 2.87 (m, 4H), 2.68 (m, 4H), 2.39 (s, 3H), 2.19 (s, 3H).

LC-MS (ESI): 460.2 (M+H)⁺.

### Example 118

### Preparation of 1-{5-chloro-2-[3-(4-methyl-piperazin-1-yl)-phenylamino]-pyrimidin-4-yl}-1H-indole-3-carboxamide (Compound 118)

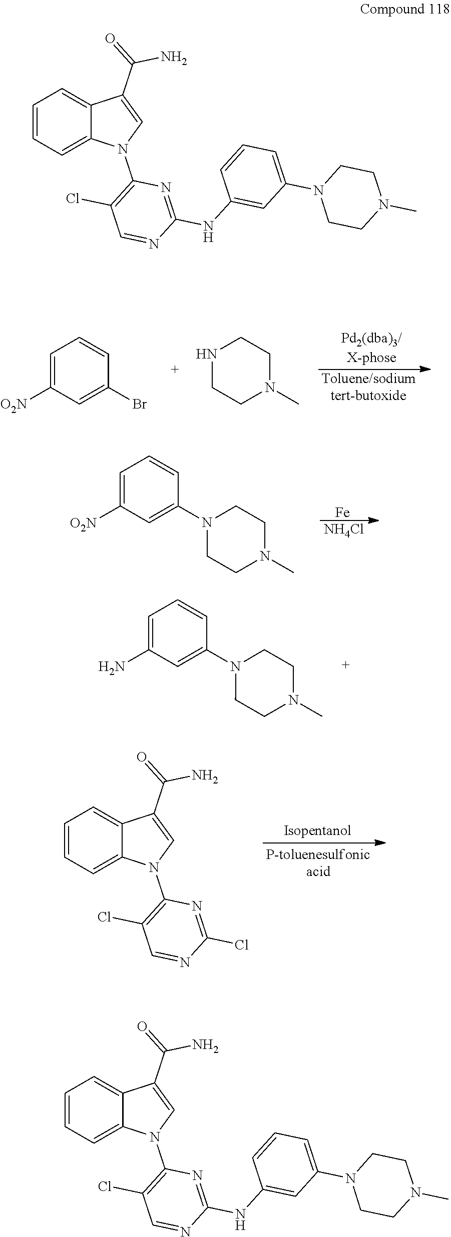

1-{5-Chloro-2-[3-(4-methyl-piperazin-1-yl)-phenylamino]-pyrimidin-4-yl}-1H-indole-3-carboxamide was obtained in accordance with the same preparation method of Step 2 of Example 34 except for replacing the tert-butyl 4-(4-amino-phenyl)-piperazine-1-carboxylate in Step 2 of Example 34 with 3-(4-methyl-piperazin-1-yl)-aniline (prepared in Step 2 of Example 115).

¹HNMR (DMSO-d6, 400 MHz) δ: 10.05 (s, 1H), 8.81 (s, 1H), 8.54 (s, 1H), 8.28-8.29 (m, 1H), 7.73-7.74 (m, 2H), 7.48 (s, 1H), 7.29-7.30 (m, 2H), 7.11-7.15 (m, 3H), 6.60 (m, 1H), 3.09 (m, 4H), 2.63 (m, 4H), 2.38 (s, 3H).

LC-MS (ESI): 462.1 (M+H)⁺.

### Example 119

### Preparation of 1-{5-chloro-2-[4-fluoro-3-(4-methyl-piperazin-1-yl)-phenylamino]-pyrimidin-4-yl}-1H-indole-3-carboxamide (Compound 119)

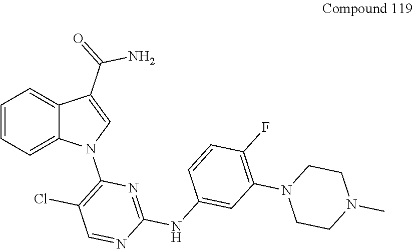 [SMILES: CN1CCN(c2cc(Nc3ncc(Cl)c(-n4cc(C(N)=O)c5ccccc54)n3)ccc2F)CC1]

1-{5-Chloro-2-[4-fluoro-3-(4-methyl-piperazin-1-yl)-phenylamino]-pyrimidin-4-yl}-1H-indole-3-carboxamide was obtained in accordance with the same preparation method of Example 118 except for replacing the m-bromonitrobenzene in Example 118 with 3-bromo-4-fluoronitrobenzene.

¹HNMR (DMSO-d6, 400 MHz) δ: 10.12 (s, 1H), 8.81 (s, 1H), 8.52 (s, 1H), 8.27 (m, 1H), 7.72 (m, 2H), 7.51 (m, 1H), 7.28 (m, 3H), 7.14 (br, 1H), 7.06 (m, 1H), 2.97 (m, 4H), 2.65 (m, 4H), 2.37 (s, 3H).

LC-MS (ESI): 480.1 (M+H)⁺.

### Example 120

### Preparation of 1-{5-chloro-2-[4-chloro-3-(4-methyl-piperazin-1-yl)-phenylamino]-pyrimidin-4-yl}-1H-indole-3-carboxamide (Compound 120)

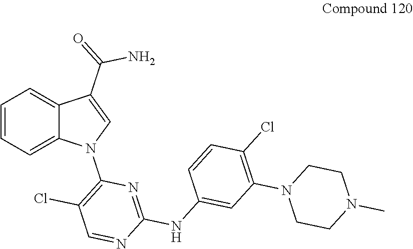 [SMILES: CN1CCN(c2cc(Nc3ncc(Cl)c(-n4cc(C(N)=O)c5ccccc54)n3)ccc2Cl)CC1]

1-{5-Chloro-2-[4-chloro-3-(4-methyl-piperazin-1-yl)-phenylamino]-pyrimidin-4-yl}-1H-indole-3-carboxamide was obtained in accordance with the same preparation method of Example 118 except for replacing the m-bromonitrobenzene in Example 118 with 3-bromo-4-chloronitrobenzene.

¹HNMR (DMSO-d6, 400 MHz) δ: 10.27 (s, 1H), 8.85 (s, 1H), 8.53 (s, 1H), 8.27 (m, 1H), 7.67-7.84 (m, 3H), 7.41 (m, 1H), 7.30 (m, 3H), 7.15 (br, 1H), 2.94 (m, 4H), 2.68 (m, 4H), 2.42 (s, 3H).

LC-MS (ESI): 496.1 (M+H)⁺.

### Example 121

### Preparation of 1-{5-chloro-2-[4-methyl-3-(4-methyl-piperazin-1-yl)-phenylamino]-pyrimidin-4-yl}-1H-indole-3-carboxamide (Compound 121)

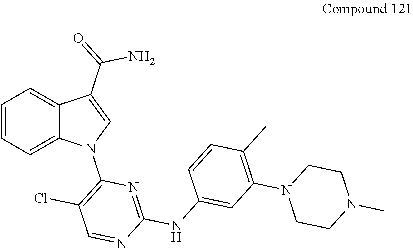 [SMILES: Cc1ccc(Nc2ncc(Cl)c(-n3cc(C(N)=O)c4ccccc43)n2)cc1N1CCN(C)CC1]

1-{5-Chloro-2-[4-methyl-3-(4-methyl-piperazin-1-yl)-phenylamino]-pyrimidin-4-yl}-1H-indole-3-carboxamide was obtained in accordance with the same preparation method of Example 118 except for replacing the m-bromonitrobenzene in Example 118 with 2-bromo-4-nitrotoluene.

¹HNMR (DMSO-d6, 400 MHz) δ: 10.06 (s, 1H), 8.80 (s, 1H), 8.53 (s, 1H), 8.28 (m, 1H), 7.73 (m, 2H), 7.48 (s, 1H), 7.30 (m, 3H), 7.15 (br, 1H), 7.04-7.06 (d, 1H), 2.79 (m, 4H), 2.62 (m, 4H), 2.36 (s, 3H), 2.16 (s, 3H).

LC-MS (ESI): 476.1 (M+H)⁺.

### Example 122

### Preparation of 1-{5-chloro-2-[4-methoxy-3-(4-methyl-piperazin-1-yl)-phenylamino]-pyrimidin-4-yl}-1H-indole-3-carboxamide (Compound 122)

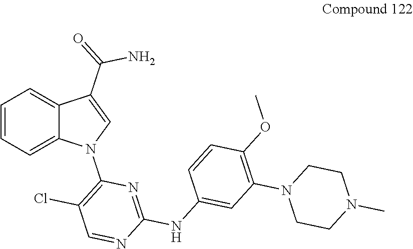 [SMILES: COc1ccc(Nc2ncc(Cl)c(-n3cc(C(N)=O)c4ccccc43)n2)cc1N1CCN(C)CC1]

1-{5-Chloro-2-[4-methoxy-3-(4-methyl-piperazin-1-yl)-phenylamino]-pyrimidin-4-yl}-1H-indole-3-carboxamide was obtained in accordance with the same preparation method of Example 118 except for replacing the m-bromonitrobenzene in Example 118 with 2-bromo-4-nitroanisole.

¹HNMR (DMSO-d6, 400 MHz) δ: 9.93 (s, 1H), 8.76 (s, 1H), 8.50 (s, 1H), 8.27 (m, 1H), 7.71 (m, 2H), 7.28 (m, 4H), 7.14 (br, 1H), 6.86 (m, 1H), 3.73 (s, 3H), 2.95 (m, 4H), 2.70 (m, 4H), 2.41 (s, 3H).

LC-MS (ESI): 492.1 (M+H)⁺.

### Example 123

### Preparation of 1-{5-methoxy-2-[3-(4-methyl-piperazin-1-yl)-phenylamino]-pyrimidin-4-yl}-1H-indole-3-carboxamide (Compound 123)

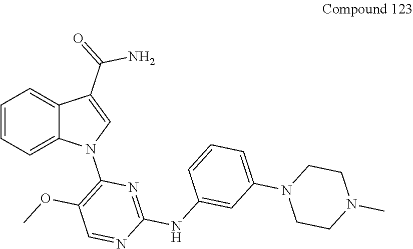 [SMILES: COc1cnc(Nc2cccc(N3CCN(C)CC3)c2)nc1-n1cc(C(N)=O)c2ccccc21]

1-{5-Methoxy-2-[3-(4-methyl-piperazin-1-yl)-phenylamino]-pyrimidin-4-yl}-1H-indole-3-carboxamide was obtained in accordance with the same preparation method of Step 3 of Example 115 except for replacing the 1-(2-chloro-5-fluoro-pyrimidin-4-yl)-1H-indole-3-carboxamide in Step 3 of Example 115 with 1-(2-chloro-5-methoxy-pyrimidin-4-yl)-1H-indole-3-carboxamide (prepared in Example 36).

¹HNMR (DMSO-d6, 400 MHz) δ: 9.52 (s, 1H), 8.62 (s, 1H), 8.58 (s, 1H), 8.26 (m, 1H), 8.00 (m, 1H), 7.76 (br, 1H), 7.50 (s, 1H), 7.27 (m, 2H), 7.06-7.16 (m, 3H), 6.52-6.54 (d, 1H), 3.92 (s, 3H), 3.09 (m, 4H), 2.57 (m, 4H), 2.33 (s, 3H).

LC-MS (ESI): 458.3 (M+H)⁺.

### Example 124

### Preparation of 1-{5-methyl-2-[3-(4-methyl-piperazin-1-yl)-phenylamino]-pyrimidin-4-yl}-1H-indole-3-carboxamide (Compound 124)

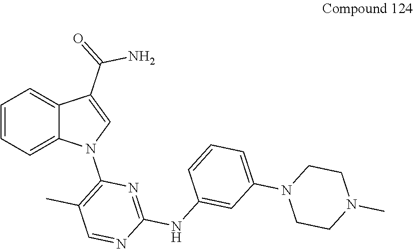 [SMILES: Cc1cnc(Nc2cccc(N3CCN(C)CC3)c2)nc1-n1cc(C(N)=O)c2ccccc21]

1-{5-Methyl-2-[3-(4-methyl-piperazin-1-yl)-phenylamino]-pyrimidin-4-yl}-1H-indole-3-carboxamide was obtained in accordance with the same preparation method of Step 3 of Example 115 except for replacing the 1-(2-chloro-5-fluoro-pyrimidin-4-yl)-1H-indole-3-carboxamide in Step 3 of Example 115 with 1-(2-chloro-5-methyl-pyrimidin-4-yl)-1H-indole-3-carboxamide (prepared in Example 37).

¹HNMR (DMSO-d6, 400 MHz) δ: 9.69 (s, 1H), 8.62 (s, 1H), 8.43 (s, 1H), 8.28-8.30 (m, 1H), 7.65-7.70 (m, 2H), 7.55 (br, 1H), 7.25-7.28 (m, 2H), 7.05-7.11 (m, 3H), 6.52-6.53 (d, 1H), 3.03 (m, 4H), 2.51 (m, 4H), 2.32 (s, 3H), 2.20 (s, 3H).

LC-MS (ESI): 442.2 (M+H)⁺.

### Example 125

### Preparation of 1-(2-{4-[4-(2-hydroxy-ethyl)-piperazin-1-yl]-phenylamino}-pyrimidin-4-yl)-1H-indole-3-carboxamide (Compound 125

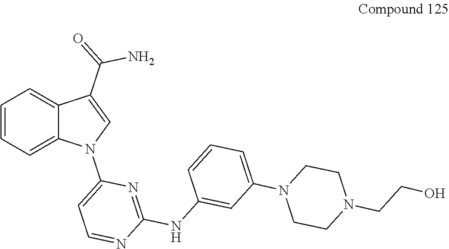 [SMILES: NC(=O)c1cn(-c2ccnc(Nc3cccc(N4CCN(CCO)CC4)c3)n2)c2ccccc12]

1-(2-{4-[4-(2-Hydroxy-ethyl)-piperazin-1-yl]-phenylamino}-pyrimidin-4-yl)-1H-indole-3-carboxamide was obtained in accordance with the same preparation method of Example 109 except for replacing the dimethylamine hydrochloride in Step 1 of Example 109 with 1-(2-hydroxyethyl)piperazine (Darui).

¹HNMR (DMSO-d6, 400 MHz) δ: 2.74 (4H, br), 3.23 (4H, br), 3.63 (4H, m), 6.66 (1H, d), 7.14-7.34 (6H, m), 7.40 (1H, s), 7.78 (1H, br), 8.24-8.27 (1H, m), 8.59 (1H, d), 8.73 (1H, br), 8.88 (1H, s), 9.70 (1H, s).

LC-MS (ESI): 458.2 (M+H)⁺.

### Example 126

### Preparation of 1-(2-{3-[4-(2-methoxy-ethyl)-piperazin-1-yl]-phenylamino]-pyrimidin-4-yl}-1H-indole-3-carboxamide (Compound 126)

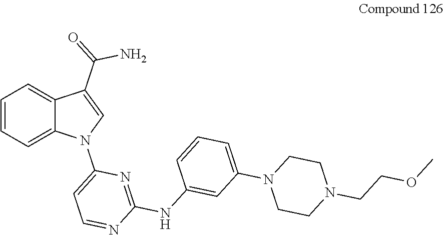 [SMILES: COCCN1CCN(c2cccc(Nc3nccc(-n4cc(C(N)=O)c5ccccc54)n3)c2)CC1]

1-(2-{3-[4-(2-Methoxy-ethyl)-piperazin-1-yl]-phenylamino]-pyrimidin-4-yl}-1H-indole-3-carboxamide was obtained in accordance with the same preparation method of Example 109 except for replacing the dimethylamine hydrochloride in Step 1 of Example 109 with 1-(2-methoxyethyl)piperazine (Darui).

¹HNMR (DMSO-d6, 400 MHz) δ: 2.52-2.58 (6H, m), 3.12 (4H, br), 3.25 (3H, s), 3.47 (2H, t), 6.63 (1H, d), 7.10 (1H, d), 7.14-7.23 (2H, m), 7.24-7.27 (1H, m), 7.29-7.35 (2H, m), 7.39 (1H, s), 7.69 (1H, br), 8.24-8.27 (1H, m), 8.59 (1H, d), 8.72 (1H, br), 8.79 (1H, s), 9.69 (1H, s).

LC-MS (ESI): 472.2 (M+H)⁺.

### Example 127

### Preparation of 1-{2-[3-(4-acryloyl-piperazin-1-yl)-phenylamino]-pyrimidin-4-yl}-1H-indole-3-carboxamide (Compound 127)

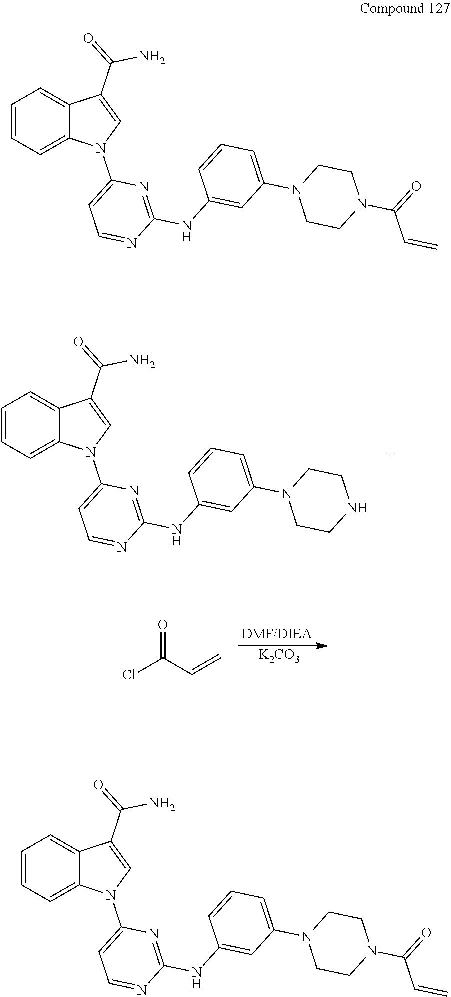

1-[2-(3-Piperazin-1-yl-phenylamino)-pyrimidin-4-yl]-1H-indole-3-carboxamide (Compound 111) (120 mg, 0.29 mmol) and N,N-diisopropylethylamine (112 mg, 0.87 mol) were dissolved in DMF (10 ml). Potassium carbonate (40 mg, 0.29 mmol) was added at room temperature, and then acryloyl chloride (31.7 mg, 0.35 mmol) was slowly added dropwise under an ice bath. After completion of the addition, the ice bath was removed, and the reaction solution was slowly warmed to room temperature and reacted for 1 hour. TLC showed that the reaction was completed. The reaction solution was poured into water, and extracted with ethyl acetate (30 ml*2). The organic phase was washed with saturated NaCl solution twice, dried over anhydrous sodium sulfate, filtrated, and concentrated under reduced pressure. The resulting residues were purified by column chromatography (eluent: dichloromethane/methanol) to obtain 1-{2-[3-(4-acryloyl-piperazin-1-yl)-phenylamino]-pyrimidin-4-yl}-1H-indole-3-carboxamide (40 mg, white solid).

¹HNMR (DMSO-d6, 400 MHz) δ: 9.76 (s, 1H), 8.84 (d, 1H), 8.75 (br, 1H), 8.63-8.64 (d, 1H), 8.30-8.31 (d, 1H), 7.74 (br, 1H), 7.49 (s, 1H), 7.15-7.35 (m, 6H), 6.84-6.91 (m, 1H), 6.70-6.72 (d, 1H), 6.16-6.21 (d, 1H), 5.74-5.76 (d, 1H), 3.72 (m, 4H), 3.18 (m, 4H).

LC-MS (ESI): 468.2 (M+H)⁺.

### Example 128

### Preparation of 1-{2-[3-(4-propionyl-piperazin-1-yl)-phenylamino]-pyrimidin-4-yl}-1H-indole-3-carboxamide (Compound 128)

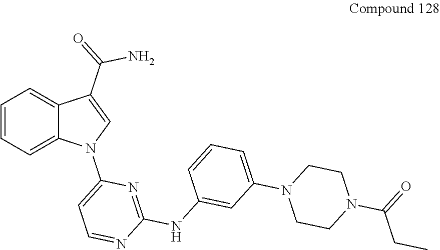 [SMILES: CCC(=O)N1CCN(c2cccc(Nc3nccc(-n4cc(C(N)=O)c5ccccc54)n3)c2)CC1]

1-{2-[3-(4-propionyl-piperazin-1-yl)-phenylamino]-pyrimidin-4-yl}-1H-indole-3-carboxamide was obtained in accordance with the same preparation method of Example 127 except for replacing the acryloyl chloride in Example 127 with propionyl chloride (Darui).

¹HNMR (DMSO-d6, 400 MHz) δ: 9.72 (s, 1H), 8.90 (d, 1H), 8.72 (br, 1H), 8.57-8.59 (d, 1H), 8.25-8.28 (m, 1H), 7.79 (br, 1H), 7.44 (s, 1H), 7.15-7.31 (m, 6H), 6.63-6.65 (d, 1H), 3.53-3.56 (m, 4H), 3.04-3.11 (m, 4H), 2.30-2.35 (q, 2H), 0.97-1.01 (t, 3H).

LC-MS (ESI): 470.2 (M+H)⁺.

### Example 129

### Preparation of 1-{2-[3-(2-dimethylamino-ethoxy)-phenylamino]-pyrimidin-4-yl}-1H-indole-3-carboxamide (Compound 129)

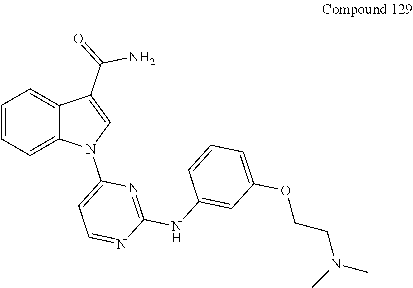 [SMILES: CN(C)CCOc1cccc(Nc2nccc(-n3cc(C(N)=O)c4ccccc43)n2)c1]

1-{2-[3-(2-Dimethylamino-ethoxy)-phenylamino]-pyrimidin-4-yl}-1H-indole-3-carboxamide was obtained in accordance with the same preparation method of Example 19 except for replacing the 4-fluoronitrobenzene in Example 19 with m-fluoronitrobenzene (Darui).

¹HNMR (DMSO-d6, 400 MHz) δ: 9.84 (1H, s), 8.81 (1H, s), 8.77 (1H, d), 8.63 (1H, d), 7.71 (1H, s), 7.53 (1H, s), 7.35-7.29 (3H, m), 7.25-7.21 (2H, m) 7.15 (1H, d), 6.63 (1H, dd), 4.03 (2H, t), 2.63 (2H, t), 2.21 (6H, s).

LC-MS (ESI): 417.1 (M+H)⁺.

### Example 130

### Preparation of 1-[2-(pyridin-2-ylamino)-pyrimidin-4-yl]-1H-indole-3-carboxamide (Compound 130)

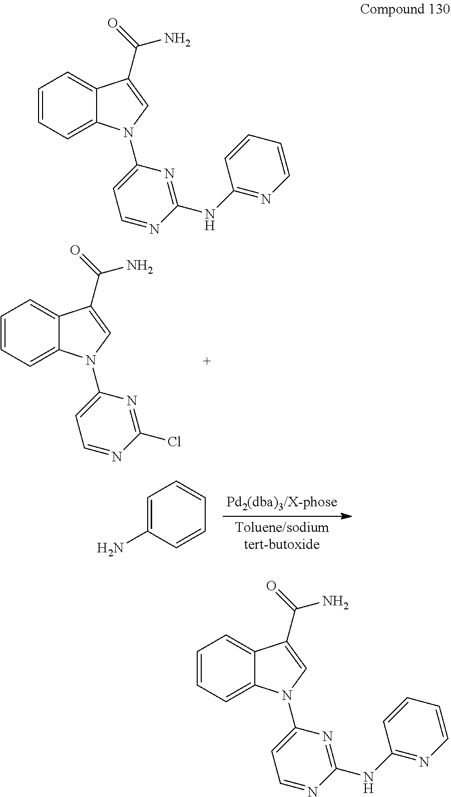

1-(2-Chloro-pyrimidin-4-yl)-1H-indole-3-carboxamide (136 mg, 0.5 mmol), 2-aminopyridine (56.0 mg, 0.6 mmol), Xphos (23 mg), Pd₂(dba)₃ (22 mg) and sodium tert-butoxide (96 mg, 1 mmol) were dissolved in 20 ml of toluene under a nitrogen atmosphere, and the resulting reaction solution was reacted at 90° C. overnight. The reaction solution was cooled to room temperature, to which 30 ml of dichloromethane was added. The solution was stirred at room temperature for 5 minutes and filtrated, the filtrate was concentrated under reduced pressure. The resulting residues were purified by column chromatography (eluent: dichloromethane/methanol) to obtain 1-[2-(pyridin-2-ylamino)-pyrimidin-4-yl]-1H-indole-3-carboxamide (12 mg, white solid).

¹HNMR (DMSO-d6, 400 MHz) δ: 10.41 (1H, s), 9.08 (1H, d), 8.90 (1H, s), 8.67 (1H, d), 8.37 (1H, dd), 8.24 (2H, m), 7.80 (2H, m), 7.34 (2H, m), 7.26 (2H, m), 7.06 (1H, m).

LC-MS (ESI): 331.1 (M+H)⁺.

### Example 131

### Preparation of 1-[2-(4-methoxy-pyridin-2-ylamino)-pyrimidin-4-yl]-1H-indole-3-carboxamide

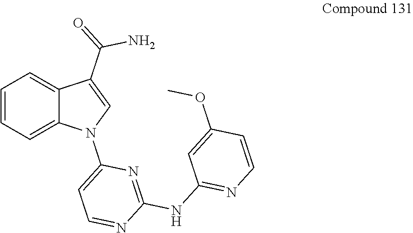 [SMILES: COc1ccnc(Nc2nccc(-n3cc(C(N)=O)c4ccccc43)n2)c1]

1-[2-(4-Methoxy-pyridin-2-ylamino)-pyrimidin-4-yl]-1H-indole-3-carboxamide was obtained in accordance with the same preparation method of Example 130 except for replacing the 2-aminopyridine in Example 130 with 2-amino-4-methoxypyridine (Darui).

¹HNMR (DMSO-d6, 400 MHz) δ: 10.28 (1H, s), 8.99 (1H, d), 8.82 (1H, s), 8.68 (1H, d), 8.24 (1H, dd), 8.19 (1H, d), 7.89 (1H, d), 7.66 (1H, s), 7.31 (2H, m), 7.22 (2H, m), 6.68 (1H, dd), 3.82 (3H, s).

LC-MS (ESI): 361.2 (M+H)⁺.

### Example 132

### Preparation of 1-[2-(4,6-dimethyl-pyridin-2-ylamino)-pyrimidin-4-yl]-1H-indole-3-carboxamide (Compound 132)

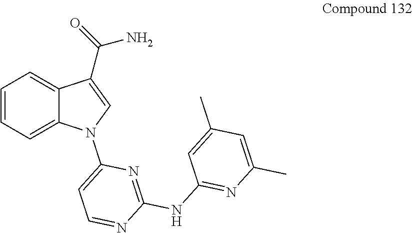 [SMILES: Cc1cc(C)nc(Nc2nccc(-n3cc(C(N)=O)c4ccccc43)n2)c1]

1-[2-(4,6-Dimethyl-pyridin-2-ylamino)-pyrimidin-4-yl]-1H-indole-3-carboxamide was obtained in accordance with the same preparation method of Example 130 except for replacing the 2-aminopyridine in Example 130 with 2-amino-4,6-dimethyl pyridine (Darui).

¹HNMR (DMSO-d6, 400 MHz) δ: 10.18 (1H, s), 9.03 (1H, d), 8.84 (1H, s), 8.66 (1H, d), 8.25 (1H, s), 7.92 (1H, s), 7.69 (1H, s), 7.32 (2H, br), 7.22 (2H, br), 6.79 (1H, s), 2.42 (3H, m), 2.30 (3H, m).

LC-MS (ESI): 359.1 (M+H)⁺.

### Example 133

### Preparation of 1-[2-(5-piperazin-1-yl-pyridin-2-ylamino)-pyrimidin-4-yl]-1H-indole-3-carboxamide (Compound 133)

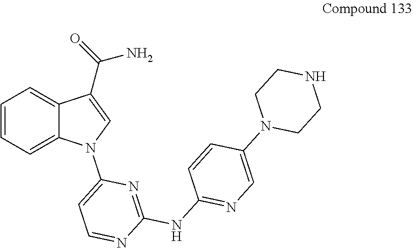 [SMILES: NC(=O)c1cn(-c2ccnc(Nc3ccc(N4CCNCC4)cn3)n2)c2ccccc12]

1-[2-(5-Piperazin-1-yl-pyridin-2-ylamino)-pyrimidin-4-yl]-1H-indole-3-carboxamide was obtained in accordance with the same preparation method of Example 27 except for replacing the 4-fluoronitrobenzene in Step 1 of Example 27 with 5-bromo-nitropyridine (Darui).

¹HNMR (DMSO-d6, 400 MHz) δ: 8.87 (s, 1H), 8.52-8.54 (d, 1H), 8.42-8.44 (d, 1H), 8.27-8.29 (d, 1H), 7.80-7.82 (m, 2H), 7.51 (m, 1H), 7.34-7.42 (m, 2H), 7.28-7.31 (m, 2H), 7.12 (br, 1H), 7.02-7.03 (d, 1H), 6.84-6.86 (d, 1H), 3.99 (m, 4H), 3.13 (m, 4H).

LC-MS (ESI): 415.2 (M+H)⁺.

### Example 134

### Preparation of 1-[5-chloro-2-(5-piperazin-1-yl-pyridin-2-ylamino)-pyrimidin-4-yl]-1H-indole-3-carboxamide (Compound 134)

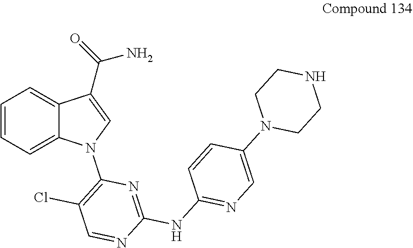 [SMILES: NC(=O)c1cn(-c2nc(Nc3ccc(N4CCNCC4)cn3)ncc2Cl)c2ccccc12]

1-[5-Chloro-2-(5-piperazin-1-yl-pyridin-2-ylamino)-pyrimidin-4-yl]-1H-indole-3-carboxamide was obtained in accordance with the same preparation method of Example 34 except for replacing the tert-butyl 4-(4-amino-phenyl)-piperazine-1-carboxylate in Step 2 of Example 34 with tert-butyl 4-(6-amino-pyridin-3-yl)-piperazine-1-carboxylate (prepared in Example 133).

¹HNMR (DMSO-d6, 400 MHz) δ: 8.72 (s, 1H), 8.55 (d, 1H), 8.26-8.28 (m, 1H), 7.85 (br, 1H), 7.70-7.75 (m, 2H), 7.50 (m, 1H), 7.42 (m, 1H), 7.28-7.34 (m, 2H), 7.13 (br, 1H), 6.82-6.85 (d, 1H), 3.90 (m, 4H), 3.07 (m, 4H).

LC-MS (ESI): 449.1 (M+H)⁺.

### Example 135

### Preparation of 1-[2-(4-piperazin-1-yl-phenylamino)-pyrimidin-4-yl]-1H-pyrrolo[2,3-b]pyridine-3-carboxamide (Compound 135)

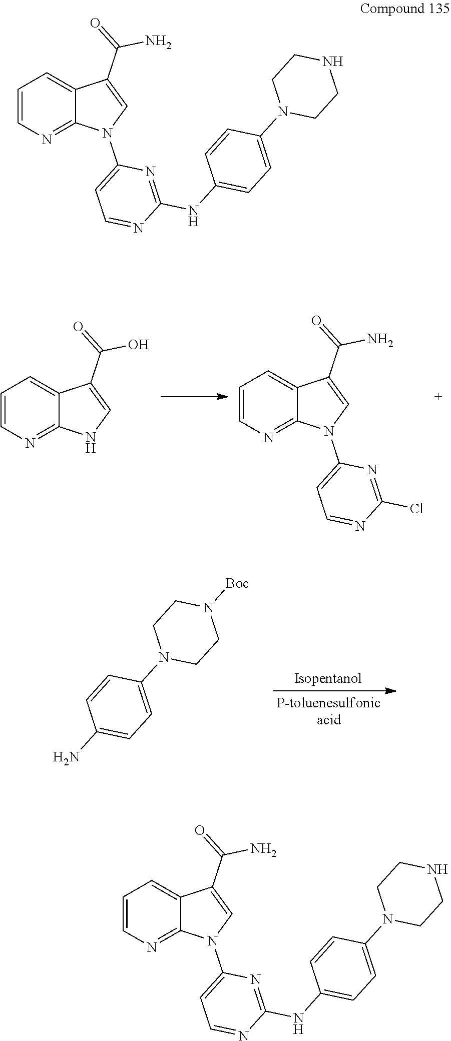 [SMILES: NC(=O)c1cn(-c2ccnc(Nc3ccc(N4CCNCC4)cc3)n2)c2ncccc12 ; O=C(O)c1c[nH]c2ncccc12 ; NC(=O)c1cn(-c2ccnc(Cl)n2)c2ncccc12 ; CC(C)(C)OC(=O)N1CCN(c2ccc(N)cc2)CC1 ; NC(=O)c1cn(-c2ccnc(Nc3ccc(N4CCNCC4)cc3)n2)c2ncccc12]

### Step 1: Preparation of 1-(2-chloro-pyrimidin-4-yl)-1H-pyrrolo[2,3-b]pyridine-3-carboxamide

1-(2-Chloro-pyrimidin-4-yl)-1H-pyrrolo[2,3-b]pyridine-3-carboxamide was obtained in accordance with the same preparation method of Steps 1 to 3 of Example 1 except for replacing the 3-indole carboxylic acid in Step 1 of Example 1 with 7-azaindole-3-carboxylic acid (Darui).

### Step 2: Preparation of 1-[2-(4-piperazin-1-yl-phenylamino)-pyrimidin-4-yl]-1H-pyrrolo[2,3-b]pyridine-3-carboxamide

1-[2-(4-Piperazin-1-yl-phenylamino)-pyrimidin-4-yl]-1H-pyrrolo[2,3-b]pyridine-3-carboxamide was obtained in accordance with the same preparation method of Step 2 of Example 34 except for replacing the 1-(2,5-dichloro-pyrimidin-4-yl)-1H-indole-3-carboxamide in Step 2 of Example 34 with 1-(2-chloro-pyrimidin-4-yl)-1H-pyrrolo[2,3-b]pyridine-3-carboxamide (prepared in Step 1).

¹HNMR (DMSO-d6, 400 MHz) δ: 9.60 (s, 1H), 8.98 (s, 1H), 8.58-8.60 (m, 2H), 8.48-8.50 (dd, 1H), 8.28-8.29 (d, 1H), 8.06 (br, 1H), 7.66-7.68 (d, 2H), 7.40-7.43 (m, 1H), 7.30 (br, 1H), 6.97-6.99 (d, 2H), 3.16-3.18 (m, 4H), 3.06-3.08 (m, 4H).

LC-MS (ESI): 415.2 (M+H)⁺.

### Example 136

### Preparation of 1-[2-(4-piperazin-1-yl-phenylamino)-pyrimidin-4-yl]-1H-pyrrolo[2,3-b]pyridine-3-carboxamide (Compound 136)

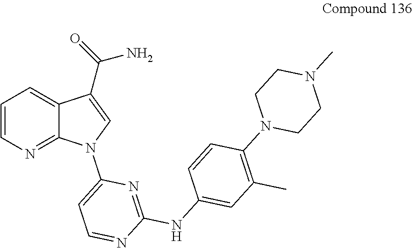 [SMILES: Cc1cc(Nc2nccc(-n3cc(C(N)=O)c4cccnc43)n2)ccc1N1CCN(C)CC1]

1-[2-(4-Piperazin-1-yl-phenylamino)-pyrimidin-4-yl]-1H-pyrrolo[2,3-b]pyridine-3-carboxamide was obtained in accordance with the same preparation method of of Example 135 except for replacing the tert-butyl 4-(4-amino-phenyl)-piperazine-1-carboxylate in Example 136 with 3-methyl-4-(4-methyl-piperazin-1-yl)-aniline (prepared in Step 50).

¹HNMR (DMSO-d6, 400 MHz) δ: 9.60 (s, 1H), 8.99 (s, 1H), 8.57-8.61 (m, 2H), 8.48-8.49 (d, 1H), 8.28-8.30 (d, 1H), 8.02 (br, 1H), 7.61 (s, 1H), 7.55-7.57 (d, 1H), 7.39-7.42 (m, 1H), 7.28 (br, 1H), 7.01-7.03 (d, 1H), 3.37 (m, 4H), 2.82 (m, 4H), 2.26 (s, 3H), 2.25 (s, 3H).

LC-MS (ESI): 443.2 (M+H)⁺.

### Example 137

### Preparation of 1-[5-fluoro-2-(3-morpholin-4-yl-phenylamino)-pyrimidin-4-yl]-1H-indole-3-carboxamide (Compound 137)

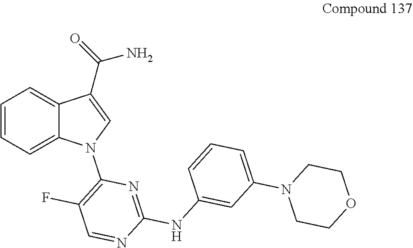 [SMILES: NC(=O)c1cn(-c2nc(Nc3cccc(N4CCOCC4)c3)ncc2F)c2ccccc12]

1-[5-Fluoro-2-(3-morpholin-4-yl-phenylamino)-pyrimidin-4-yl]-1H-indole-3-carboxamide was obtained in accordance with the same preparation method of Example 115 except for replacing the N-methylpiperazine in Step 1 of Example 115 with morpholine.

¹HNMR (DMSO-d6, 400 MHz) δ: 9.87 (s, 1H), 8.80 (m, 1H), 8.58 (m, 1H), 8.21-8.31 (m, 2H), 7.89 (br, 1H), 7.16-7.47 (m, 6H), 6.61 (m, 1H), 3.67 (m, 4H), 3.03 (m, 4H).

LC-MS (ESI): 433.1 (M+H)⁺.

### Example 138

### Preparation of 1-[2-(3-piperazin-1-yl-phenylamino)-pyrimidin-4-yl]-1H-indazole-3-carboxamide (Compound 138)

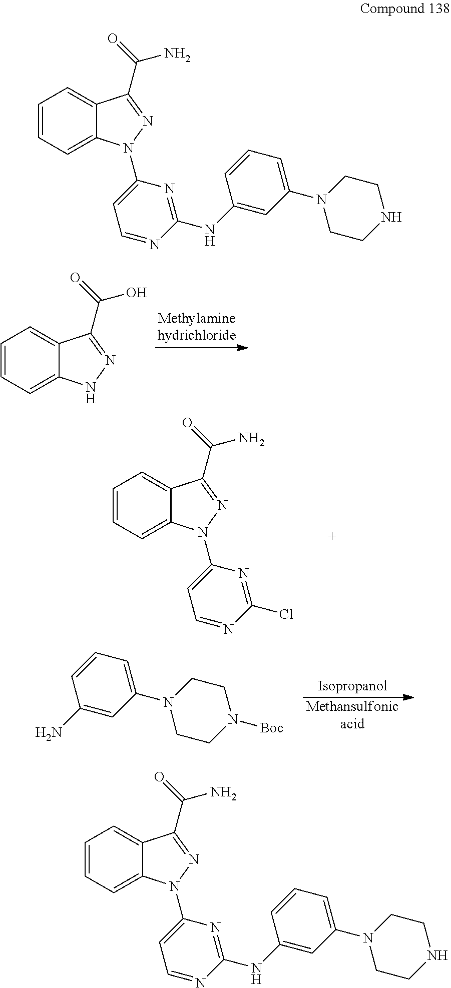

### Step 1: Preparation of 1-(2-chloro-pyrimidin-4-yl)-1H-indazole-3-carboxamide

1-(2-Chloro-pyrimidin-4-yl)-1H-indazole-3-carboxamide was obtained in accordance with the same preparation method of Steps 1 to 3 of Example 1 except for replacing the 3-indole carboxylic acid in Step 1 of Example 1 with indazole-3-carboxylic acid (Darui).

### Step 2: Preparation of 1-[2-(3-piperazin-1-yl-phenylamino)-pyrimidin-4-yl]-1H-indazole-3-carboxamide

1-[2-(3-Piperazin-1-yl-phenylamino)-pyrimidin-4-yl]-1H-indazole-3-carboxamide was obtained in accordance with the same preparation method of Example 111 except for replacing the 1-(2-chloro-pyrimidin-4-yl)-1H-indole-3-carboxamide in Example 111 with 1-(2-chloro-pyrimidin-4-yl)-1H-indazole-3-carboxamide (prepared in Step 1).

¹HNMR (DMSO-d6, 400 MHz) δ: 2.86 (4H, t), 3.03 (4H, t), 6.96 (2H, d), 7.43 (1H, t), 7.50-7.59 (4H, m), 7.72 (1H, s), 8.20 (1H, s), 8.29 (1H, d), 8.53 (1H, d), 8.90 (1H, br), 9.58 (1H, s).

LC-MS (ESI): 415.1 (M+H)⁺.

### Example 139

### Preparation of 1-{5-fluoro-2-[3-(4-morpholin-4-yl-piperidin-1-yl)-phenylamino]-pyrimidin-4-yl}-1H-indole-3-carboxamide (Compound 139)

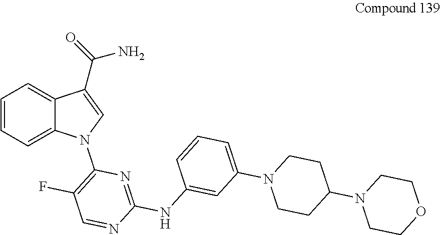 [SMILES: NC(=O)c1cn(-c2nc(Nc3cccc(N4CCC(N5CCOCC5)CC4)c3)ncc2F)c2ccccc12]

1-{5-Fluoro-2-[3-(4-morpholin-4-yl-piperidin-1-yl)-phenylamino]-pyrimidin-4-yl}-1H-indole-3-carboxamide was obtained in accordance with the same preparation method of Example 115 except for replacing the N-methylpiperazine in Step 1 of Example 115 with 4-(4-piperidinyl)morpholine.

¹HNMR (DMSO-d6, 400 MHz) δ: 9.78 (s, 1H), 8.77-8.78 (d, 1H), 8.56 (d, 1H), 8.28-8.31 (m, 1H), 8.21 (br, 1H), 7.85 (br, 1H), 7.44 (s, 1H), 7.30-7.34 (m, 2H), 7.09-7.16 (m, 3H), 6.59-6.61 (d, 1H), 3.58-3.65 (m, 6H), 2.58-2.64 (t, 2H), 2.45-2.5 (m, 4H), 2.21 (br, 1H), 1.77 (s, 2H), 1.44 (s, 2H).

LC-MS (ESI): 516.0 (M+H)⁺.

### Example 140

### Preparation of 1-(5-fluoro-2-{3-[4-(tetrahydro-pyran-4-yl)-piperazin-1-yl]-phenylamino}-pyrimidin-4-yl)-1H-indole-3-carboxamide (Compound 140)

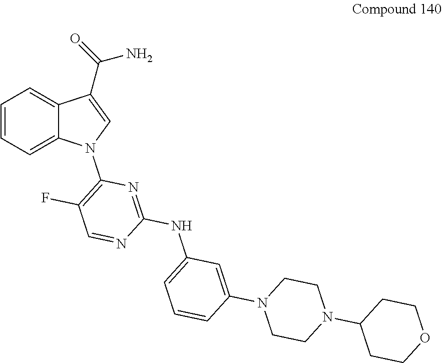 [SMILES: NC(=O)c1cn(-c2nc(Nc3cccc(N4CCN(C5CCOCC5)CC4)c3)ncc2F)c2ccccc12]

1-(5-Fluoro-2-{3-[4-(tetrahydro-pyran-4-yl)-piperazin-1-yl]-phenylamino}-pyrimidin-4-yl)-1H-indole-3-carboxamide was obtained in accordance with the same preparation method of Example 115 except for replacing the N-methylpiperazine in Step 1 of Example 115 with 1-(tetrahydropyran-4-yl)piperazine.

¹HNMR (DMSO-d6, 400 MHz) δ: 9.82 (s, 1H), 8.78-8.79 (d, 1H), 8.56-8.57 (d, 1H), 8.29-8.31 (m, 1H), 8.20-8.21 (m, 1H), 7.85 (br, 1H), 7.45 (s, 1H), 7.31-7.34 (m, 2H), 7.14-7.16 (m, 3H), 6.60-6.62 (d, 1H), 3.91-3.93 (m, 2H), 3.27-3.29 (m, 2H), 3.07 (s, 4H), 2.55-2.58 (br, 2H), 1.98-2.01 (m, 1H), 1.76-1.77 (br, 2H), 1.44-1.48 (br, 2H), 1.24-1.30 (m, 2H).

LC-MS (ESI): 516.2 (M+H)⁺.

### Example 141

### Preparation of 1-{5-fluoro-2-[3-(4-methyl-piperazin-1-yl)-phenylamino]-pyrimidin-4-yl}-5-methoxy-1H-indole-3-carboxamide (Compound 141)

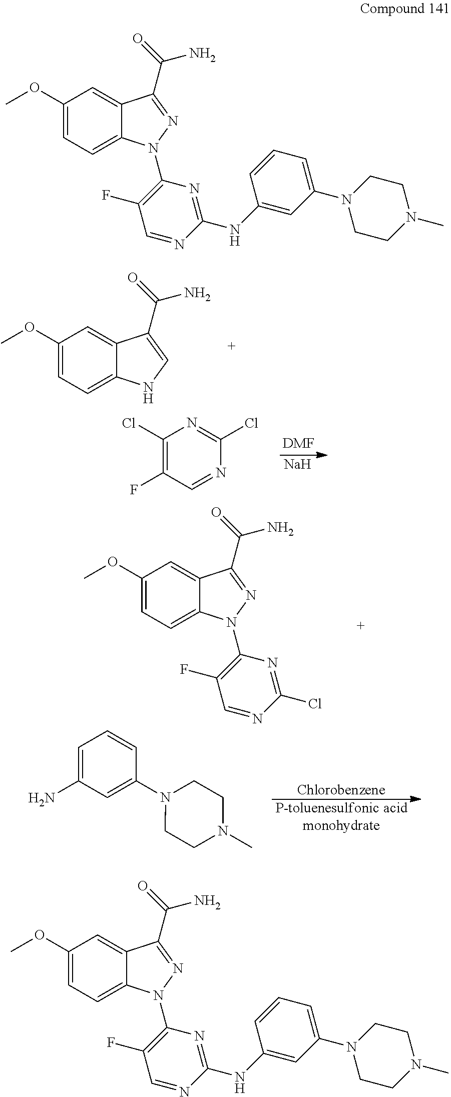

### Step 1: Preparation of 1-(2-chloro-5-fluoro-pyrimidin-4-yl)-5-methoxy-1H-indole-3-carboxamide

5-Methoxy-1H-indole-3-carboxamide (230 mg, 1.21 mmol) (prepared in Example 33) was dissolved in 10 ml of DMF at room temperature. Sodium hydride (50 mg, 1.21 mmol) was added at room temperature, and the reaction solution was reacted at room temperature for 30 minutes to obtain mixed system A. 2,4-Dichloro-5-fluoropyrimidine (303 mg, 1.82 mmol) was dissolved in 8 ml of DMF and added with above mixed system A slowly at room temperature, and the reaction solution was reacted at room temperature for 1 hour after completion of the addition. TLC showed that the reaction was completed. The reaction system was poured into water (80 ml), and extracted with ethyl acetate (60 ml×2). The organic phase was washed with saturated NaCl solution twice, dried over anhydrous sodium sulfate, filtrated, and concentrated under reduced pressure to obtain 1-(2-chloro-5-fluoro-pyrimidin-4-yl)-5-methoxy-1H-indole-3-carboxamide (400 mg). The product was used directly in the next step without purification.

### Step 2: Preparation of 1-{5-fluoro-2-[3-(4-methyl-piperazin-1-yl)-phenylamino]-pyrimidin-4-yl}-5-methoxy-1H-indole-3-carboxamide

The product 1-(2-chloro-5-fluoro-pyrimidin-4-yl)-5-methoxy-H-indole-3-carboxamide (320 mg, 1 mmol) obtained in Step 1, 3-(4-methyl-piperazin-1-yl)-aniline (191 mg, 1 mmol) (prepared in Example 115) and p-toluenesulfonic acid monohydrate (230 mg, 1.2 mmol) were dissolved in 40 ml of chlorobenzene. The reaction solution was heated to 130° C. and reacted for 2 hours. The reaction solution was cooled to room temperature, and the supernatant was removed. The viscous oil at the bottom of the flask was dissolved in a mixed solvent of dichloromethane/methanol (10:1). The solution was poured into saturated aqueous sodium bicarbonate solution (100 ml), and extracted with dichloromethane (60 ml×2). The organic phase was washed with saturated NaCl solution twice, dried over anhydrous sodium sulfate, filtrated and concentrated under reduced pressure. The resulting residues were purified by column chromatography (eluent: dichloromethane/methanol (containing 5% of aqueous ammonia)) to obtain 1-{5-fluoro-2-[3-(4-methyl-piperazin-1-yl)-phenylamino]-pyrimidin-4-yl}-5-methoxy-1H-indole-3-carboxamide (44 mg, solid).

¹HNMR (DMSO-d6, 400 MHz) δ: 9.74 (s, 1H), 8.73-8.74 (d, 1H), 8.54 (d, 1H), 8.19-8.21 (d, 1H), 7.84 (s, 1H), 7.79-7.80 (d, 1H), 7.40 (s, 1H), 7.11-7.20 (m, 3H), 6.90-6.93 (dd, 1H), 6.59-6.61 (d, 1H), 3.83 (s, 3H), 3.06-3.08 (m, 4H), 2.41 (m, 4H), 2.22 (s, 3H).

LC-MS (ESI): 476.2 (M+H)⁺.

### Example 142

### Preparation of 1-{2-[3-(4-ethyl-piperazin-1-yl)-phenylamino]-5-fluoro-pyrimidin-4-yl}-1H-indole-3-carboxamide (Compound 142)

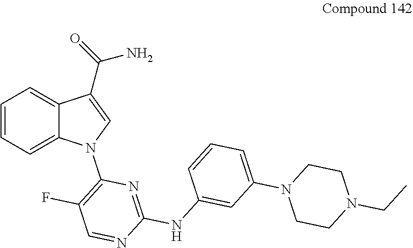 [SMILES: CCN1CCN(c2cccc(Nc3ncc(F)c(-n4cc(C(N)=O)c5ccccc54)n3)c2)CC1]

1-{2-[3-(4-Ethyl-piperazin-1-yl)-phenylamino]-5-fluoro-pyrimidin-4-yl}-1H-indole-3-carboxamide was obtained in accordance with the same preparation method of Example 115 except for replacing the N-methylpiperazine in Step 1 of Example 115 with N-ethylpiperazine.

¹HNMR (DMSO-d6, 400 MHz) δ: 9.78 (s, 1H), 8.77-8.78 (d, 1H), 8.55-8.56 (d, 1H), 8.28-8.31 (m, 1H), 8.21-8.22 (br, 1H), 7.84 (br, 1H), 7.42 (s, 1H), 7.31-7.33 (m, 2H), 7.10-7.18 (m, 3H), 6.59-6.61 (d, 1H), 3.06-3.08 (m, 4H), 2.43-2.44 (m, 4H), 2.32-2.37 (q, 2H), 1.00-1.04 (t, 3H).

LC-MS (ESI): 460.2 (M+H)⁺.

### Example 143

### Preparation of 1-{5-fluoro-2-[3-(4-methyl-piperazin-1-ylmethyl)-phenylamino]-pyrimidin-4-yl}-1H-indole-3-carboxamide (Compound 143)

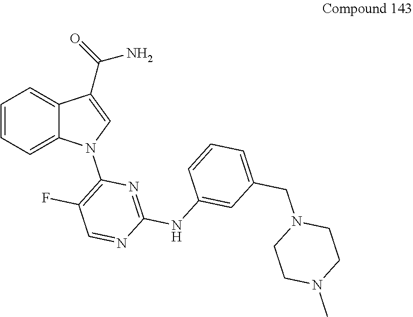 [SMILES: CN1CCN(Cc2cccc(Nc3ncc(F)c(-n4cc(C(N)=O)c5ccccc54)n3)c2)CC1]

1-{5-Fluoro-2-[3-(4-methyl-piperazin-1-ylmethyl)-phenylamino]-pyrimidin-4-yl}-1H-indole-3-carboxamide was obtained in accordance with the same preparation method of Example 115 except for replacing the 3-bromonitrobenzene in Step 1 of Example 115 with 3-nitrobenzyl bromide.

¹HNMR (DMSO-d6, 400 MHz) δ: 9.90 (s, 1H), 8.77-8.78 (d, 1H), 8.55 (d, 1H), 8.28-8.31 (m, 2H), 7.85-7.86 (br, 1H), 7.64-7.67 (m, 2H), 7.30-7.35 (m, 2H), 7.23-7.27 (t, 1H), 7.16 (br, 1H), 6.93-6.95 (d, 1H), 3.41 (m, 2H), 2.29-2.33 (m, 6H), 2.13 (s, 2H), 1.24 (s, 3H).

LC-MS (ESI): 460.2 (M+H)⁺.

### Example 144

### Preparation of 1-{5-fluoro-2-[3-(4-isopropyl-piperazin-1-yl)-phenylamino]-pyrimidin-4-yl}-1H-indole-3-carboxamide (Compound 144)

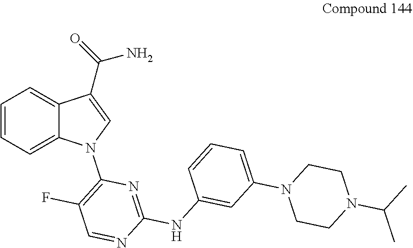 [SMILES: CC(C)N1CCN(c2cccc(Nc3ncc(F)c(-n4cc(C(N)=O)c5ccccc54)n3)c2)CC1]

1-{5-Fluoro-2-[3-(4-isopropyl-piperazin-1-yl)-phenylamino]-pyrimidin-4-yl}-1H-indole-3-carboxamide was obtained in accordance with the same preparation method of Example 115 except for replacing the N-methylpiperazine in Step 1 of Example 115 with N-isopropylpiperazine.

¹HNMR (DMSO-d6, 400 MHz) δ: 9.79 (s, 1H), 8.77-8.78 (d, 1H), 8.55 (d, 1H), 8.29-8.31 (m, 1H), 8.21-8.22 (br, 1H), 7.84 (br, 1H), 7.43 (s, 1H), 7.30-7.35 (m, 2H), 7.11-7.19 (m, 3H), 6.58-6.60 (d, 1H), 3.06 (s, 4H), 2.55-2.68 (m, 4H), 1.02 (s, 3H), 1.00 (s, 3H), 0.84-0.88 (m, 1H).

LC-MS (ESI): 474.2 (M+H)⁺.

### Example 145

### Preparation of 1-{2-[3-(4-sec-butyl-piperazin-1-yl)-phenylamino]-5-fluoro-pyrimidin-4-yl}-1H-indole-3-carboxamide (Compound 145)

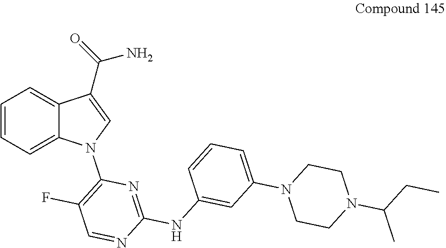 [SMILES: CCC(C)N1CCN(c2cccc(Nc3ncc(F)c(-n4cc(C(N)=O)c5ccccc54)n3)c2)CC1]

1-{2-[3-(4-Sec-butyl-piperazin-1-yl)-phenylamino]-5-fluoro-pyrimidin-4-yl}-1H-indole-3-carboxamide was obtained in accordance with the same preparation method of Example 115 except for replacing the N-methylpiperazine in Step 1 of Example 115 with N-sec-butylpiperazine.

¹HNMR (DMSO-d6, 400 MHz) δ: 9.78 (s, 1H), 8.77-8.78 (d, 1H), 8.55 (d, 1H), 8.29-8.31 (m, 1H), 8.21-8.22 (br, 1H), 7.84 (br, 1H), 7.43 (s, 1H), 7.30-7.33 (m, 2H), 7.11-7.17 (m, 3H), 6.58-6.60 (d, 1H), 3.05 (s, 4H), 2.50-2.54 (m, 4H), 1.49-1.51 (m, 1H), 1.24 (s, 3H), 0.93-0.95 (m, 2H), 0.85-0.89 (t, 3H).

LC-MS (ESI): 488.2 (M+H)⁺.

### Example 146

### Preparation of 1-[5-fluoro-2-(3-piperazin-1-yl-phenylamino)-pyrimidin-4-yl]-1H-indole-3-carboxylic acid (Compound 146)

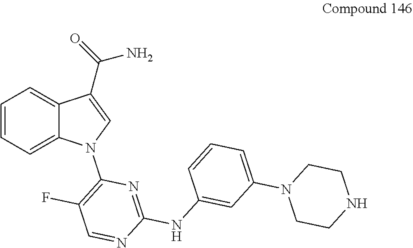 [SMILES: NC(=O)c1cn(-c2nc(Nc3cccc(N4CCNCC4)c3)ncc2F)c2ccccc12]

1-[5-Fluoro-2-(3-piperazin-1-yl-phenylamino)-pyrimidin-4-yl]-1H-indole-3-carboxylic acid was obtained in accordance with the same preparation method of Example 115 except for replacing the N-methylpiperazine in Step 1 of Example 115 with N-Boc-piperazine.

¹HNMR (DMSO-d6, 400 MHz) δ: 9.86 (s, 1H), 8.77-8.78 (d, 1H), 8.59 (d, 1H), 8.29-8.31 (m, 1H), 8.21 (br, 1H), 7.84 (br, 1H), 7.46 (s, 1H), 7.32-7.34 (m, 2H), 7.16-7.24 (m, 3H), 6.64-6.67 (d, 1H), 3.30-3.32 (m, 4H), 3.11-3.14 (m, 4H).

LC-MS (ESI): 432.0 (M+H)⁺.

### Example 147

### Preparation of 1-{2-[3-(4-tert-butyl-piperazin-1-yl)-phenylamino]-5-fluoro-pyrimidin-4-yl}-1H-indole-3-carboxamide (Compound 147)

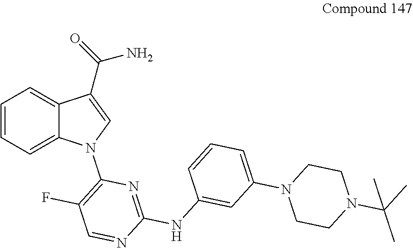 [SMILES: CC(C)(C)N1CCN(c2cccc(Nc3ncc(F)c(-n4cc(C(N)=O)c5ccccc54)n3)c2)CC1]

1-{2-[3-(4-Tert-butyl-piperazin-1-yl)-phenylamino]-5-fluoro-pyrimidin-4-yl}-1H-indole-3-carboxamide was obtained in accordance with the same preparation method of Example 115 except for replacing the N-methylpiperazine in Step 1 of Example 115 with N-Boc-piperazine.

¹HNMR (DMSO-d6, 400 MHz) δ: 9.78 (s, 1H), 8.77-8.78 (d, 1H), 8.55 (d, 1H), 8.29-8.31 (m, 1H), 8.21 (br, 1H), 7.84 (br, 1H), 7.43 (s, 1H), 7.31-7.34 (m, 2H), 7.05-7.14 (m, 3H), 6.57-6.58 (d, 1H), 3.03-3.05 (m, 4H), 2.57 (m, 4H), 1.03 (s, 9H).

LC-MS (ESI): 488.2 (M+H)⁺.

### Example 148

### Preparation of 1-(5-fluoro-2-{3-[4-(1-methyl-piperidin-4-yl)-piperazin-1-yl]-phenylamino}-pyrimidin-4-yl)-1H-indole-3-carboxamide (Compound 148)

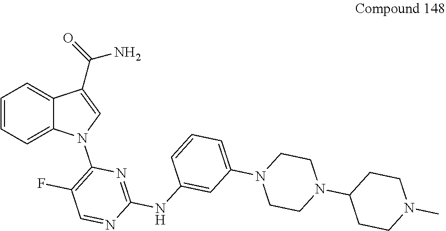 [SMILES: CN1CCC(N2CCN(c3cccc(Nc4ncc(F)c(-n5cc(C(N)=O)c6ccccc65)n4)c3)CC2)CC1]

1-(5-Fluoro-2-{3-[4-(1-methyl-piperidin-4-yl)-piperazin-1-yl]-phenylamino}-pyrimidin-4-yl)-1H-indole-3-carboxamide was obtained in accordance with the same preparation method of Example 115 except for replacing the N-methylpiperazine in Step 1 of Example 115 with 1-(1-methyl-4-piperidinyl)piperazine.

¹HNMR (DMSO-d6, 400 MHz) δ: 9.78 (s, 1H), 8.77-8.78 (d, 1H), 8.55-8.56 (d, 1H), 8.29-8.31 (m, 1H), 8.20-8.21 (m, 1H), 7.83 (br, 1H), 7.42 (s, 1H), 7.30-7.34 (m, 2H), 7.11-7.16 (m, 3H), 6.57-6.59 (d, 1H), 3.03-3.08 (m, 4H), 2.79-2.81 (m, 2H), 2.53-2.55 (m, 4H), 2.15 (s, 3H), 1.83-1.89 (t, 2H), 1.71-1.75 (m, 2H), 1.36-1.48 (m, 2H), 1.24-1.26 (m, 1H).

LC-MS (ESI): 529.2 (M+H)⁺.

### Example 149

### Preparation of 1-(5-fluoro-2-{3-[4-(tetrahydro-pyran-4-yl)-piperazin-1-yl]-phenylamino}-pyrimidin-4-yl)-5-methoxy-1H-indole-3-carboxamide (Compound 149)

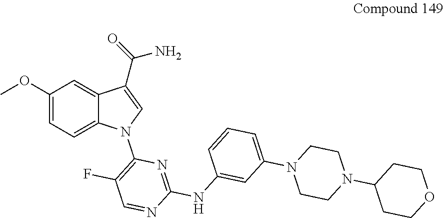 [SMILES: COc1ccc2c(c1)c(C(N)=O)cn2-c1nc(Nc2cccc(N3CCN(C4CCOCC4)CC3)c2)ncc1F]

1-(5-Fluoro-2-{3-[4-(tetrahydro-pyran-4-yl)-piperazin-1-yl]-phenylamino}-pyrimidin-4-yl)-5-methoxy-1H-indole-3-carboxamide was obtained in accordance with the same preparation method of Example 141 except for replacing the 3-(4-methyl-piperazin-1-yl)-aniline in Step 2 of Example 141 with 3-[4-(tetrahydro-pyran-4-yl)-piperazin-1-yl]-aniline (prepared in Example 140).

¹HNMR (DMSO-d6, 400 MHz) δ: 9.76 (s, 1H), 8.73-8.74 (d, 1H), 8.53 (d, 1H), 8.17-8.19 (d, 1H), 7.84 (br, 1H), 7.79-7.80 (d, 1H), 7.45 (s, 1H), 7.12-7.15 (m, 3H), 6.90-6.93 (dd, 1H), 6.57-6.59 (m, 1H), 3.88-3.92 (m, 2H), 3.83 (s, 3H), 3.26-3.3 (m, 2H), 2.99-3.04 (s, 4H), 2.55 (s, 4H), 2.38 (br, 1H), 1.71-1.74 (m, 2H), 1.36-1.45 (m, 2H).

LC-MS (ESI): 546.2 (M+H)⁺.

### Example 150

### Preparation of 1-(5-fluoro-2-{3-[4-(tetrahydro-furan-3-yl)-piperazin-1-yl]-phenylamino}-pyrimidin-4-yl)-1H-indole-3-carboxamide (Compound 150)

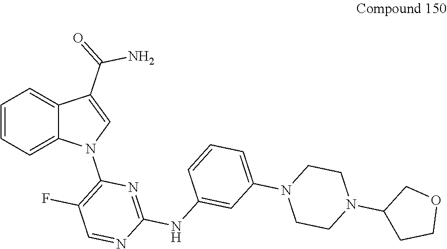 [SMILES: NC(=O)c1cn(-c2nc(Nc3cccc(N4CCN(C5CCOC5)CC4)c3)ncc2F)c2ccccc12]

1-(5-Fluoro-2-{3-[4-(tetrahydro-furan-3-yl)-piperazin-1-yl]-phenylamino}-pyrimidin-4-yl)-1H-indole-3-carboxamide was obtained in accordance with the same preparation method of Example 115 except for replacing the N-methylpiperazine in Step 1 of Example 115 with 1-(tetrahydrofuran-3-yl)piperazine hydrochloride.

¹HNMR (DMSO-d6, 400 MHz) δ: 9.81 (s, 1H), 8.77-8.78 (d, 1H), 8.56-8.57 (d, 1H), 8.28-8.30 (m, 1H), 8.19-8.23 (m, 1H), 7.79 (s, 1H), 7.43 (s, 1H), 7.31-7.37 (m, 2H), 7.12-7.27 (m, 3H), 6.60-6.62 (d, 1H), 3.76-3.79 (m, 2H), 3.63-3.69 (m, 2H), 3.06-3.08 (m, 4H), 2.65 (br, 1H), 2.49-2.50 (m, 4H), 1.97-2.05 (br, 2H).

LC-MS (ESI): 502.2 (M+H)⁺.

### Example 151

### Preparation of 1-(5-fluoro-2-{3-[4-(tetrahydro-furan-3-yl)-piperazin-1-yl]-phenylamino}-pyrimidin-4-yl)-5-methoxy-1H-indole-3-carboxamide (Compound 151

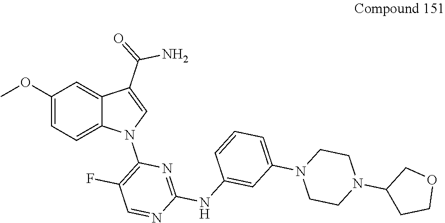 [SMILES: COc1ccc2c(c1)c(C(N)=O)cn2-c1nc(Nc2cccc(N3CCN(C4CCOC4)CC3)c2)ncc1F]

1-(5-Fluoro-2-(3-[4-(tetrahydro-furan-3-yl)-piperazin-1-yl]-phenylamino)-pyrimidin-4-yl)-5-methoxy-H-indole-3-carboxamide was obtained in accordance with the same preparation method of Example 141 except for replacing the 3-(4-methyl-piperazin-1-yl)-aniline in Step 2 of Example 141 with 3-[4-(tetrahydro-furan-3-yl)-piperazin-1-yl]-aniline (prepared in Example 150).

¹HNMR (DMSO-d6, 400 MHz) δ: 9.77 (s, 1H), 8.74-8.75 (d, 1H), 8.53 (d, 1H), 8.17-8.19 (d, 1H), 7.87 (br, 1H), 7.79-7.80 (d, 1H), 7.44 (s, 1H), 7.12-7.15 (m, 3H), 6.91-6.94 (dd, 1H), 6.59-6.60 (m, 1H), 3.74-3.832 (s, 3H), 3.76-3.80 (m, 2H), 3.63-3.69 (m, 2H), 3.06 (s, 4H), 2.60 (br, 1H), 2.49-2.47 (m, 4H), 2.00-2.02 (br, 2H).

LC-MS (ESI): 532.2 (M+H)⁺.

### Example 152

### Preparation of 1-{2-[3-(4-ethyl-piperazin-1-yl)-phenylamino]-5-fluoro-pyrimidin-4-yl}-5-methoxy-1H-indole-3-carboxamide (Compound 152)

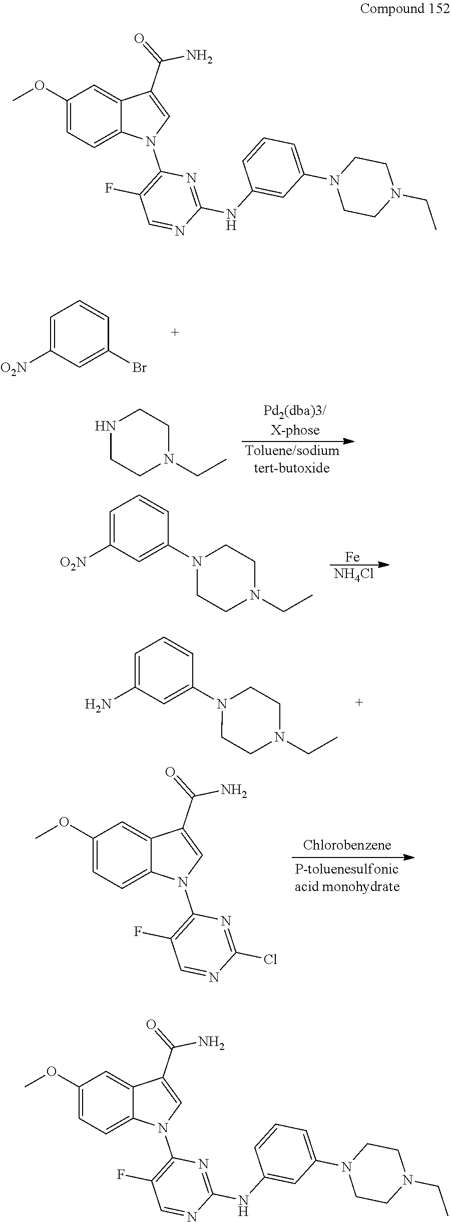

### Steps 1 and 2: Preparation of 3-(4-ethyl-piperazin-1-yl)-aniline

3-(4-Ethyl-piperazin-1-yl)-aniline was obtained in accordance with the same preparation method of Steps 1 and 2 of Example 115 except for replacing the N-methylpiperazine in Step 1 of Example 115 with N-ethylpiperazine.

### Step 3: Preparation of 1-{2-[3-(4-ethyl-piperazin-1-yl)-phenylamino]-5-fluoro-pyrimidin-4-yl}-5-methoxy-1H-indole-3-carboxamide

1-(2-Chloro-5-fluoro-pyrimidin-4-yl)-5-methoxy-1H-indole-3-carboxamide (192 mg, 0.6 mmol) (prepared in Step 1 of Example 141), 3-(4-ethyl-piperazin-1-yl)-aniline (102.5 mg, 0.5 mmol) (prepared in Steps 1 and 2) and p-toluenesulfonic acid monohydrate (114 mg, 0.6 mmol) were reacted in 12 ml of chlorobenzene at 130° C. for 15 hours. TLC showed that the reaction was completed. The reaction solution was cooled to room temperature, and the supernatant was removed. The viscous oil at the bottom of the flask was dissolved in a mixed solvent of dichloromethane/methanol (10:1). The solution was poured into saturated aqueous sodium bicarbonate solution (100 ml), and extracted with dichloromethane (60 ml×2). The organic phase was washed with saturated NaCl solution twice, dried over anhydrous sodium sulfate, filtrated and concentrated under reduced pressure. The resulting residues were purified by column chromatography (eluent: dichloromethane/methanol (containing 5% of aqueous ammonia)) to obtain 1-{2-[3-(4-ethyl-piperazin-1-yl)-phenylamino]-5-fluoro-pyrimidin-4-yl}-5-methoxy-1H-indole-3-carboxamide (39 mg, solid).

¹HNMR (DMSO-d6, 400 MHz) δ: 9.76 (s, 1H), 8.73-8.74 (d, 1H), 8.53 (d, 1H), 8.17-8.19 (d, 1H), 7.84 (br, 1H), 7.79-7.80 (d, 1H), 7.45 (s, 1H), 7.12-7.15 (m, 3H), 6.90-6.93 (dd, 1H), 6.57-6.59 (m, 1H), 3.83 (s, 3H), 3.07 (m, 4H), 2.46 (br, 4H), 2.36 (m, 2H), 1.01-1.05 (t, 3H).

LC-MS (ESI): 490.2 (M+H)⁺.

### Example 153

### Preparation of 1-[5-fluoro-2-(3-pyrazol-1-yl-phenylamino)-pyrimidin-4-yl]-1H-indole-3-carboxamide (Compound 153)

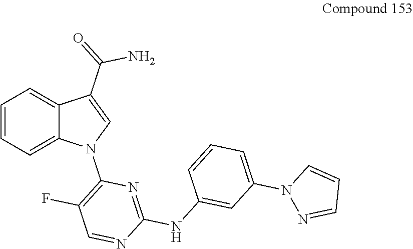 [SMILES: NC(=O)c1cn(-c2nc(Nc3cccc(-n4cccn4)c3)ncc2F)c2ccccc12]

1-[5-Fluoro-2-(3-pyrazol-1-yl-phenylamino)-pyrimidin-4-yl]-1H-indole-3-carboxamide was obtained in accordance with the same preparation method of Example 115 except for replacing the N-methylpiperazine in Step 1 of Example 115 with pyrazole.

¹HNMR (DMSO-d6, 400 MHz) δ: 10.14 (s, 1H), 8.83-8.84 (d, 1H), 8.58 (d, 1H), 8.38 (d, 11H), 8.29-8.33 (m, 2H), 7.78 (br, 1H), 7.73-7.74 (m, 1H), 7.66-7.68 (d, 1H), 7.18-7.46 (m, 4H), 6.66 (br, 1H), 6.52-6.53 (m, 1H), 5.32-5.34 (m, 1H).

LC-MS (ESI): 414.1 (M+H)⁺.

### Example 154

### Preparation of 1-{5-fluoro-2-[3-(4-methyl-pyrazol-1-yl)-phenylamino]-pyrimidin-4-yl}-1H-indole-3-carboxamide (Compound 154)

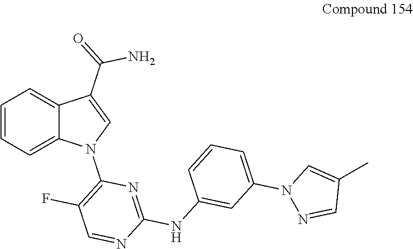 [SMILES: Cc1cnn(-c2cccc(Nc3ncc(F)c(-n4cc(C(N)=O)c5ccccc54)n3)c2)c1]

1-{5-Fluoro-2-[3-(4-methyl-pyrazol-1-yl)-phenylamino]-pyrimidin-4-yl}-1H-indole-3-carboxamide was obtained in accordance with the same preparation method of Example 115 except for replacing the N-methylpiperazine in Step 1 of Example 115 with 4-methylpyrazole.

¹HNMR (DMSO-d6, 400 MHz) δ: 10.13 (s, 1H), 8.83-8.84 (d, 1H), 8.58 (d, 1H), 8.27-8.31 (m, 3H), 8.05 (s, 1H), 7.78 (br, 1H), 7.59-7.62 (m, 1H), 7.54 (s, 1H), 7.36-7.42 (m, 2H), 7.29-7.34 (m, 2H), 7.19 (br, 1H), 2.07 (s, 3H).

LC-MS (ESI): 428.1 (M+H)⁺.

### Example 155

### Preparation of 1-(5-fluoro-2-{3-[4-(2-hydroxy-propyl)-piperazin-1-yl]-phenylamino}-pyrimidin-4-yl)-1H-indole-3-carboxamide (Compound 155)

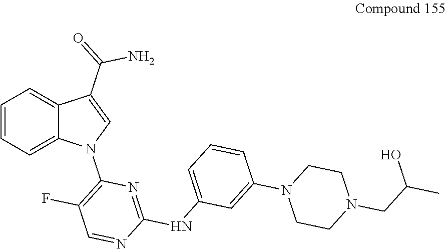 [SMILES: CC(O)CN1CCN(c2cccc(Nc3ncc(F)c(-n4cc(C(N)=O)c5ccccc54)n3)c2)CC1]

1-(5-Fluoro-2-{3-[4-(2-hydroxy-propyl)-piperazin-1-yl]-phenylamino}-pyrimidin-4-yl)-1H-indole-3-carboxamide was obtained in accordance with the same preparation method of Example 115 except for replacing the N-methylpiperazine in Step 1 of Example 115 with 1-piperazin-1-yl-propan-2-ol.

¹HNMR (DMSO-d6, 400 MHz) δ: 9.78 (s, 1H), 8.77-8.78 (d, 1H), 8.55 (d, 1H), 8.29-8.31 (m, 1H), 8.21 (m, 1H), 7.85 (br, 1H), 7.437 (s, 1H), 7.31-7.33 (m, 2H), 7.11-7.18 (m, 3H), 6.59-6.61 (d, 1H), 3.37-3.80 (m, 1H), 3.07-3.18 (m, 4H), 2.5 (m, 4H), 2.17-2.28 (m, 2H), 1.06-1.07 (d, 3H).

LC-MS (ESI): 490.0 (M+H)⁺.

### Example 156

### Preparation of 1-{5-fluoro-2-[3-(4-oxiranylmethyl-piperazin-1-yl)-phenylamino]-pyrimidin-4-yl}-1H-indole-3-carboxamide (Compound 156

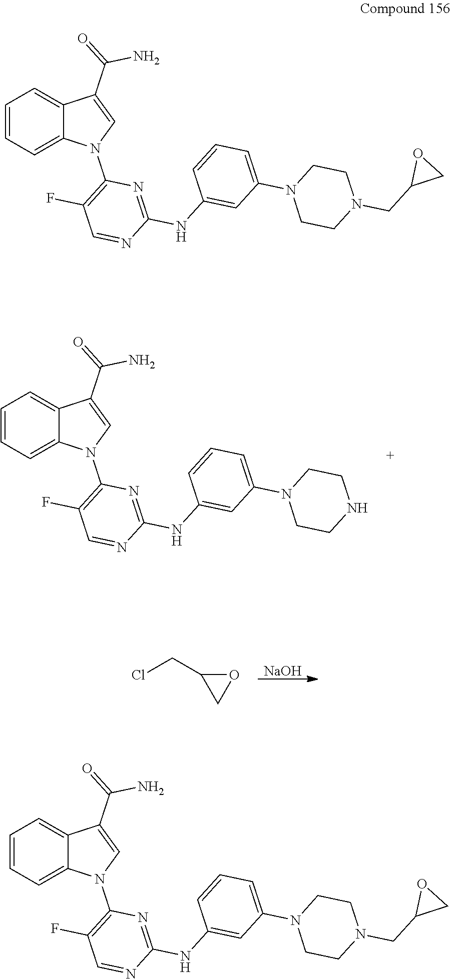

### Step 1: Preparation of 1-{5-fluoro-2-[3-(4-oxiranylmethyl-piperazin-1-yl)-phenylamino]-pyrimidin-4-yl}-1H-indole-3-carboxamide

1-[5-Fluoro-2-(3-piperazin-1-yl-phenylamino)-pyrimidin-4-yl]-1H-indole-3-carboxylic acid (100 mg, 0.23 mmol) (prepared in Example 146) was dissolved in 10 ml of acetone. Sodium hydroxide (43 mg, 0.46 mmol), epichlorohydrin (230 mg, 0.58 mmol) and a catalytic amount of potassium iodide were added successively at room temperature. The reaction system was heated to 50° C. and reacted for 6 hours. TLC showed that the reaction was completed. The reaction system was poured into water (80 ml), and extracted with dichloromethane (50 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol) to obtain 1-{5-fluoro-2-[3-(4-oxiranylmethyl-piperazin-1-yl)-phenylamino]-pyrimidin-4-yl}-1H-indole-3-carboxamide (11 mg, solid).

¹HNMR (DMSO-d6, 400 MHz) δ: 9.79 (s, 1H), 8.77-8.78 (d, 1H), 8.55 (d, 1H), 8.29-8.31 (m, 1H), 8.24 (m, 1H), 7.82-7.89 (br, 1H), 7.42 (m, 1H), 7.32-7.33 (m, 2H), 7.11-7.18 (m, 3H), 6.60-6.62 (d, 1H), 3.40-3.41 (m, 3H), 3.06-3.10 (m, 4H), 2.48-2.50 (m, 4H), 2.22-2.27 (m, 2H).

LC-MS (ESI): 488.0 (M+H)⁺.

### Example 157

### Preparation of 1-(5-fluoro-2-{3-[4-(tetrahydro-pyran-4-ylmethyl)-piperazin-1-yl]-phenylamino}-pyrimidin-4-yl)-1H-indole-3-carboxamide (Compound 157)

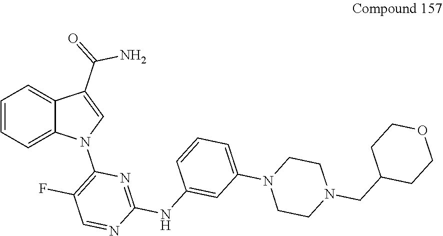 [SMILES: NC(=O)c1cn(-c2nc(Nc3cccc(N4CCN(CC5CCOCC5)CC4)c3)ncc2F)c2ccccc12]

1-(5-Fluoro-2-{3-[4-(tetrahydro-pyran-4-ylmethyl)-piperazin-1-yl]-phenylamino}-pyrimidin-4-yl)-1H-indole-3-carboxamide was obtained in accordance with the same preparation method of Example 156 except for replacing the epichlorohydrin in Step 1 of Example 156 with 4-bromomethyltetrahydropyran.

¹HNMR (DMSO-d6, 400 MHz) δ: 9.78 (s, 1H), 8.77-8.78 (d, 1H), 8.55 (d, 1H), 8.30-8.31 (m, 1H), 8.20-8.22 (m, 1H), 7.84 (br, 1H), 7.43 (s, 1H), 7.29-7.34 (m, 2H), 7.10-7.18 (m, 3H), 6.59-6.60 (d, 1H), 3.82-3.85 (dd, 2H), 3.28-3.30 (m, 2H), 3.05 (br, 4H), 2.37-2.47 (br, 4H), 2.15-2.18 (d, 2H), 1.76 (br, 1H), 1.60-1.62 (d, 2H), 1.08-1.17 (m, 2H).

LC-MS (ESI): 530.2 (M+H)⁺.

### Example 158

### Preparation of 1-(5-fluoro-2-{3-[4-(tetrahydro-furan-2-carbonyl)-piperazin-1-yl]-phenylamino}-pyrimidin-4-yl)-1H-indole-3-carboxamide (Compound 158)

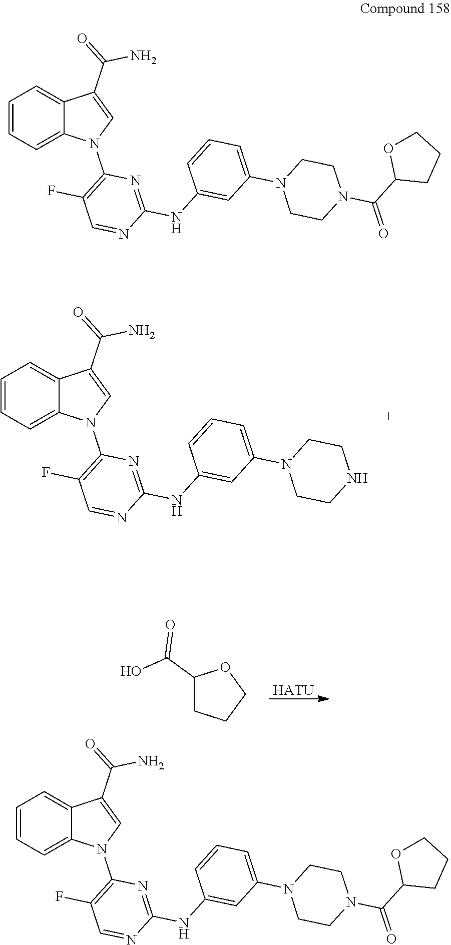

### Step 1: Preparation of 1-(5-fluoro-2-{3-[4-(tetrahydro-furan-2-carbonyl)-piperazin-1-yl]-phenylamino}-pyrimidin-4-yl)-1H-indole-3-carboxamide

1-[5-Fluoro-2-(3-piperazin-1-yl-phenylamino)-pyrimidin-4-yl]-1H-indole-3-carboxamide (200 mg, 0.46 mmol) (prepared in Example 146) was dissolved in 5 ml of DMF. 2-Tetrahydrofuroic acid (65 mg, 0.56 mmol), HATU (270 mg, 0.70 mmol) and N-methylmorpholine (140 mg, 1.38 mmol) were added successively at room temperature. The reaction system was reacted at room temperature for 1 hour. TLC showed that the reaction was completed. The reaction system was poured into water (80 ml), and extracted with dichloromethane (50 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol) to obtain 1-(5-fluoro-2-{3-[4-(tetrahydro-furan-2-carbonyl)-piperazin-1-yl]-phenylamino}-pyrimidin-4-yl)-1H-indole-3-carboxamide (15 mg, solid).

¹HNMR (DMSO-d6, 400 MHz) δ: 9.83 (s, 1H), 8.78-8.79 (d, 1H), 8.56 (d, 1H), 8.29-8.32 (m, 1H), 8.21-8.23 (m, 1H), 7.86 (br, 1H), 7.46 (s, 1H), 7.32-7.35 (m, 2H), 7.15-7.19 (m, 3H), 6.62-6.64 (d, 1H), 4.66-4.69 (m 1H), 3.72-3.81 (m, 2H), 3.50-3.63 (m, 4H), 3.04-3.07 (m, 4H), 1.97-2.05 (m, 2H), 1.81-1.87 (m, 2H).

LC-MS (ESI): 530.0 (M+H)⁺.

### Example 159

### Preparation of 1-{5-fluoro-2-[3-(4-isopropyl-piperazin-1-yl)-phenylamino]-pyrimidin-4-yl}-5-methoxy-1H-indole-3-carboxamide (Compound 159)

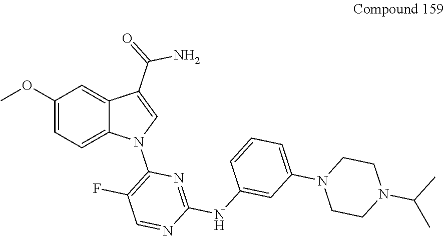 [SMILES: COc1ccc2c(c1)c(C(N)=O)cn2-c1nc(Nc2cccc(N3CCN(C(C)C)CC3)c2)ncc1F]

1-{5-Fluoro-2-[3-(4-isopropyl-piperazin-1-yl)-phenylamino]-pyrimidin-4-yl}-5-methoxy-1H-indole-3-carboxamide was obtained in accordance with the same preparation method of Example 152 except for replacing the N-ethylpiperazine in Steps 1 and 2 of Example 152 with N-isopropylpiperazine.

¹HNMR (DMSO-d6, 400 MHz) δ: 9.78 (s, 1H), 8.74-8.75 (d, 1H), 8.54 (d, 1H), 8.19-8.21 (d, 1H), 7.85 (br, 1H), 7.80-7.81 (d, 1H), 7.44 (s, 1H), 7.12-7.15 (m, 3H), 6.91-6.94 (dd, 1H), 6.59-6.61 (m, 1H), 3.83 (s, 3H), 3.10-3.18 (m, 4H), 2.64 (br, 4H), 1.23 (br, 1H), 1.05 (m, 6H).

LC-MS (ESI): 504.0 (M+H)⁺.

### Example 160

### Preparation of 1-(5-fluoro-2-{3-[4-(tetrahydro-furan-2-ylmethyl)-piperazin-1-yl]-phenylamino}-pyrimidin-4-yl)-1H-indole-3-carboxamide (Compound 160)

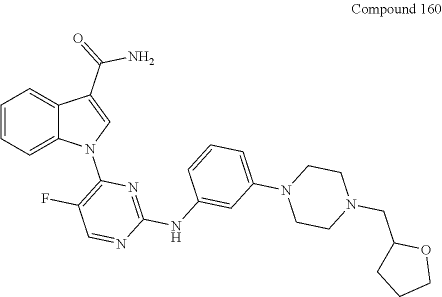 [SMILES: NC(=O)c1cn(-c2nc(Nc3cccc(N4CCN(CC5CCCO5)CC4)c3)ncc2F)c2ccccc12]

1-(5-Fluoro-2-{3-[4-(tetrahydro-furan-2-ylmethyl)-piperazin-1-yl]-phenylamino}-pyrimidin-4-yl)-1H-indole-3-carboxamide was obtained in accordance with the same preparation method of Example 156 except for replacing the epichlorohydrin in Step 1 of Example 156 with 2-chloromethyltetrahydrofuran.

¹HNMR (DMSO-d6, 400 MHz) δ: 9.78 (s, 1H), 8.77-8.78 (d, 1H), 8.55 (d, 1H), 8.30-8.31 (m, 1H), 8.21-8.22 (m, 1H), 7.84 (br, 1H), 7.41 (s, 1H), 7.30-7.34 (m, 2H), 7.11-7.19 (m, 3H), 6.59-6.61 (d, 1H), 3.98-4.04 (m 1H), 3.61-3.79 (m, 2H), 3.02-3.08 (m, 4H), 2.52-2.67 (m, 6H), 1.44-1.99 (m, 4H).

LC-MS (ESI): 516.0 (M+H)⁺.

### Example 161

### Preparation of 1-{2-[3-(4-ethyl-piperazin-1-yl)-phenylamino]-5-fluoro-pyrimidin-4-yl}-5-fluoro-1H-indole-3-carboxamide (Compound 161)

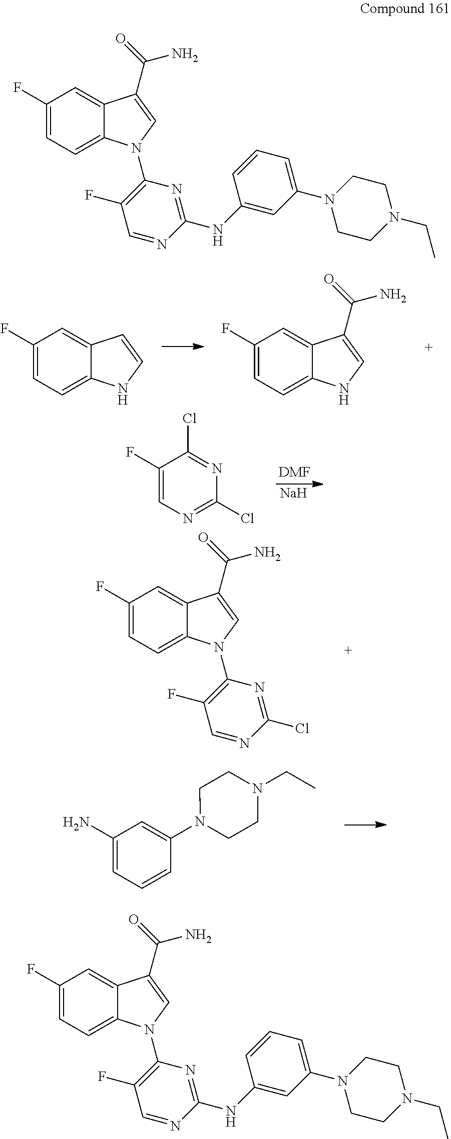

### Step 1: Preparation of 5-fluoro-H-indole-3-carboxamide

5-Fluoro-H-indole-3-carboxamide was obtained in accordance with the same preparation method of Steps 1 to 4 of Example 29 except for replacing the 3-fluoroindole in Step 1 of Example 29 with 5-fluoroindole.

### Step 2: Preparation of 1-(2-chloro-5-fluoro-pyrimidin-4-yl)-5-fluoro-1H-indole-3-carboxamide

5-Fluoro-1H-indole-3-carboxamide (1.9 g, 10.7 mmol) was dissolved in 20 ml of DMF at room temperature. The solution was cooled in an ice water bath to 0-5° C., to which sodium hydride (427 mg, 10.7 mmol) was slowly added. After completion of the addition, the reaction solution was warmed and reacted for 30 minutes to obtain mixed system A. 2,4-Dichloro-5-fluoropyrimidine (2.67 g, 16.0 mmol) was dissolved in 20 ml of DMF and added with the above mixed system A slowly at room temperature. After completion of the addition, the reaction solution was reacted at room temperature for 2 hours. TLC showed that the reaction was completed. The reaction system was poured into water (300 ml), and solid was precipitated. The solution was filtrated, and the filter cake was washed with water. The resulting solid was dried by blowing (60° C.) for 12 hours to obtain 1-(2-chloro-5-fluoro-pyrimidin-4-yl)-5-fluoro-1H-indole-3-carboxamide (2.2 g, solid). The product was used directly in the next step without purification.

### Step 3: Preparation of 1-{2-[3-(4-ethyl-piperazin-1-yl)-phenylamino]-5-fluoro-pyrimidin-4-yl}-5-fluoro-1H-indole-3-carboxamide

1-(2-Chloro-5-fluoro-pyrimidin-4-yl)-5-fluoro-1H-indole-3-carboxamide (185 mg, 0.6 mmol) (prepared in Step 2), 3-(4-ethyl-piperazin-1-yl)-aniline (102.5 mg, 0.5 mmol) (prepared in Steps 1 and 2 of Example 152) and p-toluenesulfonic acid monohydrate (114 mg, 0.6 mmol) were reacted in 12 ml of chlorobenzene at 130° C. for 15 hours. TLC showed that the reaction was completed. The reaction solution was cooled to room temperature, and the supernatant was removed. The viscous oil at the bottom of the flask was dissolved in a mixed solvent of dichloromethane/methanol (10:1). The solution was poured into saturated aqueous sodium bicarbonate solution (100 ml), and extracted with dichloromethane (60 ml×2). The organic phase was washed with saturated NaCl solution twice, dried over anhydrous sodium sulfate, filtrated and concentrated under reduced pressure. The resulting residues were purified by column chromatography (eluent: dichloromethane/methanol (containing 5% of aqueous ammonia)) to obtain 1-{2-[3-(4-ethyl-piperazin-1-yl)-phenylamino]-5-fluoro-pyrimidin-4-yl}-5-fluoro-1H-indole-3-carboxamide (70 mg, solid).

¹HNMR (DMSO-d6, 400 MHz) δ: 9.80 (s, 1H), 8.77-8.78 (d, 1H), 8.65 (d, 1H), 8.28-8.31 (m, 1H), 7.98-8.02 (dd, 1H), 7.93-7.91 (br, 1H), 7.40 (s, 1H), 7.19-7.20 (m, 1H), 7.12-7.18 (m, 3H), 6.59-6.62 (m, 1H), 3.09 (br, 4H), 2.43-2.51 (m, 6H), 1.03-1.06 (t, 3H).

LC-MS (ESI): 478.0 (M+H)⁺.

### Example 162

### Preparation of 5-amino-1-{2-[3-(4-ethyl-piperazin-1-yl)-phenylamino]-5-fluoro-pyrimidin-4-yl}-1H-indole-3-carboxamide (Compound 162)

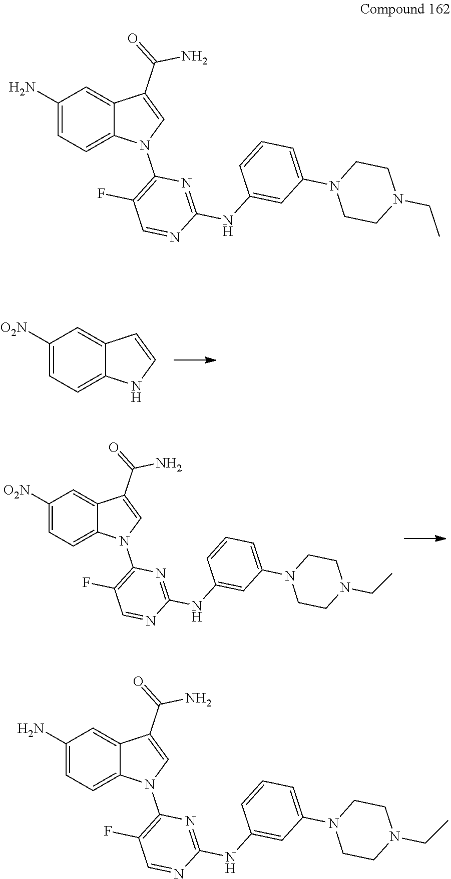 [SMILES: CCN1CCN(c2cccc(Nc3ncc(F)c(-n4cc(C(N)=O)c5cc(N)ccc54)n3)c2)CC1 ; O=[N+]([O-])c1ccc2[nH]ccc2c1 ; CCN1CCN(c2cccc(Nc3ncc(F)c(-n4cc(C(N)=O)c5cc([N+](=O)[O-])ccc54)n3)c2)CC1 ; CCN1CCN(c2cccc(Nc3ncc(F)c(-n4cc(C(N)=O)c5cc(N)ccc54)n3)c2)CC1]

### Step 1: Preparation of 1-{2-[3-(4-ethyl-piperazin-1-yl)phenylamino]-5-fluoro-pyrimidin-4-yl}-5-nitro-1H-indole-3-carboxamide

1-{2-[3-(4-Ethyl-piperazin-1-yl)phenylamino]-5-fluoro-pyrimidin-4-yl}-5-nitro-1H-indole-3-carboxamide was obtained in accordance with the same preparation method of Example 161 except for replacing the 5-fluoroindole in Step 1 of Example 161 with 5-nitroindole.

### Step 2: Preparation of 5-amino-1-{2-[3-(4-ethyl-piperazin-1-yl)-phenylamino]-5-fluoro-pyrimidin-4-yl}-1H-indole-3-carboxamide

1-{2-[3-(4-Ethyl-piperazin-1-yl)phenylamino]-5-fluoro-pyrimidin-4-yl}-5-nitro-1H-indole-3-carboxamide (252 mg, 0.5 mmol) (prepared in Step 1), reduced iron powder (112 mg, 2 mmol) and ammonium chloride (188 mg, 3.5 mmol) were dissolved in a mixed solvent of ethanol (12 ml) and water (4 ml). The reaction solution was heated to 90° C. and reacted for 1 hour. TLC showed that the reaction was completed. The reaction system was cooled to room temperature, poured into saturated aqueous sodium bicarbonate solution (100 ml), and extracted with ethyl acetate (60 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol (containing 5% of aqueous ammonia)) to obtain 5-amino-1-{2-[3-(4-ethyl-piperazin-1-yl)-phenylamino]-5-fluoro-pyrimidin-4-yl}-1H-indole-3-carboxamide (20 mg, solid).

¹HNMR (DMSO-d6, 400 MHz) δ: 9.67 (s, 1H), 8.67-8.78 (d, 1H), 8.39 (d, 1H), 8.01-8.03 (d, 1H), 7.69-7.73 (br, 1H), 7.47 (d, 1H), 7.39 (br, 1H), 7.10-7.19 (m, 2H), 6.98-7.04 (m, 1H), 6.59-6.64 (m, 2H), 4.92-5.06 (br, 2H), 3.09 (br, 4H), 2.34-2.51 (m, 6H), 1.02-1.06 (t, 3H).

LC-MS (ESI): 475.1 (M+H)⁺.

### Example 163

### Preparation of 1-{2-[3-(4-ethyl-piperazin-1-yl)-phenylamino]-5-fluoro-pyrimidin-4-yl}-5-hydroxy-1H-indole-3-carboxamide (Compound 163)

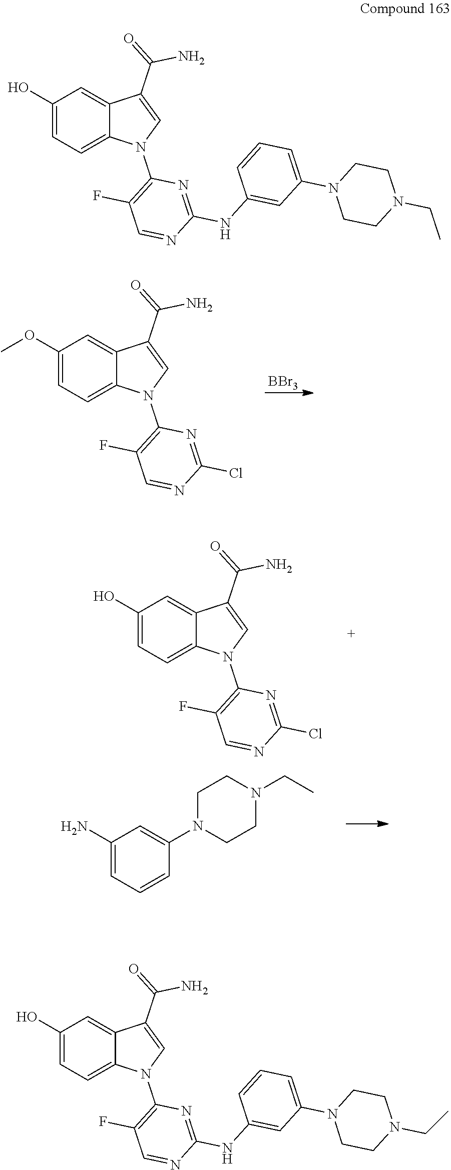

### Step 1: Preparation of 1-(2-chloro-5-fluoro-pyrimidin-4-yl)-5-hydroxy-1H-indole-3-carboxamide

1-(2-Chloro-5-fluoro-pyrimidin-4-yl)-5-methoxy-H-indole-3-carboxamide (2.5 g, 7.8 mmol) (prepared in Step 1 of Example 141) was stirred in 100 ml of dichloromethane at room temperature to obtain a turbid system. The system was cooled to below −20° C. by dry ice/ethanol under a nitrogen atmosphere (purged with nitrogen three times), to which 40 ml of a solution (1M) of boron tribromide in dichloromethane was slowly added dropwise. After completion of the addition, the reaction solution was warmed to 0° C. and reacted for 4 hours. TLC showed that the reaction was completed. The reaction solution was slowly poured into water (800 ml), and the pH was adjusted to around 7 by saturated aqueous sodium bicarbonate solution. The solution was filtrated, and the filter cake was washed with water. The resulting solid was dried by blowing (60° C.) for 12 hours to obtain 1-(2-chloro-5-fluoro-pyrimidin-4-yl)-5-hydroxy-1H-indole-3-carboxamide (2.3 g, yellowish white solid).

### Step 2: Preparation of 1-{2-[3-(4-ethyl-piperazin-1-yl)-phenylamino]-5-fluoro-pyrimidin-4-yl}-5-hydroxy-1H-indole-3-carboxamide

1-{2-[3-(4-Ethyl-piperazin-1-yl)-phenylamino]-5-fluoro-pyrimidin-4-yl}-5-hydroxy-1H-indole-3-carboxamide was obtained in accordance with the same preparation method of Step 3 of Example 161 except for replacing the 1-(2-chloro-5-fluoro-pyrimidin-4-yl)-5-fluoro-1H-indole-3-carboxamide in Step 3 of Example 161 with 1-(2-chloro-5-fluoro-pyrimidin-4-yl)-5-hydroxy-1H-indole-3-carboxamide (prepared in Step 1).

¹HNMR (DMSO-d6, 400 MHz) δ: 9.27 (s, 1H), 8.71-8.72 (d, 1H), 8.48 (d, 1H), 8.12-8.14 (d, 1H), 7.80 (br, 1H), 7.69-7.70 (d, 1H), 7.39 (s, 1H), 7.08-7.18 (m, 3H), 6.76-6.78 (dd, 1H), 6.58-6.60 (d, 1H), 3.06-3.08 (m, 4H), 2.42-2.45 (m, 4H), 2.32-2.37 (q, 2H), 1.01-1.04 (t, 3H).

LC-MS (ESI): 476.0 (M+H)⁺.

### Example 164

### Preparation of 1-{2-[3-(4-ethyl-piperazin-1-yl)-phenylamino]-5-fluoro-pyrimidin-4-yl}-5-(2-methoxy-ethoxy)-1H-indole-3-carboxamide (Compound 164)

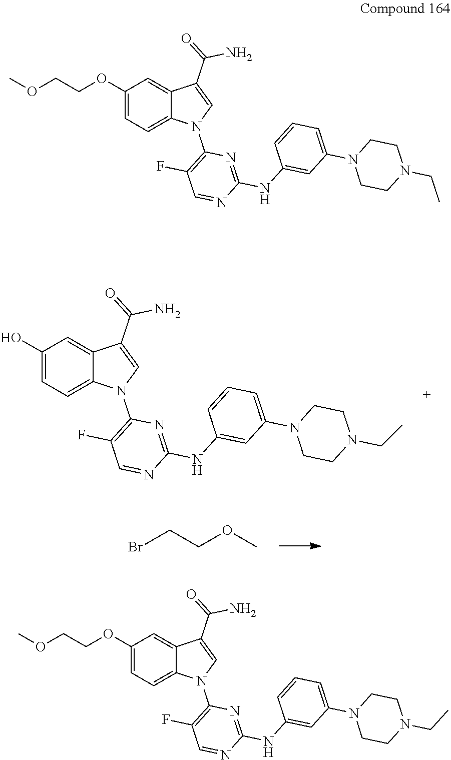

### Step 1: Preparation of 1-{2-[3-(4-ethyl-piperazin-1-yl)-phenylamino]-5-fluoro-pyrimidin-4-yl}-5-(2-methoxy-ethoxy)-1H-indole-3-carboxamide

1-{2-[3-(4-Ethyl-piperazin-1-yl)-phenylamino]-5-fluoro-pyrimidin-4-yl}-5-hydroxy-1H-indole-3-carboxamide (111 mg, 0.234 mmol) (prepared in Example 163) was dissolved in 10 ml of DMF at room temperature. 2-Bromoethyl methyl ether (49 mg, 0.351 mmol), cesium carbonate (229 mg, 0.701 mmol) and a catalytic amount of potassium iodide were added successively. The reaction system was heated to 90° C. and reacted for 3 hours. TLC showed that the reaction was completed. The reaction system was cooled to room temperature, poured into water (100 ml), and extracted with ethyl acetate (60 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol (containing 5% of aqueous ammonia)) to obtain 1-{2-[3-(4-ethyl-piperazin-1-yl)-phenylamino]-5-fluoro-pyrimidin-4-yl}-5-(2-methoxy-ethoxy)-1H-indole-3-carboxamide (22 mg, solid).

¹HNMR (DMSO-d6, 400 MHz) δ: 9.77 (s, 1H), 8.73-8.74 (d, 1H), 8.54 (d, 1H), 8.19-8.22 (d, 1H), 7.84 (br, 1H), 7.79-7.80 (d, 1H), 7.42 (s, 1H), 7.13-7.19 (m, 3H), 6.92-6.95 (dd, 1H), 6.61-6.63 (d, 1H), 4.13-4.16 (t, 2H), 3.70-3.73 (t, 2H), 3.36 (s, 3H), 3.13-3.16 (br, 4H), 2.55-2.61 (m, 6H), 1.24 (s, 3H).

LC-MS (ESI): 534.1 (M+H)⁺.

### Example 165

### Preparation of 1-{2-[3-(4-acetyl-piperazin-1-yl)-phenylamino]-5-fluoro-pyrimidin-4-yl}-1H-indole-3-carboxamide (Compound 165)

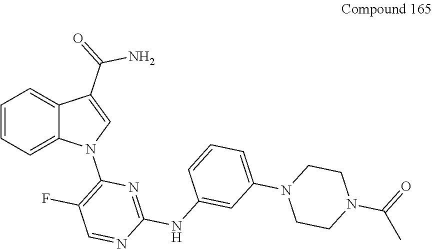 [SMILES: CC(=O)N1CCN(c2cccc(Nc3ncc(F)c(-n4cc(C(N)=O)c5ccccc54)n3)c2)CC1]

1-{2-[3-(4-Acetyl-piperazin-1-yl)-phenylamino]-5-fluoro-pyrimidin-4-yl}-1H-indole-3-carboxamide was obtained in accordance with the same preparation method of Example 115 except for replacing the N-methylpiperazine in Step 1 of Example 115 with 1-acetylpiperazine.

¹HNMR (DMSO-d6, 400 MHz) δ: 9.82 (s, 1H), 8.77-8.78 (d, 1H), 8.56 (d, 1H), 8.30-8.31 (m, 1H), 8.21 (m, 1H), 7.84 (br, 1H), 7.46 (s, 1H), 7.32-7.35 (m, 2H), 7.14-7.20 (m, 3H), 6.62-6.64 (d, 1H), 3.50-3.55 (m, 4H), 3.01-3.09 (m, 4H), 2.02 (s, 3H).

LC-MS (ESI): 474.1 (M+H)⁺.

### Example 166

### Preparation of 1-(5-fluoro-2-{3-[4-(tetrahydro-pyran-4-carbonyl)-piperazin-1-yl]-phenylamino}-pyrimidin-4-yl)-1H-indole-3-carb

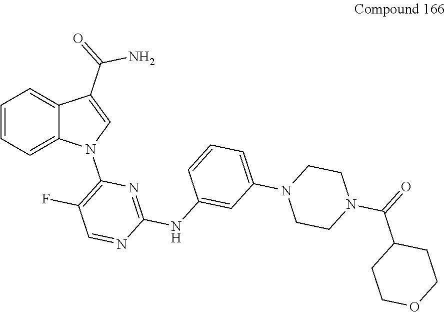 [SMILES: NC(=O)c1cn(-c2nc(Nc3cccc(N4CCN(C(=O)C5CCOCC5)CC4)c3)ncc2F)c2ccccc12]

1-(5-Fluoro-2-{3-[4-(tetrahydro-pyran-4-carbonyl)-piperazin-1-yl]-phenylamino}-pyrimidin-4-yl)-1H-indole-3-carboxamide was obtained in accordance with the same preparation method of Example 158 except for replacing the 2-tetrahydrofuroic acid in Step 1 of Example 158 with tetrahydropyran-4-carboxylic acid.

¹HNMR (DMSO-d6, 400 MHz) δ: 9.84 (s, 1H), 8.78-8.79 (d, 1H), 8.56 (d, 1H), 8.30-8.31 (m, 1H), 8.21 (m, 1H), 7.84 (br, 1H), 7.49 (s, 1H), 7.31-7.33 (m, 2H), 7.14-7.21 (m, 3H), 6.63-6.64 (d, 1H), 3.84-3.87 (m, 2H), 3.54-3.65 (m, 6H), 3.04-3.31 (m, 4H), 2.85-2.88 (m, 1H), 1.91-2.01 (m, 2H), 1.50-1.61 (m, 2H).

LC-MS (ESI): 544.2 (M+H)⁺.

### Example 167

### Preparation of 1-(5-fluoro-2-{3-[4-(morpholin-4-carbonyl)-piperazin-1-yl]-phenylamino}-pyrimidin-4-yl)-1H-indole-3-carboxamide (Compound 167)

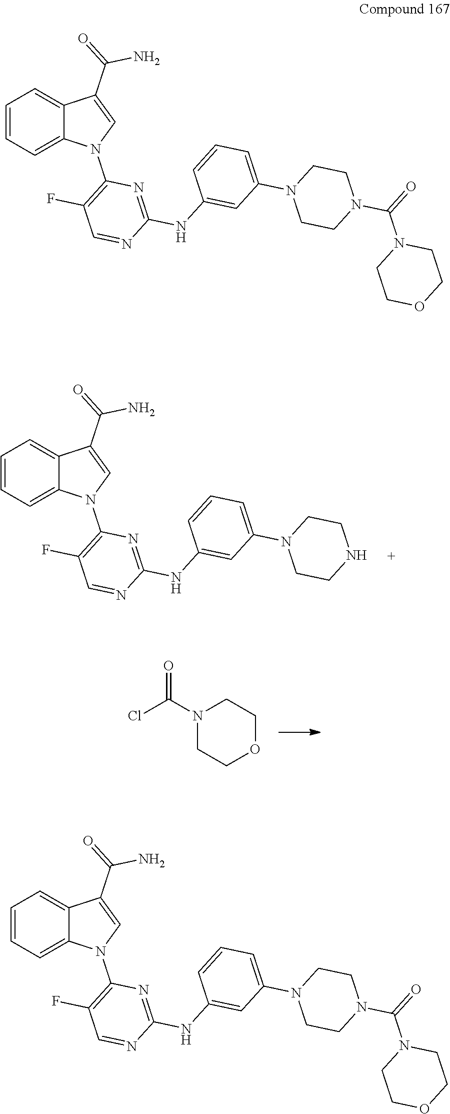 [SMILES: NC(=O)c1cn(-c2nc(Nc3cccc(N4CCN(C(=O)N5CCOCC5)CC4)c3)ncc2F)c2ccccc12 ; NC(=O)c1cn(-c2nc(Nc3cccc(N4CCNCC4)c3)ncc2F)c2ccccc12 ; O=C(Cl)N1CCOCC1 ; NC(=O)c1cn(-c2nc(Nc3cccc(N4CCN(C(=O)N5CCOCC5)CC4)c3)ncc2F)c2ccccc12]

1-[5-Fluoro-2-(3-piperazin-1-yl-phenylamino)-pyrimidin-4-yl]-1H-indole-3-carboxylic acid (100 mg, 0.23 mmol) (prepared in Example 146) and N,N-dimethylethylamine (90 mg, 0.698 mmol) were dissolved in 10 ml of DMF at room temperature. The reaction solution was cooled in an ice water bath to 0-5° C., to which morpholine-4-carbonyl chloride (52 mg, 0.349 mmol) was added dropwise. After completion of the addition, the reaction solution was warmed to room temperature and reacted for 1 hour. TLC showed that the reaction was completed. The reaction system was poured into water (100 ml), and extracted with ethyl acetate (60 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol (containing 5% of aqueous ammonia)) to obtain 1-(5-fluoro-2-{3-[4-(morpholin-4-carbonyl)-piperazin-1-yl]-phenylamino}-pyrimidin-4-yl)-1H-indole-3-carboxamide (67 mg, solid).

¹HNMR (DMSO-d6, 400 MHz) δ: 9.82 (s, 1H), 8.78-8.79 (d, 1H), 8.56 (d, 1H), 8.30-8.32 (m, 1H), 8.21 (m, 1H), 7.84 (br, 1H), 7.49 (s, 1H), 7.30-7.33 (m, 2H), 7.12-7.16 (m, 3H), 6.61-6.63 (m, 1H), 3.57-3.59 (m, 4H), 3.21-3.24 (m, 4H), 3.14-3.16 (m, 4H), 3.05-3.06 (m, 4H).

LC-MS (ESI): 545.2 (M+H)⁺.

### Example 168

### Preparation of 1-{2-[3-(4-ethyl-piperazin-1-yl)-phenylamino]-5-fluoro-pyrimidin-4-yl}-5-(2-morpholin-4-yl-ethoxy)-1H-indole-3-carboxamide (Compound 168)

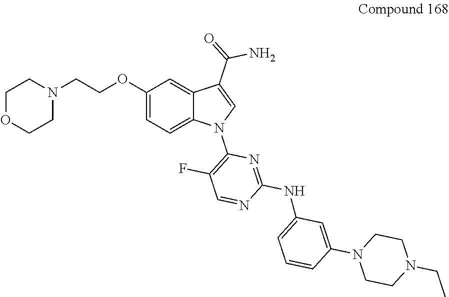 [SMILES: CCN1CCN(c2cccc(Nc3ncc(F)c(-n4cc(C(N)=O)c5cc(OCCN6CCOCC6)ccc54)n3)c2)CC1]

1-{2-[3-(4-Ethyl-piperazin-1-yl)-phenylamino]-5-fluoro-pyrimidin-4-yl}-5-(2-morpholin-4-yl-ethoxy)-H-indole-3-carboxamide was obtained in accordance with the same preparation method of Example 164 except for replacing the 2-bromoethyl methyl ether in Step 1 of Example 164 with 4-(2-chloroethyl)morpholine hydrochloride.

¹HNMR (DMSO-d6, 400 MHz) δ: 9.81 (s, 1H), 8.74-8.75 (d, 1H), 8.54 (d, 1H), 8.17-8.20 (d, 1H), 7.85 (br, 1H), 7.81-7.82 (d, 1H), 7.74 (s, 1H), 7.16-7.23 (m, 3H), 6.93-6.95 (dd, 1H), 6.65-6.67 (d, 1H), 4.18 (m, 2H), 3.63 (m, 4H), 3.51 (m, 4H), 2.58-3.08 (m, 10H), 1.97-2.01 (q, 2H), 1.19-1.26 (m, 3H).

LC-MS (ESI): 589.0 (M+H)⁺.

### Example 169

### Preparation of 1-(5-fluoro-2-{3-[4-(pyrrolidin-2-carbonyl)-1-piperazin-1-yl]-phenylamino}-pyrimidin-4-yl)-1H-indole-3-carboxamide (Compound 169)

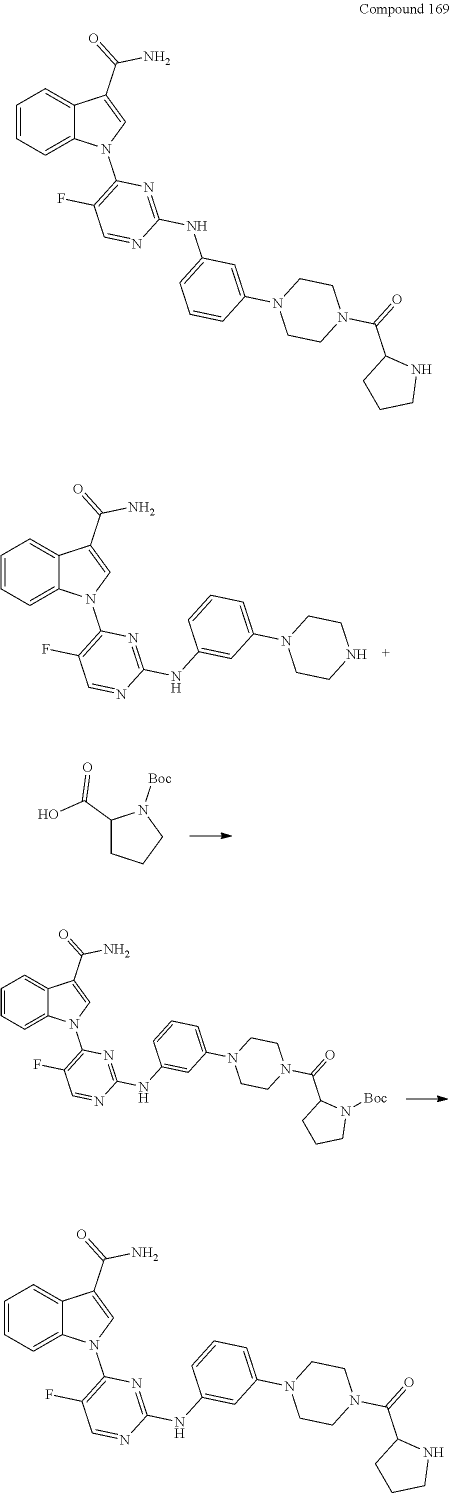 [SMILES: NC(=O)c1cn(-c2nc(Nc3cccc(N4CCN(C(=O)C5CCCN5)CC4)c3)ncc2F)c2ccccc12 ; NC(=O)c1cn(-c2nc(Nc3cccc(N4CCNCC4)c3)ncc2F)c2ccccc12 ; CC(C)(C)OC(=O)N1CCCC1C(=O)O ; CC(C)(C)OC(=O)N1CCCC1C(=O)N1CCN(c2cccc(Nc3ncc(F)c(-n4cc(C(N)=O)c5ccccc54)n3)c2)CC1 ; NC(=O)c1cn(-c2nc(Nc3cccc(N4CCN(C(=O)C5CCCN5)CC4)c3)ncc2F)c2ccccc12]

### Step 1: Preparation of tert-butyl 2-(4-(3-[4-(3-carbamoyl-indol-1-yl)-5-fluoro-pyrimidin-2-ylamino]-phenyl)-piperazine-1-carbonyl)-pyrrolidine-1-carboxylate

Tert-butyl 2-(4-(3-[4-(3-carbamoyl-indol-1-yl)-5-fluoro-pyrimidin-2-ylamino]-phenyl)-piperazine-1-carbonyl)-pyrrolidine-1-carboxylate was obtained in accordance with the same preparation method of Example 158 except for replacing the 2-tetrahydrofuroic acid in Step 1 of Example 158 with N-Boc-DL-proline.

### Step 2: Preparation of 1-(5-fluoro-2-{3-[4-(pyrrolidin-2-carbonyl)-1-piperazin-1-yl]-phenylamino}-pyrimidin-4-yl)-1H-indole-3-carboxamide

Tert-butyl 2-(4-{3-[4-(3-carbamoyl-indol-1-yl)-5-fluoro-pyrimidin-2-ylamino]-phenyl}-piperazine-1-carbonyl)-pyrrolidine-1-carboxylate (80 mg, 0.127 mmol) (prepared in Step 1) was dissolved in 10 ml of dichloromethane at room temperature, and stirred at room temperature. 1 ml of trifluoroacetic acid was added, and the reaction solution was stirred at room temperature for 1 hour. TLC showed that the reaction was completed. The reaction system was poured into saturated aqueous sodium bicarbonate solution (100 ml), and extracted with dichloromethane (60 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol (containing 5% of aqueous ammonia)) to obtain 1-(5-fluoro-2-{3-[4-(pyrrolidin-2-carbonyl)-1-piperazin-1-yl]-phenylamino}-pyrimidin-4-yl)-1H-indole-3-carboxamide (21 mg, solid).

¹HNMR (DMSO-d6, 400 MHz) δ: 9.83 (s, 1H), 8.76-8.79 (d, 1H), 8.59 (d, 1H), 8.30-8.33 (m, 1H), 8.21 (m, 1H), 7.90 (br, 1H), 7.50 (s, 1H), 7.30-7.35 (m, 2H), 7.14-7.21 (m, 3H), 6.61-6.63 (m, 1H), 4.174.21 (t, 1H), 3.74 (br, 4H), 3.09-3.15 (m, 4H), 2.86-2.90 (m, 1H), 2.14-2.19 (m, 1H), 1.63-1.83 (m, 4H).

LC-MS (ESI): 529.2 (M+H)⁺.

### Example 170

### Preparation of 1-(5-fluoro-2-{3-[4-(tetrahydro-furan-3-carbonyl)-piperazin-1-yl]-phenylamino}-pyrimidin-4-yl)-1H-indole-3-carboxamide (Compound 170)

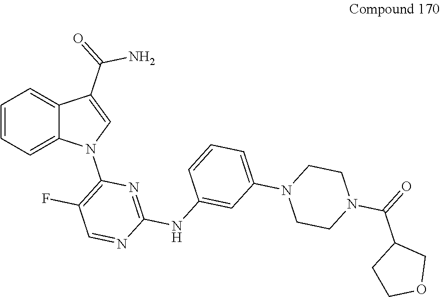 [SMILES: NC(=O)c1cn(-c2nc(Nc3cccc(N4CCN(C(=O)C5CCOC5)CC4)c3)ncc2F)c2ccccc12]

1-(5-Fluoro-2-{3-[4-(tetrahydro-furan-3-carbonyl)-piperazin-1-yl]-phenylamino}-pyrimidin-4-yl)-1H-indole-3-carboxamide was obtained in accordance with the same preparation method of Example 158 except for replacing the 2-tetrahydrofuroic acid in Step 1 of Example 158 with 3-tetrahydrofuroic acid.

¹HNMR (DMSO-d6, 400 MHz) δ: 9.83 (s, 1H), 8.78-8.79 (d, 1H), 8.57 (d, 1H), 8.30-8.32 (m, 1H), 8.21 (m, 1H), 7.85 (br, 1H), 7.47 (s, 1H), 7.31-7.35 (m, 2H), 7.14-7.23 (m, 3H), 6.62-6.64 (d, 1H), 3.66-3.89 (m, 4H), 3.56 (br, 4H), 3.35 (m, 1H), 3.04-3.07 (m, 4H), 1.96-2.05 (m, 2H).

LC-MS (ESI): 530.0 (M+H)⁺.

### Example 171

### Preparation of 1-{2-[3-(4-cycloheptanecarbonyl-piperazin-1-yl)-phenylamino]-5-fluoro-pyrimidin-4-yl}-1H-indole-3-carboxamide (Compound 171)

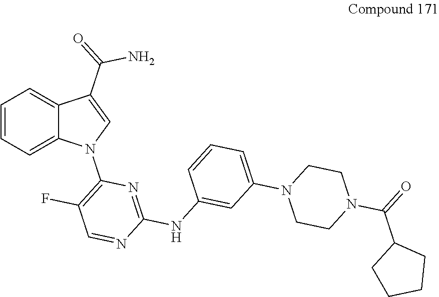 [SMILES: NC(=O)c1cn(-c2nc(Nc3cccc(N4CCN(C(=O)C5CCCC5)CC4)c3)ncc2F)c2ccccc12]

1-{2-[3-(4-Cyclopentanecarbonyl-piperazin-1-yl)-phenylamino]-5-fluoro-pyrimidin-4-yl}-1H-indole-3-carboxamide was obtained in accordance with the same preparation method of Example 158 except for replacing the 2-tetrahydrofuroic acid in Step 1 of Example 158 with cyclopentanoic acid.

¹HNMR (DMSO-d6, 400 MHz) δ: 9.83 (s, 1H), 8.77-8.78 (d, 1H), 8.55 (d, 1H), 8.30-8.32 (m, 1H), 8.21 (m, 1H), 7.84 (br, 1H), 7.47 (s, 1H), 7.29-7.34 (m, 2H), 7.12-7.16 (m, 3H), 6.61-6.63 (d, 1H), 3.54 (br, 4H), 2.93-3.04 (m, 5H), 1.51-1.76 (m, 8H).

LC-MS (ESI): 528.3 (M+H)⁺.

### Example 172

### Preparation of 1-{5-fluoro-2-[3-(4-methylcarbamoyl-piperazin-1-yl)-phenylamino]-pyrimidin-4-yl}-1H-indole-3-carboxamide (Compound 172)

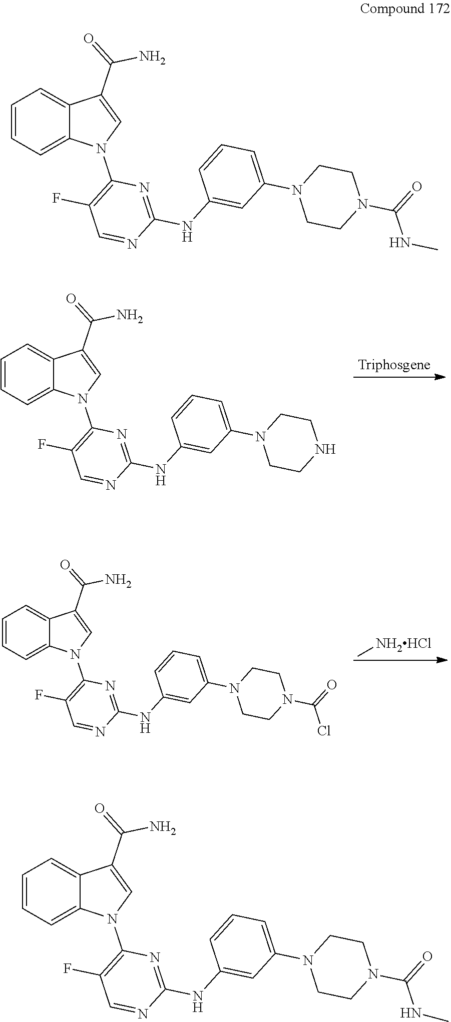

### Step 1: Preparation of 4-{3-[4-(3-carbamoyl-indol-1-yl)-5-fluoro-pyrimidin-2-ylamino]-phenyl}-piperazine-1-carbonyl chloride

Triphosgene (207 mg, 0.696 mmol) was dissolved in 10 ml of dichloromethane at room temperature, and cooled to 0-5° C. in an ice water bath. 1-[5-Fluoro-2-(3-piperazin-1-yl-phenylamino)-pyrimidin-4-yl]-1H-indole-3-carboxylic acid (200 mg, 0.464 mmol) (prepared in Example 146) was dissolved in a mixed solvent of dichloromethane (20 ml) and tetrahydrofuran (10 ml), the resulting solution was slowly added dropwise to the above solution of triphosgene in dichloromethane, and the temperature was kept at 0-5° C. After completion of the addition, triethylamine (85 mg, 0.837 mmol) was added dropwise at this temperature. After completion of the addition, the reaction solution was warmed to room temperature and reacted for 6 hours. TLC showed that the reaction was completed. The reaction system was poured into water (100 ml), and extracted with dichloromethane (60 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol) to obtain 4-{3-[4-(3-carbamoyl-indol-1-yl)-5-fluoro-pyrimidin-2-ylamino]-phenyl}-piperazine-1-carbonyl chloride (190 mg).

### Step 2: Preparation of 1-{5-fluoro-2-[3-(4-methylcarbamoyl-piperazin-1-yl)-phenylamino]-pyrimidin-4-yl}-1H-indole-3-carboxamide

Methylamine hydrochloride (39 mg, 0.578 mmol) was stirred in 10 ml of DMF at room temperature, which could not dissolve completely. N,N-Dimethylethylamine (248 mg, 1.925 mmol) was added at room temperature. The system became clear and was cooled to 0-5° C. in an ice water bath. 4-{3-[4-(3-Carbamoyl-indol-1-yl)-5-fluoro-pyrimidin-2-ylamino]-phenyl}-piperazine-1-carbonyl chloride (190 mg, 0.385 mmol) (prepared in Step 1) was dissolved in 5 ml of tetrahydrofuran, and the resulting solution was added dropwise to the above solution. After completion of the addition, the reaction solution was warmed to room temperature and reacted for 4 hours. TLC showed that the reaction was completed. The reaction system was poured into water (100 ml), and extracted with ethyl acetate (60 ml×2). The organic phase was washed with saturated NaCl solution twice, dried over anhydrous sodium sulfate, filtrated, and concentrated under reduced pressure. The resulting residues were purified by column chromatography (eluent: dichloromethane/methanol (containing 5% of aqueous ammonia)) to obtain 1-{5-fluoro-2-[3-(4-methylcarbamoyl-piperazin-1-yl)-phenylamino]-pyrimidin-4-yl}-1H-indole-3-carboxamide (20 mg, solid).

¹HNMR (DMSO-d6, 400 MHz) δ: 9.78 (s, 1H), 8.76-8.77 (d, 1H), 8.55 (d, 1H), 8.29-8.30 (m, 1H), 8.27 (m, 1H), 7.84 (br, 1H), 7.38 (s, 1H), 7.30-7.33 (m, 2H), 7.12-7.25 (m, 3H), 6.62-6.64 (m, 1H), 6.47-6.48 (d, 1H), 3.01-3.03 (m, 4H), 3.57-2.58 (m, 4H), 1.23 (s, 3H).

LC-MS (ESI): 489.1 (M+H)⁺.

### Biological Assay

### Test Example 1. Measurement of the 50% Inhibitory Concentration (IC₅₀) of the Present Compound on the Growth of CDK9-Positive Cell Line MOLM13 In Vitro

Experimental Materials and Methods

1. MOLM13 cell line and cell culture MOLM13 is a CDK9-positive human acute myeloid leukemia cell line from DMSZ. Suspension culture was carried out in RPMI1640 (Gibco) medium with 10% fetal bovine serum (Gibco), 1% penicillin-streptomycin and 2 mM glutamine.

2. Drug Treatment

Suspended MOLM-13 cells in logarithmic growth phase were collected by centrifugation (1700 rpm, 3 minutes), the supernatant was discarded, and the cells were counted. A solution with a cell concentration of 2×10⁵ cells per milliliter was formulated and inoculated in a 96-well plate (Corning) with 100 microliters per well. The plate was incubated at 37° C., 5% CO₂ overnight. On the next day, the test compound was added to the cultured cells in two parallel wells. The final concentration of organic solvent did not exceed one thousandth. The cells were cultured for 3 to 6 days, and MTT assay was carried out.

Two CDK9 inhibitors in clinical trial phase, Dinaciclib (SCH727965) and BAY1251125 (Table 1), were selected as the control compounds. The present compound and the control compounds were respectively dissolved in DMSO (Sigma). The purity of the compound was above 98%. The storage concentration of the compound was 10 mM, and the solution was stored at −20° C. A two-fold or 10-fold serial dilution was carried out before use.

TABLE 1
Control compounds

| | In vitro CDKs | | |
|---|---|---|---|
| Compound | (IC₅₀) | Structural formula | Source |
| Dinaciclib | CDK2(1 nM) CDK5(1 nM) CDK1(3 nM) CDK9(4 nM) | 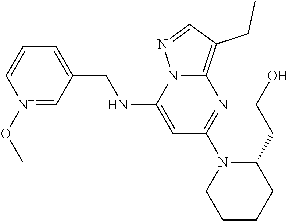 [SMILES: CCc1cnn2c(NCc3ccc[n+](OC)c3)cc(N3CCCC[C@H]3CCO)nc12] | Purchased from Selleck |
| BAY1251125 | CDK9 (4 nM) | 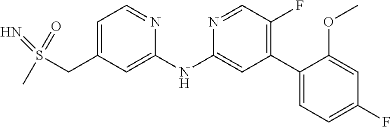 [SMILES: COc1cc(F)ccc1-c1cc(Nc2cc(CS(C)(=N)=O)ccn2)ncc1F] | Prepared according to the method of Example 1 of WO2014/076091 |

3. MTT Assay and IC₅₀ Calculation

The MTT assay reagent was Dojindo CCK8 kit, and the microplate reader was THERMO MULTISKAN FC.

CCK8 reagent was added directly to the suspended MOLM-13 cells with a final concentration of 10%, and the cells were cultured for 1 to 4 hours. When the solvent control well became dark yellow, the OD450 nm absorption value was measured, and the cell growth rate was calculated according to the following formula. Cell growth rate %=100*(T−T₀)/(C−T₀), wherein T=the optical density value of the drug-treated well—the optical density value of the blank control well, T₀=the optical density value of the well before drug treatment—the optical density value of the blank control well, C=the optical density value of the solvent control well—the optical density value of the blank control well. The 50% inhibitory concentration of cell growth, i.e. IC₅₀, was calculated by GraphPad Prism7 software. The assay was repeated three times, and the data was subjected to a biological statistical analysis. Table 2 summarizes the results of the IC₅₀ of the present compound for inhibiting the growth of MOLM-13 cells (or inducing apoptosis) in vitro.

### Test Example 2. Measurement of CDK9 Protein Kinase Activity

The inhibitory activity (IC₅₀) of the present compound on CDK9 kinase was determined with ADP-Glo™ CDK9/CyclinK kinase detection kit (V4105, Promega Corporation) and GloMax™ 196 microplate luminescence detector.

The present compound was dissolved in DMSO (Sigma Aldrich), and the initial concentration of the compound was 1000 nM. A two-fold serial dilution was carried out. The specific operation of CDK9 kinase activity determination was carried out according to the ADP-Go™ CDK9/CyclinK kinase detection experimental procedure. DMSO was used as the solvent control, and Dinaciclib was used as the positive control. Each test sample was tested in duplicate, and the test was repeated once. The inhibitory activity (IC₅₀) of the compound on CDK9 protein kinase was calculated from the dose-inhibition curve of the compound. Table 2 shows the results of the inhibitory activity (IC₅₀) of the present compound on CDK9 protein kinase.

TABLE 2
IC₅₀ of the compound for inhibiting cell growth
and CDK9 protein kinase activity

| | | CDK9/ |
|---|---|---|
| | MOLM13 | CyclinK |
| Compounds | cells | kinase |
| 1 | * | — |
| 2 | * | — |
| 3 | * | — |
| 4 | * | — |
| 5 | * | — |
| 6 | * | — |
| 7 | *** | — |
| 8 | *** | — |
| 9 | *** | — |
| 10 | *** | — |
| 11 | *** | — |
| 12 | *** | — |
| 13 | *** | — |
| 14 | *** | — |
| 15 | *** | — |
| 16 | *** | — |
| 17 | ** | — |
| 18 | *** | — |
| 19 | *** | — |
| 20 | *** | — |
| 21 | *** | — |
| 22 | *** | — |
| 23 | *** | — |
| 24 | *** | — |
| 25 | *** | — |
| 26 | *** | — |
| 27 | **** | — |
| 28 | **** | — |
| 29 | *** | — |
| 30 | **** | — |
| 31 | *** | — |
| 32 | *** | — |
| 33 | **** | — |
| 34 | ***** | — |
| 35 | ***** | — |
| 36 | **** | — |
| 37 | **** | — |
| 38 | ** | — |
| 39 | ***** | **** |
| 40 | ***** | — |
| 41 | **** | — |
| 42 | ***** | **** |
| 43 | ***** | — |
| 44 | **** | — |
| 45 | *** | — |
| 46 | **** | — |
| 47 | *** | — |
| 48 | *** | — |
| 49 | **** | — |
| 50 | **** | — |
| 51 | **** | — |
| 52 | **** | — |
| 53 | **** | — |
| 54 | **** | — |
| 55 | ***** | **** |
| 56 | ***** | **** |
| 57 | **** | — |
| 58 | ***** | — |
| 59 | **** | — |
| 60 | ***** | **** |
| 61 | **** | — |
| 62 | *** | — |
| 63 | **** | — |
| 64 | *** | — |
| 65 | *** | — |
| 66 | *** | — |
| 67 | *** | — |
| 68 | **** | — |
| 69 | **** | — |
| 70 | *** | — |
| 71 | *** | — |
| 72 | *** | — |
| 73 | **** | — |
| 74 | *** | — |
| 75 | ** | — |
| 76 | *** | — |
| 77 | *** | — |
| 78 | ** | — |
| 79 | **** | — |
| 80 | **** | — |
| 81 | **** | — |
| 82 | *** | — |
| 83 | *** | — |
| 84 | **** | — |
| 85 | **** | — |
| 86 | **** | — |
| 87 | **** | — |
| 88 | ***** | *** |
| 89 | ***** | *** |
| 90 | **** | — |
| 91 | **** | — |
| 92 | ** | — |
| 93 | **** | — |
| 94 | **** | — |
| 95 | *** | — |
| 96 | **** | — |
| 97 | **** | — |
| 98 | **** | — |
| 99 | **** | — |
| 100 | **** | — |
| 101 | ** | — |
| 102 | * | — |
| 103 | ** | — |
| 104 | *** | — |
| 105 | *** | — |
| 106 | *** | — |
| 107 | *** | — |
| 108 | * | — |
| 109 | *** | — |
| 110 | **** | — |
| 111 | **** | — |
| 112 | **** | — |
| 113 | ***** | — |
| 114 | **** | — |
| 115 | ***** | ***** |
| 116 | *** | — |
| 117 | *** | — |
| 118 | **** | — |
| 119 | *** | — |
| 120 | *** | — |
| 121 | *** | — |
| 122 | *** | — |
| 123 | **** | — |
| 124 | **** | — |
| 125 | **** | — |
| 126 | **** | — |
| 127 | *** | — |
| 128 | *** | — |
| 129 | *** | — |
| 130 | * | — |
| 131 | * | — |
| 132 | * | — |
| 133 | * | — |
| 134 | * | — |
| 135 | ** | — |
| 136 | ** | — |
| 137 | **** | — |
| 138 | **** | — |
| 139 | **** | — |
| 140 | ***** | — |
| 141 | **** | — |
| 142 | ***** | — |
| 143 | *** | — |
| 144 | ***** | — |
| 145 | *** | — |
| 146 | *** | — |
| 147 | ***** | — |
| 148 | **** | — |
| 149 | **** | — |
| 150 | **** | — |
| 151 | **** | — |
| 152 | ***** | ***** |
| 153 | ** | — |
| 154 | ** | — |
| 155 | **** | — |
| 156 | *** | — |
| 157 | *** | — |
| 158 | *** | — |
| 159 | **** | — |
| 160 | *** | — |
| 161 | ***** | ***** |
| 162 | **** | — |
| 163 | ***** | ***** |
| 164 | **** | — |
| 165 | *** | — |
| 166 | *** | — |
| 167 | ***** | ***** |
| 168 | *** | — |
| 169 | **** | — |
| 170 | **** | — |
| 171 | *** | — |
| 172 | *** | — |
| Dinaciclib | **** | **** |
| BAY1251125 | *** | — |

The smaller the IC₅₀ value, the more active the compound.
In the table, “*****” means that the IC₅₀ value is <1 nM; “****” means that the IC₅₀ value is in the range of 1 nM to <10 nM; “***” means that the IC₅₀ value is in the range of 10 nM to <100 nM; “**” means that the IC₅₀ value is in the range of 100 nM to <1000 nM; “*” means that the IC₅₀ value is >1000 nM; and “—” means not determined.

The results in Table 2 show that the present compound has a high inhibitory activity on the growth of MOLM-13 cells in vitro, and the IC₅₀ thereof can reach a sub-nanomolar level. The test compound can directly inhibit the CDK9/CyclinK protein kinase activity in vitro, and the IC₅₀ thereof can be less than 1 nM.

### Test Example 3. Measurement of the Inhibitory Activity of the Present Compound on the Growth of Various Types of CDK9-Positive Tumor Cell Lines In Vitro

Tumor cell line is an effective cell model for studying the inhibitory activity of a drug on tumor growth in vitro. The inventors selected representative CDK9-positive tumor cell lines for further determination of the compound activity. All cell lines used came from ATCC, JCRB, DSMZ and Chinese Academy of Sciences Cell Bank (ZK). Cell culture condition and method were based on the requirements of each cell line. No more than 3 passages were carried out in each culture in vitro. Monoclonal purification and STR identification of the cell line were conducted if necessary.

Drug treatment, MTT assay and IC₅₀ calculation were performed in accordance with Test Example 1.

TABLE 3
Inhibitory activity (IC₅₀) of the present compound on the growth of the CDK9-positive tumor cell lines in vitro

| | | Compounds | | | | | |
|---|---|---|---|---|---|---|---|
| Tumor type | Cell line | 60 | 161 | 140 | 144 | 152 | Dinaciclib |
| Human | HO8910PM | *** | ***** | ***** | ***** | ***** | **** |
| ovarian | Sk-ov-3 | *** | ***** | ***** | ***** | ***** | *** |
| cancer | | | | | | | |
| Human | BT-549 | *** | ***** | ***** | ***** | ***** | **** |
| breast | BT-474 | *** | ***** | ***** | ***** | ***** | **** |
| cancer | CAL-120 | **** | ***** | ***** | ***** | ***** | **** |
| | HCC1954 | **** | ***** | ***** | ***** | ***** | **** |
| | JIMT-1 | *** | ***** | ***** | ***** | ***** | **** |
| | MCF7 | *** | ***** | ***** | ***** | ***** | **** |
| | MDA-MB-231 | — | ***** | ***** | ***** | ***** | **** |
| | MDA-MB-436 | — | ***** | ***** | ***** | ***** | **** |
| | MDA-MB-453 | — | ***** | ***** | ***** | ***** | **** |
| | MFM-223 | *** | ***** | ***** | ***** | ***** | **** |
| | MX-1 | **** | ***** | ***** | ***** | ***** | **** |
| | SK-BR-3 | **** | ***** | ***** | ***** | ***** | **** |
| Human | A549 | — | **** | **** | **** | ****— | **** |
| lung | NCI-H1581 | *** | ***** | ***** | ***** | ***** | **** |
| cancer | NCI-H1703 | *** | ***** | ***** | ***** | ***** | **** |
| | NCI-H1781 | *** | ***** | ***** | ***** | ***** | **** |
| | NCI-H1975 | ** | **** | **** | **** | **** | *** |
| | PC-9 | ** | **** | **** | **** | **** | **** |
| | NCI-H358 | ** | **** | **** | **** | **** | **** |
| | NCI-H1993 | — | *** | **** | **** | **** | **** |
| | NCI-H460 | ** | **** | **** | **** | **** | **** |
| Human | HCT-116 | ** | **** | **** | **** | **** | **** |
| intestinal | Colo205 | ** | **** | **** | **** | **** | **** |
| cancer | | | | | | | |
| Human | HuCCT-1 | ** | **** | **** | **** | **** | **** |
| cholangioca | | | | | | | |
| rcinoma | | | | | | | |
| Human | NCI-N87 | ** | **** | **** | **** | **** | **** |
| stomach | AGS | ** | **** | **** | **** | **** | **** |
| cancer | | | | | | | |
| Human | KYSE70 | ** | **** | **** | **** | **** | **** |
| esophageal | | | | | | | |
| cancer | | | | | | | |
| Human | CA Ski | *** | ***** | ***** | ***** | ***** | **** |
| cervical | | | | | | | |
| cancer | | | | | | | |
| Human | AN3-CA | — | — | ***** | ***** | ***** | — |
| endometrial | | | | | | | |
| cancer | | | | | | | |
| Human | SK-MEL-28 | ** | **** | **** | **** | **** | **** |
| melanoma | A375 | ** | **** | **** | **** | **** | **** |
| Human | LN229 | ** | **** | **** | ***** | **** | **** |
| glioma | | | | | | | |
| Human B | Pfeiffer | *** | ***** | ***** | ***** | ***** | **** |
| lymphocyte | | | | | | | |
| tumor | | | | | | | |
| Human | Kasumi-1 | **** | **** | **** | ***** | **** | **** |
| acute | KG-1 | *** | **** | ***** | ***** | **** | **** |
| myeloid | | | | | | | |
| leukemia | | | | | | | |
| Human | DU-145 | *** | **** | ***** | ***** | **** | **** |
| prostate | | | | | | | |
| cancer | | | | | | | |
| Human | TPC-1 | *** | **** | ***** | ***** | **** | **** |
| thyroid | KHM-5M | *** | **** | ***** | ***** | **** | **** |
| cancer | 8305C | *** | **** | ***** | ***** | **** | **** |
| | B-CPAP | *** | **** | ***** | ***** | **** | **** |

Table 3 summarizes the results of the IC₅₀ of representative compounds of the present invention for inhibiting the growth of various types of CDK9-positive tumor cell lines (or inducing apoptosis) in vitro.
The smaller the IC₅₀ value, the more active the compound.
“*****” means that the IC₅₀ value is <1 nM,
“****” means that the IC₅₀ value is in the range of 1 nM to <10 nM,
“***” means that the IC₅₀ value is in the range of 10 nM to <100 nM;
“**” means that the IC₅₀ value is in the range of 100 nM to <1000 nM;
“*” means that the IC₅₀ value is >1000 nM; and
“—” means not determined.

The results in Table 3 show that representative compounds of the present invention have a high inhibitory activity on the growth of various types of tumor cells in vitro, and the IC₅₀ thereof can reach a sub-nanomolar level.

### Test Example 4. In Vivo Growth Inhibition Test of Tumor Cell

Experimental animals: Bab/c immunodeficiency mice, female, 6 weeks old (weighing about 20 grams), purchased from Shanghai Sippr-BK laboratory animal Co. Ltd., raised by the Animal Center of Shanghai Fudan University, approved by the Ethics Committee of Shanghai Fudan University. The breeding environment is SPF level.

Test samples: Compound 140 of the present invention (purity: 99%), solid powder, stored at 4-8° C.

Cell and animal modelling: human MOLM13 leukemia cells were cultured in RPMI 1640 medium containing 10% fetal bovine serum. Cells in the exponential growth phase were collected (centrifuge at 1700 rpm for 3 minutes), washed once with 1×PBS, and resuspended to obtain a final concentration of 5×10⁷/ml cells. Single-point inoculation of 0.1 ml was carried out subcutaneously on the right back.

When the average tumor volume reached ˜400 mm³, the mice were randomly grouped according to tumor size. The test was divided into solvent control group and administration group, with 3 mice in each group. 0.01 ml of solution per gram of mouse weight was intragastrically administered (p.o.) once a day. The tumor was measured three times a week. Calculation formula for tumor volume: long diameter×short diameter²/2. When the tumor volume of the control group reached 2000 mm³, the experiment was ended, and molecular pathological analysis of tumor tissue was performed. The efficacy was evaluated based on the relative tumor growth inhibition rate (TGI), and the safety was evaluated based on the change of animal weight.

Formulation of test samples: An appropriate amount of compound 140 was weighed. An appropriate amount of ultrapure water was added and mixed well.

Methanesulfonic acid was then added to obtain a clear solution, and 4M NaOH was added dropwise to adjust pH to 4.3. Ultrapure water was added to obtain a constant volume and a final concentration of 5 mg/ml.

The solvent control group was an aqueous methanesulfonic acid solution (pH 4.3).

Criteria to evaluate the result: Relative tumor growth inhibition rate TGI (%), i.e. TGI=1−T/C (%).

T/C % represents the relative tumor growth rate, that is, the percentage ratio of relative tumor volume or tumor weight of the administration group to that of the control group at a certain time point. T and C respectively represent the relative tumor volume (RTV) of the administration group and the control group at a certain time point. T/C % is calculated as follows:

T/C %=T_{RTV}/C_{RTV}*100%

wherein T_{RTV}: the average RTV of the administration group; C_{RTV}: the average RTV of the solvent control group: RTV=Vₜ/V₀, V₀ represents the tumor volume of the animal at the time of grouping, and Vₜ represents the tumor volume of the animal after administration.

Statistical analysis: All test results were expressed as mean tumor volume±SEM (mean standard error). The tumor volume data on Day 9 after the start of administration was selected for statistical analysis among different groups. The significant difference between the relative tumor volume of the administration group and that of the control group was determined by independent sample T test method. All data were analyzed with SPSS 18.0. p<0.05 means that there is a significant difference.

TABLE 4
TGI and T/C values on Day 9 after the start of administration

| | Compound | Tumor | | | P value |
|---|---|---|---|---|---|
| Tumor | dose | volume | | | (relative to the |
| model | (mg/kg) | (mm³) | TGI (%) | TIC (%) | control group) |
| Solvent | — | 1893.4 ± 98 | — | — | — |
| control | | | | | |
| group | | | | | |
| MOLM13 | 50 | 164.8 ± 12 | 91.29 | 8.71 | 0.0061 |

The results in Table 4 show that the compound 140 of the present invention can effectively inhibit the growth of human acute myeloid leukemia MOLM13 cells in vivo at a dose of 50 mg/kg. During the administration, the weight of the mouse remained stable, and the drug was well tolerated.

1. A compound of formula (I) or a pharmaceutically acceptable salt thereof, 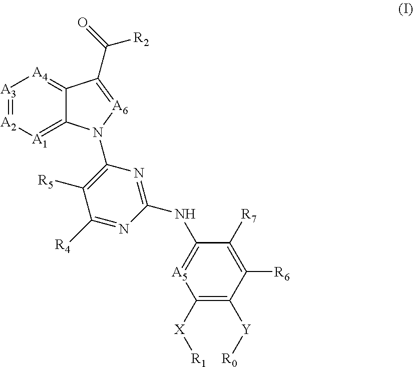
wherein:
A₁, A₂, A₃, A₄ and A₅ are identical or different and are each independently selected from the group consisting of N and CQ;
A₆ is selected from the group consisting of CR₃ and N;
R₂ is selected from the group consisting of alkoxy, hydroxy and amino, wherein the amino is optionally substituted with one or two alkyl(s);
R₃, R₄, R₅, R₆ and R₇ are each independently selected from group Q;
X and Y are identical or different and are each independently selected from the group consisting of —NR₈—, —O—, —S—, —CH₂—, —C(O)—, —S(O)ₙ— and group Q;
when X and Y are each independently selected from —NR₈—, R₁ and R₀ are identical or different and are each independently selected from the group consisting of hydrogen, alkyl, alkenyl, alkynyl, cycloalkyl, heterocyclyl, aryl, heteroaryl, —R^{u}OR^{x}, —R^{u}N(R^{y})(R^{z}), —R^{u}C(O)OR^{x}, —C(O)N(R^{y})(R^{z}), —R^{u}S(O)ₙN(R^{y})(R^{z}) and —R^{u}S(O)ₙR, wherein the alkyl, alkenyl, alkynyl, cycloalkyl, heterocyclyl, aryl and heteroaryl are each independently optionally substituted with one or more substituents selected from the group consisting of halogen, cyano, amino, hydroxy, alkyl, alkoxy, amido, cycloalkyl, heterocyclyl, aryl, haloaryl and heteroaryl; R₈ is selected from the group consisting of hydrogen, alkyl, alkenyl, alkynyl and heterocyclyl; or, R₁ and R₈ or R₀ and R₈ together with the nitrogen attached to them form a heterocyclyl or heteroaryl, wherein the heterocyclyl and heteroaryl are each independently optionally substituted with one or more substituents selected from the group consisting of halogen, alkyl, haloalkyl, alkoxy, haloalkoxy, —C(O)-alkenyl, —C(O)-alkyl, hydroxyalkyl, -alkylene-O-alkyl, heterocyclyl, -alkylene-heterocyclyl, —C(O)-heterocyclyl, —C(O)-cycloalkyl, —C(O)—N(R^{y})(R^{z}) and —R^{u}N(R^{y})(R^{z};
when X and Y are each independently selected from the group consisting of —O—, —S—, —CH₂—, —C(O)— and —S(O)ₙ—, R₁ and R₀ are identical or different and are each independently selected from the group consisting of —R^{u}N(R^{y})(R^{z}), —C(O)N(R^{y})(R^{z}) and —R^{u}S(O)ₙN(R^{y})(R^{z});
when X is selected from group Q, R₁ is absent;
when Y is selected from group Q, R₀ is absent;
each R^{u} is independently selected from the group consisting of a bond, alkylene, alkenylene and alkynylene;
each R^{u} is independently selected from the group consisting of hydrogen, alkyl, hydroxyalkyl, haloalkyl, alkenyl and alkynyl; or
the oxygen in —R^{u}OR^{x}— together with the attached R^{u} and R^{x} form a 3 to 7 membered oxygen-containing heterocyclyl, wherein the heterocyclyl is optionally substituted with one or more group Q;
R^{y} and R^{z} are identical or different and are each independently selected from the group consisting of hydrogen, alkyl, alkoxy, alkenyl, alkynyl, cycloalkyl, haloalkyl and haloalkoxy; or
R^{y} and R^{z} together with the nitrogen attached to them form a heterocyclyl or heteroaryl, wherein the heterocyclyl and heteroaryl are each independently optionally substituted with one or more substituents selected from the group consisting of halogen, alkyl, haloalkyl, alkoxy, haloalkoxy, —C(O)-alkyl, alkyl, alkenyl and alkynyl;
each group Q is independently selected from the group consisting of hydrogen, halogen, hydroxy, alkyl, amino, alkoxy, cycloalkyl, alkenyl, alkynyl, cyano, nitro, amido, aryl, heterocyclyl, heteroaryl, —O-(alkylene)-O-alkyl and —O-(alkylene)-heterocyclyl, wherein the alkyl, amino, alkoxy, cycloalkyl, alkenyl, alkynyl, amido, aryl, heterocyclyl and heteroaryl are each independently optionally substituted with one or more substituents selected from the group consisting of hydroxy, halogen and alkyl; and
n is 0, 1 or 2.

2. The compound of formula (I) or a pharmaceutically acceptable salt thereof according to claim 1, wherein:
A₁, A₂, A₃, A₄ and A₅ are identical or different and are each independently selected from the group consisting of N and CQ;
each group Q is independently selected from the group consisting of hydrogen, halogen, nitro, hydroxy, C₁-C₆ alkyl, C₁-C₆ alkoxy, C₁-C₆ haloalkyl, C₁-C₆ haloalkoxy, C₃-C₆ cycloalkyl, amido, —O—(C₁-C₆ alkylene)-O—C₁-C₆ alkyl and —O—(C₁-C₆ alkylene)-3 to 7 membered heterocyclyl.

3. The compound of formula (I) or a pharmaceutically acceptable salt thereof according to claim 1, wherein A₁, A₂, A₃ and A₄ are CH.

4. The compound of formula (I) or a pharmaceutically acceptable salt thereof according to claim 1, wherein A₁ is N, and A₂, A₃ and A₄ are CH.

5. The compound of formula (I) or a pharmaceutically acceptable salt thereof according to claim 1, wherein A₅ is selected from the group consisting of N and CH.

6. The compound of formula (I) or a pharmaceutically acceptable salt thereof according to claim 1, wherein A₆ is selected from the group consisting of N and CH.

7. The compound of formula (I) or a pharmaceutically acceptable salt thereof according to claim 1, wherein:
X is selected from —NR₈—; R₈ is selected from the group consisting of hydrogen and alkyl; and R₁ is selected from the group consisting of hydrogen, C₁-C₆ alkyl, C₃-C₆ cycloalkyl, 3 to 7 membered heterocyclyl, —R^{u}OR^{x} and —R^{u}N(R^{y})(R^{z}), wherein the C₁-C₆ alkyl, C₃-C₆ cycloalkyl and 3 to 7 membered heterocyclyl are each independently optionally substituted with one or more substituents selected from the group consisting of halogen, cyano, hydroxy, C₁-C₆ alkoxy, 3 to 7 membered heterocyclyl preferably 3 to 7 membered oxygen-containing or nitrogen-containing heterocyclyl, C₅-C₇ aryl preferably phenyl, C₅-C₇ haloaryl preferably halophenyl, 5 to 7 membered heteroaryl and C₃-C₆ cycloalkyl;
Y is selected from group Q; and R₀ is absent;
R^{u}, R^{y}, R^{z} and Q are as defined in claim 1.

8. The compound of formula (I) or a pharmaceutically acceptable salt thereof according to claim 1, wherein:
X is selected from —NR₈—; and R₁ and R₈ together with the nitrogen attached to them form a heterocyclyl, wherein the heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)-alkenyl, —C(O)-alkyl, hydroxyalkyl, -alkylene-O-alkyl, heterocyclyl, -alkylene-heterocyclyl, —C(O)-heterocyclyl, —C(O)-cycloalkyl, —C(O)—N(R^{y})(R^{z}) and —R^{u}N(R^{y})(R^{z});
Y is selected from group Q; and R₀ is absent;
R^{u}, R^{y}, R^{z} and Q are as defined in claim 1.

9. The compound of formula (I) or a pharmaceutically acceptable salt thereof according to claim 1, wherein:
X is selected from the group consisting of —O—, —S—, —CH₂—, —C(O)— and —S(O)ₙ—; and R₁ is selected from —R^{u}N(R^{y})(R^{z});
Y is selected from group Q; and R₀ is absent;
R^{u}, R^{y}, R^{z}, n and Q are as defined in claim 1.

10. The compound of formula (I) or a pharmaceutically acceptable salt thereof according to claim 1, wherein:
Y is selected from —NR₈—; R₈ is selected from the group consisting of hydrogen and alkyl; and R₀ is selected from the group consisting of hydrogen, C₁-C₆ alkyl, C₃-C₆ cycloalkyl, 3 to 7 membered heterocyclyl, —R^{u}OR^{x} and —R^{u}N(R^{y})(R^{z}), wherein the C₁-C₆ alkyl, C₃-C₆ cycloalkyl and 3 to 7 membered heterocyclyl are each independently optionally substituted with one or more substituents selected from the group consisting of halogen, cyano, hydroxy, C₁-C₆ alkoxy, 3 to 7 membered heterocyclyl preferably 3 to 7 membered oxygen-containing or nitrogen-containing heterocyclyl, C₅-C₇ aryl preferably phenyl, C₅-C₇ haloaryl preferably halophenyl, 5 to 7 membered heteroaryl and C₃-C₆ cycloalkyl;
X is selected from group Q; and R₁ is absent;
R^{u}, R^{y}, R^{z} and Q are as defined in claim 1.

11. The compound of formula (I) or a pharmaceutically acceptable salt thereof according to claim 1, wherein:
Y is selected from —NR₈—; and R₀ and R₈ together with the nitrogen attached to them form a heterocyclyl, wherein the heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)-alkenyl, —C(O)-alkyl, hydroxyalkyl, -alkylene-O-alkyl, heterocyclyl, -alkylene-heterocyclyl, —C(O)-heterocyclyl, —C(O)-cycloalkyl, —C(O)—N(R^{y})(R^{z}) and —R^{u}N(R_{y})(R^{z});
X is selected from group Q; and R₁ is absent;
R^{u}, R^{y}, R^{z} and Q are as defined in claim 1.

12. The compound of formula (I) or a pharmaceutically acceptable salt thereof according to claim 1, wherein:
Y is selected from the group consisting of —O—, —S—, —CH₂—, —C(O)— and —S(O)ᵤ—; and R₀ is selected from —R^{u}N(R^{y})(R^{z});
X is selected from group Q; and R₁ is absent;
R^{u}, R^{y}, R^{z}, n and Q are as defined in claim 1.

13. The compound of formula (I) or a pharmaceutically acceptable salt thereof according to claim 1, wherein:
X is selected from the group consisting of hydrogen, halogen, hydroxy, alkyl, haloalkyl, amino, alkoxy, haloalkoxy, cycloalkyl, cyano and nitro; and R₁ is absent;
Y is selected from the group consisting of hydrogen, halogen, hydroxy, alkyl, haloalkyl, amino, alkoxy, haloalkoxy, cycloalkyl, cyano and nitro; and R₀ is absent.

14. The compound of formula (I) or a pharmaceutically acceptable salt thereof according to claim 1, wherein:
X is selected from —NR₈—; and R₁ is selected from the group consisting of hydrogen, C₁-C₆ alkyl and —R^{u}N(R^{y})(R^{z});
Y is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₀ is absent;
R₈ is selected from the group consisting of hydrogen and C₁-C₆ alkyl;
R^{u} is selected from C₁-C₆ alkylene;
R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or
R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

15. The compound of formula (I) or a pharmaceutically acceptable salt thereof according to claim 1, wherein:
X is selected from —NR₈—; and R₁ and R₈ together with the nitrogen attached to them form a 5 to 7 membered heterocyclyl, preferably a morpholinyl, piperidinyl, piperazinyl, pyrrolidinyl or azepanyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)—C₂-C₆ alkenyl, —C(O)—C₁-C₆ alkyl, C₁-C₆ hydroxyalkyl, —C₁-C₆ alkylene-O—C₁-C₆ alkyl, 3 to 7 membered heterocyclyl, —C₁-C₆ alkylene-3 to 7 membered heterocyclyl, —C(O)-3 to 7 membered heterocyclyl, —C(O)—C₃-C₆ cycloalkyl, —C(O)—N(R^{y})(R^{z}) and —R^{u}N(R^{y})(R^{z});
Y is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₀ is absent;
R^{u} is selected from C₁-C₆ alkylene;
R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or
R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

16. The compound of formula (I) or a pharmaceutically acceptable salt thereof according to claim 1, wherein:
X is selected from the group consisting of —O—, —S—, —CH₂—, —C(O)— and —S(O)₂—; and R₁ is selected from —R^{u}N(R^{y})(R^{z});
Y is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₀ is absent;
R^{u} is selected from the group consisting of a bond and C₁-C₆ alkylene;
R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or
R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

17. The compound of formula (I) or a pharmaceutically acceptable salt thereof according claim 1, wherein:
Y is selected from —NR₈—; and R₀ is selected from the group consisting of hydrogen, C₁-C₆ alkyl and —R^{u}N(R^{y})(R^{z});
X is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₁ is absent;
R₈ is selected from the group consisting of hydrogen and C₁-C₆ alkyl;
R^{u} is selected from C₁-C₆ alkylene;
R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or
R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

18. The compound of formula (I) or a pharmaceutically acceptable salt thereof according to claim 1, wherein:
Y is selected from —NR₈—; and R₀ and R₈ together with the nitrogen attached to them form a 5 to 7 membered heterocyclyl, preferably a morpholinyl, piperidinyl, piperazinyl, pyrrolidinyl or azepanyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy, —C(O)—C₂-C₆ alkenyl, —C(O)—C₁-C₆ alkyl, C₁-C₆ hydroxyalkyl, —C₁-C₆ alkylene-O—C₁-C₆ alkyl, 3 to 7 membered heterocyclyl, —C₁-C₆ alkylene-3 to 7 membered heterocyclyl, —C(O)-3 to 7 membered heterocyclyl, —C(O)—C₃-C₆ cycloalkyl, —C(O)—N(R^{y})(R^{z}) and —R^{u}N(R^{y})(R^{z});
X is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₁ is absent;
R^{u} is selected from C₁-C₆ alkylene;
R^{x} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or
R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

19. The compound of formula (I) or a pharmaceutically acceptable salt thereof according to claim 1, wherein:
Y is selected from the group consisting of —O—, —S—, —CH₂—, —C(O)— and —S(O)₂—; and R₀ is selected from —R^{u}N(R^{y})(R^{z});
X is selected from the group consisting of hydrogen, halogen, hydroxy, cyano, nitro, C₁-C₆ alkyl, C₁-C₆ alkoxy, C₃-C₆ cycloalkyl and C₁-C₆ haloalkoxy; and R₁ is absent;
R^{u} is selected from the group consisting of a bond and C₁-C₆ alkylene;
R^{y} and R^{z} are identical or different and are each independently selected from the group consisting of hydrogen, C₁-C₆ alkyl, C₁-C₆ alkoxy, C₁-C₆ haloalkyl, C₁-C₆ haloalkoxy and C₃-C₇ cycloalkyl; or
R^{y} and R^{z} together with the nitrogen attached to them form a 5 to 7 membered heterocyclyl, preferably a morpholinyl, piperidinyl, piperazinyl, azepanyl or pyrrolidinyl, wherein the 5 to 7 membered heterocyclyl is optionally substituted with one or more substituents selected from the group consisting of halogen, C₁-C₆ alkyl, C₁-C₆ haloalkyl, C₁-C₆ alkoxy, C₁-C₆ haloalkoxy and —C(O)—C₁-C₆ alkyl.

20. The compound of formula (I) or a pharmaceutically acceptable salt thereof according to claim 1, wherein:
R₂ is selected from the group consisting of hydroxy, amino and methylamino.

21. The compound of formula (I) or a pharmaceutically acceptable salt thereof according to claim 1, wherein:
R₃, R₄, R₅, R₆ and R₇ are each independently selected from the group consisting of hydrogen, halogen, hydroxy, C₁-C₆ alkyl, C₁-C₆ alkoxy, C₁-C₆ haloalkyl, C₁-C₆ haloalkoxy, C₃-C₆ cycloalkyl, nitro, cyano and amino.

22. The compound of formula (I) or a pharmaceutically acceptable salt thereof according to claim 1, selected from the group consisting of:

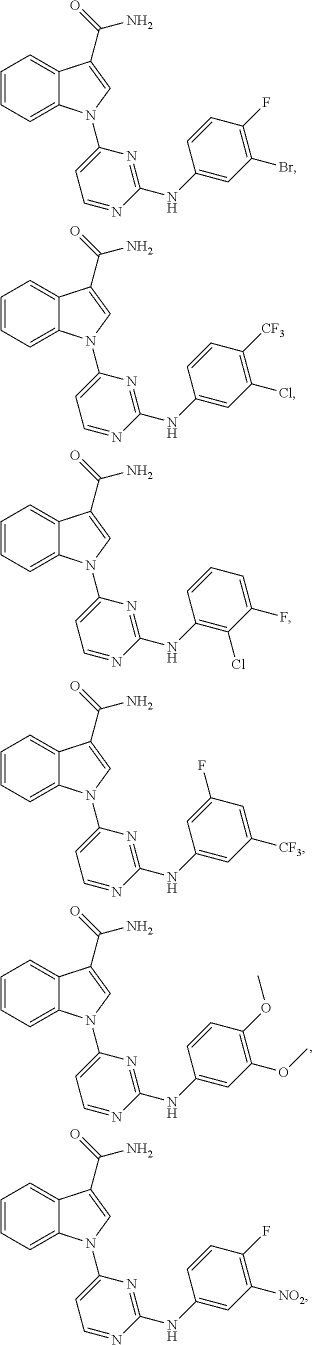

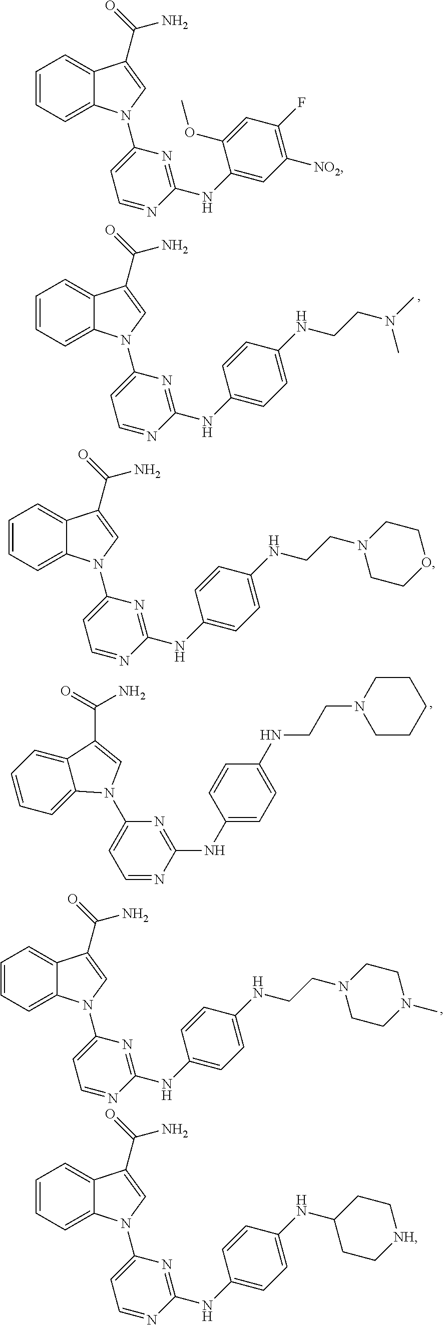

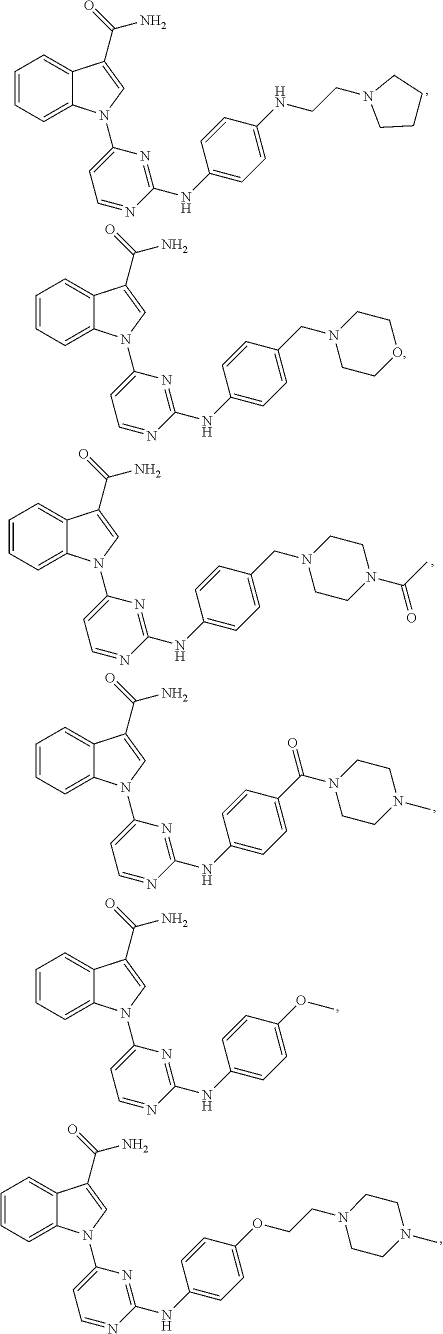

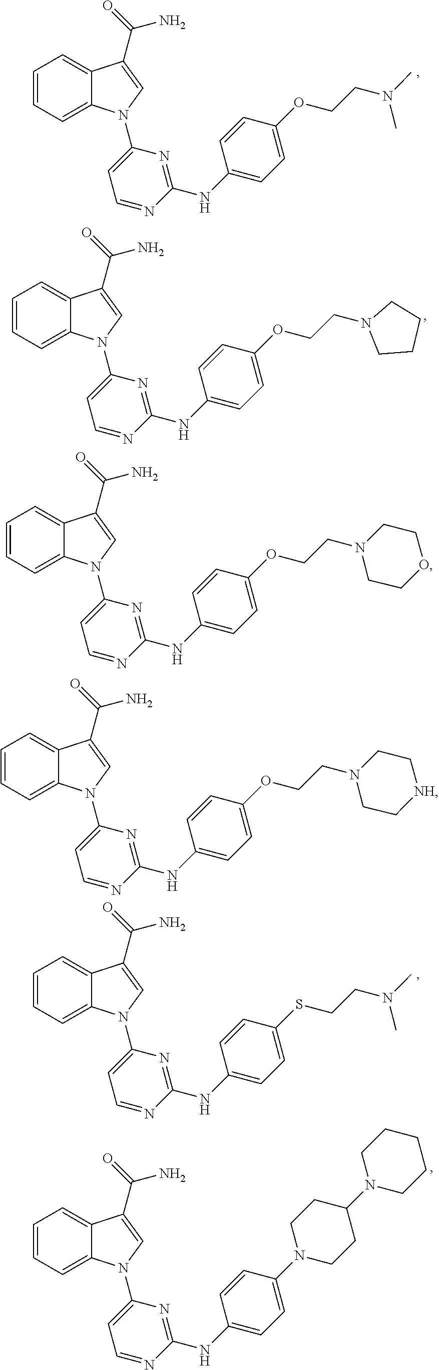

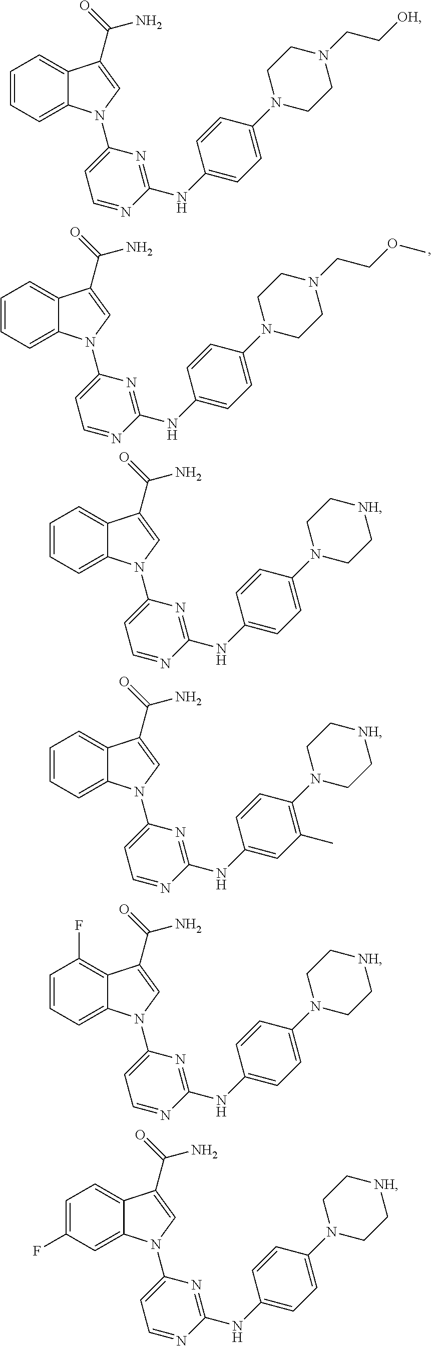

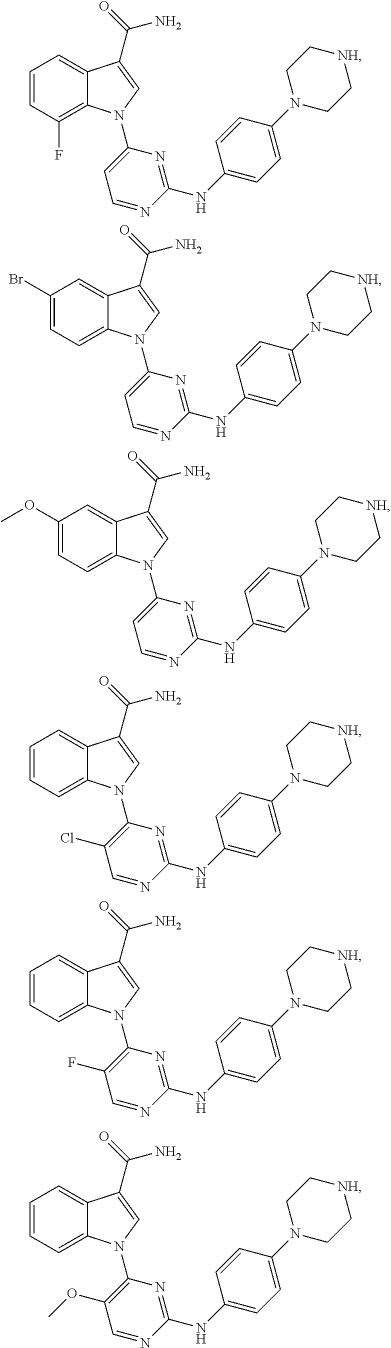

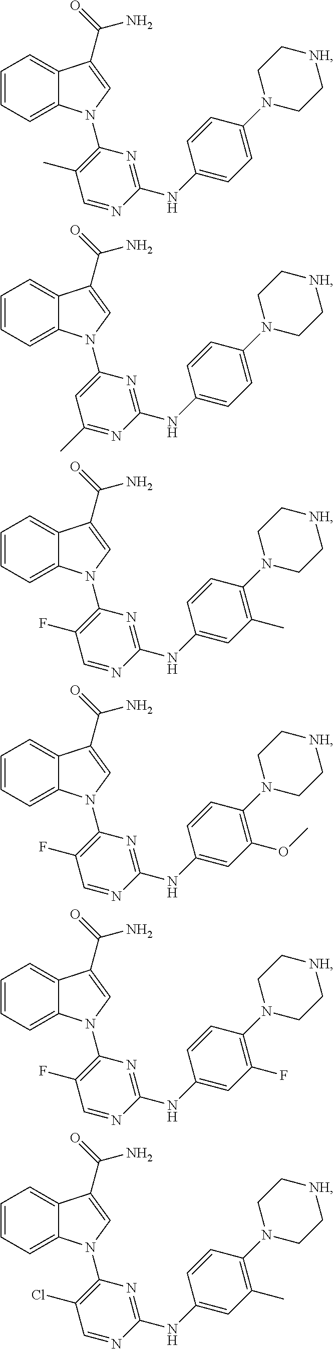

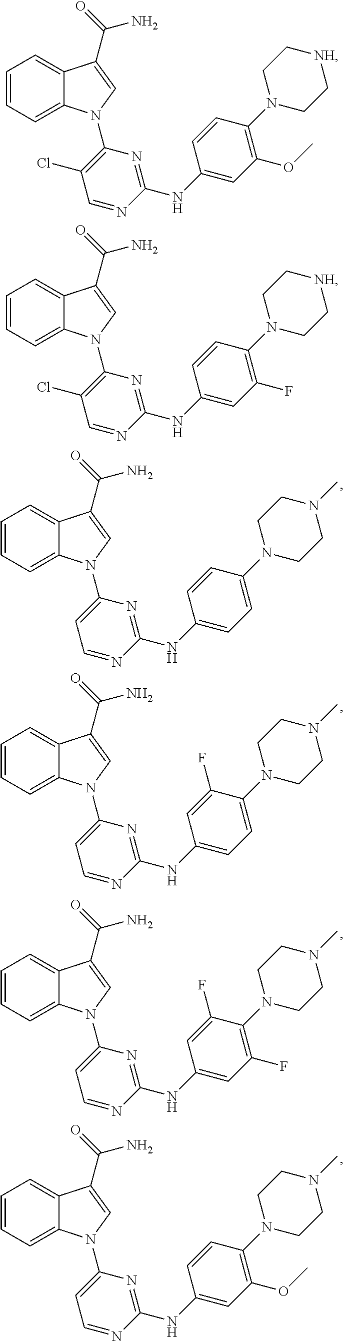

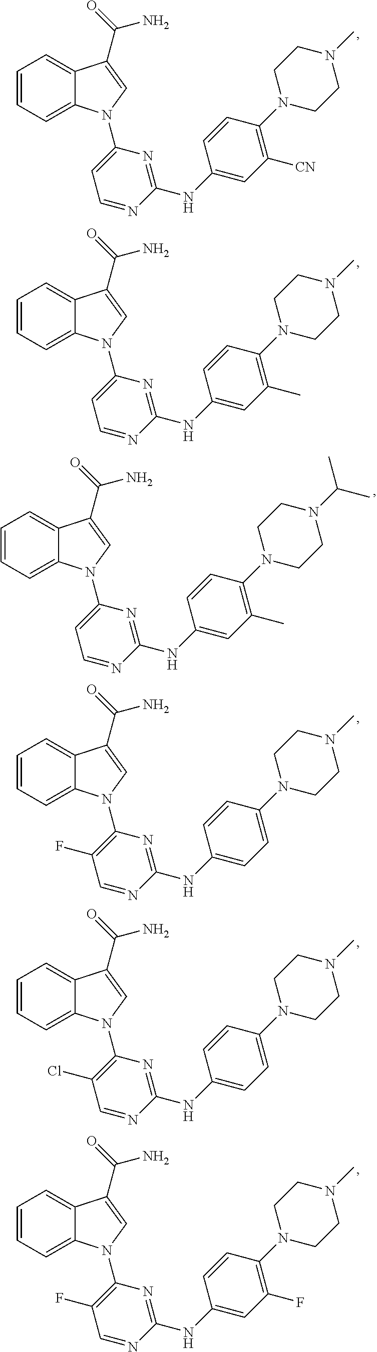

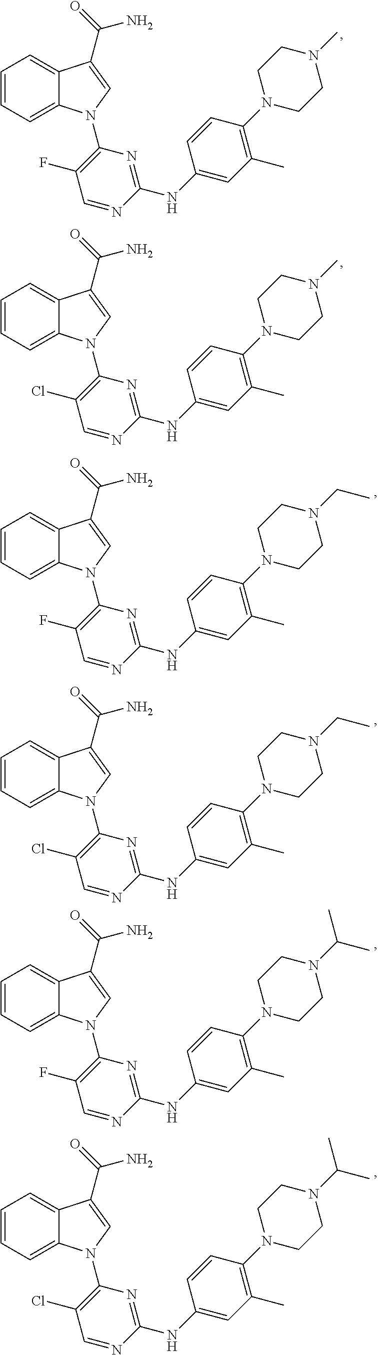

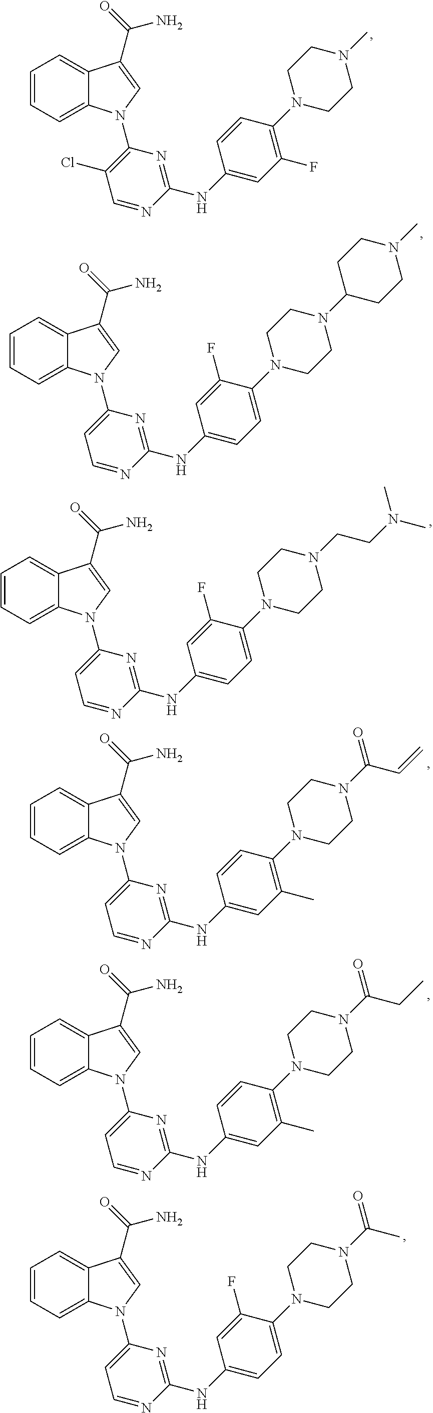

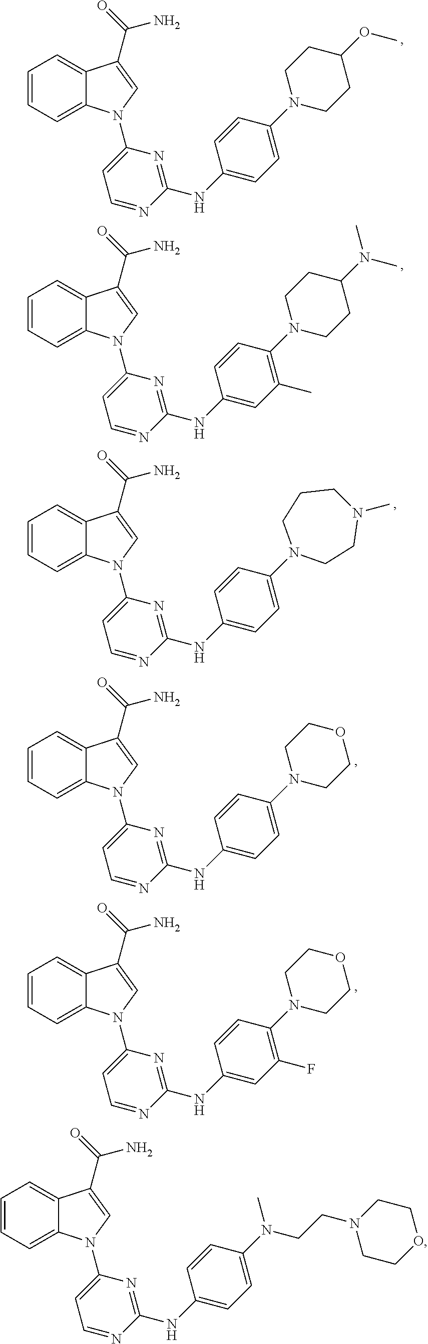

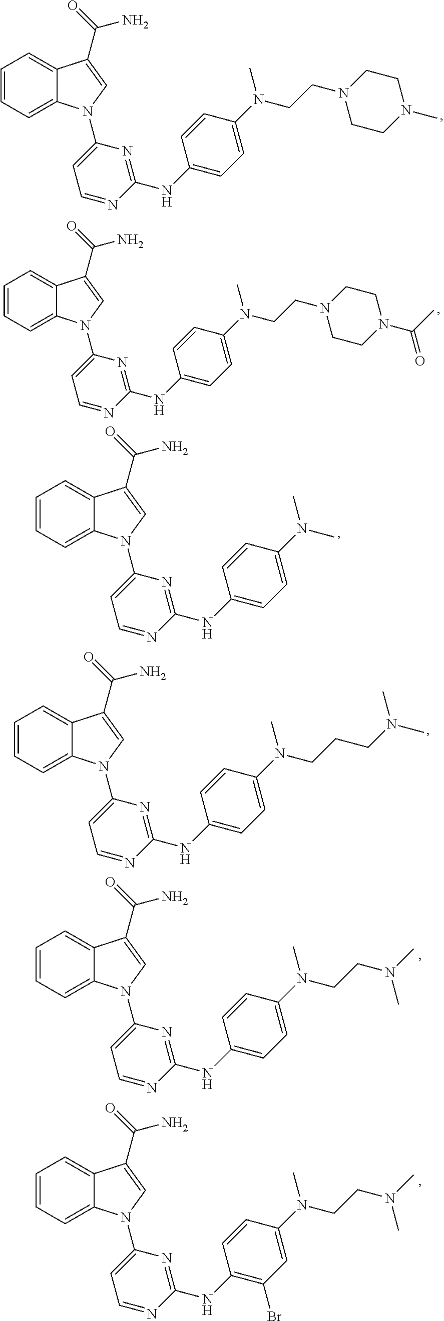

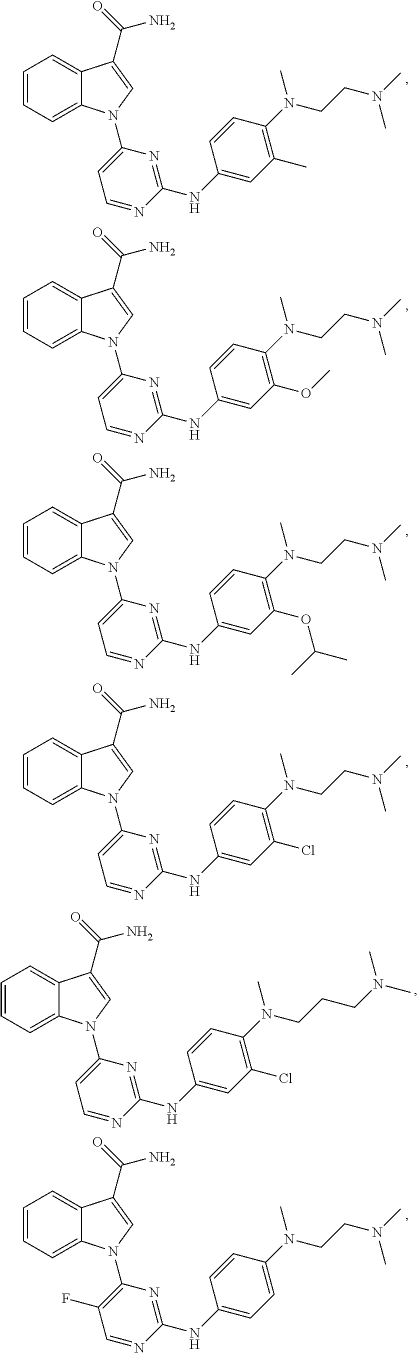

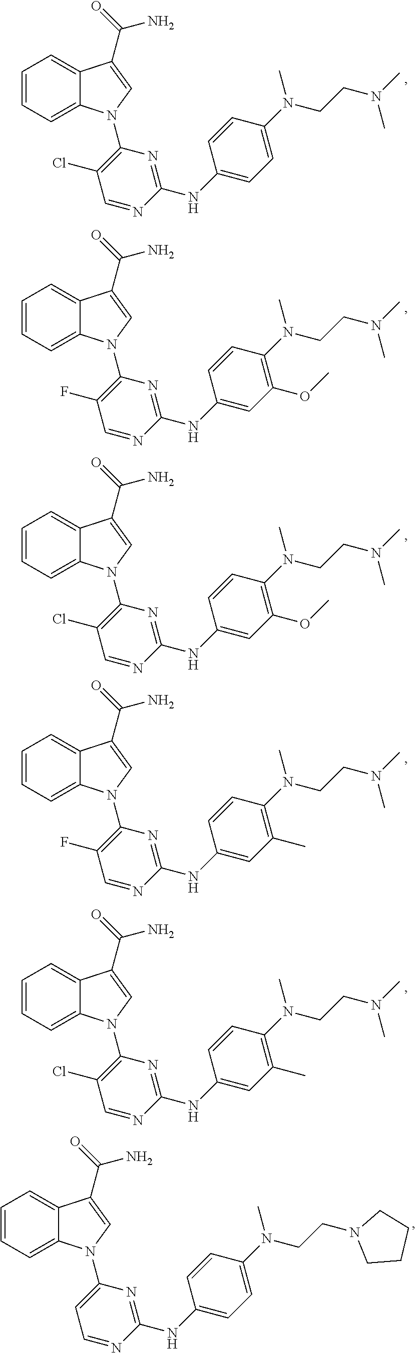

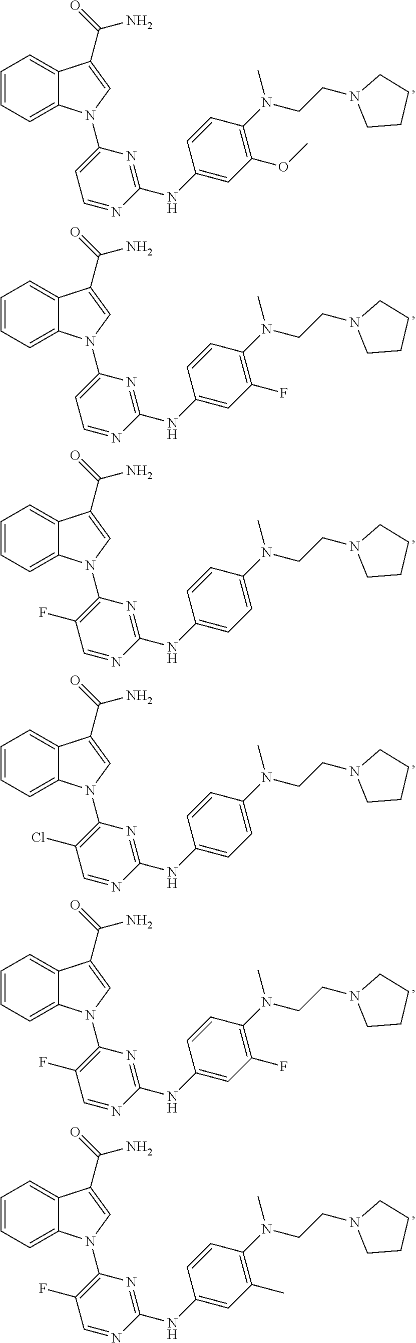

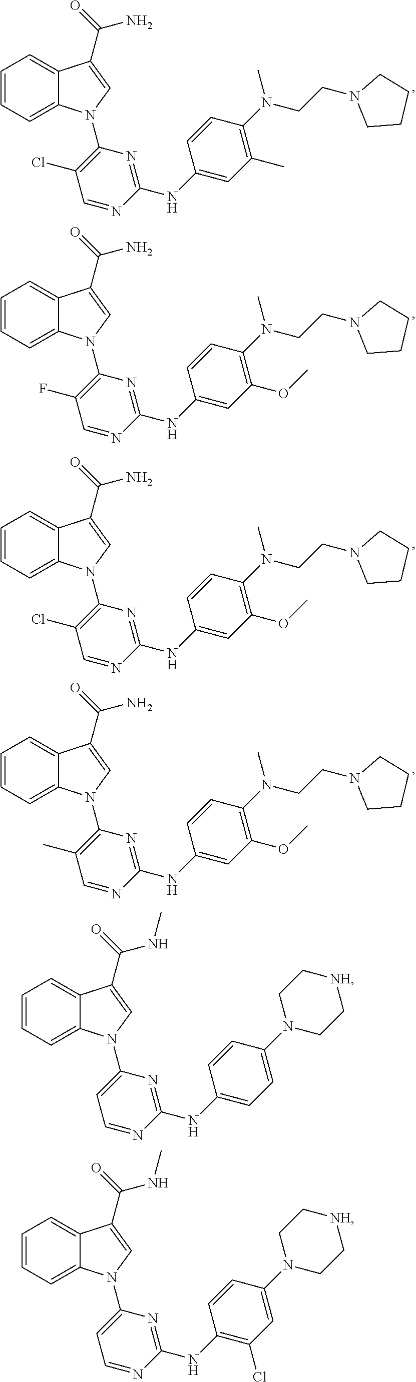

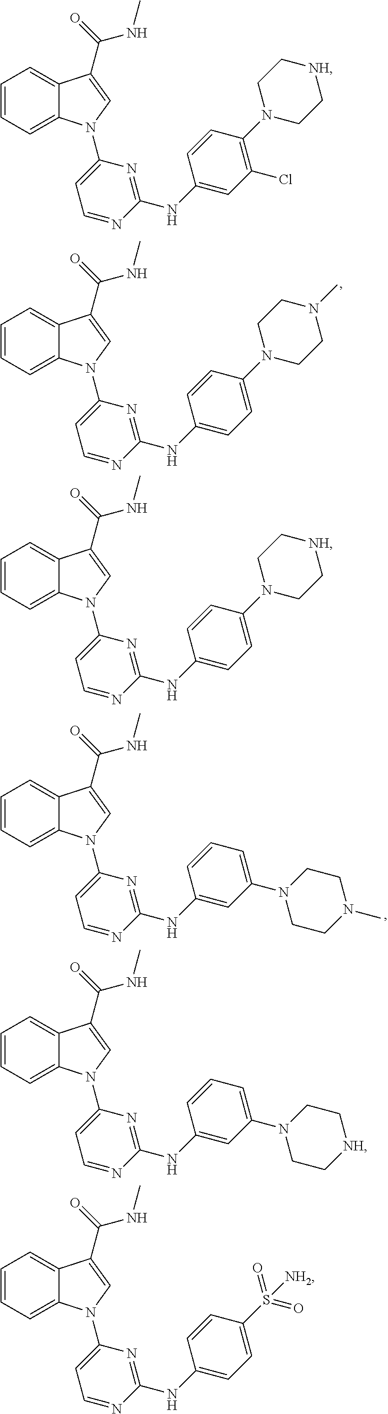

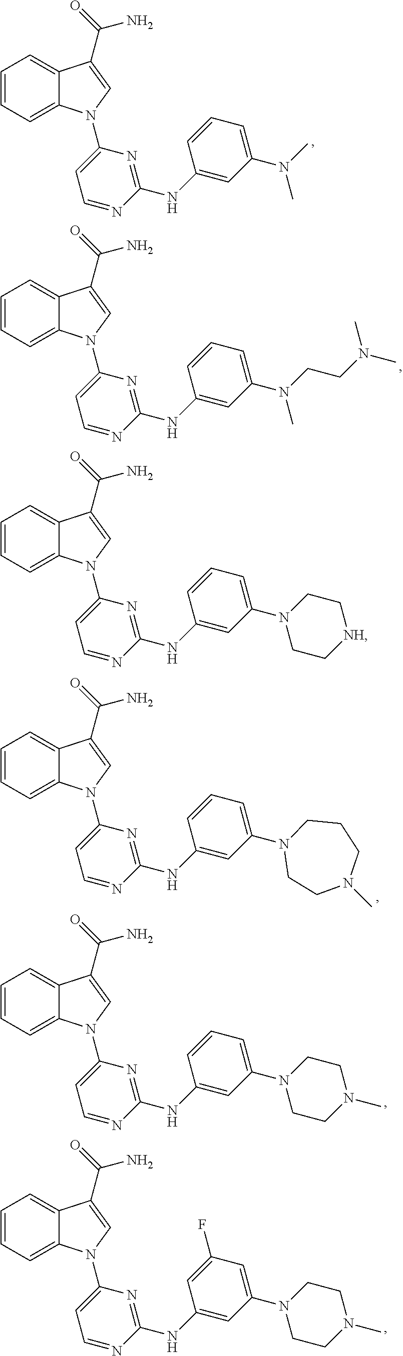

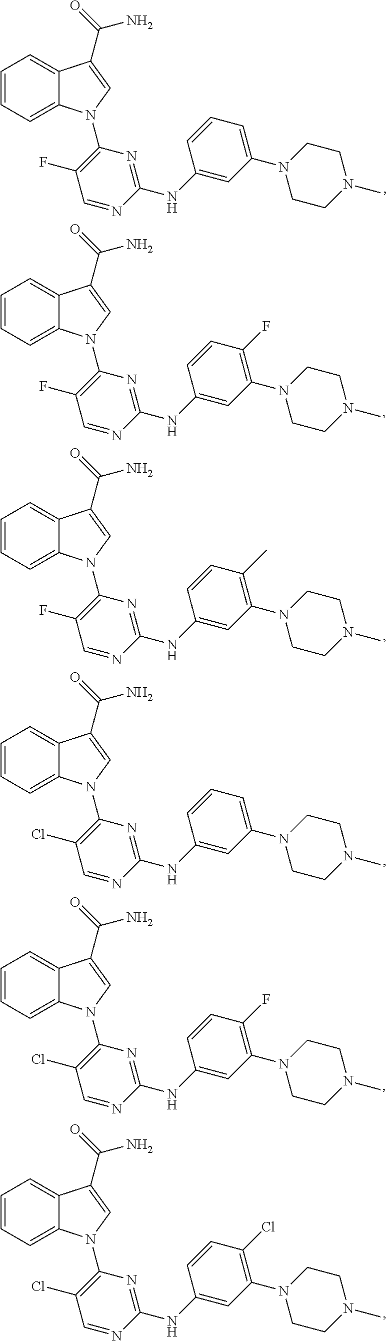

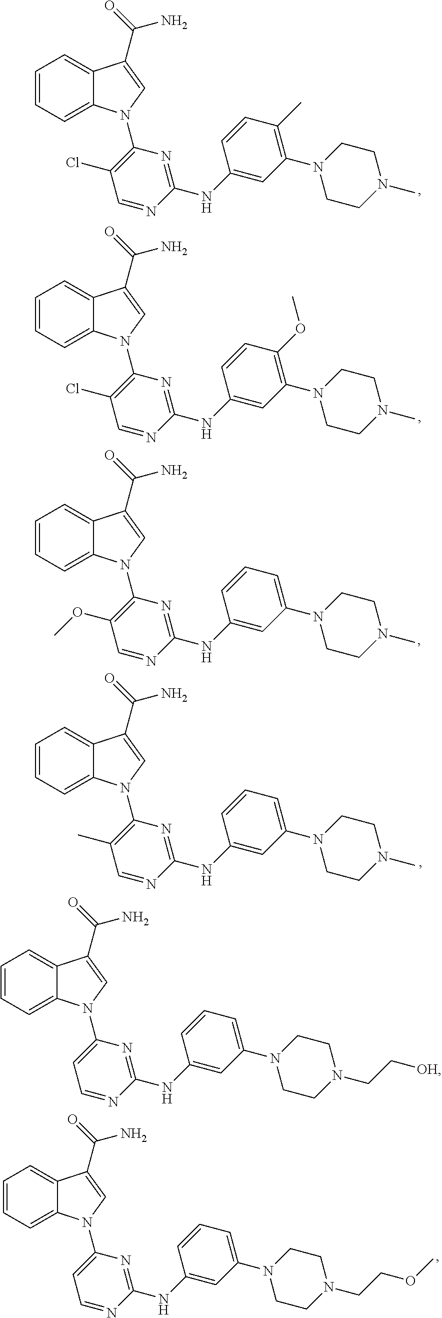

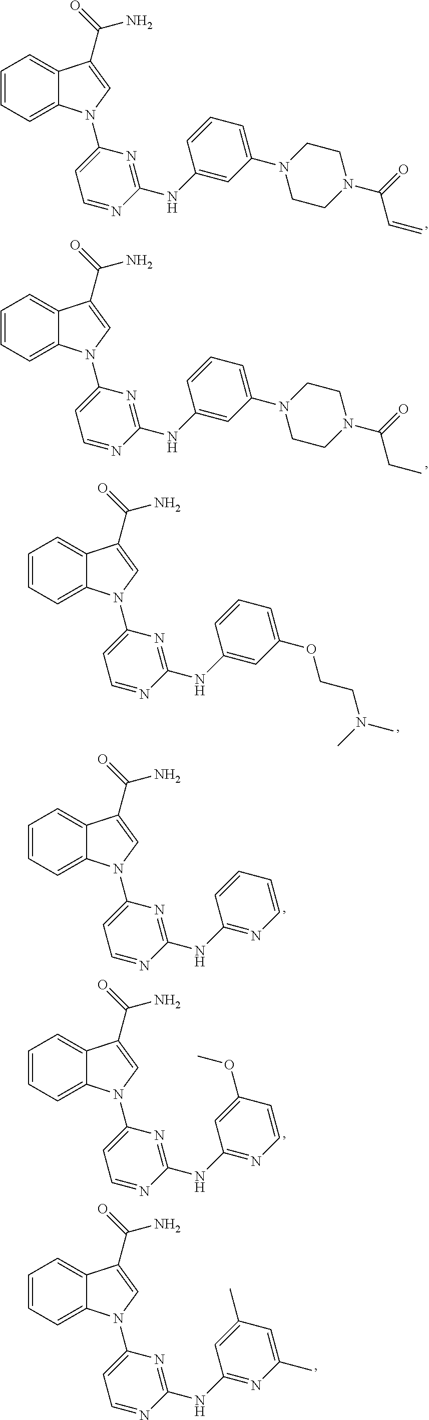

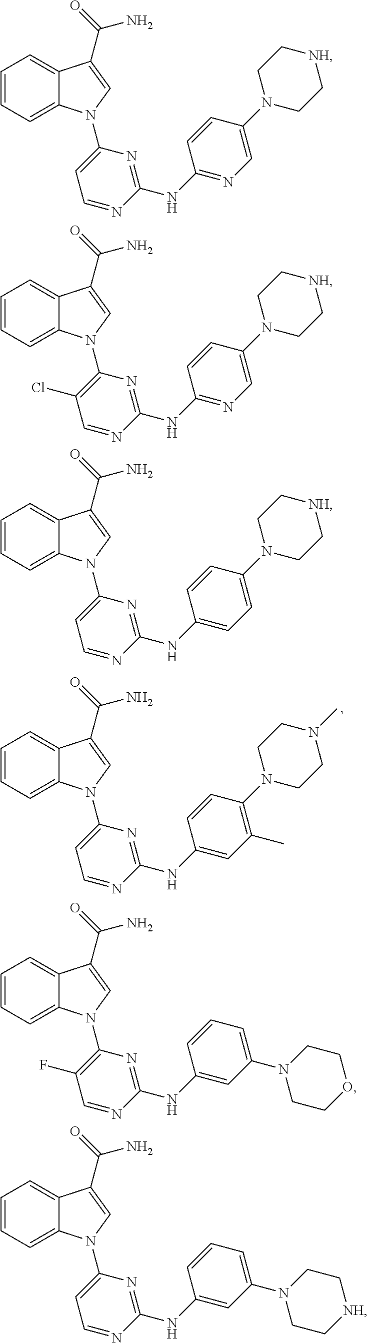

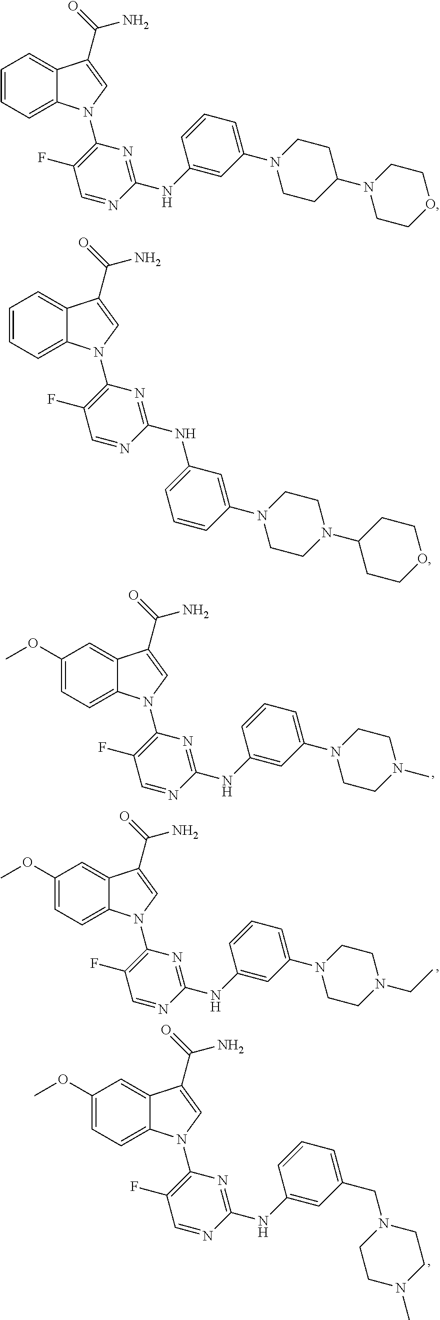

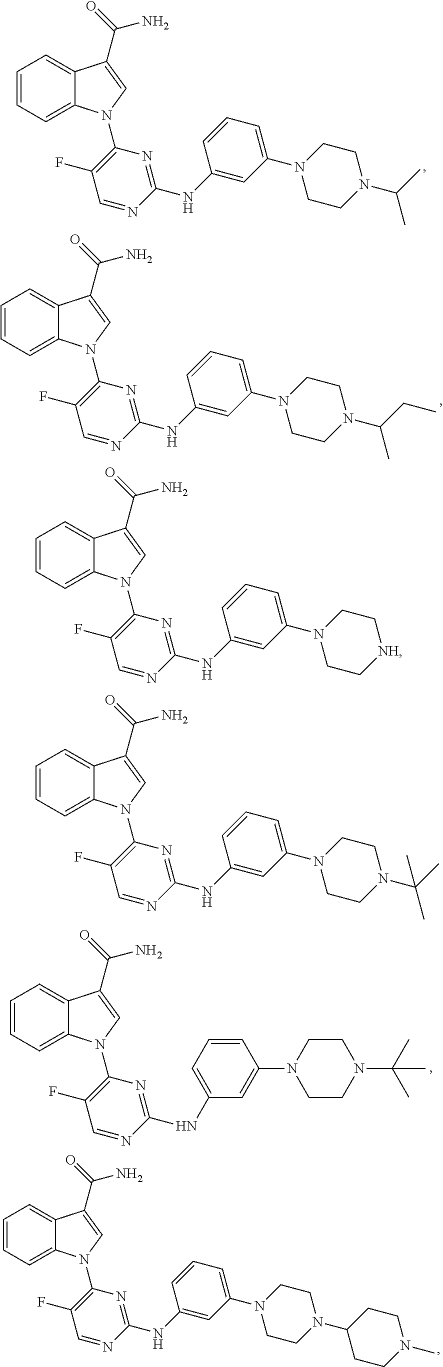

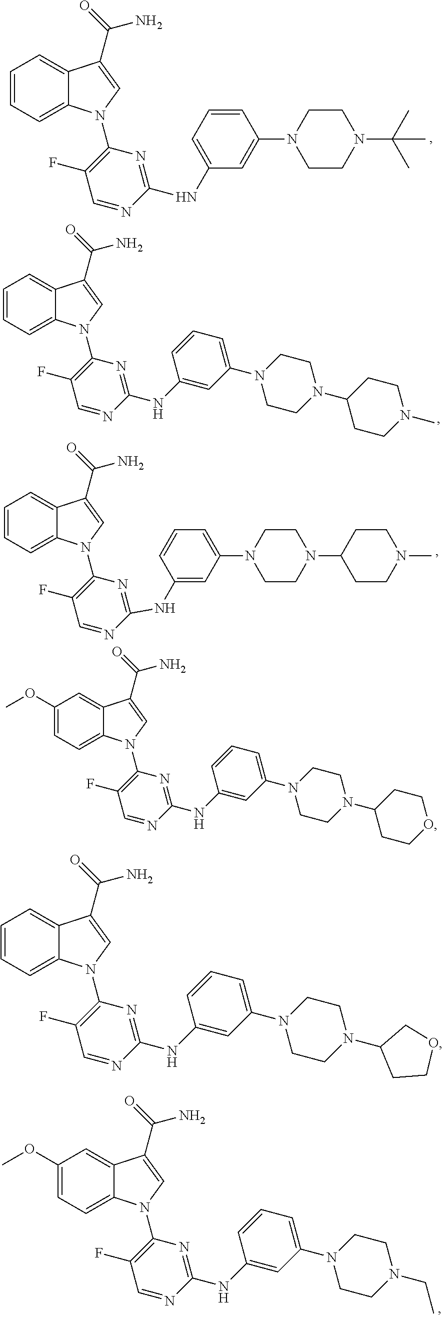

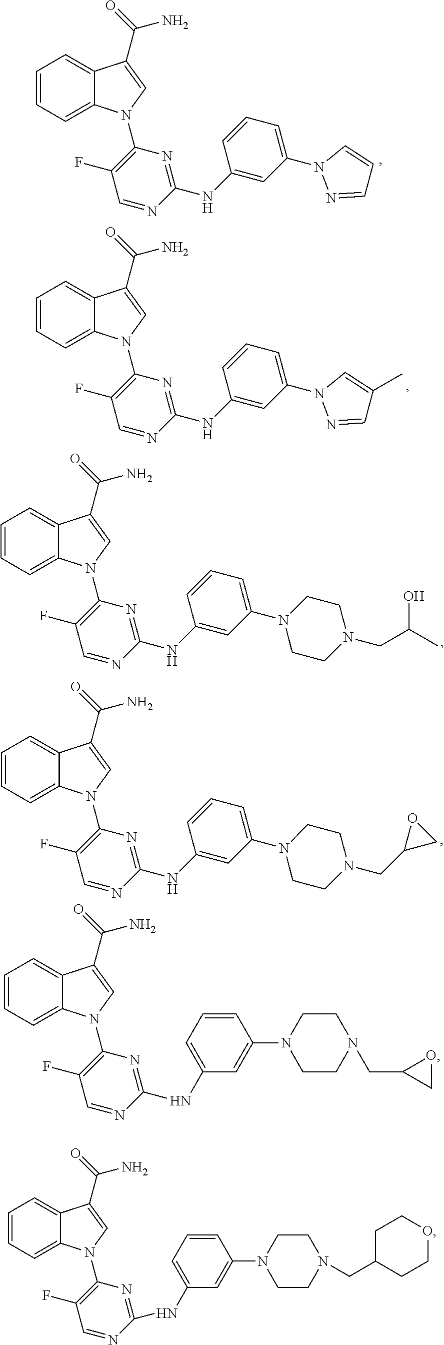

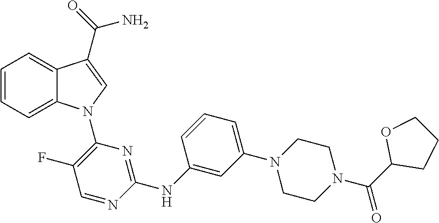 [SMILES: NC(=O)c1cn(-c2nc(Nc3cccc(N4CCN(C(=O)C5CCCO5)CC4)c3)ncc2F)c2ccccc12]

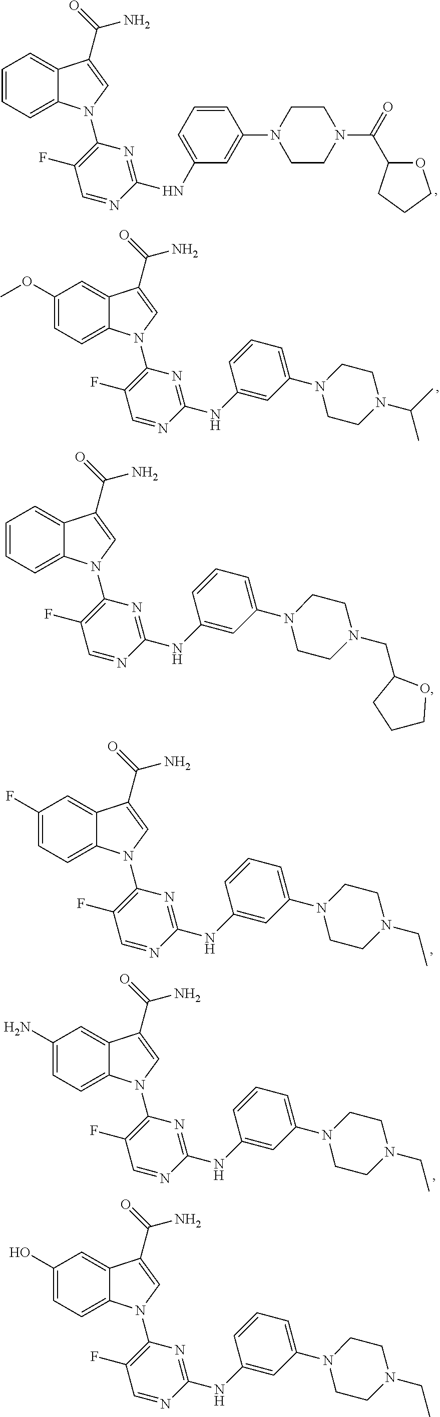

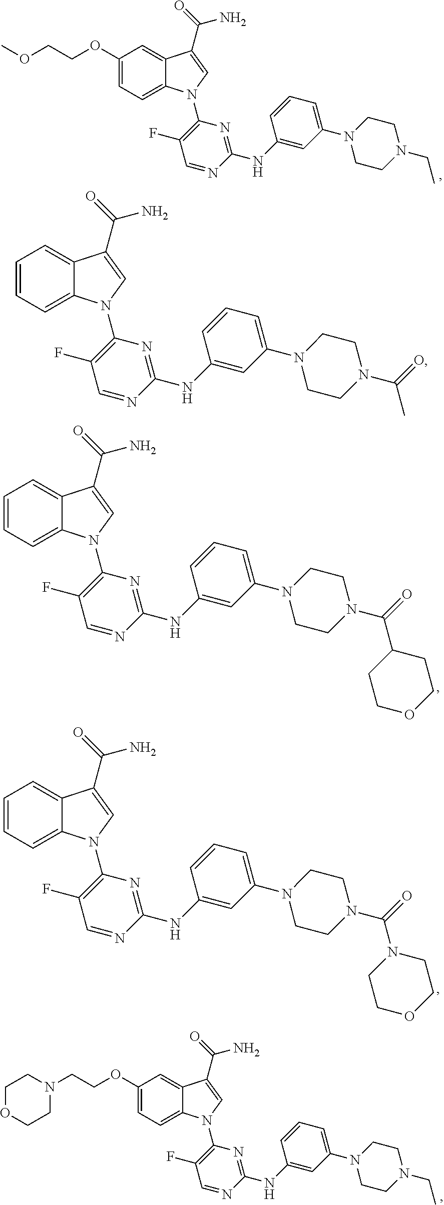

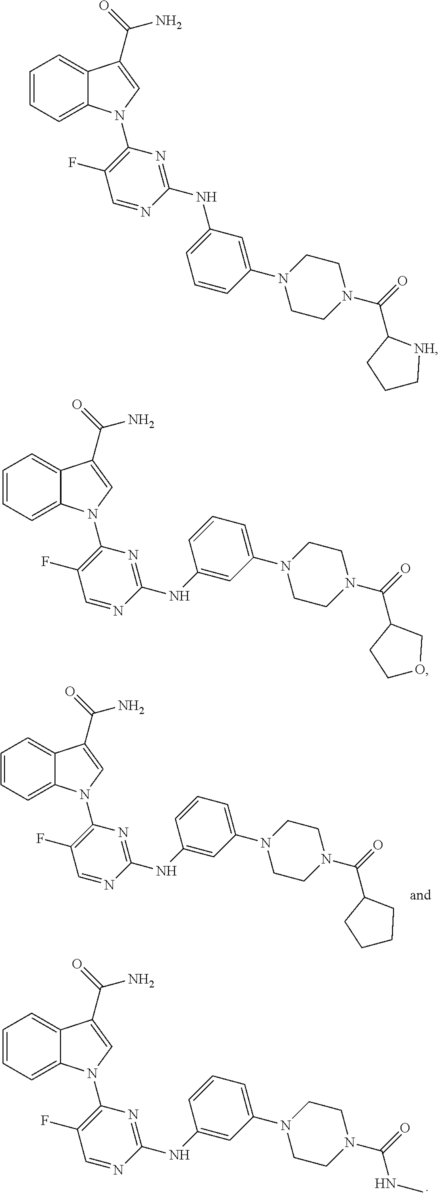 [SMILES: NC(=O)c1cn(-c2nc(Nc3cccc(N4CCN(C(=O)C5CCCN5)CC4)c3)ncc2F)c2ccccc12 ; NC(=O)c1cn(-c2nc(Nc3cccc(N4CCN(C(=O)C5CCOC5)CC4)c3)ncc2F)c2ccccc12 ; NC(=O)c1cn(-c2nc(Nc3cccc(N4CCN(C(=O)C5CCCC5)CC4)c3)ncc2F)c2ccccc12 ; CNC(=O)N1CCN(c2cccc(Nc3ncc(F)c(-n4cc(C(N)=O)c5ccccc54)n3)c2)CC1]

23. A method for preparing the compound of formula (I) or a pharmaceutically acceptable salt thereof according to claim 1, comprising the following steps of: 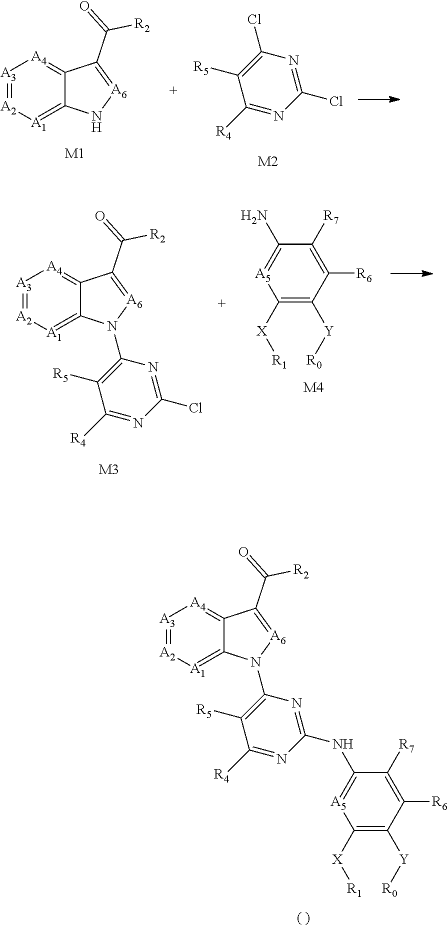
intermediate M1 is reacted with intermediate M2 in a solvent in the presence of a base and a catalyst to give intermediate M3; said solvent is preferably N,N dimethylformamide (DMF) or N-methylpyrrolidone (NMP); said base is preferably potassium carbonate or cesium carbonate; and said catalyst is preferably 1-hydroxybenzotriazole (HOBT);
intermediate M3 is reacted with intermediate M4 in a solvent under acid catalysis to give the compound of formula (I); said solvent is preferably isopropanol, isopentanol, sec-pentanol or dioxane; and said acid is preferably hydrochloric acid, sulfuric acid, methanesulfonic acid, p-toluenesulfonic acid or benzenesulfonic acid;
wherein X, Y, A₁, A₂, A₃, A₄, A₅, A₆, R₀, R₁, R₂, R₄, R₅, R₆ and R₇ are as defined in claim 1.

24. A pharmaceutical composition comprising a therapeutically effective amount of the compound of formula (I) or a pharmaceutically acceptable salt thereof according t claim 1, and a pharmaceutically acceptable carrier.

25.-27. (canceled)

28. A method for inhibiting CDK9 comprising administering an inhibitory effective amount of the compound of formula (I) or a pharmaceutically acceptable salt thereof according to claim 1 or the pharmaceutical composition according to claim 24 to a patient in need thereof.

29. A method for treating cancers in mammals, including human, comprising administering a therapeutically effective amount of the compound of formula (I) or a pharmaceutically acceptable salt thereof according to claim 1 or the pharmaceutical composition according to claim 24 to a patient in need thereof, wherein the cancer is selected from the group consisting of non-solid tumors such as leukemia, and solid tumors such as skin cancer, melanoma, lung cancer, gastric cancer, breast cancer or intestinal cancer.

30. A method for treating cancers in mammals, including human, comprising administering the compound of formula (I) or a pharmaceutically acceptable salt thereof or a metabolite thereof according to claim 1 or the pharmaceutical composition according to claim 24 in combination with other drugs or cancer therapies to a patient in need thereof, wherein the cancer is selected from the group consisting of non-solid tumors such as leukemia, and solid tumors such as skin cancer, melanoma, lung cancer, gastric cancer, breast cancer or intestinal cancer.
